(19) United States
(12) Patent Application Publication
(10) Pub. No.: US 2008/0318803 A1
(43) Pub. Date: Dec. 25, 2008

(54) Biomarkers for Monitoring Impdh Pathway Inhibition

(75) Inventors: Jugnu Jain, Natick, MA (US); Matthew W. Harding, Acton, MA (US); Ravi Ramachandran, Acton, MA (US); Martyn Botfield, Boston, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: VERTEX PHARMACEUTICALS, Cambridge, MA (US)

(21) Appl. No.: 11/597,464

(22) PCT Filed: May 27, 2005
(86) PCT No.: PCT/US05/18551

§ 371 Date: Sep. 28, 2007

Related U.S. Application Data
(60) Provisional application No. 60/575,076, filed on May 27, 2004.

(51) Int. Cl. C40B 30/06; C40B 40/08; C07H 21/00

(52) U.S. Cl. 506/10; 506/17; 536/24.3

(57) ABSTRACT

The present invention is directed to methods for ameliorating reproductive disorders. More specifically, the present invention describes methods and compositions for using IL-17 in the treatment of various infertility-related defects.

The present application claims the benefit of priority of 60/575,076 filed on May 27, 2004. The entire text of this priority application and its appendices is incorporated herein by reference.

### FIELD OF THE INVENTION

The present invention relates to biomarkers useful for monitoring the effects of inhibition of IMPDH in a patient.

### BACKGROUND

The synthesis of nucleotides in organisms is required for the cells in those organisms to divide and replicate. Nucleotide synthesis in mammals may be achieved through one of two pathways: the de novo synthesis pathway or the salvage pathway. Different cell types use these pathways to a different extent.

Inosine-5′-monophosphate dehydrogenase (IMPDH; EC 1.1.1.205) is an enzyme involved in the de novo synthesis of guanine nucleotides. IMPDH catalyzes the NAD-dependent oxidation of inosine-5′-monophosphate (IMP) to xanthosine-5′-monophosphate (XMP) [Jackson R. C. et. al., Nature, 256, pp. 331-333, (1975)].

IMPDH is ubiquitous in eukaryotes, bacteria and protozoa [Y. Natsumeda & S. F. Carr, Ann. N.Y. Acad., 696, pp. 88-93 (1993)]. The prokaryotic forms share 30-40% sequence identity with the human enzyme. Two isoforms of human IMPDH, designated type I and type II, have been identified and sequenced [F. R. Collart and E. Huberman, J. Biol. Chem., 263, pp. 15769-15772, (1988); Y. Natsumeda et. al., J. Biol. Chem., 265, pp. 5292-5295, (1990)]. Each is 514 amino acids, and they share 84% sequence identity. Both IMPDH type I and type II form active tetramers in solution, with subunit molecular weights of 56 kDa [Y. Yamada et. al., Biochemistry, 27, pp. 2737-2745 (1988)].

The de novo synthesis of guanosine nucleotides, and thus the activity of IMPDH, is particularly important in B and T-lymphocytes. These cells depend on the de novo, rather than salvage pathway to generate sufficient levels of nucleotides necessary to initiate a proliferative response to mitogen or antigen [A. C. Allison et. al., Lancet II, 1179, (1975) and A. C. Allison et. al., Ciba Found. Symp., 48, 207, (1977)]. Thus, IMPDH is an attractive target for selectively inhibiting the immune system without also inhibiting the proliferation of other cells. Although resting lymphocytes can utilize the alternate salvage nucleotide synthesis pathway (Fairbanks et al, 1995), rapidly proliferating lymphocytes rely predominantly on the de novo pathway for satisfying their requirement for nucleotides. This observation makes enzymes of the de novo pathway attractive targets for pharmacological intervention aimed at inhibiting lymphocyte proliferation (Allison and Eugui, 2000). Blocking IMPDH enzyme activity results in a decrease of cellular guanine nucleotide levels, thereby inhibiting DNA and RNA synthesis, resulting ultimately in antiproliferative, immunosuppressive and antiviral effects (reviewed in Franchetti and Grifantini, 1999).

It is also known that IMPDH plays a role in other metabolic events. Increased IMPDH activity has been observed in rapidly proliferating human leukemia cell lines and other tumor cell lines, indicating IMPDH as a target for anti-cancer as well as immunosuppressive chemotherapy [M. Nagai et. al., Cancer Res., 51, pp. 3886-3890, (1991)].

Inhibitors of IMPDH have been extensively investigated to treat various diseases, e.g., tumors and cancers, immunosuppression, and viral diseases, See, e.g., WO 00/56331. Mycophenolic acid (“MPA”) and VX-944 are two known IMPDH inhibitors. VX-944 is currently being investigated for potential use as an anti-cancer agent. Several IMPDH inhibitors have been used clinically and more are being developed for the treatment of immune cell-mediated, chronic inflammatory, antineoplatic and antiviral indications (Jain et al, 2001, Jain et al, 2002, Dhar et al, 2002). In addition, several new, non-nucleoside IMPDH inhibitors have been designed using a rational drug design approach (Sintchak et al, 1996, reviewed in Saunders and Raybuck, 2000). Of these, VX-497, is currently being investigated in a Phase II trial in combination with pegylated IFN and ribavirin for the treatment of Hepatitis C (Markland et al, 2000, Jain et al, 2001). VX-944 inhibits both IMPDH isozymes with Ki values of 7-10 nM (Jain et al, 2003, ASH poster). VX-944 is also very potent in inhibiting the proliferation of human peripheral lymphocytes stimulated with either T or B-cell mitogens with IC50 values ranging from 20-100 nM, and is more potent than VX-497 or MPA (Eugui et al, 1991a, Jain et al, 2001, Jain et al, 2003 ASH poster, Jain et al, 2004 ASH poster). The inhibition of lymphocyte proliferation by VX-944 is alleviated by the addition of 50 μM guanosine, demonstrating its specificity towards the IMPDH enzyme.

There is a need to develop a reliable biomarker that can be readily used for tracking and monitoring the efficacy of such an IMPDH inhibitor. Such a biomarker should be sensitive to inhibition of IMPDH and be readily detectable by methods that are not unduly burdensome.

### SUMMARY OF THE INVENTION

The present invention provides biomarkers that are useful in monitoring the effect of inhibition of IMPDH in a patient. Applicants have identified a group of genes that are up- or downregulated as a result of IMPDH inhibition. Exemplary expression data showing genes that are up- or down-regulated in response to IMPDH inhibition after 24, 48 and 72 hours of exposure to exemplary IMPDH inhibitors are provided in Table IX. Determining the expression of these genes in a biological sample obtained from a subject treated with an IMPDH inhibitor will therefore allow an assessment of whether the individual will be responsive to IMPDH inhibitor therapy.

The invention describes a nucleic acid array consisting essentially of at least 4 polynucleotides selected from the polynucleotides listed in any one or more of Tables I through VIII, wherein said polynucleotides are immobilized on a solid surface, and wherein said array further contains one or more calibration points and one or more housekeeping genes. In some embodiments the nucleic acid array consists of at least 4 polynucleotides selected from the polynucleotides listed in any one or more of Tables I through VIII. The array may have between 1 and 10 calibration spots and also or alternatively may have between 1 to 10 housekeeping genes to serve as controls for the array. The polynucleotides may be cDNAs or oligonucleotides. The arrays may consist of at least 4, 5, 10, 15, 20, 25, 30, 35, 40, 50, 60, 70, 80, 90, 100, 110, 120, 130, 140, 150, 200, 300 or more polynucleotides. The polynucleotides may be preferred polynucleotides presented herein in Tables I through Table VIII or alternatively may be fragments or variants of those polynucleotides. In one preferred aspect the microarray is one which comprises all of the polynucleotides set forth in Table VI. Another preferred microarray contains all of the polynucleotides set forth in Table V. Another preferred microarray contains all of the polynucleotides set forth in Table IV. In preferred embodiments, the array contains more than one polynucleotide hybridizing to the same gene.

Also encompassed by the invention is a nucleic acid array consisting essentially of at least 4 distinct nucleic acid sequences selected from the group consisting of the polynucleotides listed in Table I, immobilized on the surface at discrete and known positions, wherein said nucleic acids hybridize to nucleic acids in a sample of a subject that are either up-regulated or down-regulated in response to inhibition of IMPDH. In other embodiments, there is provided a nucleic acid array consisting of at least 4 distinct nucleic acid sequences selected from the group consisting of the polynucleotides listed in Table I. Another embodiment is directed to a nucleic acid array consisting essentially of at least 4 distinct nucleic acid sequences selected from the group consisting of the polynucleotides listed in Table IV, immobilized on the surface at discrete and known positions, wherein said nucleic acids hybridize to nucleic acids in a sample of a subject that are either up-regulated or down-regulated in response to inhibition of IMPDH. Another embodiment is directed to a nucleic acid array consisting of at least 4 distinct nucleic acid sequences selected from the group consisting of the polynucleotides listed in Table IV. Still more preferably, the nucleic acids on said microarray are selected from the group consisting, or consisting essentially, of the genes from Table V. Alternatively, the nucleic acids on said microarray are selected from the group consisting, or consisting essentially, of the genes from Table VI.

Other exemplary nucleic acid arrays are those that consist, or consist essentially, of at least 4 distinct nucleic acid sequences selected from the group consisting of the polynucleotides listed in Table VII, immobilized on the surface at discrete and known positions, wherein said nucleic acids hybridize to nucleic acids in a sample of a subject that are either up-regulated or down-regulated in response to inhibition of IMPDH. In the arrays the surface is typically selected from the group consisting of a metal, silicon, a polymer plastic, paper, ceramic, quartz, gallium arsenide, metal, metalloid, cellulose, celluose acetate, nitrocellulose, and a glass. More specifically, the plastic is selected from the group consisting of nylon, polycarbonate, polyethylene, polystyrene, teflon, polypropylene, poly(4-methylbutene), polystyrene/latex, polymethacrylate, poly(ethylene terephthalate), rayon, polyvinylbutyrate, and polyvinylidene difluoride. Preferably, the arrays have at least one control spot consisting of one or more nucleic acids that are known not to be modulated with IMPDH inhibition.

In specific embodiments the array surface comprises a plurality of microarrays separated from each other with a hydrophobic polymer strip. In preferred aspects the hydrophobic polymer strip is selected from the group of polyethylene, silicone, paraffin, and Teflon®.

Also contemplated by the invention is a set of polynucleotides for use in the detection of IMPDH inhibition, wherein said polynucleotides hybridize to 4 to 314 genes selected from the group consisting of the genes set forth in any one or more of Table I, Table II, Table III, Table IV, Table V, Table VI, Table VII and Table VIII wherein the expression of each said nucleic acid is either up- or down-regulated in response to inhibition of IMPDH.

Another aspect of the invention is related to a set of polynucleotides for use in the prediction of efficacy of an IMPDH inhibitor in non-proliferating cells, wherein the set of polynucleotides hybridize to 4 to 38 genes selected from the group consisting of the genes set forth in Table I. Still another aspect is directed to a set of polynucleotides for use in the prediction of efficacy of an IMPDH inhibitor in a proliferating cell, wherein the set of polynucleotides hybridize to 4 to 300 genes selected from the group consisting of the genes set forth in Table II, Table III, and Table IV. Preferably, the proliferating cell is from a hematological cancer and said genes are selected from the group of genes set forth in Table V. Alternatively, the genes are selected from the group of genes set forth in Table VI.

Also part of this invention is a set of nucleic acids for use in the prediction of anti-viral efficacy of an IMPDH inhibitor, wherein the set of polynucleotides hybridize to 4 to 9 genes selected from the group consisting of the genes set forth in Table VII. These are not the only genes identified that may predict antiviral activity of VX-944. The genes listed in Table VII are from the intersection of the genes of Table I and IFN-responsive genes. The skilled person may identify additional anti-viral genes by obtaining the intersection between Table IV and IFN-responsive genes.

Another aspect of the invention is a set of nucleic acids for use in the prediction of efficacy of an IMPDH inhibitor as an anti-cancer agent, wherein the set of polynucleotides hybridize to the 4 genes set forth in Table VIII.

The invention specifically contemplates a method for predicting whether a candidate IMPDH inhibitory agent will produce a therapeutic effect in a subject comprising: contacting a biological sample with said inhibitory agent and determining the expression level of four or more prognostic genes selected from the group consisting of the genes set forth in one or more of Gene Table I, Gene Table II, Gene Table III, Gene Table IV, Gene Table V, Gene Table VI, Gene Table VII and Gene Table VIII in said biological sample, wherein a modulation of the expression level of four or more of genes similar to the modulation resulting from a known IMPDH inhibitor is indicative that said agent is a therapeutically effective IMPDH inhibitory agent.

A candidate IMPDH inhibitor produces an expression pattern that is “similar” to a known IMPDH inhibitor, e.g., VX-944 or MPA, if the qualitative, but not necessarily quantitative, effect on gene expression of the candidate and known inhibitors reliably resemble or mimic each other from the perspective of one of skill in the art. The qualitative effect on expression (i.e., the number of genes exhibiting modulated expression and whether that modulation reflects up- or down-regulation) is distinct from the quantitative effect on expression (i.e., the magnitude of a given change in expression). Preferably, a candidate IMPDH inhibitor is identified as producing an expression pattern similar to a known IMPDH inhibitor if the two modulators exert the same qualitative effect (i.e., up-regulated or down-regulated) on a percentage of the modulated genes that is at least 50%, 60%, 70%, 75%, 80%, 90%, 95% or 99%.

Preferably, the effect is a clinical response. More particularly, the subject is a mammal, more specifically a human patient. The response also may be monitored in vitro in tissues or cells lines from a cancer patient. In the methods described herein the biological sample is preferably a sample from a cancer patient, and wherein said cancer is selected from the group consisting of myeloproliferative diseases, leukemia, breast cancer, ovarian cancer, gastric cancer, colorectal cancer, prostate cancer; pancreatic cancer, lung cancer, etc. The methods are such that the alteration of expression in response to said IMPDH inhibitory agent is preferably similar to the alteration in expression seen in response to administration of VX-944.

Preferably, the subject is a human patient. Or human cell lines or tissues or cells derived from cancers. More preferably, the subject is one who is suffering form a proliferative disorder. Where the proliferative disorder is cancer, the biological sample is a sample from a cancer patient, wherein said cancer is selected from the group consisting of myeloproliferative diseases, leukemia, breast cancer, ovarian cancer, gastric cancer, colorectal cancer, prostate cancer; pancreatic cancer, lung cancer, etc. The method may involve comparing the alteration of expression in response to said IMPDH agent to the level of alteration in expression seen in response to administration of VX-944.

Preferably, the sample is a tissue sample comprising cancer cells. The tissue or cells may be fixed, paraffin-embedded, fresh, or frozen. Preferably, the tissue is obtained through a biopsy, such as for example, a fine needle aspiration, bronchial lavage, or transbronchial biopsy. In specific embodiments, the expression level of said prognostic RNA transcript or transcripts is determined by PCR, immunohistochemistry, in situ hybridization, and the like. The assay for the measurement of said prognostic RNA transcripts or their expression products is provided in the form of a kit or kits.

Also contemplated is a method of preparing a prognostic profile for a subject's response to an IMPDH inhibitor comprising the steps of exposing ex vivo cells of said subject to an IMPDH inhibitor, subjecting RNA extracted from the cells of step (a) to gene expression profiling; determining the expression level of at least four genes selected from the group consisting of the genes set forth in Table I, Table II, Table III, Table IV, Table V, Table VI, Table VII and Table VIII in said cells; and comparing the expression levels obtained in step (c) to expression levels obtained in the absence of said IMPDH inhibitor, wherein a modulation of the expression level of said four or more of genes in response to said IMPDH inhibitor indicates that said subject is likely to be responsive to said inhibitor.

Also contemplated is a method of preparing a prognostic profile for a subject's response to an IMPDH inhibitor comprising the steps of administering to said subject to an IMPDH inhibitor; subjecting RNA extracted from the cells of said subject to gene expression profiling; determining the expression level of at least four genes selected from the group consisting of the genes set forth in Table I, Table II, Table III, Table IV, Table V, Table VI, Table VII and Table VIII in said cells of said subject; and comparing the expression levels obtained in step (c) to expression levels obtained in the absence of said administration of said IMPDH inhibitor, wherein a modulation of the expression level of said four or more of genes in response to said IMPDH inhibitor administration indicates that said IMPDH inhibitor will likely predict the subject's response to said IMPDH inhibitor administration. These methods can be used to monitor a response such as a therapeutic response, toxicity, dose range, and patient stratification.

In these methods, the cells may be cancer cells, e.g., cells selected from the group consisting of breast cancer, ovarian cancer, hematological cancer, gastric cancer, colorectal cancer, pancreatic cancer, and lung cancer. The cells can be fixed or may be from a fresh biopsy or may be from a cancer cell culture. In preferred embodiments, the cancer cells are cultured ex vivo. In the above methods the expression profile may be compiled into a report that includes recommendation for a treatment for said subject with an IMPDH inhibitor. In further embodiments, if altered expression of one or more of the genes selected from the genes set forth in Tables I, II, III, IV, V, VI, VII, and VIII, or the corresponding expression product is determined, said report includes a prediction that said subject is a suitable candidate for IMPDH inhibition-based therapy. In certain embodiments, the method may further comprise treating said patient with an IMPDH inhibitory agent. The biological sample used may be a blood sample, a tissue biopsy, or a tumor cell isolated from a tumor biopsy. The subset of genes could also be useful in conducting animal models or defining biomarkers in animal models used to determine efficacy or PK-PD correlation for IMPDH inhibitors.

The treatment method is one in which subject has been treated with any IMPDH inhibitor. In this regard, several IMPDH inhibitors have been used clinically and more are being developed for the treatment of immune cell-mediated, chronic inflammatory, antineoplatic and antiviral indications (Jain et al, 2001, Jain et al, 2002, Dhar et al, 2002). Mycophenolic acid (MPA), has been demonstrated to inhibit the proliferation of lymphocytes in vitro and in vivo (Eugui et al, 1991a, Allison and Eugui, 2000). An ester pro-drug of MPA, mycophenolate mofetil (CellCept®) is prescribed for the prevention of acute rejection in kidney, heart and liver transplantation (reviewed in Mele and Halloran, 2000), in combination with steroids and cyclosporine A (CsA). Mizoribine (Bredinin®) and ribavirin (Virazole®, Rebetol®) are nucleoside analogs which following intracellular phosphorylation are competitive IMPDH inhibitors (Franchetti and Grifantini, 1999, Saunders and Raybuck, 2000). Mizoribine is approved in Japan for multiple indications including prevention of rejection after renal transplantation, idiopathic glomerulonephritis, lupus nephritis, and rheumatoid arthritis (Ishikawa 1999). Ribavirin is used extensively as an inhaled antiviral agent for treatment of respiratory syncytial virus (RSV) and, orally in combination with interferon-a, for the treatment of chronic hepatitis C viral (HCV) infection (Davis et al 1998, McHutchison et al, 1998, Poynard et al 1998). Antineoplastic and antiviral activities of other nucleoside and NAD analogs, such as tiazofurin for the treatment of CML (reviewed in Jayaram et al, 1999), have been described but these are not yet clinically approved (reviewed in Saunders and Raybuck, 2000). The diverse therapeutic indications for these and other IMPDH inhibitors that are being developed illustrate the unmet clinical need for potent, reversible and lymphocyte-selective inhibitors that are safe, well-tolerated, and suitable for long-term dosing. The methods of the invention will facilitate such evaluations.

Also part of the invention are kits which comprise the nucleic acid arrays described herein in combination with appropriate containers, buffers, reagents and instructions for use in diagnostic and/or therapeutic methods. Such kits may further comprise nucleic acids of a reference subject as calibration standards. Further, the kits also may comprise detectable labels for the compositions of the invention.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWING(S)

The following drawings form part of the present specification and are included to further illustrate aspects of the present invention. The invention may be better understood by reference to the drawings in combination with the detailed description of the specific embodiments presented herein.

FIG. 1 shows treatment comparisons in which it is observed that there are differences in gene expression (scatter) enhanced at 30 h compared to the 14 h time-point for VX-944 and Mycophenolic acid (MPA).

FIGS. 2A-2C provides % cv's for certain genes for various treatment groups (n=3). There is a heirarchical clustering of certain transcript profiles (data not shown).

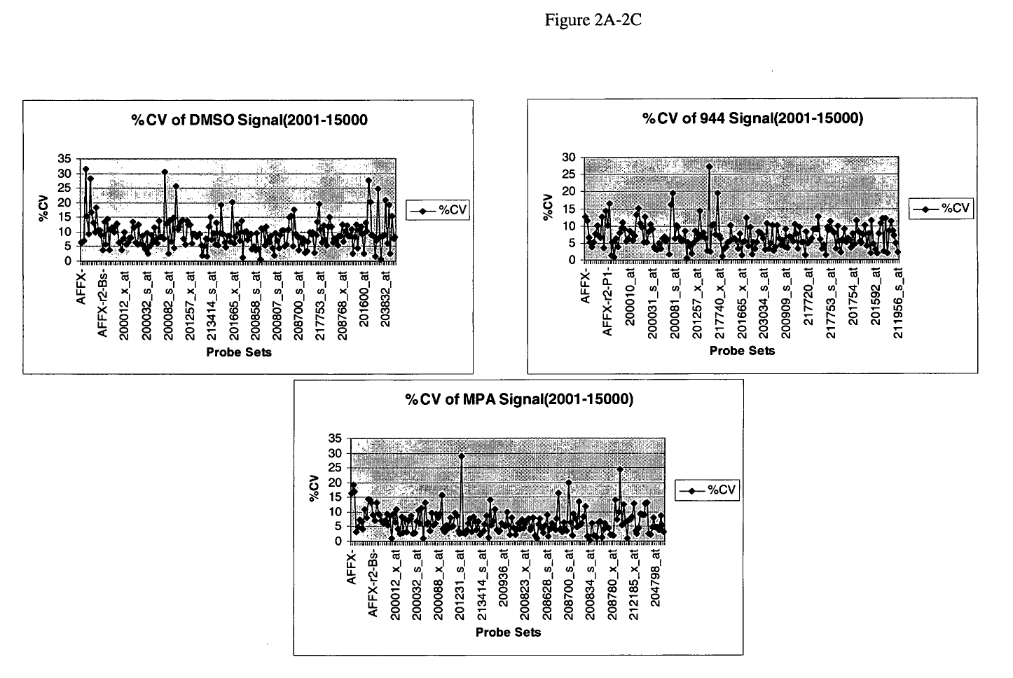

FIG. 3 shows exemplary genes co-regulated by VX-944 and MPA.

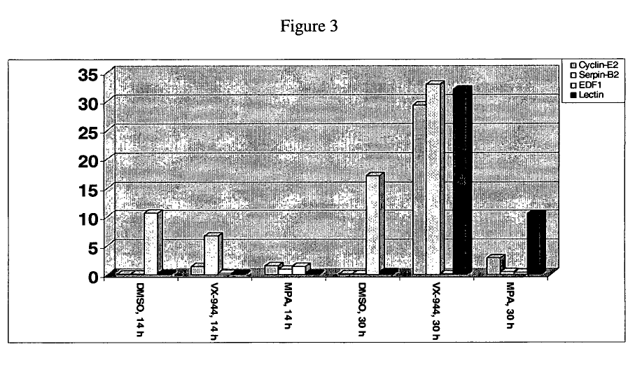

FIG. 4 shows exemplary genes down-regulated by VX-944 at various time points of exposure.

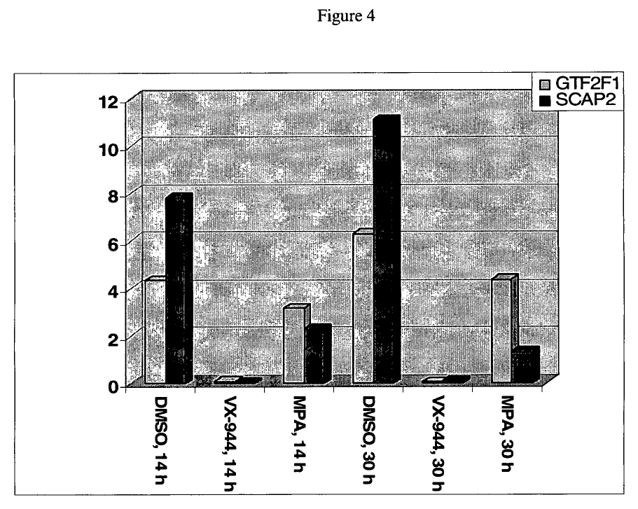

FIG. 5 shows exemplary genes upregulated by MPA at various time points of exposure.

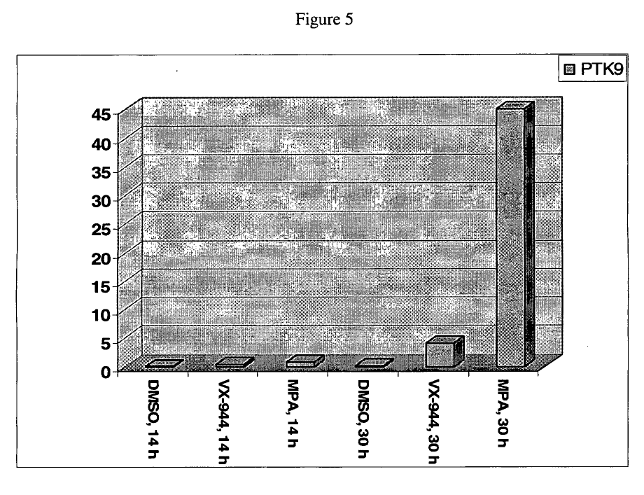

FIG. 6 shows additional exemplary genes upregulated by VX-944 at various time points of exposure.

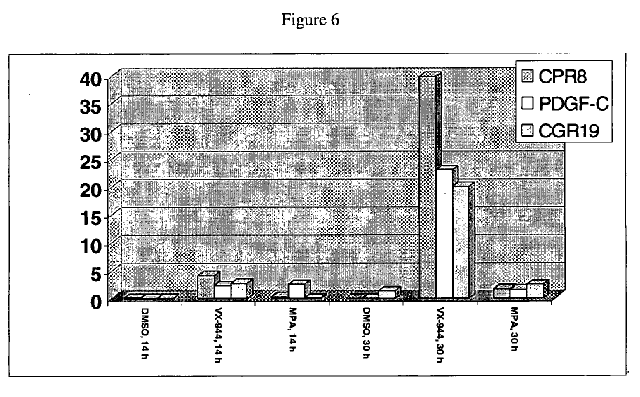

FIG. 7 shows exemplary genes upregulated by both VX-944 and MPA at various time points of exposure.

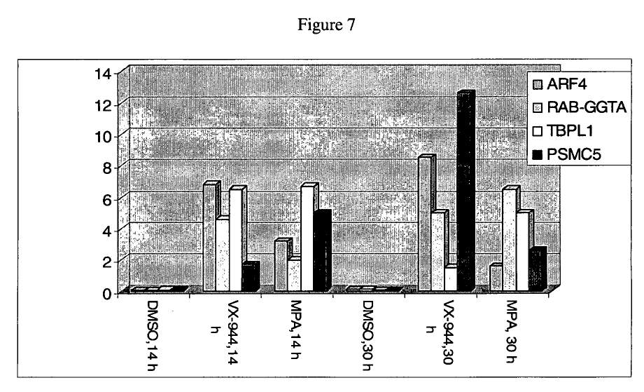

FIG. 8 shows exemplary biomarkers of IMPDH inhibition.

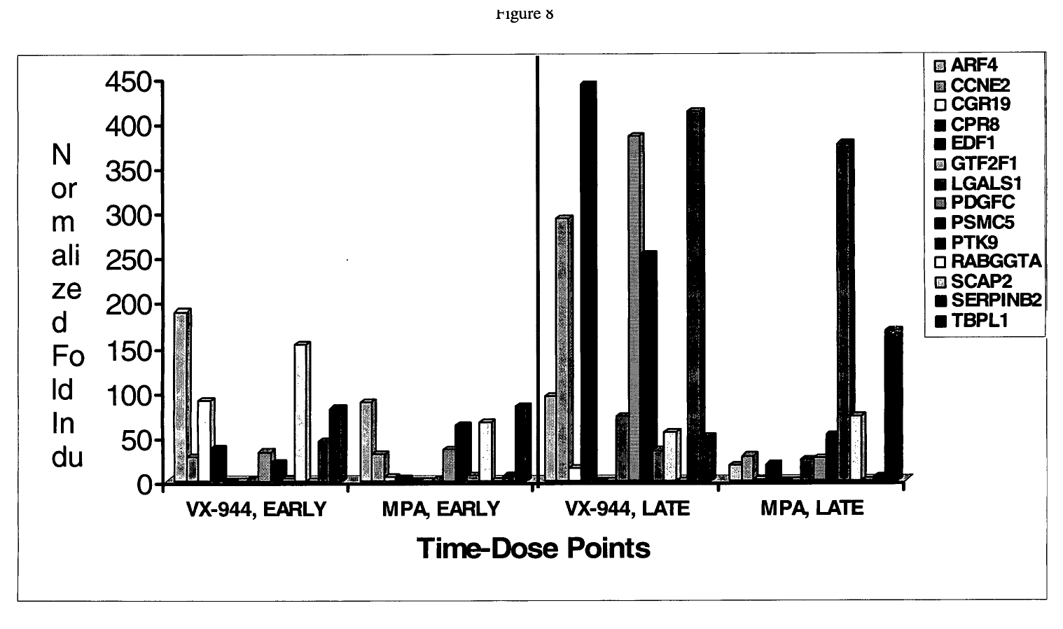

FIG. 9 shows exemplary biomarkers of IMPDH inhibition segregated according to early and late biomarkers.

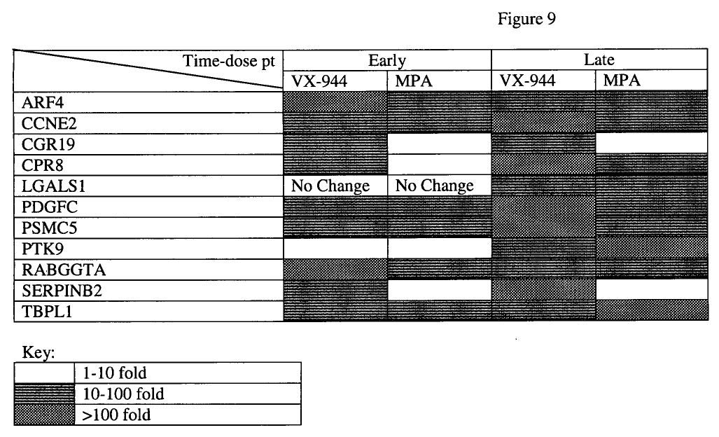

FIG. 10 shows QRT-PCR data that validates the data obtained using the gene chip data disclosed. These data were generated using expression patterns, impact of the inhibitors on specific pathways, high dynamic range, mechanistic relevance and cancer patient literature to select that candidate biomarker panel that will be useful. From these studies IL1RN (Interleukin 1 receptor antagonist), SPP1 (Secreted phosphoprotein 1), NCF1 (Autosomal chronic granulomatous disease-associated gene) and BCL2 (B-cell CLL/lymphoma 2—Anti-apoptosis marker) were selected as suitable candidate markers for IMPDH inhibition in HL-60 cells.

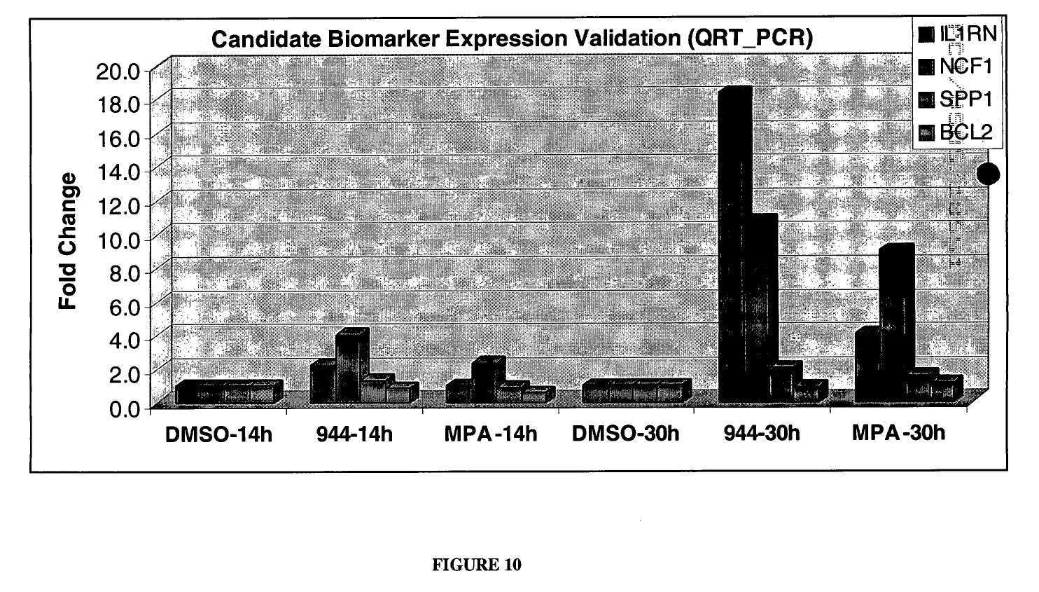

FIG. 11 shows that candidate marker responses to VX-944 can be monitored in human PBMCs. The same markers discussed above were used in the studies shown in this figure.

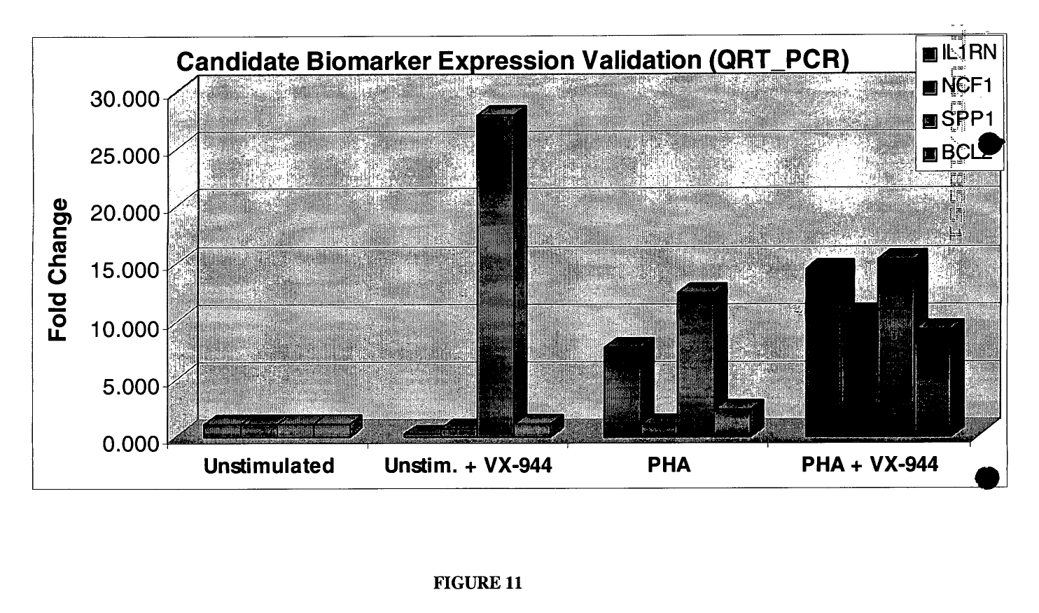

### DETAILED DESCRIPTION OF THE PREFERRED EMBODIMENTS OF THE INVENTION

Traditional therapeutic monitoring of drugs involves the measurement of drug concentrations in patient blood. Several biochemical approaches to measure the biological effect of MPA or MMF (Langman et al 1995, Langman et al 1996, Griesmacher et al, 1997, Gummert et al, 1999, Albrecht et al, 2000, Budde et al, 2000, Qiu et al, 2000, Daxecker et al, 2001), mizoribine (Hirohata and Yamagida, 1995), and ribavirin (Fairbanks et al, 1995, Montero et al, 1995) have been described. In the present invention, the inventors have evaluated gene expression changes in response to VX-944 as an example of a potent and specific IMPDH inhibitor. MPA, a structurally unrelated but mechanistically similar IMPDH inhibitor, was included in some studies to compare its effect with VX-944. Given the similar mechanism of action of the two compounds, genes affected by both VX-944 and MPA are more likely to represent biomarkers for IMPDH inhibition. These results will be valuable in optimizing the dosing regimen and therapeutic benefit of IMPDH-based therapies in a broad range of therapeutic indication. Monitoring these patterns of gene expression changes are also likely to be predictive of a beneficial outcome with other unknown IMPDH pathway inhibitors. The gene expression changes observed with VX-944 and MPA may also allow selection of drugs that may enhance the effect of IMPDH inhibitors in combination treatments. Examples of gene expression changes have been published for Gleevec/STI-571. Based on the two compounds targeting two complementary pathways, we predict that a combination of the two agents is likely to be synergisitic in treating cancer.

The present invention is directed to methods and compositions for using biomarkers that are responsive to changes in IMPDH activity levels. More particularly, in certain preferred embodiments, the invention is directed to biomarkers that are responsive to IMPDH inhibition. In preferred aspects of the present invention there are provided nucleic acid arrays or sets of nucleic acids that are specifically up-regulated or down-regulated in response to IMPDH inhibition. Having identified these specific nucleic acids, it is now possible to prepare microarrays and specific collections of nucleic acids that may be used in order to identify additional IMPDH inhibitors, to verify that a known IMPDH inhibitor is exerting a desired effect in vitro or in vivo, and to provide a genetic profile of a subject under investigation in order to determine whether the individual is responsive to IMPDH inhibition. In one embodiment, the inventors obtained 18 gene chips that contained a total of 8793 genes on the arrays. Out of these 8793 genes there were 3421 genes that were common to all 18 gene chips. Of these, the inventors identified 1355 genes that were responsive to IMPDH inhibition. Gene sets 2 and 3 identified in the examples provided below contain the preferred subset of genes that are responsive to IMPDH inhibition. The following table summarizes the response of those genes to such inhibition and from these analyses the genes that were identified as particularly useful exemplary biomarkers for IMPDH inhibition are listed immediately beneath the table.

| | |
|---|---|
| No. genes modulated by DMSO | 32 |
| No. genes modulated by MPA | 75 |
| Increased | 39 |
| Decreased | 36 |
| No. genes modulated by VX-944 | 106 |
| Increased | 46 |
| Decreased | 60 |
| No. genes increased by both | 62 |
| No. genes decreased by both | 63 |

Of the above genes, particularly useful exemplary biomarkers include, but are not limited to: ARF4; CCNE2; CGR19; CPR8; EDF1; GTF2F1; LGALS1; PDGFC; PSMC5; PTK9; RABGGTA; SCAP2; SERPINB2; TBPL1.

Monitoring gene expression patterns offers an alternative approach for optimizing the therapeutic window as compared with the traditional method of measuring drug concentrations in blood and correlating either the AUC or Cmax levels with a clinical response. Towards that end, the inventors have comprehensively evaluated several genes affected by treatment with two distinct IMPDH inhibitors in vitro, as potential readouts for assessing their biological activity. The genes that are listed in Tables I through VIII are particularly useful in the present invention. These genes were identified using the specific methods as follows: Most cell culture reagents, unless specified otherwise, were obtained from Gibco-BRL or JRH Biologicals. Complete RPMI was prepared by adding up to a final of 10% heat-inactivated fetal bovine serum (FBS), 55 μM β mercaptoethanol, 50 units/mL penicillin with 50 μg/mL streptomycin, 300 μg/ml L-glutamine and 10 mM HEPES pH 7.5 to RPMI1640. Stock solutions of Phyto-hemagglutinin (PHA-P, Difco) was prepared in FBS-free RPMI and stored at −20° C. VX-944, and MPA (Sigma-Aldrich or Calbiochem), were dissolved in DMSO at a concentration of 20 mM and stored at −20° C.

Peripheral blood mononuclear cells (PBMCs) and T cells were isolated as follows: Human venous blood was drawn from healthy volunteers using heparin as an anti-coagulant. PBMCs were isolated from blood by centrifugation over Ficoll-paque gradient or CPT tubes (Becton-Dickinson, Calif.), using standard conditions. PBMCs were harvested, washed and resuspended in complete RPMI, counted and diluted to 1×10⁶ cells/ml.

In order to monitor PBMC proliferation assay, human PBMC derived T cells (5×10⁴ cells/well), were added to 96-well plates. PHA was added up to a final concentration of 10-20 μg/mL per well for stimulating T cells. The mitogen SPAS was used at a final concentration of 2 mg/mL. DMSO was maintained at a final concentration of 0.1%.

The methods described here are standard techniques that can be implemented at many clinical laboratories. The readouts can be adapted further for pharmacodynamic monitoring of IMPDH inhibitors in a variety of therapeutic indications, ultimately helping to optimize their therapeutic window in patients.

The methods and compositions of the present invention will typically employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 3rd edition (Sambrook et al., 2000); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4.sup.th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymnerase Chain Reaction”, (Mullis et al., eds., 1994); ); Current Protocols in Immunology.

Exemplary aspects of the present invention will involve partial and/or complete gene expression profiling of a patient in order to assess whether the patient has or will be responsive to IMPDH inhibition. Such gene expression profiling methods include methods based on hybridization analysis of polynucleotides, methods based on sequencing of polynucleotides, and proteomics-based methods. Common methods for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

PCR-based methods of expression profiling include, for example, RT-PCR, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex¹⁰⁰ LabMAP system and multiple color-coded microspheres (Luminex Corp., Austin, Tex.) in a rapid assay for gene expression (Yang et al., Genome Res. 11:1888-1898 (2001)); high coverage expression profiling (HiCEP) analysis (Fukumura et al., Nucl. Acids. Res. 31(16) e94 (2003)), and MassARRAY-based gene expression profiling method, which employs a mass spectrometry detection system.

A TaqMan assay is used to detect and optionally measure specific sequences in Polymerase Chain Reaction (PCR) products by employing the 5′Δ3′ exonuclease activity of Taq DNA polymerase. The TaqMan probe disabled from extension at the 3′ end, consists of a site-specific sequence labeled with a fluorescent reporter dye and a fluorescent quencher dye. During PCR the TaqMan probe set are derived from the genes of Tables I through VIII hybridizes to its complementary single stranded DNA sequence within the PCR target. When amplification occurs the TaqMan probe is degraded due to the 5′→3′ exonuclease activity of Taq DNA polymerase, thereby separating the quencher from the reporter during extension. Due to the release of the quenching effect on the reporter, the fluorescence intensity of the reporter dye increases. During the entire amplification process this light emission increases exponentially, the final level being measured by spectrophotometry after termination of the PCR. Because increase of the fluorescence intensity of the reporter dye is only achieved when probe hybridization and amplification of the target sequence has occurred, the TaqMan assay offers a sensitive method to determine the presence or absence of specific sequences. Therefore, this technique is particularly useful in diagnostic applications, such as the screening of samples for the presence or incorporation of favorable traits and the detection of pathogens and diseases. The TaqMan assay allows high sample throughput because no gel-electrophoresis is required for detection. Each TaqMan® Gene Expression Assay consists minimally of two unlabeled primers and a labeled probe. A more detailed description of this technique is contained in e.g., Bustin, S A. (2000). Journal of Molecular Endocrinology, 25: 169-193.

Sample preparation for use in analysis. In order to conduct sample analysis in the above gene expression profiling methods, a sample containing target polynucleotides is provided. The samples derived from can be any source containing target polynucleotides and obtained from any bodily fluid (blood, urine, saliva, phlegm, gastric juices, etc.), cultured cells, biopsies, or other tissue preparations. In preferred embodiments the target nucleic acid is obtained from a biological sample obtained from an individual that has undergone therapy based on IMPDH inhibition. In some embodiments, the expression profile of such a patient is obtained before and after IMPDH inhibition in order to assess the change in gene expression as a result of IMPDH therapy. Such expression profiling may be ongoing throughout the IMPDH therapy in order to assess whether the inhibition is having a sustained effect throughout the therapy.

DNA or RNA can be isolated from the biological sample according to any of a number of methods well known to those of skill in the art. In certain embodiments, the nucleic acid is isolated from a variety of primary tumors, including breast, lung, colorectal, prostate, brain, liver, kidney, pancreas, spleen, thymus, testis, ovary, uterus, etc., tumor, or tumor cell lines, with pooled DNA from healthy donors. More generally, the nucleic acid is isolated from hyperproliferative cells such as those found in immune system disorders such as transplant rejection and autoimmune diseases, such as rheumatoid arthritis, multiple sclerosis, juvenile diabetes, asthma, inflammatory bowel disease, as well as in the treatment of cancer and tumors, such as lymphomas and leukemia, vascular diseases, such as restenosis, and viral replication diseases, such as retroviral diseases, hepatitis C, and herpes. If the source of mRNA is a primary tumor or primary tissue from a hyperproliferative disorder, mRNA can be extracted, for example, from frozen or archived paraffin-embedded and fixed (e.g. formalin-fixed) tissue samples. Other methods of purification of nucleic acids are described in Tijssen (1993) Laboratory Techniques in Biochemistry and Molecular Biology: Hybridization With Nucleic Acid Probes, Part I. Theory and Nucleic Acid Preparation, Elsevier, New York N.Y. For example, total RNA is can be isolated using the TRIZOL reagent (Life Technologies, Gaithersburg Md.), and mRNA is isolated using oligo d(T) column chromatography or glass beads. Alternatively, when target polynucleotides are derived from an mRNA, the target polynucleotides can be a cDNA reverse transcribed from an mRNA to generate more stable cDNAs, an RNA transcribed from that cDNA, a DNA amplified from that cDNA, an RNA transcribed from the amplified DNA, and the like. When the target polynucleotide is DNA, it can be derived from a DNA source or from an RNA source by reverse transcription. In yet another embodiment, the targets are target polynucleotides prepared by more than one method.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrs et al., BioTechniques 18:42044 (1995). In particular, RNA isolation can be performed using purification kit, buffer set and protease from commercial manufacturers, such as Qiagen™, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit, (Ambion, Inc.). Total RNA from tissue samples can be isolated using RNA Stat-60 (Tel-Test). RNA prepared from tumor can be isolated, for example, by cesium chloride density gradient centrifugation. Note that as RNA cannot serve as a template for PCR, the first step in gene expression profiling by RT-PCR is the reverse transcription of the RNA template into cDNA, followed by its exponential amplification in a PCR reaction.

When target polynucleotides are amplified it is desirable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single-stranded DNA template. The second DNA strand is polymerized using a DNA polymerase and a RNAse (e.g., RNAse H) which assists in breaking up the DNA/RNA hybrid. After synthesis of the double-stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine U.S. Pat. No. 5,514,545).

Controls to quantitate the product may be included within the sample to assure that amplification and labeling procedures do not change the relative representation of target polynucleotides in a sample. For this purpose, a sample is spiked with a known amount of a control target polynucleotide and the composition of polynucleotide probes includes reference polynucleotide probes which specifically hybridize with the control target polynucleotides. After hybridization and processing, the hybridization signals obtained should reflect accurately the amounts of control target polynucleotide added to the sample.

Prior to hybridization, it may be desirable to fragment the nucleic acid target polynucleotides. Fragmentation improves hybridization by minimizing secondary structure and cross-hybridization to other nucleic acid target polynucleotides in the sample or noncomplementary polynucleotide probes. Fragmentation can be performed by mechanical or chemical means.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent. markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes and the like. Preferably, fluorescent markers absorb light above about 300 nm, preferably above 400 nm, and usually emit light at wavelengths at least greater-than 10 nm above the wavelength of the light absorbed. Preferred fluorescent markers include fluorescein, phycoerythrin, rhodamine, lissamine, and C3 and C5 available from Amersham Pharmacia Biotech (Piscataway N.J.).

Labeling can be carried out during an amplification reaction, such as polymerase chain and in vitro transcription reactions, or by nick translation or 5′ or 3′-end-labeling reactions. When the label is incorporated after or without an amplification step, the label is incorporated by using terminal transferase or by kinasing the 5′ end of the target polynucleotide and then incubating overnight with a labeled oligonucleotide in the presence of T4 RNA ligase.

Alternatively, the labeling moiety can be incorporated after hybridization once a probe/target complex has formed.

Nucleic Acid Microarrays: The samples prepared above are hybridized to nucleic acid microarrays of the invention. An array of the invention typically will be a nucleic acid array consisting essentially of at least 4 polynucleotides selected from the polynucleotides listed in any one or more of Gene Tables I through Gene Table VIII, wherein said polynucleotides are immobilized on a solid surface, and wherein the array further contains one or more calibration points and one or more housekeeping genes.

The housekeeping genes are provided as controls for the chip so as to provide signals corresponding to nucleic acids that are constitutively expressed at a known level. These genes are presumed to produce the minimally essential transcripts necessary for normal cellular physiology, and are expressed at a similar level in almost all cells. These controls are useful in normalizing the data, for example to quantify expression levels. As a control gene, any known reference gene can be used, including, for example, glyceraldehyde-3-phosphate dehydrogenase (GAPDH), β-actin, U-snRNP-associated cyclophilin (USA-CYP), ribosomal protein LPO; 18S ribosomal RNA; Glucoronidase beta; and Ribosomal protein L32. Alternatively, normalization can be achieved by correcting for differences between the total of all signals of the tested gene sets (global normalization strategy). The report may include a prognosis for the outcome of the treatment of the patient. The method may additionally comprise the step of treating the subject, e.g. a human patient, if a good prognosis is indicated.

The calibration spot is a signal that is contained on the microarray that will be used to ensure that the microarray is approprotately aligned and being read by a detection device. Typically, such a calibration spot comprises a mixture of the plurality of nucleic acids isolated from the sample that are known not to be modulated with IMPDH inhibition.

In the present invention, 5 or more nucleic acids selected from the distinct sequences of listed in Gene Tables I-VIII may be presented in a DNA microarray for the analysis and expression of these genes in various cell types. Microarray chips are well known to those of skill in the art (e.g., see U.S. Pat. No. 6,308,170; U.S. Pat. No. 6,183,698; U.S. Pat. No. 6,306,643; U.S. Pat. No. 6,297,018; U.S. Pat. No. 6,287,850; U.S. Pat. No. 6,291,183, each incorporated herein by reference. These are merely exemplary patents that disclose nucleic acid microarrays and those of skill in the art will be aware of numerous other methods and compositions for producing microarrays). Given the identification of the above nucleic acids that are specifically responsive to IMPDH inhibition it is possible to use commercially available microarray chips that contain probes that will hybridize to such nucleic acids regardless of any other nucleic acid probes contained on such chips. In this regard a preferred chip is the Affymetrix HG-FOCUS arrays (Affymetrix, CA catalog #900377).

The present invention provides for a composition comprising a plurality of polynucleotide probes for use in detecting changes in expression of a large number of genes from cells in response to IMPDH inhibition. As used in this section, the term “polynucleotide probe” refers to a nucleic acid that hybridize to any one of the nucleic acids listed in Tables I-VIII or hybridizes to any fragment thereof or any nucleic acid that hybridizes specifically to one such nucleic acid. Particularly preferred exemplary markers of IMPDH inhibition are provided in gene sets I through VIII. Other exemplary markers include ARF4; CCNE2; CGR19;CPR8; EDF1; GTF2F1; LGALS1; PDGFC; PSMC5; PTK9; RABGGTA; SCAP2; SERPINB2; TBPL1. Preferably the fragment is longer than 20 base pairs. Such a composition can be employed for the diagnosis and for monitoring the treatment of any disease in which abnormal IMPDH is found, and preferably diseases in which overexpression or increased activity of IMPDH is implicated. IMPDH-mediated disease refers to any disease state in which the IMPDH enzyme plays a regulatory role in the metabolic pathway of that disease. Examples of IMPDH-mediated diseases include transplant rejection and autoimmune diseases, such as rheumatoid arthritis, multiple sclerosis, juvenile diabetes, asthma, and inflammatory bowel disease, as well as inflammatory diseases, cancer, viral replication diseases and vascular diseases. In particular embodiments, the compositions of the invention may be used to monitor/track the treatment of transplant rejection (e.g., kidney, liver, heart, lung, pancreas (islet cells), bone marrow, cornea, small bowel and skin allografts and heart valve xenografts), rheumatoid arthritis, multiple sclerosis, juvenile diabetes, asthma, inflammatory bowel disease (Crohn's disease, ulcerative colitis), lupus, diabetes, mellitus myasthenia gravis, psoriasis, dermatitis, eczema, seborrhea, pulmonary inflammation, eye uveitis, hepatitis, Grave's disease, Hashimoto's thyroiditis, Behcet's or Sjorgen's syndrome (dry eyes/mouth), pernicious or immunohaemolytic anaemia, idiopathic adrenal insufficiency, polyglandular autoimmune syndrome, and glomerulonephritis, scleroderma, lichen planus, viteligo (depigmentation of the skin), autoimmune thyroiditis, and alveolitis, inflammatory diseases such as osteoarthritis, acute pancreatitis, chronic pancreatitis, asthma and adult respiratory distress syndrome, as well as in the treatment of cancer and tumors, such as solid tumors, lymphomas and leukemia, vascular diseases, such as restenosis, stenosis and atherosclerosis, and DNA and RNA viral replication diseases, such as retroviral diseases, and herpes.

A particularly useful composition of the present invention is a collection of hybridizable array elements in a microarray for monitoring the expression of a plurality of target polynucleofides. The microarray typically comprises a solid substrate and hybridizable array elements positioned on said substrate. The microarray can be used, for example, in the assessment of the efficacy of IMPDH inhibition on the treatment of a cancer, in immune disorders, in neuropathologies, and the like.

Tables I through VIII herein list the exemplary sequences disclosed herein as being responsive to IMPDH inhibition. In set I there are listed specific genes that are responsive to VX-944 in resting PBMC cells, i.e., genes from normal non-proliferating lymphocytes that were found to be specifically responsive (i.e., their expression levels were either increased or decreased) to VX-944. Gene set I can be described as a set of genes whose expression changes in changes in blood obtained from patients suffering from cancer, autoimmune, inflammatory diseases upon treatment with IMPDH inhibitors. These genes were: thrombospondin 1; interleukin 18 receptor 1; aldehyde dehydrogenase 1 family, member A1; complement component 1, q subcomponent, beta polypeptide; chitinase 3-like 1 (cartilage glycoprotein-39); BCR downstream signaling 1; XIAP associated factor-1; protein kinase N2; interferon-induced protein with tetratricopeptide repeats 5; CD38 antigen (p45); peroxisomal biogenesis factor 19; defensin, alpha 1, myeloid-related sequence; cleavage and polyadenylation specific factor 1, 160 kDa; hemochromatosis; chromosome 1 open reading frame 29; apolipoprotein B mRNA editing enzyme, catalytic polypeptide-like 3B; interferon, alpha-inducible protein 27; ubiquitin specific protease 18; interferon-induced protein with tetratricopeptide repeats 1; tissue inhibitor of metalloproteinase 2; myxovirus (influenza virus) resistance 1, interferon-inducible protein p78 (mouse); chemokine (C-C motif) ligand 8; interferon regulatory factor 7; 2′-5′-oligoadenylate synthetase 3, 100 kDa; sialoadhesin; interferon-induced protein 44; myxovirus (influenza virus) resistance 2 (mouse); phospholipid scramblase 1; hect domain and RLD 5; interferon, alpha-inducible protein (clone IFI-6-16); interferon, alpha-inducible protein (clone IFI-15K); suppressor of cytokine signaling 1; chromosome 6 open reading frame 11; chemokine (C-C motif) ligand 13; chemokine (C-C motif) ligand 2; interleukin 1 receptor antagonist; chemokine (C-X-C motif) ligand 10; chemokine (C-X-C motif) ligand 9. The sequences of these genes are provided below in Table 1.

In set II there are listed specific genes that are responsive to VX-944 in resting or non-proliferating and proliferating cells. As such, these genes are those whose gene expression changes in normal human lymphocytes upon treatment with VX-944 or a mitogen or both mitogen and VX-944. This set was determined as follows: the genes altered by VX-944 administration alone in resting lymphocytes were identified by comparing genes altered in VX-944 treated cells as compared to untreated cells. These genes from resting cells were compared with gene expression profile seen in proliferating cells by comparing genes altered in HL60 cells treated with VX-944 compared to untreated HL60 cells. Subsequently, gene expression pattern changes attenuated in proliferating human lymphocytes in the presence of with VX-944 treatment were determined by comparing genes altered in PHA-treated cells compared to untreated cells as compared with genes altered by PHA+VX-944 treated cells. These genes were: chitinase 3-like 1 (cartilage glycoprotein-39) and interleukin 1 receptor antagonist. In gene set III, an assessment was made of the genes that are responsive to VX-944 in resting PBMC cells as well as in HL-60 cells and also genes that were responsive to MPA, another IMPDH inhibitor. These studies again showed that both chitinase 3-like 1 (cartilage glycoprotein-39) and interleukin 1 receptor antagonist are particularly responsive. As such, these two genes will be particularly predictive biomarkers of responsiveness of a given cell to IMPDH inhibition. Gene set II can be monitored in lymphocytes or blood obtained from cancer patients that have been treated with an IMPDH inhibitor such as VX-944.

Set IV shows the genes that could be monitored in proliferating cells from patients treated with VX-944. This set is typical of the gene expression changes that can be monitored in lymphocytes or blood obtained from cancer, autoimmune, transplant-patients, virally infected or otherwise diseased subjects that have been treated with VX-944. These genes represent a subset of genes that are up- or down-regulated in lymphocytes upon stimulation with a mitogen such as PHA (T1), that are modulated in the presence of VX-944.

These genes were identified by first inducing PBMC cells to proliferate, e.g., by stimulating the cells with PHA and determining which genes have an altered expression upon proliferation alone. The PBMC's also were analyzed to obtain the differential expression seen in response to both stimulation with PHA and treatment with VX-944. The overlap between the genes that were expressed in both sets of experiments is listed in Set IV as genes that should preferably be monitored in proliferating lymphocytes or cells of patients treated with VX-944. These genes are: RAD54 homolog B (homolog of S. cerevisiae); chemokine (C-C motif) ligand 2; kinesin family member 4A; spleen focus forming virus (SFFV) proviral integration oncogene spi1; chromosome condensation 1; Fc fragment of IgG, high affinity Ia, receptor for (CD64); asialoglycoprotein receptor 2; aurora kinase B; a disintegrin and metalloproteinase domain 28; growth arrest-specific 7; mucolipin 1; activator of S phase kinase; hexokinase 3 (white cell); nudix (nucleoside diphosphate linked moiety X)-type motif 1; Fc fragment of IgE, low affinity II, receptor for (CD23A); hemopoietic cell kinase; kinesin family member 11; SNARE protein Ykt6; 3-hydroxyisobutyryl-Coenzyme A hydrolase; cytochrome P450, family 1, subfamily B, polypeptide 1; chemokine (C motif) ligand 1; cyclin F; primase, polypeptide 2A, 58 kDa; Charcot-Leyden crystal protein; alpha-2-macroglobulin; exonuclease 1; CDC6 cell division cycle 6 homolog (S. cerevisiae); matrix metalloproteinase 1 (interstitial collagenase); protein regulator of cytokinesis 1; leukocyte immunoglobulin-like receptor, subfamily B (with TM and ITIM domains), member 1; KIAA0146 protein; CDC28 protein kinase regulatory subunit 1B; baculoviral IAP repeat-containing 5 (survivin); Rac GTPase activating protein 1; zinc finger homeobox 1b; glycoprotein (transmembrane) nmb; IGF-II mRNA-binding protein 2; solute carrier family 27 (fatty acid transporter), member 2; cyclin-dependent kinase inhibitor 3 (CDK2-associated dual specificity phosphatase); chemokine (C-X3-C motif) receptor 1; sperm associated antigen 5; formyl peptide receptor 1; cadherin 3, type 1, P-cadherin (placental); origin recognition complex, subunit 1-like (homolog of yeast); chromosome condensation 1-like; protein tyrosine phosphatase, non-receptor type 12; myxovirus (influenza virus) resistance 1, interferon-inducible protein p78 (homolog of mouse); solute carrier family 11 (proton-coupled divalent metal ion transporters), member 1; solute carrier family 7 (cationic amino acid transporter, y+ system), member 7; serine hydroxymethyltransferase 2 (mitochondrial); nuclear receptor subfamily 4, group A, member 2; CDC28 protein kinase regulatory subunit 2; aldehyde dehydrogenase 2 family (mitochondrial); matrix metalloproteinase 19; development and differentiation enhancing factor 2; dipeptidylpeptidase 3; aryl hydrocarbon receptor; chemokine (C-C motif) ligand 8; chromosome 1 open reading frame 29; tensin-like SH2 domain containing 1; killer cell lectin-like receptor subfamily F, member 1; tissue inhibitor of metalloproteinase 1 (erythroid potentiating activity, collagenase inhibitor); cat eye syndrome chromosome region, candidate 1; splicing factor 1 [BLAST]; v-fos FBJ murine osteosarcoma viral oncogene homolog; pleckstrin homology-like domain, family A, member 1; geminin, DNA replication inhibitor; hyaluronan-mediated motility receptor (RHAMM); chromosome 1 open reading frame 33; regulator of G-protein signalling 2, 24 kDa; transforming growth factor, beta-induced, 68 kDa; serine/threonine kinase 6; Fc fragment of IgG, low affinity IIa, receptor for (CD32); ribonuclease T2; arachidonate 5-lipoxygenase; cytokine receptor-like factor 2; purinergic receptor P2Y, G-protein coupled, 5; chromobox homolog 5 (HP1 alpha homolog of Drosophila); inositol(myo)-1 (or 4)-monophosphatase 2; 2′-5′-oligoadenylate synthetase 3, 100 kDa; transcription factor EC; chondroitin sulfate proteoglycan 2 (versican); histone 1, H2ac; metallothionein 1X; aquaporin 3; apolipoprotein A-II; chemokine (C motif) ligand 1; RAD51 homolog (RecA homolog of E. coli) (homolog of S. cerevisiae); C-type (calcium dependent, carbohydrate-recognition domain) lectin, superfamily member 5; TYRO protein tyrosine kinase binding protein; polymerase (DNA directed), epsilon 2 (p59 subunit); FBJ murine osteosarcoma viral oncogene homolog B; fucosyltransferase 8 (alpha (1,6) fucosyltransferase); CD14 antigen; GLI pathogenesis-related 1 (glioma); CD209 antigen; thymidine kinase 1, soluble; interleukin 9; dihydropyrimidine dehydrogenase; lectin, galactoside-binding, soluble, 2 (galectin 2); Kruppel-like factor 4 (gut); ribonucleotide reductase M2 polypeptide; proliferating cell nuclear antigen; cyclin B2; killer cell lectin-like receptor subfamily B, member 1; chemokine (C-C motif) receptor 1; syntaxin 11; mannose receptor, C type 1; lung type-I cell membrane-associated glycoprotein; guanidinoacetate N-methyltransferase; alanyl (membrane) aminopeptidase (aminopeptidase N, aminopeptidase M, microsomal aminopeptidase, CD13, p150); zinc finger protein 36, C3H type-like 1; 24-dehydrocholesterol reductase; hematological and neurological expressed 1; M-phase phosphoprotein 1; activation-induced cytidine deaminase; interleukin 1 receptor, type II; transforming growth factor, alpha; collagen, type VI, alpha 3; deoxythymidylate kinase (thymidylate kinase); pleckstrin homology domain containing, family A (phosphoinositide binding specific) member 1; TTK protein kinase; ataxia telangiectasia mutated (includes complementation groups A, C and D); histidine decarboxylase; acyloxyacyl hydrolase (neutrophil); G protein-coupled receptor 77; serine (or cysteine) proteinase inhibitor, clade B (ovalbumin), member 2; solute carrier family 7, (cationic amino acid transporter, y+ system) member 11; splicing factor, arginine/serine-rich 1 (splicing factor 2, alternate splicing factor); glutaminyl-peptide cyclotransferase (glutaminyl cyclase); solute carrier family 16 (monocarboxylic acid transporters), member 6; CCAAT/enhancer binding protein (C/EBP), alpha; SKB1 homolog (S. pombe); v-myb myeloblastosis viral oncogene homolog (avian)-like 2; CDC42 effector protein (Rho GTPase binding) 1; integrin, alpha M (complement component receptor 3, alpha; also known as CD11b (p170), macrophage antigen alpha polypeptide); centromere protein F, 350/400 ka (mitosin); cystatin C (amyloid angiopathy and cerebral hemorrhage); sialic acid binding Ig-like lectin 7; C-type (calcium dependent, carbohydrate-recognition domain) lectin, superfamily member 14 (macrophage-derived); serum/glucocorticoid regulated kinase; uridine monophosphate kinase; Kruppel-like factor 2 (lung); fructose-1,6-bisphosphatase 1; neutrophil cytosolic factor 2 (65 kDa, chronic granulomatous disease, autosomal 2); frizzled homolog 2 (Drosophila); integrin, beta 7; interleukin 12 receptor, beta 2; colony stimulating factor 1 receptor, formerly McDonough feline sarcoma viral (v-fms) oncogene homolog; kinesin family member C3; N-acetylneuraminate pyruvate lyase (dihydrodipicolinate synthase); tensin; complement component 1, q subcomponent, receptor 1; leukocyte immunoglobulin-like receptor, subfamily B (with TM and ITIM domains), member 4; kinesin-like 7; chemokine (C-C motif) ligand 7; myeloid cell nuclear differentiation antigen; BCL2/adenovirus E1B 19 kDa interacting protein 3; pituitary tumor-transforming 1; interferon-induced protein 44; chemokine (C-X-C motif) ligand 2; SLAM family member 8; transferrin receptor 2; integrin, alpha X (antigen CD11C (p150), alpha polypeptide); serine/threonine kinase 6; ficolin (collagen/fibrinogen domain containing) 1; chromodomain helicase DNA binding protein 1-like; Fc fragment of IgG, low affinity IIb, receptor for (CD32); secreted phosphoprotein 1 (osteopontin, bone sialoprotein I, early T-lymphocyte activation 1); CD86 antigen (CD28 antigen ligand 2, B7-2 antigen); achalasia, adrenocortical insufficiency, alacrimia (Allgrove, triple-A); polo-like kinase 1 (hormolog of Drosophila); ectonucleotide pyrophosphatase/phosphodiesterase 2 (autotaxin); nuclear receptor interacting protein 1; thymidylate synthetase; DNA replication complex GINS protein PSF2; CD9 antigen (p24); T-LAK cell-originated protein kinase; RAB31, member RAS oncogene family; centromere protein E, 312 kDa; ZW10 interactor; fibrinogen-like 2; bombesin-like receptor 3; matrix metalloproteinase 12 (macrophage elastase); dual specificity phosphatase 6; chemokine (C-X-C motif) ligand 3; MAD2 mitotic arrest deficient-like 1 (homolog of yeast); leukocyte immunoglobulin-like receptor, subfamily B (with TM and ITIM domains), member 2; CD72 antigen; cellular repressor of E1A-stimulated genes; CHK1 checkpoint homolog (homolog of S. pombe); interferon, gamma-inducible protein 30; ubiquitin-conjugating enzyme E2S; ribokinase; serine/threonine kinase 3 (STE20 homolog of yeast); v-maf musculoaponeurotic fibrosarcoma oncogene homolog B (homolog of avian); exonuclease NEF-sp; vascular endothelial growth factor; stathmin 1/oncoprotein 18; MCM2 minichromosome maintenance deficient 2, mitotin (homolog of S. cerevisiae); ribonuclease, RNase A family, k6; G-protein signalling modulator 2 (AGS3-like, homolog of C. elegans); amylase, alpha 2B; pancreatic; tribbles homolog 1 (homolog of Drosophila); C-type (calcium dependent, carbohydrate-recognition domain) lectin, superfamily member 6; Ig superfamily protein; baculoviral IAP repeat-containing 1; transgelin; kinesin family member 20A; interleukin 3 (colony-stimulating factor, multiple); oxidised low density lipoprotein (lectin-like) receptor 1; Similar to ribosomal protein L13a; 60S ribosomal protein L13a; 23 kD highly basic protein (LOC399810), mRNA [BLAST]; endoplasmic reticulum chaperone SIL1, homolog of yeast; telomerase-associated protein 1; Fc fragment of IgG, receptor, transporter, alpha; nerve growth factor receptor (TNFRSF 16) associated protein 1; BUB1 budding uninhibited by benzimidazoles 1 homolog (homolog of yeast); cyclin A2; BUB1 budding uninhibited by benzimidazoles 1 homolog beta (homolog of yeast); gamma-glutamyltransferase-like activity 1; immunoglobulin superfamily, member 6; hypothetical protein CG003; fer-1-like 3, myoferlin (homolog of C. elegans); cell division cycle 2, G1 to S and G2 to M; erythrocyte membrane protein band 4.1-like 3; cell division cycle 25C; thyroid hormone receptor interactor 13; thrombospondin 1; TPX2, microtubule-associated protein homolog (homolog of Xenopus laevis); CDC20 cell division cycle 20 homolog (homolog of S. cerevisiae); lymphotoxin beta receptor (TNFR superfamily, member 3); membrane-spanning 4-domains, subfamily A, member 4; polo-like kinase 4 (homolog of Drosophila); cyclin-dependent kinase inhibitor 2C (p18, inhibits CDK4); toll-like receptor 8; histamine N-methyltransferase; interleukin 3 receptor, alpha (low affinity); cathepsin L; transforming, acidic coiled-coil containing protein 3; sialyltransferase 4A (beta-galactoside alpha-2,3-sialyltransferase); exosome component 9; BRCA1 associated RING domain 1; reticulon 1; leukocyte immunoglobulin-like receptor, subfamily B (with TM and ITIM domains), member. 1; Transcribed sequences; H2A histone family, member Z; RAD54-like (homolog of S. cerevisiae); chromosome condensation protein G; glutathione transferase zeta 1 (maleylacetoacetate isomerase); kinesin family member 2C; ubiquitin-conjugating enzyme E2C; spinocerebellar ataxia 1 (olivopontocerebellar ataxia 1, autosomal dominant, ataxin 1); v-myb myeloblastosis viral oncogene homolog (homolog of avian); thrombomodulin; centromere protein A, 17 kDa; cytochrome b-245, beta polypeptide (chronic granulomatous disease); endoglin (Osler-Rendu-Weber syndrome 1); formyl peptide receptor-like 2; nucleolar and spindle associated protein 1; tripartite motif-containing 26; BRCA2 and CDKN1A interacting protein; ubiquitin specific protease 18; acid phosphatase 5, tartrate resistant; metallothionein 1H; NIMA (never in mitosis gene a)-related kinase 2; dual specificity phosphatase 1; breast cancer 1, early onset; fatty acid desaturase 2; topoisomerase (DNA) II alpha 170 kDa; neuropilin 1; LIM domain only 2 (rhombotin-like 1); tissue inhibitor of metalloproteinase 2; oncostatin M; S100 calcium binding protein A9 (calgranulin B); chemokine (C-C motif) ligand 24; BTB and CNC homology 1, basic leucine zipper transcription factor 1; leukocyte immunoglobulin-like receptor, subfamily B (with TM and ITIM domains), member 3; ribonuclease, RNase A family, 1 (pancreatic); pro-platelet basic protein (chemokine (C-X-C motif) ligand 7); phospholipase A2, group VII (platelet-activating factor acetylhydrolase, plasma). These genes are listed in Table IV. These genes will be useful in assessing treatment of cancer, autoimmune diseases, inflammatory diseases and the like by VX-944 and are likely to represent attenuation of proliferation by VX-944 in diseases where cell proliferation is implicated. A combination of gene set I and gene set IV reveals markers that can be monitored in hematological malignancies. By hematological malignancies the present application refers to any hematological cancer or hematological disorder that manifests in the proliferation or hyperproliferation of blood cells.

Set V provides a more select marker set of genes from set IV whose expression also is altered in response to VX-944 in AML or PML cancers or other diseases in which the proliferation of lymphocytes is implicated. Thus, gene expression changes that can be monitored in lymphocytes (PBMCs, blood) obtained from hematological cancers, or other diseases in which proliferation of lymphocytes is implicated in the presence of VX-944 to provide an assessment of the efficacy of VX-944 or IMPDH inhibition in general. This subset of genes includes: kinesin family member 4A; Fc fragment of IgG, high affinity Ia, receptor for (CD64); aurora kinase B; growth arrest-specific 7; solute carrier family 27 (fatty acid transporter), member 2; cyclin-dependent kinase inhibitor 3 (CDK2-associated dual specificity phosphatase); sperm associated antigen 5; formyl peptide receptor 1; chromosome condensation 1-like; tissue inhibitor of metalloproteinase 1 (erythroid potentiating activity, collagenase inhibitor); pleckstrin homology-like domain, family A, member 1; hyaluronan-mediated motility receptor (RHAMM); regulator of G-protein signalling 2, 24 kDa; serine/threonine kinase 6; arachidonate 5-lipoxygenase; inositol(myo)-1(or 4)-monophosphatase 2; metallothionein IX; C-type (calcium dependent, carbohydrate-recognition domain) lectin, superfamily member 5; ribonucleotide reductase M2 polypeptide; cyclin B2; chemokine (C-C motif) receptor 1; mannose receptor, C type 1; collagen, type VI, alpha 3; histidine decarboxylase; G protein-coupled receptor 77; serine (or cysteine) proteinase inhibitor, clade B (ovalbumin), member 2; CCAAT/enhancer binding protein (C/EBP), alpha; integrin, alpha M (complement component receptor 3, alpha; also known as CD11b (p170), macrophage antigen alpha polypeptide); serum/glucocorticoid regulated kinase; neutrophil cytosolic factor 2 (65 kDa, chronic granulomatous disease, autosomal 2); N-acetylneuraminate pyruvate lyase (dihydrodipicolinate synthase); pituitary tumor-transforming 1; integrin, alpha X (antigen CD11C (p150), alpha polypeptide); serine/threonine kinase 6; polo-like kinase 1 (homolog of Drosophila); RAB31, member RAS oncogene family; CD72 antigen; interferon, gamma-inducible protein 30; v-maf musculoaponeurotic fibrosarcoma oncogene homolog B (homolog of avian); tribbles homolog 1 (homolog of Drosophila); oxidised low density lipoprotein (lectin-like) receptor 1; BUB1 budding uninhibited by benzimidazoles 1 homolog beta (homolog of yeast); hypothetical protein CG003; CDC20 cell division cycle 20 homolog (homolog of S. cerevisiae); toll-like receptor 8; kinesin family member 2C; ubiquitin-conjugating enzyme E2C; v-myb myeloblastosis viral oncogene homolog (homolog of avian); centromere protein A, 17 kDa; cytochrome b-245, beta polypeptide (chronic granulomatous disease); nucleolar and spindle associated protein 1; metallothionein 1H; S100 calcium binding protein A9 (calgranulin B); and are listed herein below in Table V.

The genes in Table IV were further analysed and narrowed to those genes that are responsive to VX-944 and to the unrelated IMPDH inhibitor MPA to provide a subset of IV, that is further narrower than subset V by using genes commonly affected by both VX-944 and MPA in HL60 cells. This subset of genes included the following genes: kinesin family member 4A; Fc fragment of IgG, high affinity Ia, receptor for (CD64); growth arrest-specific 7; solute carrier family 27 (fatty acid transporter), member 2; cyclin-dependent kinase inhibitor 3 (CDK2-associated dual specificity phosphatase); sperm associated antigen 5; formyl peptide receptor 1; chromosome condensation 1-like; tissue inhibitor of metalloproteinase 1 (erythroid potentiating activity, collagenase inhibitor); pleckstrin homology-like domain, family A, member 1; regulator of G-protein signalling 2, 24 kDa; arachidonate 5-lipoxygenase; inositol(myo)-1(or 4)-monophosphatase 2; metallothionein 1X; C-type (calcium dependent, carbohydrate-recognition domain) lectin, superfamily member 5; ribonucleotide reductase M2 polypeptide; chemokine (C-C motif) receptor 1; collagen, type VI, alpha 3; histidine decarboxylase; serine (or cysteine) proteinase inhibitor, clade B (ovalbumin), member 2; integrin, alpha M (complement component receptor 3, alpha; also known as CD11b (p170), macrophage antigen alpha polypeptide); serum/glucocorticoid regulated kinase; neutrophil cytosolic factor 2 (65 kDa, chronic granulomatous disease, autosomal 2); N-acetylneuraminate pyruvate lyase (dihydrodipicolinate synthase); integrin, alpha X (antigen CD11C (p150), alpha polypeptide); RAB31, member RAS oncogene family; CD72 antigen; interferon, gamma-inducible protein 30; v-maf musculoaponeurotic fibrosarcoma oncogene homolog B (homolog of avian); tribbles homolog 1 (homolog of Drosophila); v-myb myeloblastosis viral oncogene homolog (homolog of avian); cytochrome b-245, beta polypeptide (chronic granulomatous disease); nucleolar and spindle associated protein 1; metallothionein 1H; S100 calcium binding protein A9 (calgranulin B); and the sequences of these genes are listed in Table VI.

VX-944 also has been used as an antiviral agent. Genes that are predictive of the antiviral efficacy of VX-944 are found in Set VII (see Table VII), which were identified as the subset of genes from set I which are also modified in response to interferon. These genes will be useful for monitoring gene expression changes that in lymphocytes (PBMCs, blood) obtained from patients suffering from viral diseases such as HCV, or other diseases in which interferon treatment is useful. These genes were: interferon, alpha-inducible protein (clone IFI-6-16); interferon, alpha-inducible protein (clone IFI-15K); interferon-induced protein with tetratricopeptide repeats 5; interferon-induced protein with tetratricopeptide repeats 1; interferon-induced protein 44; myxovirus (influenza virus) resistance 1, interferon-inducible protein p78 (mouse); chemokine (C-C motif) ligand 2; chemokine (C-X-C motif) ligand 10; chemokine (C-X-C motif) ligand 9 and the sequences of these genes is provided in Table VII.

In set VIII there are listed the apoptosis genes that are responsive to VX-944. This is a narrower subset of genes from subset IV whose gene expression changes can predict beneficial response to VX-944 and be monitored in lymphocytes (PBMCs, blood) obtained from cancer patients. Out of the 67 genes implicated in cancers, 4 are down-regulated by VX-944 in proliferating lymphocytes. These genes will be predictive of the anti-cancer cancer efficacy of VX-944 in leukemias and/or solid tumors. The sequences of these genes are listed below in Table VIII.

Alternatively, the above subsets of genes may be segregated according to function and it may be desirable to prepare microarrays that will be useful in providing an indication of the effects of VX-944 or any other IMPDH inhibitor on a specific functional pathway. In this regard, genes involved in apoptosis and whose expression is altered in response to IMPDH inhibition are: nuclear factor of kappa light polypeptide gene enhancer in B-cells 1 (p105); TNF receptor-associated factor 2; TRK-fused gene; lymphotoxin alpha (TNF superfamily, member 1); inhibitor of kappa light polypeptide gene enhancer in B-cells, kinase epsilon; nuclear factor of kappa light polypeptide gene enhancer in B-cells inhibitor, alpha; tumor necrosis factor receptor superfamily, member 5; tumor necrosis factor (ligand) superfamily, member 6; ADP-ribosyltransferase (NAD+; poly (ADP-ribose) polymerase); Fas (TNFRSF6)-associated via death domain; nerve growth factor, beta polypeptide; TNFRSF1A-associated via death domain; BH3 interacting domain death agonist; receptor (TNFRSF)-interacting serine-threonine kinase 1; tumor protein p53 (Li-Fraumeni syndrome); Mdm2, transformed 3T3 cell double minute 2, p53 binding protein (homolog of mouse); caspase 7, apoptosis-related cysteine protease; tumor necrosis factor (TNF superfamily, member 2); myeloid cell leukemia sequence 1 (BCL2-related); caspase 10, apoptosis-related cysteine protease; BCL2-related ovarian killer; tumor necrosis factor receptor superfamily, member 1A; baculoviral IAP repeat-containing 3; B-cell CLL/lymphoma 2; BCL2-associated X protein; TRK-fused gene; tumor necrosis factor receptor superfamily, member 1B; caspase 6, apoptosis-related cysteine protease; tumor necrosis factor (ligand) superfamily, member 10; caspase 9, apoptosis-related cysteine protease; protein tyrosine phosphatase, non-receptor type 13 (APO-1/CD95 (Fas)-associated phosphatase); TNF receptor-associated factor 6; cytochrome c, somatic; mitogen-activated protein kinase kinase 4; v-myc myelocytomatosis viral oncogene homolog (homolog of avian); BCL2-like 2; v-jun sarcoma virus 17 oncogene homolog (homolog of avian); BCL2-antagonist/killer 1; mitogen-activated protein kinase kinase kinase 14;splicing factor, arginine/serine-rich 2, interacting protein; caspase 2, apoptosis-related cysteine protease (neural precursor cell expressed, developmentally down-regulated 2); apoptotic protease activating factor; perforin 1 (pore forming protein); BCL2-antagonist of cell death; BCL2-related protein A1; harakiri, BCL2 interacting protein (contains only BH3 domain); tumor necrosis factor receptor superfamily, member 6; tumor necrosis factor (ligand) superfamily, member 5 (hyper-IgM syndrome); mitogen-activated protein kinase 10; TNF receptor-associated factor 3; TNF receptor-associated factor 1; caspase 8, apoptosis-related cysteine protease; granzyme B (granzyme 2, cytotoxic T-lymphocyte-associated serine esterase 1); baculoviral IAP repeat-containing 2; caspase 4, apoptosis-related cysteine protease; CASP2 and RIPK1 domain containing adaptor with death domain; caspase 1, apoptosis-related cysteine protease (interleukin 1, beta, convertase); DNA fragmentation factor, 45 kDa, alpha polypeptide; DNA fragmentation factor, 40 kDa, beta polypeptide (caspase-activated DNase); baculoviral IAP repeat-containing 4; inhibitor of kappa light polypeptide gene enhancer in B-cells, kinase gamma; caspase 3, apoptosis-related cysteine protease; nuclear receptor subfamily 3, group C, member 1 (glucocorticoid receptor); v-rel reticuloendotheliosis viral oncogene homolog A, nuclear factor of kappa light polypeptide gene enhancer in B-cells 3, p65 (homolog of avian); nerve growth factor receptor (TNFR superfamily, member 16). Thus a microarray may be prepared to determine the efficacy of IMPDH inhibition by monitoring genes involved in apoptosis wherein the microarray comprises at least two or more of the above VX-944-responsive apoptosis-associated genes.

Genes involved in cellular metabolism that are responsive to VX-944 and IMPDH inhibition include: dehydrogenase/reductase (SDR family) member 4; dehydrogenase/reductase (SDR family) member 10; prion protein (p27-30) (Creutzfeld-Jakob disease, Gerstmann-Strausler-Scheinker syndrome, fatal familial insomnia); phosphomannomutase 1; arylsulfatase B; UDP glycosyltransferase 1 family, polypeptide A10; branched chain keto acid dehydrogenase E1, alpha polypeptide (maple syrup urine disease); 6-phosphofructo-2-kinase/fructose-2,6-biphosphatase 1; chitinase 1 (chitotriosidase); ATPase, Ca++ transporting, plasma membrane 3; tyrosinase-related protein 1; 1-acylglycerol-3-phosphate O-acyltransferase 1 (lysophosphatidic acid acyltransferase, alpha); aldehyde dehydrogenase 1 family, member B1; dehydrogenase/reductase (SDR family) member 6; 3-hydroxyisobutyryl-Coenzyme A hydrolase; isocitrate dehydrogenase 3 (NAD+) alpha; pyruvate dehydrogenase complex, component X; 6-phosphofructo-2-kinase/fructose-2,6-biphosphatase 2; galactokinase 2; pyruvate dehydrogenase (lipoamide) alpha 1; hydroxyprostaglandin dehydrogenase 15-(NAD); flightless I homolog (homolog of Drosophila); glutathione S-transferase M3 (brain); ATPase, H+/K+ exchanging, alpha polypeptide; ATPase, Ca++ transporting, type 2C, member 1; N-sulfoglucosamine sulfohydrolase(sulfamidase); aldehyde dehydrogenase 1 family, member A3; 5,10-methenyltetrahydrofolate synthetase(5-formyltetrahydrofolate cyclo-ligase); succinate-CoA ligase, ADP-forming, beta subunit; carboxypeptidase E; alkaline phosphatase, intestinal; chromodomain protein, Y-like; aldehyde dehydrogenase 5 family, member A1 (succinate-semialdehyde dehydrogenase); aldehyde dehydrogenase 4 family, member A1; branched chain aminotransferase 2, mitochondrial; isocitrate dehydrogenase 3 (NAD+) beta; acyl-CoA synthetase long-chain family member 3; hydroxysteroid (17-beta) dehydrogenase 4; ankyrin 2, neuronal; tryptophan hydroxylase 1 (tryptophan 5-monooxygenase); fibroblast growth factor 16; N-acetyltransferase 2 (arylamine N-acetyltransferase); solute carrier family 27 (fatty acid transporter), member 2; peroxisomal trans-2-enoyl-CoA reductase; phosphoserine aminotransferase 1; alanine-glyoxylate aminotransferase (oxalosis I; hyperoxaluria I; glycolicaciduria; serine-pyruvate aminotransferase); tafazzin (cardiomyopathy, dilated 3A (X-linked); endocardial fibroelastosis 2; Barth syndrome); liver-specific bHLH-Zip transcription factor; UDP glycosyltransferase 8 (UDP-galactose ceramide galactosyltransferase); pyruvate dehydrogenase (lipoamide) alpha 2; arylsulfatase A; alanine-glyoxylate aminotransferase (oxalosis I; hyperoxaluria I; glycolicaciduria; serine-pyruvate aminotransferase); ATPase, Na+/K+ transporting, alpha 1 polypeptide; arylsulfatase F; hydroxysteroid (17-beta) dehydrogenase 12; G protein-coupled receptor 56; phosphoribosyl pyrophosphate amidotransferase; eyes absent homolog 4 (homolog of Drosophila); hydroxyacyl-Coenzyme A dehydrogenase/3-ketoacyl-Coenzyme A thiolase/enoyl-Coenzyme A hydratase (trifunctional protein), alpha subunit; lanosterol synthase (2,3-oxidosqualene-lanosterol cyclase); family with sequence similarity 16, member A, X-linked; alkaline phosphatase, placental (Regan isozyme); acyl-CoA synthetase long-chain family member 1; alkaline phosphatase, liver/bone/kidney; eosinophil chemotactic cytokine; hydroxysteroid (11-beta) dehydrogenase 1; isocitrate dehydrogenase 2 (NADP+), mitochondrial; pyruvate carboxylase; galactosamine (N-acetyl)-6-sulfate sulfatase (Morquio syndrome, mucopolysaccharidosis type IVA); phenylalanine hydroxylase; matrix metalloproteinase 24 (membrane-inserted); nucleolar protein 5A (56 kDa with KKE/D repeat); phosphoglycerate mutase 2 (muscle); AU RNA binding protein/enoyl-Coenzyme A hydratase; retinol dehydrogenase 8 (all-trans); glutathione S-transferase A1; glutamine-fructose-6-phosphate transaminase 1; phosphatidylglycerophosphate synthase; WW domain containing oxidoreductase; serine (or cysteine) proteinase inhibitor, clade A (alpha-1 antiproteinase, antitrypsin), member 4; glutamine-fructose-6-phosphate transaminase 2; phosphoserine phosphatase; aspartoacylase (aminoacylase 2, Canavan disease); oxoglutarate (alpha-ketoglutarate) dehydrogenase (lipoamide); eyes absent homolog 3 (homolog of Drosophila); glyoxylate reductase/hydroxypyruvate reductase; aldehyde dehydrogenase 3 family, member B1; glutathione S-transferase M4; hydroxysteroid (17-beta) dehydrogenase 1; ATP citrate lyase; iduronate 2-sulfatase (Hunter syndrome); WW domain containing oxidoreductase; chitinase 3-like 1 (cartilage glycoprotein-39); propionyl Coenzyme A carboxylase, alpha polypeptide; dopachrome tautomerase (dopachrome delta-isomerase, tyrosine-related protein 2); inorganic pyrophosphatase 2; methylmalonyl Coenzyme A mutase; aldehyde dehydrogenase 1 family, member A2; hydroxysteroid (17-beta) dehydrogenase 3; chitobiase, di-N-acetyl-; ATPase, Ca++ transporting, cardiac muscle, slow twitch 2; UDP glycosyltransferase 2 family, polypeptide B17; selenocysteine lyase; ATPase, Ca++ transporting, ubiquitous; quinoid dihydropteridine reductase; peroxisomal D3,D2-enoyl-CoA isomerase; SA hypertension-associated homolog (homolog of rat); chromosome 1 open reading frame 10; insulin induced gene 1; NEL-like 1 (homolog of chicken); rTS beta protein; sepiapterin reductase (7,8-dihydrobiopterin:NADP+ oxidoreductase); phosphoserine phosphatase; pyrophosphatase (inorganic); branched chain aminotransferase 1, cytosolic; UDP glycosyltransferase 1 family, polypeptide A10; oviductal glycoprotein 1, 120 kDa (mucin 9, oviductin); chromosome 5 open reading frame 4; retinol dehydrogenase 5 (11-cis and 9-cis); chromosome 5 open reading frame 4; ATPase, Na+/K+ transporting, alpha 3 polypeptide; arylsulfatase D; dehydrogenase/reductase (SDR family) member 2; UDP glycosyltransferase 2 family, polypeptide A1; solute carrier family 27 (fatty acid transporter), member 5; glyceronephosphate O-acyltransferase; steroid sulfatase (microsomal), arylsulfatase C, isozyme S; acyl-CoA synthetase long-chain family member 1; glutathione S-transferase pi; aldehyde dehydrogenase 8 family, member A1; 6-phosphofructo-2-kinase/fructose-2,6-biphosphatase 4; ATPase, Ca++ transporting, cardiac muscle, fast twitch 1; aconitase 1, soluble; hydroxysteroid (17-beta) dehydrogenase 7; 1-acylglycerol-3-phosphate O-acyltransferase 3; UDP-glucose pyrophosphorylase 2; chromosome 12 open reading frame 5; glutathione S-transferase M5; phospholipase D1, phophatidylcholine-specific; dehydrogenase/reductase (SDR family) member 8; phospholipase D2; fumarylacetoacetate hydrolase (fumarylacetoacetase); glutathione S-transferase omega 1; 3-hydroxysteroid epimerase; ATPase, Na+/K+ transporting, alpha 2 (+) polypeptide; 2,4-dienoyl CoA reductase 1, mitochondrial; arylsulfatase E (chondrodysplasia punctata 1); 2,4-dienoyl CoA reductase 2, peroxisomal; E-1 enzyme; chitinase 3-like 2; solute carrier family 27 (fatty acid transporter), member 6; phosphoglycerate mutase 1 (brain); hydroxysteroid (17-beta) dehydrogenase 2; 1-acylglycerol-3-phosphate O-acyltransferase 4 (lysophosphatidic acid acyltransferase, delta); aldehyde dehydrogenase 3 family, member A1; iduronate 2-sulfatase (Hunter syndrome); UDP glycosyltransferase 1 family, polypeptide A10; dehydrogenase/reductase (SDR family) member 3; ATPase, Cu++ transporting, alpha polypeptide (Menkes syndrome); matrix metalloproteinase 24 (membrane-inserted); galactokinase 1; ATPase, Ca++ transporting, plasma membrane 1; isocitrate dehydrogenase 1 (NADP+), soluble; 1-acylglycerol-3-phosphate O-acyltransferase 2 (lysophosphatidic acid acyltransferase, beta); succinate-CoA ligase, GDP-forming, alpha subunit; glutathione S-transferase M1; glutathione S-transferase A4; UDP-N-acteylglucosamine pyrophosphorylase 1; glucosamine (N-acetyl)-6-sulfatase (Sanfilippo disease IIID); ATPase, Ca++ transporting, plasma membrane 4; glutathione S-transferase A3; fatty acid 2-hydroxylase; 1-acylglycerol-3-phosphate O-acyltransferase 1 (lysophosphatidic acid acyltransferase, alpha); dehydrogenase/reductase (SDR family) member 9; glutathione S-transferase M4; 3-oxoacid CoA transferase 1; sterol-C4-methyl oxidase-like; carboxylesterase 1 (monocyte/macrophage serine esterase 1); acetyl-Coenzyme A carboxylase alpha; acyl-CoA synthetase long-chain family member 5; methylcrotonoyl-Coenzyme A carboxylase 1 (alpha); UDP glycosyltransferase 1 family, polypeptide A10; hydroxysteroid (11-beta) dehydrogenase 2; COP9 constitutive photomorphogenic homolog subunit 8 (Arabidopsis); ATPase, H+/K+ transporting, nongastric, alpha polypeptide; integrin beta 4 binding protein; ATPase, Cu++ transporting, beta polypeptide (Wilson disease); eyes absent homolog 1 (homolog of Drosophila); 6-phosphofructo-2-kinase/fructose-2,6-biphosphatase 3; asparagine synthetase; formiminotransferase cyclodeaminase; eyes absent homolog 2 (homolog of Drosophila); 2,3-bisphosphoglycerate mutase; amiloride binding protein 1 (amine oxidase (copper-containing)); alpha-methylacyl-CoA racemase; ubiquitin associated and SH3 domain containing, A; acetyl-Coenzyme A carboxylase beta; proprotein convertase subtilisin/kexin type 1; tyrosinase (oculocutaneous albinism IA); ATPase, Ca++ transporting, plasma membrane 2; dicarbonyl/L-xylulose reductase; phosphomannomutase 2; carbonyl reductase 3; arylacetamide deacetylase (esterase); carbonyl reductase 1; NFS1 nitrogen fixation 1 (homolog of S. cerevisiae). As such another contemplated microarray would be one which comprises two or more of the above genes to specifically monitor the efficacy of IMPDH inhibition on metabolic pathways in general.

Genes involved in the citric acid cycle that are responsive to VX-944 and IMPDH inhibition include: malate dehydrogenase 2, NAD (mitochondrial); isocitrate dehydrogenase 3 (NAD+) alpha; succinate dehydrogenase complex, subunit A, flavoprotein (Fp); isocitrate dehydrogenase 3 (NAD+) beta; dihydrolipoamide dehydrogenase (E3 component of pyruvate dehydrogenase complex, 2-oxo-glutarate complex, branched chain keto acid dehydrogenase complex); aconitase 2, mitochondrial; isocitrate dehydrogenase 3 (NAD+) gamma; succinate dehydrogenase complex, subunit B, iron sulfur (Ip); isocitrate dehydrogenase 2 (NADP+), mitochondrial; pyruvate carboxylase; fumarate hydratase; phosphoenolpyruvate carboxykinase 1 (soluble); oxoglutarate (alpha-ketoglutarate) dehydrogenase (lipoamide); aconitase 1, soluble; isocitrate dehydrogenase 1 (NADP+), soluble; malate dehydrogenase 1, NAD (soluble); succinate-CoA ligase, GDP-forming, alpha subunit. As such another contemplated microarray would be one which comprises two or more of the above genes to specifically monitor the efficacy of IMPDH inhibition on glycolysis and the citric acid cycle in general.

An array designed to monitor the IMPDH inhibition on genes involved in purine metabolism will advantageously determine the expression levels of two or more genes selected from the group consisting of adenosine monophosphate deaminase 2 (isoform L); hypoxanthine phosphoribosyltransferase 1 (Lesch-Nyhan syndrome); methylenetetrahydrofolate dehydrogenase (NADP+ dependent), methenyltetrahydrofolate cyclohydrolase, formyltetrahydrofolate synthetase; adenosine kinase; adenylosuccinate synthase; IMP (inosine monophosphate) dehydrogenase 1; ATP-binding cassette, sub-family G (WHITE), member 1; phosphoribosyl pyrophosphate amidotransferase; cat eye syndrome chromosome region, candidate 1; thiopurine S-methyltransferase; solute carrier family 28 (sodium-coupled nucleoside transporter), member 2; phosphoribosylaminoimidazole carboxylase, phosphoribosylaminoimidazole succinocarboxamide synthetase; adenosine monophosphate deaminase (isoform E); IMP (inosine monophosphate) dehydrogenase 2; adenosine monophosphate deaminase 1 (isoform M); phosphoribosylglycinamide formyltransferase, phosphoribosylglycinamide synthetase, phosphoribosylaminoimidazole synthetase; adenylosuccinate lyase; nucleoside phosphorylase; 5-aminoimidazole-4-carboxamide ribonucleotide formyltransferase/IMP cyclohydrolase; 8-oxoguanine DNA glycosylase; guanine monphosphate synthetase; adenosine deaminase; adenine phosphoribosyltransferase. Specific microarrays comprising these genes are contemplated as part of the invention.

Another advantage array may be one which can be used to determine the expression levels of lysosomal enzymes that are altered in response to IMPDH inhibition. Such specific arrays may comprise two or more of the following genes: arylsulfatase B; prolylcarboxypeptidase (angiotensinase C); scavenger receptor class B, member 2; acid phosphatase 2, lysosomal; cystinosis, nephropathic; coronin, actin binding protein, 1A; lysosomal-associated membrane protein 2; CD63 antigen (melanoma 1 antigen); lysosomal-associated membrane protein 3.

An array for monitoring chemokines that are responsive to IMPDH inhibition will advantageously comprise a plurality of genes selected from the group consisting of: chemokine (C-X-C motif) ligand 14;chemokine (C-X-C motif) ligand 11; chemokine (C motif) ligand 1; chemokine-like factor; chemokine (C-X-C motif) ligand 6 (granulocyte chemotactic protein 2); platelet factor 4 (chemokine (C-X-C motif) ligand 4); chemokine (C-C motif) receptor-like 1; chemokine (C-X3-C motif) ligand 1; Burkitt lymphoma receptor 1, GTP binding protein (chemokine (C-X-C motif) receptor 5); chemokine (C-X3-C motif) receptor 1; chemokine-like receptor 1; chemokine (C-X-C motif) receptor 6; chemokine (C-C motif) ligand 8; chemokine (C-C motif) ligand 25; chemokine (C motif) ligand 1; tumor necrosis factor receptor superfamily, member 1B; chemokine (C-C motif) ligand 18 (pulmonary and activation-regulated); chemokine (C-X-C motif) ligand 13 (B-cell chemoattractant); G protein-coupled receptor 17; chemokine binding protein 2; chemokine (C-C motif) ligand 3; chemokine (C-X-C motif) ligand 5; chemokine (C-C motif) ligand 1; chemokine (C-C motif) ligand 17; chemokine (C-C motif) receptor-like 2; chemokine(C-X-C motif) ligand 2; chemokine (C-C motif) ligand 20; cardiotrophin-like cytokine; chemokine (C-C motif) ligand 16; G protein-coupled receptor 2; chemokine (C-X-C motif) ligand 3; colony stimulating factor 2 receptor, beta, low-affinity (granulocyte-macrophage); chemokine (C-C motif) ligand 27; chemokine (C-C motif) ligand 22; chemokine (C-C motif) ligand 15 [BLAST]; chemokine (C-C motif) ligand 21; chemokine (C-X-C motif) ligand 9; chemokine (C-C motif) ligand 19; chemokine (C-C motif) ligand 13; platelet factor 4 variant 1; chemokine (C-C motif) ligand 24; and interleukin 18 (interferon-gamma-inducing factor).

A cell-adhesion microarray that contains genes responsive to IMPDH inhibition also is part of the invention and will comprise a plurality of genes selected from the group consisting of: chemokine (C-C motif) ligand 2; protocadherin gamma subfamily C, 3; brain-specific angiogenesis inhibitor 1; cell adhesion molecule with homology to L1CAM (close homolog of L1); catenin (cadherin-associated protein), alpha 1, 102 kDa; sushi-repeat-containing protein, X-linked; EGF-like repeats and discoidin I-like domains 3; cadherin 1, type 1, E-cadherin (epithelial); Clone IMAGE:5301388, mRNA; Rho GTPase activating protein 5; retinal outer segment membrane protein 1; bystin-like; myelin associated glycoprotein; protein disulfide isomerase, pancreatic; limbic system-associated membrane protein; cadherin 17, LI cadherin (liver-intestine); poliovirus receptor-related 1 (herpesvirus entry mediator C; nectin); cadherin 9, type 2 (T1-cadherin); CD226 antigen; parvin, alpha; trophinin associated protein (tastin); protocadherin gamma subfamily C, 3; selectin P (granule membrane protein 140 kDa, antigen CD62); ephrin-B1; plakophilin 2; sema domain, immunoglobulin domain (Ig), transmembrane domain (TM) and short cytoplasmic domain, (semaphorin) 4D; desmocollin 2; selectin E (endothelial adhesion molecule 1); cadherin, EGF LAG seven-pass G-type receptor 2 (flamingo homolog of Drosophila); calsyntenin 2; myosin binding protein C, cardiac; ARS component B; sema domain, seven thrombospondin repeats (type 1 and type 1-like), transmembrane domain (TM) and short cytoplasmic domain, (semaphorin) 5A; neural precursor cell expressed, developmentally down-regulated 9; intercellular adhesion molecule 2; alpha-1-microglobulinlbikunin precursor; sialic acid binding Ig-like lectin 5; plakophilin 3; laminin, gamma 1 (formerly LAMB2); flotillin 2; integrin, alpha E (antigen CD103, human mucosal lymphocyte antigen 1; alpha polypeptide); myosin binding protein C, slow type; retinoschisis (X-linked, juvenile) 1; desmocollin 1; CD44 antigen (homing function and Indian blood group system); thrombospondin 2; fasciculation and elongation protein zeta 1 (zygin I); BH-protocadherin (brain-heart); protocadherin beta 12; leupaxin; insulin-like growth factor binding protein, acid labile subunit; pinin, desmosome associated protein; parvin, beta; chemokine (C-X3-C motif) ligand 1; scavenger receptor class B, member 2; amyloid beta (A4) precursor-like protein 1; Rho GDP dissociation inhibitor (GDI) alpha; parvin, beta; collagen, type V, alpha 3; sialophorin (gpL115, leukosialin, CD43); laminin, gamma 3; hyaluronan and proteoglycan link protein 2; collagen, type XIX, alpha 1; cadherin 15, M-cadherin (myotubule); chemokine (C-X3-C motif) receptor 1; laminin, alpha 2 (merosin, congenital muscular dystrophy); cadherin 3, type 1, P-cadherin (placental); bone gamma-carboxyglutamate (gla) protein (osteocalcin); lectin, galactoside-binding, soluble, 7 (galectin 7); protocadherin 11 X-linked; protocadherin alpha 2; chondroitin sulfate proteoglycan 4 (melanoma-associated); tumor necrosis factor (TNF superfamily, member 2); myosin binding protein H; TYRO3 protein tyrosine kinase; plakophilin 1 (ectodermal dysplasia/skin fragility syndrome); neuropilin 2; vascular cell adhesion molecule 1; acetylcholinesterase (YT blood group); erbb2 interacting protein; a disintegrin and metalloproteinase domain 2 (fertilin beta); polycystic kidney disease 1 (autosomal dominant); thrombospondin 4; multimerin 1; laminin, beta 3; cadherin 11, type 2, OB-cadherin (osteoblast); contactin 1; Lutheran blood group (Auberger b antigen included); protein tyrosine phosphatase, receptor type, U; catenin (cadherin-associated protein), alpha 2; desmoglein 3 (pemphigus vulgaris antigen); collagen, type XVI, alpha 1; chemokine (C-X-C motif) receptor 3; desmoglein 2; tenascin R (restrictin, janusin); growth arrest-specific 6; comeodesmosin; transforming growth factor, beta-induced, 68 kDa; collagen, type VIII, alpha 1; FXYD domain containing ion transport regulator 5; transforming growth factor beta 1 induced transcript 1; spondin 2, extracellular matrix protein; dystonin; intercellular adhesion molecule 3; DiGeorge syndrome critical region gene 2; collagen, type VII, alpha 1 (epidermolysis bullosa, dystrophic, dominant and recessive); CD99 antigen; L1 cell adhesion molecule (hydrocephalus, stenosis of aqueduct of Sylvius 1, MASA (mental retardation, aphasia, shuffling gait and adducted thumbs) syndrome, spastic paraplegia 1); laminin, beta 1; calcium and integrin binding 1 (calmyrin); collagen, type V, alpha 1; WNT1 inducible signaling pathway protein 1; cadherin 7, type 2; tissue specific transplantation antigen P35B; collagen, type XVIII, alpha 1; vinexin beta (SH3-containing adaptor molecule-1); desmocollin 3; brevican; protocadherin beta 6; collagen, type XV, alpha 1; cadherin, EGF LAG seven-pass G-type receptor 1 (flamingo homolog of Drosophila); amine oxidase, copper containing 3 (vascular adhesion protein 1); glycoprotein Ib (platelet), alpha polypeptide; CD209 antigen; integrin, alpha 7; trophoblast glycoprotein; pancreatitis-associated protein; crumbs homolog 1 (homolog of Drosophila); oligodendrocyte myelin glycoprotein; sialic acid binding Ig-like lectin 7; integrin, alpha 2b (platelet glycoprotein IIb of IIb/IIIa complex, antigen CD41B); discs, large homolog 5 (homolog of Drosophila); slit homolog 2 (homolog of Drosophila); NEL-like 2 (homolog of chicken); protocadherin beta 11; protocadherin alpha 2; nephronophthisis 1 (juvenile); neuroligin 3; intercellular adhesion molecule 5, telencephalin; protocadherin beta 5; collagen, type VI, alpha 3; phosphoglucomutase 5; contactin 2 (axonal); deleted in liver cancer 1; cadherin 5, type 2, VE-cadherin (vascular epithelium); slit homolog 3 (homolog of Drosophila); AE binding protein 1; ATPase, Ca++ transporting, cardiac muscle, slow twitch 2; chemokine (C-C motif) receptor 3; spastic paraplegia 7, paraplegin (pure and complicated autosomal recessive); myeloid/lymphoid or mixed-lineage leukemia (trithorax homolog of Drosophila); translocated to, 4; CD209 antigen-like; heparan sulfate proteoglycan 2 (perlecan); activated leukocyte cell adhesion molecule; cadherin 12, type 2 (N-cadherin 2); PTK2B protein tyrosine kinase 2 beta; nidogen (enactin); chemokine (C-X-C motif) ligand 12 (stromal cell-derived factor 1); glycoprotein V (platelet); discoidin domain receptor family, member 1; epithelial V-like antigen 1; chromosome 1 open reading frame 10; lectin, galactoside-binding, soluble, 3 binding protein; NEL-like 1 (homolog of chicken); neuronal cell adhesion molecule; amyloid beta (A4) precursor protein-binding, family A, member 1 (X11); melanoma cell adhesion molecule; sialic acid binding Ig-like lectin 7; protocadherin alpha 2; CD84 antigen (leukocyte antigen); colony stimulating factor 3 receptor (granulocyte); protocadherin 8; sialoadhesin; protein tyrosine phosphatase, receptor type, S; neural cell adhesion molecule 2; ectonucleoside triphosphate diphosphohydrolase 1; vitronectin (serum spreading factor, somatomedin B, complement S-protein); mesothelin; adhesion regulating molecule 1; coagulation factor V (proaccelerin, labile factor); intercellular adhesion molecule 1 (CD54), human rhinovirus receptor; protocadherin alpha 2; junction plakoglobin; chemokine (C-C motif) receptor 8; CD22 antigen; complement component 1, q subcomponent, receptor 1; neural cell adhesion molecule 1; protocadherin beta 1; aggrecan 1 (chondroitin sulfate proteoglycan 1, large aggregating proteoglycan, antigen identified by monoclonal antibody A0122); protocadherin alpha 2; integrin-binding sialoprotein (bone sialoprotein, bone sialoprotein II); CD33 antigen (gp67); stromal interaction molecule 1; armadillo repeat gene deletes in velocardiofacial syndrome; laminin, beta 2 (laminin S); signal-induced proliferation-associated gene 1; tetraspan 2; von Willebrand factor; elastin microfibril interfacer 1; reelin; endothelial differentiation, sphingolipid G-protein-coupled receptor, 1; protein tyrosine phosphatase, receptor type, F; protocadherin 9; cadherin 16, KSP-cadherin; alpha-2-glycoprotein 1, zinc; CD36 antigen (collagen type I receptor, thrombospondin receptor); symplekin; contactin 6; sialic acid binding Ig-like lectin 8; chondroitin sulfate proteoglycan 3 (neurocan); cadherin, EGF LAG seven-pass G-type receptor 2 (flamingo homolog of Drosophila); CD9 antigen (p24); nephrosis 1, congenital, Finnish type (nephrin); selectin L (lymphocyte adhesion molecule 1); natural killer cell transcript 4; FAT tumor suppressor homolog 1 (homolog of Drosophila); cadherin 8, type 2; coagulation factor VIII, procoagulant component (hemophilia A); integrin, beta 2 (antigen CD18 (p95), lymphocyte function-associated antigen 1; macrophage antigen 1 (mac-1) beta subunit); Kallmann syndrome 1 sequence; proline-serine-threonine phosphatase interacting protein 1; mucosal vascular addressin cell adhesion molecule 1; periostin, osteoblast specific factor; sarcospan (Kras oncogene-associated gene); hyaluronan binding protein 2; Rho family GTPase 1; catenin (cadherin-associated protein), delta 1; glycoprotein Ib (platelet), beta polypeptide; amyloid beta (A4) precursor protein (protease nexin-II, Alzheimer disease); protocadherin beta 8; junctional adhesion molecule 2; a disintegrin and metalloproteinase domain 22; CD72 antigen; tenascin C (hexabrachion); collectin sub-family member 10 (C-type lectin); vinculin; catenin (cadherin-associated protein), beta 1, 88 kDa; trophinin; catenin (cadherin-associated protein), delta 2 (neural plakophilin-related arm-repeat protein); vascular endothelial growth factor; protein tyrosine phosphatase, non-receptor type substrate 1; CD44 antigen (homing function and Indian blood group system); CD2 antigen (p50), sheep red blood cell receptor; cysteine-rich secretory protein 3; C-type (calcium dependent, carbohydrate-recognition domain) lectin, superfamily member 6; cadherin 6, type 2, K-cadherin (fetal kidney); Usher syndrome 2A (autosomal recessive, mild); myosin binding protein C, fast type; cell adhesion molecule-related/down-regulated by oncogenes; fibronectin leucine rich transmembrane protein 3; scavenger receptor class B, member 1; carbohydrate (N-acetylglucosamine 6-O) sulfotransferase 4; hyaluronan synthase 1; retinal degeneration, slow; roundabout, axon guidance receptor, homolog 1 (homolog of Drosophila); WNT1 inducible signaling pathway protein 2; chromosome 1 open reading frame 38; egf-like module containing, mucin-like, hormone receptor-like 1; immunoglobulin superfamily, member 1; tumor necrosis factor (ligand) superfamily, member 5 (hyper-IgM syndrome); desmoglein 1; intercellular adhesion molecule 4, Landsteiner-Wiener blood group; selectin P ligand; spastic paraplegia 7, paraplegin (pure and complicated autosomal recessive); claudin 1; leucine rich repeat neuronal 5; likely ortholog of mouse gene rich cluster, C10 gene; protocadherin beta 3; contactin associated protein 1; glucosamine (UDP-N-acetyl)-2-epimerase/N-acetylmannosamine kinase; CD6 antigen; milk fat globule-EGF factor 8 protein; KIT ligand; calcium/calmodulin-dependent serine protein kinase (MAGUK family); zyxin; discoidin domain receptor family, member 2; adenomatosis polyposis coli; tetraspan 1; glycoprotein IX (platelet); thrombospondin 1; sialic acid binding Ig-like lectin 6; embryonal Fyn-associated substrate; protocadherin gamma subfamily C, 3; claudin 10; tumor necrosis factor, alpha-induced protein 6; lectin, galactoside-binding, soluble, 4 (galectin 4); FAT tumor suppressor homolog 2 (homolog of Drosophila); formyl peptide receptor-like 1; osteomodulin; carbohydrate sulfotransferase 10; Rho GDP dissociation inhibitor (GDI) beta; collagen, type VIII, alpha 1; neogenin homolog 1 (homolog of chicken); a disintegrin and metalloproteinase domain 10; protocadherin gamma subfamily C, 3; CD97 antigen; kininogen 1; protocadherin gamma subfamily C, 3; lymphocyte antigen 6 complex, locus D; cadherin 4, type 1, R-cadherin (retinal); lysyl oxidase-like 2; GDP-mannose 4,6-dehydratase; collagen, type XI, alpha 1; chemokine (C-C motif) ligand 11; RAB13, member RAS oncogene family; integrin beta 3 binding-protein (beta3-endonexin); protocadherin gamma subfamily C, 3; cartilage oligomeric matrix protein; DiGeorge syndrome critical region gene 6; spleen tyrosine kinase; connective tissue growth factor; cadherin, EGF LAG seven-pass G-type receptor 3 (flamingo homolog of Drosophila); scavenger receptor class F, member 1; hyaluronan and proteoglycan link protein 1; CD151 antigen; cadherin 2, type 1, N-cadherin (neuronal); neurotrimin; a disintegrin and metalloproteinase domain 12 (meltrin alpha); plexin C1; endoglin (Osler-Rendu-Weber syndrome 1); ret proto-oncogene (multiple endocrine neoplasia and medullary thyroid carcinoma 1, Hirschsprung disease); cadherin 19, type 2; immunoglobulin superfamily containing leucine-rich repeat; protocadherin gamma subfamily C, 3; protocadherin 12; dermatopontin; CD58 antigen, (lymphocyte function-associated antigen 3); ADAM-like, decysin 1; CD164 antigen, sialomucin; neurexin 3; tumor necrosis factor receptor superfamily, member 12A; PTK7 protein tyrosine kinase 7; cysteine-rich, angiogenic inducer, 61; CD34 antigen; protocadherin alpha 2; neuropilin 1; laminin, alpha 4; serum amyloid A2; nidogen 2 (osteonidogen); plakophilin 4; tuberous sclerosis 1; cadherin 18, type 2; transmembrane protein 8 (five membrane-spanning domains); macrophage erythroblast attacher; opioid binding protein/cell adhesion molecule-like; interleukin 18 (interferon-gamma-inducing factor); and ninjurin 2.

Glycosylation-associated genes that were found to be responsive to IMPDH inhibition and that could be used to prepare a microarray include: core 2 beta-1,6-N-acetylglucosaminyltransferase 3; sialyltransferase 8E (alpha-2,8-polysialyltransferase); mannosyl(alpha-1,6-)-glycoprotein beta-1,2-N-acetylglucosaminyltransferase; low density lipoprotein-related protein 2; glucosaminyl (N-acetyl) transferase 3, mucin type; mannosyl (alpha-1,3-)-glycoprotein beta-1,2-N-acetylglucosaminyltransferase; UDP-N-acetyl-alpha-D-galactosamine:polypeptide N-acetylgalactosaminyltransferase 1 (GalNAc-T1); stress-associated endoplasmic reticulum protein 1; transmembrane 4 superfamily member 5; lipase, hepatic; asparagine-linked glycosylation 3 homolog (yeast, alpha-1,3-mannosyltransferase); mannosyl(alpha-1,6-)-glycoprotein beta-1,6-N-acetyl-glucosaminyltransferase; UDP-GlcNAc:betaGal beta-1,3-N-acetylglucosaminyltransferase 3; mannosidase, alpha, class 1A, member 1; UDP-glucose ceramide glucosyltransferase-like 2; mannosyl (beta-1,4-)-glycoprotein beta-1,4-N-acetylglucosaminyltransferase; sialyltransferase 9 (CMP-NeuAc:lactosylceramide alpha-2,3-sialyltransferase; GM3 synthase); fucosyltransferase 1 (galactoside 2-alpha-L-fucosyltransferase); fucosyltransferase 9 (alpha (1,3) fucosyltransferase); sialyltransferase 8D (alpha-2,8-polysialyltransferase); fucosyltransferase 4 (alpha (1,3) fucosyltransferase, myeloid-specific); asparagine-linked glycosylation 6 homolog (homolog of yeast, alpha-1,3-glucosyltransferase); ribophorin I; Kell blood group; low density lipoprotein receptor (familial hypercholesterolemia); UDP-Gal:betaGlcNAc beta 1,3-galactosyltransferase, polypeptide 3; beta-1,3-glucuronyltransferase 1 (glucuronosyltransferase P); transmembrane 4 superfamily member 4; UDP-Gal:betaGlcNAc beta 1,3-galactosyltransferase, polypeptide 4; tissue specific transplantation antigen P35B; UDP-N-acetyl-alpha-D-galactosamine:polypeptide N-acetylgalactosaminyltransferase 2 (GaINAc-T2); phosphatidylinositol glycan, class B; protein-O-mannosyltransferase 1; sialyltransferase 8C (alpha2,3Galbeta1,4GlcNAcalpha 2,8-sialyltransferase); sialyltransferase 1 (beta-galactoside alpha-2,6-sialyltransferase); ribophorin II; interleukin 17 (cytotoxic T-lymphocyte-associated serine esterase 8); fucosyltransferase 7 (alpha (1,3) fucosyltransferase); UDP-Gal:betaGlcNAc beta 1,3-galactosyltransferase, polypeptide 2; dolichyl-phosphate mannosyltransferase polypeptide 1, catalytic subunit; component of oligomeric golgi complex 2; fucosyltransferase 5 (alpha (1,3) fucosyltransferase); stromal cell-derived factor 2; integral membrane protein 1; sialyltransferase 8A (alpha-N-acetylneuraminate: alpha-2,8-sialyltransferase, GD3 synthase); galactose-3-O-sulfotransferase 1; glucosaminyl (N-acetyl) transferase 2, I-branching enzyme; protein-O-mannosyltransferase 2; O-linked N-acetylglucosamine (GIcNAc) transferase (UDP-N-acetylglucosamine:polypeptide-N-acetylglucosaminyl transferase); mannosidase, alpha, class 1C, member 1; UDP-GlcNAc:betaGal beta-1,3-N-acetylglucosaminyltransferase 4; transmembrane 4 superfamily member 3; asparagine-linked glycosylation 5 homolog (homolog of yeast, dolichyl-phosphate beta-glucosyltransferase); UDP-glucose ceramide glucosyltransferase-like 1; transmembrane 4 superfamily member 2; mannosidase, alpha, class 1B, member 1; sialyltransferase 4C (beta-galactoside alpha-2,3-sialyltransferase); alpha-1,4-N-acetylglucosaminyltransferase; sialyltransferase 7 ((alpha-N-acetylneuraminyl-2,3-beta-galactosyl-1,3)-N-acetyl galactosaminide alpha-2,6-sialyltransferase) B; CD37 antigen; fucosyltransferase 6 (alpha (1,3) fucosyltransferase); sialyltransferase 10 (alpha-2,3-sialyltransferase VI); UDP-GlcNAc:betaGal beta-1,3-N-acetylglucosaminyltransferase 1; CDNA clone IMAGE:5093665, partial cds; sialyltransferase 4A (beta-galactoside alpha-2,3-sialyltransferase); sialyltransferase 7 ((alpha-N-acetylneuraminyl-2,3-beta-galactosyl-1,3)-N-acetyl galactosaminide alpha-2,6-sialyltransferase) E; glycophorin C (Gerbich blood group); glucosaminyl (N-acetyl) transferase 1, core 2 (beta-1,6-N-acetylglucosaminyltransferase); UDP-Gal:betaGlcNAc beta 1,3-galactosyltransferase, polypeptide 1; mannosidase, alpha, class 2B, member 1; fucosyltransferase 3 (galactoside 3(4)-L-fucosyltransferase, Lewis blood group included); sialyltransferase 7D ((alpha-N-acetylneuraminyl-2,3-beta-galactosyl-1,3)-N-acetyl galactosaminide alpha-2,6-sialyltransferase); lipase A, lysosomal acid, cholesterol esterase (Wolman disease); UDP-Gal:betaGlcNAc beta 1,3-galactosyltransferase, polypeptide 5; sialyltransferase 8B (alpha-2,8-sialyltransferase); dolichyl-diphosphooligosaccharide-protein glycosyltransferase; dolichyl-phosphate mannosyltransferase polypeptide 2, regulatory subunit; phosphomannomutase 2; and glucosidase I.

Microarrays that comprise chemotaxis-associated genes that are responsive to IMPDH inhibition would advantageously include a plurality of genes selected from the group consisting of: chemokine (C-C motif) receptor 7; chemokine (C-C motif) ligand 2; chemokine (C-X-C motif) ligand 14;chemokine (C-X-C motif) ligand 11; interleukin 8; chemokine (C-C motif) receptor 9; chemokine (C motif) ligand 1; chemokine-like factor; interleukin 4; chemokine (C-X-C motif) ligand 6 (granulocyte chemotactic protein 2); platelet factor 4 (chemokine (C-X-C motif) ligand 4); leukocyte cell-derived chemotaxin 2; chemokine (C-C motif) receptor-like 1; fibroblast growth factor 2 (basic); chemokine (C-X3-C motif) ligand 1; sialophorin (gpL115, leukosialin, CD43); plasminogen activator, urokinase receptor; chemokine (C-X3-C motif) receptor 1; chemokine-like receptor 1; chemokine (C-C motif) ligand 8; complement component 5; interleukin 8 receptor, beta; chemokine (C-C motif) ligand 25; azurocidin 1 (cationic antimicrobial protein 37); complement component 3a receptor 1; chemokine (C motif) receptor 1; chemokine (C-X-C motif) receptor 3; G protein-coupled receptor 77; phosphoinositide-3-kinase, catalytic, beta polypeptide; ribonuclease, RNase A family, 2 (liver, eosinophil-derived neurotoxin); chemokine (C motif) ligand 1; chemokine (C-C motif) receptor 5; chemokine (C-C motif) ligand 18 (pulmonary and activation-regulated); slit homolog 2 (homolog of Drosophila); chemokine (C-X-C motif) ligand 13 (B-cell chemoattractant); v-ral simian leukemia viral oncogene homolog A (ras related); chemokine (C-C motif) receptor 3; G protein-coupled receptor 77; chemokine binding protein 2; chemokine (C-X-C motif) ligand 12 (stromal cell-derived factor 1); mitogen-activated protein kinase 14;chemokine (C-X-C motif) ligand 2; chemokine (C-X-C motif) ligand 5; chemokine (C-C motif) ligand 1; chemokine (C-C motif) ligand 17; chemokine (C-X-C motif) ligand 10; serine (or cysteine) proteinase inhibitor, clade D (heparin cofactor), member 1; chemokine (C-C motif) receptor 8; endothelial cell growth factor 1 (platelet-derived); chemokine (C-X-C motif) ligand 2; FOS-like antigen 1; secretogranin III; chemokine (C-C motif) ligand 23; interleukin 10; secreted phosphoprotein 1 (osteopontin, bone sialoprotein I, early T-lymphocyte activation 1); ectonucleotide pyrophosphatase/phosphodiesterase 2 (autotaxin); platelet-activating factor receptor; chemokine (C-C motif) ligand 20; chemokine (C-C motif) receptor 6; phospholipase D1, phophatidylcholine-specific; chemokine (C-C motif) ligand 16; Kallmann syndrome 1 sequence; chemokine (C-C motif) ligand 15; chemokine (C-X-C motif) ligand 3; roundabout, axon guidance receptor, homolog 3 (homolog of Drosophila); mitogen-activated protein kinase 1; ralA binding protein 1; plasminogen activator, urokinase; defensin, beta 4; roundabout, axon guidance receptor, homolog 1 (homolog of Drosophila); interleukin 16 (lymphocyte chemoattractant factor); chemokine (C-C motif) ligand 27; chemokine (C-C motif) ligand 22; formyl peptide receptor-like 1; small inducible cytokine subfamily E, member 1 (endothelial monocyte-activating); v-Ha-ras Harvey rat sarcoma viral oncogene homolog; Transcribed sequences; chemokine (C-C motif) ligand 11; spleen tyrosine kinase; chemokine (C-C motif) receptor 4; chemokine (C-C motif) ligand 21; chemokine (C-X-C motif) ligand 9; formyl peptide receptor-like 2; chemokine (C-C motif) ligand 19; mitogen-activated protein kinase kinase 1; chemokine (C-C motif) ligand 13; cysteine-rich, angiogenic inducer, 61; serum amyloid A2; chemokine (C-C motif) ligand 24; and pro-platelet basic protein (chemokine (C-X-C motif) ligand 7).

Microarrays that are specific for cell-cell signaling pathway response to IMPDH would include a plurality of genes selected from the group consisting of G protein-coupled receptor 50; glutamyl aminopeptidase (aminopeptidase A); chemokine (C-X-C motif) ligand 14;chemokine (C-X-C motif) ligand 11; vasoactive intestinal peptide; interferon, alpha 8; ephrin-A5; gap junction protein, beta 1, 32 kDa (connexin 32, Charcot-Marie-Tooth neuropathy, X-linked); interferon, alpha 10; a disintegrin and metalloproteinase domain 17 (tumor necrosis factor, alpha, converting enzyme); S100 calcium binding protein A6 (calcyclin); poliovirus receptor-related 1 (herpesvirus entry mediator C; nectin); netrin 1; interleukin 8; carcinoembryonic antigen-related cell adhesion molecule 6 (non-specific cross reacting antigen); neuregulin 2; ephrin-B1; pan-hematopoietic expression; plakophilin 2; adrenergic, alpha-1A-, receptor; lymphotoxin alpha (TNF superfamily, member 1); interferon, gamma; fatty acid binding protein 1, liver; somatostatin receptor 3; wingless-type MMTV integration site family, member 4; chemokine (C motif) ligand 1; granulin; sema domain, seven thrombospondin repeats (type 1 and type 1-like), transmembrane domain (TM) and short cytoplasmic domain, (semaphorin) 5A; leptin (obesity homolog of mouse); TGFB inducible early growth response; intercellular adhesion molecule 2; thioredoxin; tachykinin, precursor 1 (substance K, substance P, neurokinin 1, neurokinin 2, neuromedin L, neurokinin alpha, neuropeptide K, neuropeptide gamma); sonic hedgehog homolog (homolog of, Drosophila); chemokine (C-X-C motif) ligand 6 (granulocyte chemotactic protein 2); tumor necrosis factor (ligand) superfamily, member 6; ephrin-A4; fibroblast growth factor 17; CD44 antigen (homing function and Indian blood group system); nerve growth factor, beta polypeptide; fibroblast growth factor 4 (heparin secretory transforming protein 1, Kaposi sarcoma oncogene); adrenergic, alpha-2C-, receptor; fibroblast growth factor 2 (basic); CD80 antigen (CD28 antigen ligand 1, B7-1 antigen); fibroblast growth factor 8 (androgen-induced); fibroblast growth factor 3 (murine mammary tumor virus integration site (v-int-2) oncogene homolog); myelin protein zero-like 1; fibroblast growth factor 16; gap junction protein, alpha 3, 46 kDa (connexin 46); adenosine A1 receptor; bone morphogenetic protein 10; tumor suppressor candidate 2; fibroblast growth factor 1 (acidic); thyrotropin-releasing hormone degrading ectoenzyme; tumor necrosis factor (TNF superfamily, member 2); bone morphogenetic protein 3 (osteogenic); gap junction protein, alpha 1, 43 kDa (connexin 43); colony stimulating factor 3 (granulocyte); vasoactive intestinal peptide receptor 2; proprotein convertase subtilisin/kexin type 5; vascular cell adhesion molecule 1; transforming growth factor, beta 3; tumor necrosis factor (ligand) superfamily, member 8; chemokine (C-C motif) ligand 8; ephrin-A1; somatostatin receptor 2; chorionic somatomammotropin hormone 1 (placental lactogen); IK cytokine, down-regulator of HLA II; tumor necrosis factor (ligand) superfamily, member 9; sprouty homolog 2 (homolog of Drosophila); fibroblast growth factor 6; motilin; TEK tyrosine kinase, endothelial (venous malformations, multiple cutaneous and mucosal); cardiotrophin 1; FXYD domain containing ion transport regulator 5; WNT inhibitory factor 1; intercellular adhesion molecule 3; GRB2-related adaptor protein 2; gonadotropin-releasing hormone 1 (leutinizing-releasing hormone); estrogen receptor 2 (ER beta); bone morphogenetic protein 2; WNT1 inducible signaling pathway protein 1; steroid-5-alpha-reductase, alpha polypeptide 2 (3-oxo-5 alpha-steroid delta 4-dehydrogenase alpha 2); calcitonin/calcitonin-related polypeptide, alpha; desmoplakin, tumor necrosis factor (ligand) superfamily, member 7; fibroblast growth factor 14;nitric oxide synthase 1 (neuronal); microsomal glutathione S-transferase 2; chemokine (C motif) ligand 1; thyrotropin-releasing hormone; protocadherin beta 6; interferon, alpha 4; epiregulin; proprotein convertase subtilisin/kexin type 2; CD209 antigen; chemokine (C-C motif) receptor 5; interleukin 9; nuclear receptor subfamily 5, group A, member 1; growth factor receptor-bound protein 10; crumbs homolog 1 (homolog of Drosophila); GRB2-related adaptor protein; pre-B-cell colony enhancing factor 1; chemokine (C-C motif) ligand 18 (pulmonary and activation-regulated); discs, large homolog 5 (homolog of Drosophila); angiotensinogen (serine (or cysteine) proteinase inhibitor, clade A (alpha-1 antiproteinase, antitrypsin), member 8); fibroblast growth factor 20; protocadherin beta 11; interleukin 17 (cytotoxic T-lymphocyte-associated serine esterase 8); nephronophthisis 1 (juvenile); ephrin-A2; somatostatin receptor 1; proprotein convertase subtilisin/kexin type 6; intercellular adhesion molecule 5, telencephalin; protocadherin beta 5; transforming growth factor, alpha; tumor necrosis factor (ligand) superfamily, member 10; chemokine (C-X-C motif) ligand 13 (B-cell chemoattractant); phosphoglucomutase 5; adrenergic, alpha-1D-, receptor; proopiomelanocortin (adrenocorticotropin/beta-lipotropin/alpha-melanocyte stimulating hormone/beta-melanocyte stimulating hormone/beta-endorphin); thyroid stimulating hormone, beta; agouti signaling protein, nonagouti homolog (homolog of mouse); interferon, alpha 10; phosphate regulating endopeptidase homolog, X-linked (hypophosphatemia, vitamin D resistant rickets); placental growth factor, vascular endothelial growth factor-related protein; prostaglandin I2 (prostacyclin) receptor (IP); inhibin, alpha; c-mer proto-oncogene tyrosine kinase; lymphotoxin beta (TNF superfamily, member 3); myeloid/lymphoid or mixed-lineage leukemia (trithorax homolog of Drosophila); translocated to, 4; CD209 antigen-like; endothelin 3; tumor necrosis factor receptor superfamily, member 11a, activator of NFKB; adrenergic, alpha-2B-, receptor; chemokine (C-X-C motif) ligand 12 (stromal cell-derived factor 1); wingless-type MMTV integration site family, member 8B; tumor necrosis factor (ligand) superfamily, member 18; chromosome 1 open reading frame 10; SH2 domain containing phosphatase anchor protein 1; tumor necrosis factor (ligand) superfamily, member 4 (tax-transcriptionally activated glycoprotein 1, 34 kDa); interleukin 11; interleukin 6 (interferon, beta 2); neuronal cell adhesion molecule; endometrial bleeding associated factor (left-right determination, factor A; transforming growth factor beta superfamily); chemokine (C-X-C motif) ligand 5; protocadherin 8; sialoadhesin; adrenomedullin; chemokine (C-C motif) ligand 1; ectonucleoside triphosphate diphosphohydrolase 1; wingless-type MMTV integration site family, member 5A; chemokine (C-C motif) ligand 17; chemokine (C-X-C motif) ligand 10; amphiregulin (schwannoma-derived growth factor); intercellular adhesion molecule 1 (CD54), human rhinovirus receptor; complement component 1, q subcomponent, receptor 1; furin (paired basic amino acid cleaving enzyme); erythropoietin; endothelial cell growth factor 1 (platelet-derived); neuromedin B; interleukin 22; neural cell adhesion molecule 1; thyroid stimulating hormone receptor; CD33 antigen (gp67); stromal interaction molecule 1; sema domain, immunoglobulin domain (Ig), transmembrane domain (TM) and short cytoplasmic domain, (semaphorin) 4F; ephrin-B2; growth differentiation factor 5 (cartilage-derived morphogenetic protein-1); insulin; chemokine (C-C motif) ligand 23; fibroblast growth factor binding protein 1; interleukin 10; secreted phosphoprotein 1 (osteopontin, bone sialoprotein I, early T-lymphocyte activation 1); proenkephalin; CD86 antigen (CD28 antigen ligand 2, B7-2 antigen); fibroblast growth factor 13; lactalbumin, alpha-; stanniocalcin 2; steroid-5-alpha-reductase, alpha polypeptide 1 (3-oxo-5 alpha-steroid delta 4-dehydrogenase alpha 1); wingless-type MMTV integration site family, member 3; stanniocalcin 1; chemokine (C-C motif) ligand 20; adenylate cyclase activating polypeptide 1 (pituitary); ephrin-B3; insulin-like 4 (placenta); FAT tumor suppressor homolog 1 (homolog of Drosophila); chemokine (C-C motif) ligand 16; nudix (nucleoside diphosphate linked moiety X)-type motif 3; interferon, alpha 21; integrin, beta 2 (antigen CD18 (p95), lymphocyte function-associated antigen 1; macrophage antigen 1 (mac-1) beta subunit); arginine vasopressin (neurophysin II, antidiuretic hormone, diabetes insipidus, neurohypophyseal); interferon, alpha 2; transcription factor AP-2 gamma (activating enhancer binding protein 2 gamma); interleukin 1 receptor-like 1 ligand; peptide YY; chemokine (C-C motif) ligand 15; WNT1 inducible signaling pathway protein 3; catenin (cadherin-associated protein), delta 1; junctional adhesion molecule 2; prostaglandin D2 synthase, hematopoietic; fibroblast growth factor 21; endothelin 1; CD44 antigen (homing function and Indian blood group system); CD2 antigen (p50), sheep red blood cell receptor; cysteine-rich secretory protein 3; discs, large (Drosophila) homolog-associated protein 1; endothelin 2; wingless-type MMTV integration site family, member 7A; carbohydrate (N-acetylglucosamine 6-O) sulfotransferase 4; platelet-derived growth factor alpha polypeptide; membrane metallo-endopeptidase (neutral endopeptidase, enkephalinase, CALLA, CD10); interleukin 3 (colony-stimulating factor, multiple); islet amyloid polypeptide; WNT1 inducible signaling pathway protein 2; interleukin 17B; major intrinsic protein of lens fiber; growth hormone releasing hormone; parathyroid hormone-like hormone; intercellular adhesion molecule 4, Landsteiner-Wiener blood group; ephrin-A3; protocadherin beta 3; sema domain, immunoglobulin domain,(Ig), short basic domain, secreted, (semaphorin) 3B; pro-melanin-concentrating hormone; neurotrophin 3; zyxin; interleukin 13; sialic acid binding Ig-like lectin 6; chemokine (C-C motif) ligand 27; interleukin 15; chorionic gonadotropin, beta polypeptide; Norrie disease (pseudoglioma); glycoprotein hormones, alpha polypeptide; tumor necrosis factor, alpha-induced protein 6; androgen receptor (dihydrotestosterone receptor; testicular feminization; spinal and bulbar muscular atrophy; Kennedy disease); chemokine (C-C motif) ligand 22; bone marrow stromal cell antigen 2; midkine (neurite growth-promoting factor 2); fibroblast growth factor 9 (glia-activating factor); wingless-type MMTV integration site family, member 11; SH2 domain protein 1A, Duncan's disease (lymphoproliferative syndrome); discs, large (homolog of Drosophila) homolog-associated protein 2; neogenin homolog 1 (homolog of chicken); a disintegrin and metalloproteinase domain 10; fibroblast growth factor 1 (acidic); CD97 antigen; small inducible cytokine subfamily E, member 1 (endothelial monocyte-activating); SH2 domain protein 1A, Duncan's disease (lymphoproliferative syndrome); inhibin, beta A (activin A, activin AB alpha polypeptide); parathyroid hormone; jagged 2; collagen, type XI, alpha 1; chemokine (C-C motif) ligand 11; interleukin 1 family, member 9; complement component 1, q subcomponent, alpha polypeptide; leukemia inhibitory factor (cholinergic differentiation factor); chemokine (C-C motif) ligand 21; hydroxysteroid (11-beta) dehydrogenase 2; chemokine (C-X-C motif) ligand 9; adrenergic, alpha-1B-, receptor; cocaine- and amphetamine-regulated transcript; interferon, alpha-inducible protein (clone IFI-15K); fibroblast growth factor 5; CD58 antigen, (lymphocyte function-associated antigen 3); interleukin 7; anti-Mullerian hormone; chemokine (C-C motif) ligand 13; proprotein convertase subtilisin/kexin type 1; interleukin 1, beta; interleukin 26; neuropilin 1; oncostatin M; sorbin and SH3 domain containing 1; S100 calcium binding protein A9 (calgranulin B); chemokine (C-C motif) ligand 24; interleukin 18 (interferon-gamma-inducing factor); and insulin-degrading enzyme.

Microarrays affecting translation in response to IMPDH inhibitors would include a plurality of genes selected from the group consisting of methionine-tRNA synthetase; alanyl-tRNA synthetase; solute carrier family 22 (organic cation transporter), member 17; tyrosyl-tRNA synthetase; histidyl-tRNA synthetase; seryl-tRNA synthetase; phenylalanine-tRNA synthetase 1 (mitochondrial); N-acylaminoacyl-peptide hydrolase; valyl-tRNA synthetase 2; aminoacylase 1; aspartoacylase (aminoacylase 2, Canavan disease); isoleucine-tRNA synthetase; arginyl-tRNA synthetase; transcription factor binding to IGHM enhancer 3; lysyl-tRNA synthetase; phenylalanine-tRNA synthetase-like, alpha subunit; threonyl-tRNA synthetase; cysteinyl-tRNA synthetase; tryptophanyl tRNA synthetase 2 (mitochondrial); glycyl-tRNA synthetase; asparaginyl-tRNA synthetase; glutaminyl-tRNA synthetase; solute carrier family 22 (organic cation transporter), member 17; aspartyl-tRNA synthetase; small inducible cytokine subfamily E, member 1 (endothelial monocyte-activating); glutamyl-prolyl-tRNA synthetase; lysyl-tRNA synthetase; and tryptophanyl-tRNA synthetase.

Microarrays that are specific for genes required for mitochondrial function that are responsive to IMPDH would include a plurality of geres selected from the group consisting of succinate dehydrogenase complex, subunit C, integral membrane protein, 15 kDa; mitochondrial ribosomal protein L3; malate dehydrogenase 2, NAD (mitochondrial); hexokinase 2; uncoupling protein 3 (mitochondrial, proton carrier); death associated protein 3; 4-aminobutyrate aminotransferase; ubiquinol-cytochrome c reductase binding protein; mitochondrial ribosomal protein L12; mitochondrial intermediate peptidase; ubiquinol-cytochrome c reductase complex (7.2 kD); translocase of inner mitochondrial membrane 17 homolog A (yeast); ATP-binding cassette, sub-family F (GCN20), member 2; ATP synthase, H+ transporting, mitochondrial F0 complex, subunit F6; solute carrier family 25 (mitochondrial carrier, brain), member 14; thiosulfate sulfurtransferase (rhodanese); cytochrome c oxidase subunit VIa polypeptide 2; BH3 interacting domain death agonist; aldehyde dehydrogenase 4 family, member A1; translocase of outer mitochondrial membrane 40 homolog (yeast); heat shock 60 kDa protein 1 (chaperonin); mercaptopyruvate sulfurtransferase; NADH dehydrogenase (ubiquinone) Fe—S protein 6, 13 kDa (NADH-coenzyme Q reductase); NADH dehydrogenase (ubiquinone) 1 beta subcomplex, 2, 8 kDa; mitochondrial ribosomal protein S12; glycerol kinase; NADH dehydrogenase (ubiquinone) Fe—S protein 4, 18 kDa (NADH-coenzyme Q reductase); myeloid cell leukemia sequence 1 (BCL2-related); NADH dehydrogenase (ubiquinone) 1, subcomplex unknown, 2, 14.5 kDa; translocase of inner mitochondrial membrane 22 homolog (homolog of yeast); acyl-Coenzyme A dehydrogenase, C-4 to C-12 straight chain; solute carrier family 25 (mitochondrial carrier; phosphate carrier), member 3; cytochrome c oxidase subunit Vb; popeye domain containing 2; peptidase (mitochondrial processing) beta; mitochondrial ribosomal protein L23; NADH dehydrogenase (ubiquinone) Fe—S protein 1, 75 kDa (NADH-coenzyme Q reductase); dynamin 1-like; translocase of inner mitochondrial membrane 13 homolog (yeast); NADH dehydrogenase (ubiquinone) 1 alpha subcomplex, 6, 14 kDa; cytochrome c oxidase subunit VIa polypeptide 1; sarcosine dehydrogenase; B-cell CLL/lymphoma 2; voltage-dependent anion channel 1; 3-hydroxymethyl-3-methylglutaryl-Coenzyme A lyase (hydroxymethylglutaricaciduria); BCL2-associated X protein; NADH dehydrogenase (ubiquinone) 1, subcomplex unknown, 1, 6 kDa; omithine aminotransferase (gyrate atrophy); camitine palmitoyltransferase II; solute carrier family 25 (mitochondrial oxodicarboxylate carrier), member 21; monoamine oxidase B; NADH dehydrogenase (ubiquinone) Fe—S protein 2, 49 kDa (NADH-coenzyme Q reductase); ornithine carbamoyltransferase; NADH dehydrogenase (ubiquinone) 1 beta subcomplex, 5, 16 kDa; ubiquinol-cytochrome c reductase core protein I; solute carrier family 25 (mitochondrial carrier; omithine transporter) member 15; surfeit 1; voltage-dependent anion channel 3; hydroxyacyl-Coenzyme A dehydrogenase/3-ketoacyl-Coenzyme A thiolase/enoyl-Coenzyme A hydratase (trifunctional protein), beta subunit; translocase of inner mitochondrial membrane 8 homolog B (homolog of yeast); ubiquinol-cytochrome c reductase (6.4 kD) subunit; electron-transferring-flavoprotein dehydrogenase; NADH dehydrogenase (ubiquinone) Fe—S protein 8, 23 kDa (NADH-coenzyme Q reductase); NADH dehydrogenase (ubiquinone) 1 beta subcomplex, 3, 12 kDa; phosphoprotein enriched in astrocytes 15 [BLAST]; carbonic anhydrase VA, mitochondrial; NADH dehydrogenase (ubiquinone) Fe—S protein 5, 15 kDa (NADH-coenzyme Q reductase); inner membrane protein, mitochondrial (mitofilin); translocase of inner mitochondrial membrane 9 homolog (yeast); oxidase (cytochrome c) assembly 1-like; transcription factor A, mitochondrial; translocase of outer mitochondrial membrane 20 homolog (homolog of yeast); DnaJ (Hsp40) homolog, subfamily A, member 3; NADH dehydrogenase (ubiquinone) 1 beta subcomplex, 6, 17 kDa; endothelial cell growth factor 1 (platelet-derived); hypothetical protein LOC114971; NADH dehydrogenase (ubiquinone) flavoprotein 1, 51 kDa; electron-transfer-flavoprotein, beta polypeptide; transcription factor A, mitochondrial; mitochondrial folate transporter/carrier; NADH dehydrogenase (ubiquinone) 1 alpha subcomplex, 9, 39 kDa; v-raf-1 murine leukemia viral oncogene homolog 1; ubiquinol-cytochrome c reductase core protein II; solute carrier family 25 (mitochondrial carrier; peroxisomal membrane protein, 34 kDa), member 17; NADH dehydrogenase (ubiquinone) 1 beta subcomplex, 8, 19 kDa; uncoupling protein 1 (mitochondrial, proton carrier); START domain containing 3; BCL2-antagonist/killer 1; nicotinamide nucleotide transhydrogenase; translocase of outer mitochondrial membrane 34; holocytochrome c synthase (cytochrome c heme-lyase); mitochondrial carrier family protein; ATP-binding cassette, sub-family B (MDR/TAP), member 8; BCL2-antagonist of cell death; cytochrome c oxidase subunit IV isoform 2; succinate dehydrogenase complex, subunit D, integral membrane protein; solute carrier family 25 (camitine/acylcamitine translocase), member 20; solute carrier family 25 (mitochondrial carrier; oxoglutarate carrier), member 11; translocase of inner mitochondrial membrane 17 homolog B (yeast); solute carrier family 25 (mitochondrial carrier, Aralar), member 12; uncoupling protein 2 (mitochondrial, proton carrier); NADH dehydrogenase (ubiquinone) 1 alpha subcomplex, 5, 13 kDa; cytochrome c-1; methylcrotonoyl-Coenzyme A carboxylase 1 (alpha); NADH dehydrogenase (ubiquinone) 1 beta subcomplex, 4, 15 kDa; isovaleryl Coenzyme A dehydrogenase; mitochondrial ribosomal protein S15; benzodiazapine receptor (peripheral); fracture callus 1 homolog (homolog of rat); protein (peptidyl-prolyl cis/trans isomerase) NIMA-interacting, 4 (parvulin); cytochrome c oxidase subunit VIIb; metaxin 2; mitochondrial ribosomal protein L40; NADH dehydrogenase (ubiquinone) flavoprotein 2, 24 kDa; translocase of inner mitochondrial membrane 44 homolog (yeast); translocase of inner mitochondrial membrane 23 homolog (homolog of yeast); electron-transfer-flavoprotein, alpha polypeptide (glutaric aciduria II); solute carrier family 25, and member 13 (citrin).

Microarrays that are specific for genes from cGMP biosynthesis pathway that are responsive to IMPDH inhibitors would include a plurality of genes selected from the group consisting of guanylate cyclase 1, soluble, beta 3; natriuretic peptide receptor B/guanylate cyclase B (atrionatriuretic peptide receptor B); protein kinase, cGMP-dependent, type II; phosphodiesterase 6B, cGMP-specific, rod, beta (congenital stationary night blindness 3, autosomal dominant); phosphodiesterase 6C, cGMP-specific, cone, alpha prime; phosphodiesterase 6A, cGMP-specific, rod, alpha; guanylate cyclase 2F, retinal; natriuretic peptide receptor A/guanylate cyclase A (atrionatriuretic peptide receptor A); guanylate cyclase 1, soluble, alpha 2; phosphodiesterase 3B, cGMP-inhibited; phosphodiesterase 9A; phosphodiesterase 3B, cGMP-inhibited; guanylate cyclase 2D, membrane (retina-specific); phosphodiesterase 2A, cGMP-stimulated; cyclic nucleotide gated channel alpha 3; phosphodiesterase 6G, cGMP-specific, rod, gamma; phosphodiesterase 6H, cGMP-specific, cone, gamma; cyclic nucleotide gated channel alpha 1; and phosphodiesterase 3A, cGMP-inhibited.

Microarrays that are specific for genes from cAMP biosynthesis pathway that are responsive to IMPDH inhibitors would include a plurality of genes selected from the group consisting of phosphodiesterase 4D, cAMP-specific (phosphodiesterase E3 dunce homolog of Drosophila); cholinergic receptor, muscarinic 2; adenylate cyclase 7; protein kinase, cGMP-dependent, type I; adenylate cyclase 6; Rap guanine nucleotide exchange factor (GEF) 3; glucagon-like peptide 2 receptor; protein kinase, X-linked; melanocortin 4 receptor; adrenergic, beta-3-, receptor; calcitonin/calcitonin-related polypeptide, alpha; phosphodiesterase 4B, cAMP-specific (phosphodiesterase E4 dunce homolog, Drosophila); cAMP responsive element modulator; adenylate cyclase 3; calcium-binding tyrosine-(Y)-phosphorylation regulated (fibrousheathin 2); adrenergic, alpha-1D-, receptor; protein kinase, AMP-activated, gamma 1 non-catalytic subunit; cyclic nucleotide gated channel beta 1; chemokine (C-C motif) receptor 3; testicular soluble adenylyl cyclase; adenylate cyclase 8 (brain); phosphodiesterase 7B; protein kinase, cAMP-dependent, catalytic, alpha; adrenomedullin; Rap guanine nucleotide exchange factor (GEF) 2; A kinase (PRKA) anchor protein 4; adenylate cyclase 9; Rap guanine nucleotide exchange factor (GEF) 4; phosphodiesterase 4C, cAMP-specific (phosphodiesterase E1 dunce homolog of Drosophila); prostaglandin E receptor 4 (subtype EP4); G protein-coupled receptor kinase 5; protein kinase (cAMP-dependent, catalytic) inhibitor alpha; galanin receptor 2; protein kinase, cAMP-dependent, regulatory, type I, alpha (tissue specific extinguisher 1); arginine vasopressin receptor 2 (nephrogenic diabetes insipidus); protein kinase, cAMP-dependent, catalytic, beta; corticotropin releasing hormone receptor 2; growth hormone releasing hormone; parathyroid hormone-like hormone; adenosine A2a receptor; protein kinase, cAMP-dependent, regulatory, type II, alpha; corticotropin releasing hormone receptor 1; parathyroid hormone; hyperpolarization activated cyclic nucleotide-gated potassium channel 2; adrenergic, alpha-1B-, receptor; ret proto-oncogene (multiple endocrine neoplasia and medullary thyroid carcinoma 1, Hirschsprung disease); phosphodiesterase 4A, cAMP-specific (phosphodiesterase E2 dunce homolog of Drosophila); protein kinase, cAMP-dependent, regulatory, type II, beta; and protein kinase (cAMP-dependent, catalytic) inhibitor gamma.

The term “microarray” refers to an ordered arrangement of hybridizable array elements (i.e., the above-described polynucleotide probes). The array elements are arranged so that there are preferably at least one or more different array elements, more preferably at least 100 array elements, and most preferably at least 1,000 array elements, on a 1 cm² substrate surface. The hybridization signal from each of the array elements is individually distinguishable. In a preferred embodiment, the array elements comprise polynucleotide probes.

A “polynucleotide” refers to a chain of nucleotides, preferably a single-stranded chain. Preferably, the chain has from about 75 to 10,000 nucleotides, more preferably from about 100 to 3,500 nucleotides. The term “probe” refers to a polynucleotide sequence capable of hybridizing with a target sequence to form a polynucleotide probe/target complex. A “target polynucleotide” refers to a chain of nucleotides to which a polynucleotide probe can hybridize by base pairing. In some instances, the sequences will be complementary (no mismatches) when aligned. In other instances, there may be up to a 10%. mismatch.

A “plurality” is given its ordinary meaning of 2 or more. In the present context, a plurality of polynucleotide probes (elements) refers preferably to a group of at least least 4, 5, 10, 15, 20, 25, 30, 35, 40, 50, 60, 70, 80, 90, 100, 110, 120, 130, 140, 150, 200, 300 or more polynucleotides or more elements, more preferably to a group of at least about 100, and even more preferably to a group of at least about 1,000, elements. The maximum number of members is unlimited, but is at least about 100,000 members.

The term “gene” or “genes” refers to polynucleotide sequence of a gene which may be the partial or complete gene and may comprise regulatory, untranslated, or coding regions.

The microarray can be used for large-scale genetic or gene expression analysis of a number of target polynucleotides. The microarray can also be used in the diagnosis of diseases and in the monitoring of treatments. Further, the microarray can be employed to investigate an individual's predisposition to a disease. Furthermore, the microarray can be employed to investigate cellular responses to carcinogens, mitogens, drug treatment, and the like.

The hybridizable array elements in a microarray of the invention may be organized in an ordered fashion so that each element is present at a specified location on the substrate. As such, each of the nucleic acids on the array will have its own “address” such that hybridization to that nucleic acid will allow specific identification of the complementary nucleic acid in a biological sample. Because the array elements are at specified locations on the substrate, the hybridization patterns and intensities can be interpreted in terms of expression levels of particular genes. Collectively, the expression profile obtained with the microarrays of the invention are tightly correlated to a particular disease or condition or treatment. Hence, the invention provides dramatically enhanced reliability in profiling and obtaining prognostic indicators of response to IMPDH inhibition.

The composition comprising a plurality of polynucleotide probes can also be used to purify a subpopulation of mRNAs, cDNAs, genomic fragments and the like, in a sample. This may be particularly useful in identifying subsets of the above-identified nucleic acids that are more highly indicative of modulated/abnormal IMPDH activity.

The nucleic acids identified herein as being responsive to IMPDH inhibition will be used in microarray production and can be genomic DNA or cDNA or mRNA, or any RNA-like or DNA-like material, such as peptide nucleic acids, branched DNAs and the like. The polynucleotide probes can be sense or antisense polynucleotide probes. Where target polynucleotides are double-stranded, the probes may be either sense or antisense strands. Where the target polynucleotides are single-stranded, the nucleotide probes are complementary single strands.

In one embodiment, the polynucleotide probes are cDNAs. The size of the DNA sequence of interest may vary and is preferably from 20 to 10,000 nucleotides, more preferably from 20 to 200 nucleotides. The polynucleotide probes can be prepared by a variety of synthetic or enzymatic schemes which are well known in the art. The probes can be synthesized, in whole or in part, using chemical methods well known in the art Caruthers et al. (1980) Nucleic Acids Res. Symp. Ser. 215-233). Alternatively, the probes can be generated, in whole or in part, enzymatically.

Nucleotide analogues can be incorporated into the polynucleotide probes by methods well known in the art. The only requirement is that the incorporated nucleotide analogues must serve to base pair with target polynucleotide sequences. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs than those between adenine and thymidine.

Additionally, the polynucleotide probes can include nucleotides that have been derivatized chemically or enzymatically. Typical chemical modifications include derivatization with acyl, alkyl, aryl or amino groups.

The polynucleotide probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Complementary DNA (cDNA) can be arranged and then immobilized on a substrate. The probes can be immobilized by covalent means such as by chemical bonding procedures or UV. In one such method, a cDNA is bound to a glass surface which has been modified to contain epoxide or aldehyde groups. In another case, a cDNA probe is placed on a polylysine coated surface and then UV cross-linked (Shalon et al. PCT publication WO95/35505, herein incorporated by reference). In yet another method, a DNA is actively transported from a solution to a given position on a substrate by electrical means (Heller et al. U.S. Pat. No. 5,605,662). Alternatively, individual DNA clones can be gridded on a filter.

Furthermore, the probes do not have to be directly bound to the substrate, but rather can be bound to the substrate through a linker group. The linker groups are typically about 6 to 50 atoms long to provide exposure to the attached polynucleotide probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the polynucleotide probe.

The polynucleotide probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Hybridization of Nitcleic Acids from Biological Sample to Microarray. In order to detect the presence of a given nucleic acid in a biological sample it will be desirable to hybridize the nucleic acid isolated from that sample to the microarray. Hybridization causes a denatured polynucleotide probe and a denatured complementary target to form a stable duplex through base pairing. Hybridization methods are well known to those skilled in the art (See, e.g., Ausubel (1997; Short Protocols in Molecular Biology, John Wiley & Sons, New York N.Y., units 2.8-2.11, 3.18-3.19 and 4-6-4.9). Conditions can be selected for hybridization where exactly complementary target and polynucleotide probe can hybridize, i.e., each base pair must interact with its complementary base pair. Alternatively, conditions can be selected where target and polynucleotide probes have mismatches but are still able to hybridize. Suitable conditions can be selected, for example, by varying the concentrations of salt in the prehybridization, hybridization and wash solutions, or by varying the hybridization and wash temperatures. With some membranes, the temperature can be decreased by adding formamide to the prehybridization and hybridization solutions.

Hybridization can be performed at low stringency with buffers, such as 6 x SSPE with 0.005% Triton X-100 at 37° C., which permits hybridization between target and polynucleotide probes that contain some mismatches to form target polynucleotide/probe complexes. Subsequent washes are performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl or Triton X-100, or a blocking agent, such as salmon sperm DNA.

After hybridization, the microarray is washed to remove nonhybridized nucleic acids, and complex formation between the hybridizable array elements and the target polynucleotides is detected. Methods for detecting complex formation are well known to those skilled in the art. In a preferred embodiment, the target polynucleotides are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensity. The scanned image is examined to determine the abundance/expression level of each hybridized target polynucleotide.

Typically, microarray fluorescence intensities can be normalized to take into account variations in hybridization intensities when more than one microarray is used under similar test conditions. In a preferred embodiment, individual polynucleotide probe/target complex hybridization intensities are normalized using the intensities derived from internal normalization controls contained on each microarray.

Microarray Expression Profiles. As discussed above an important aspect of the present invention is a method of obtaining an expression profile, using the microarray compositions of the invention, of a subject that has or is about to undergo therapy based on IMPDH inhibition. The expression profile can be used to detect changes in the expression of genes in response to such inhibition and to provide a prognosis of a patient's response to an IMPDH inhibitor comprising the steps of: (a) subjecting RNA extracted from the cells obtained from the patient to gene expression analysis on one of the microarrays of the invention in the presence and absence of said IMPDH inhibitor. In doing so, the expression level of at least one gene selected from the genes in Tables I through VIII is determined and compared to the amount of expression found in a corresponding reference tissue set that has not been treated with an IMPDH inhibitor. Subsequently, a report summarizing the data obtained by such gene expression analysis can be prepared and used to determine whether the patient will likely be responsive to IMPDH inhibition.

The expression profile comprises determining the level of expression the nucleic acids that have been identified herein as being responsive to IMPDH inhibition and may further involve categorizing said nucleic acids into functional categories (e.g., the gene has a cell-cycle function, a cell proliferation function, is involved in lipid metabolism some other metabolic pathway, and the like). It is contemplated that at least one of the nucleic acids identified herein, and preferably a plurality thereof, is hybridized to a complementary target polynucleotide forming at least one, and preferably a plurality, of complexes. A complex is preferably detected by incorporating at least one labeling moiety in the complex as described above. The expression profiles provide “snapshots” that can show unique expression patterns that are characteristic of that individual's response to IMPDH inhibition.

After performing hybridization experiments and interpreting detected signals from a microarray, particular polynucleotide probes can be identified and selected based on their expression patterns (e.g., those that are consistently and dramatically up- or down-regulated upon IMPDH inhibition). Such polynucleotide probe sequences can be used to clone a full length sequence of the gene for further analysis, provide an alternative diagnostic tool, or to produce the encoded polypeptide.

In one situation, the microarray is used to monitor the progression of disease and the response of that disease to IMPDH inhibition. Researchers can assess and catalog the differences in gene expression between healthy and diseased tissues or cells. By analyzing changes in patterns of gene expression, disease can be diagnosed at earlier stages before the patient is symptomatic. The invention can also be used to monitor the efficacy of treatment. For some treatments with known side effects, the microarray is employed to “fine tune” the treatment regimen. A dosage of IMPDH inhibitor is established that causes a change in genetic expression patterns indicative of successful treatment. Expression patterns associated with undesirable side effects are avoided. This approach may be more sensitive and rapid than waiting for the patient to show inadequate improvement, or to manifest side effects, before altering the course of treatment.

Alternatively, animal models which mimic a disease, rather than patients having the disease, can be used to characterize expression profiles associated with a particular inhibitor. This gene expression data may be useful in diagnosing and monitoring the course of disease in a patient, in determining gene targets for intervention, and in testing treatment regimens.

Also, researchers can use the microarray to rapidly screen large numbers of candidate IMPDH inhibitory drug molecules, looking for ones that produce an expression profile similar to those of known therapeutic drugs e.g., VX-944, MPA, Nucleoside analogs such as tiazofurin, ribavirin and mizoribine, and other agents listed in e.g., U.S. Pat. Nos. 5,807,876, 5,932,600, 6,054,472, 6,344,465, 6,395,763, 6,399,773, 6,420,403, 6,867,299, 6,826,488, 6,825,224, 6,653,309, 6,624,184, 6,617,323, 6,541,496, 6,518,291, and 6,498,178 (each specifically incorporated herein by reference in its entirety for its teaches of IMPDH inhibitor compositions and methods of administering the same for the treatment of IMPDH related disorders), with the expectation that molecules with the same expression profile will have similar therapeutic effects. Thus, the invention provides the means to determine the molecular mode of action of an IPDH inhibitor or IMPDH pathway inhibitor, as well as to facilitate identification of new such drugs.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus are considered to constitute certain aspects for its practice. However, those of skill in the art should, in light of the present disclosure, appreciate that many changes can be made in the specific embodiments which are disclosed and still obtain a like or similar result without departing from the spirit and scope of the invention.

### Example 1

### VX-944-Biomarker Identification by Transcript Profiling

Inosine monophosphate dehydrogenase (IMPDH) catalyzes a rate-limiting step in guanine nucleotide biosynthesis, the NAD-dependent reduction of IMP to XMP. Inhibition of IMPDH activity results in the cessation of DNA synthesis and is an important target for immunosuppressive, anticancer and antiviral therapy.

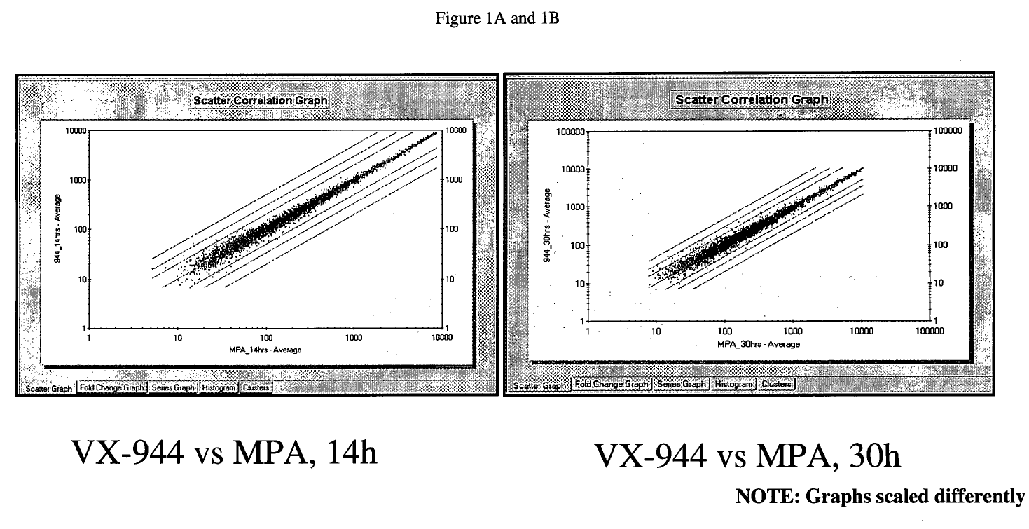

Mycophenolate mofetil, an approved immunosuppressive agent (CellCept® from Roche) used for the prevention of kidney, heart or liver transplant rejections is the 2-morpholinoethyl ester of mycophenolic acid (MPA). VX-944 is currently being developed for the treatment of hematological malignancies. Both MPA and VX-944 are selective inhibitors of IMPDH with VX-944 being 3-4 fold more potent in cell assays (see FIG. 1).

| IC50 values in uM | VX-944 | MPA | VX-497 |
|---|---|---|---|
| PBMCs PHA-T cell IC50 | 0.021 | 0.089 | 0.105 |
| PHA-T cell IC50 in the presence of | 7.8 | >20 | 6.5 |
| guanosine | | | |
| Ratio of PHA-T cell IC50 in the | 373 | >224 | 62 |
| presence and absence of guanosine | | | |

In order to arrive at the conclusion that the expression of certain nucleic acids is modulated in response to IMPDH inhibition, the following inhibitors were employed:

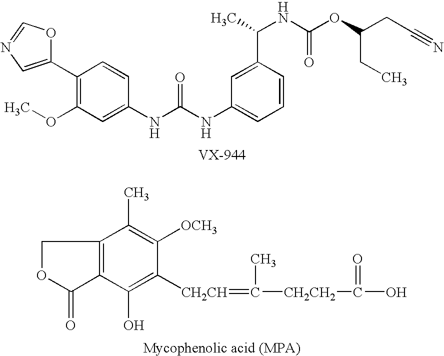

The above agents were tested in HL-60 cells at a concentration of 500 nM for VX-944 and 1000 nM for MPA or with 0.1% DMSO as the vehicle control. The cells were harvested at 14 or 30 hours post addition of compound or vehicle. Briefly, the culture medium was removed at the appropriate time-points and the cells washed with PBS. The cells were then harvested, and pelleted by-gentle centrifugation. The supernatant was discarded and the pellets snap-frozen on an ethanol/dry-ice slurry. RNA was isolated from cells by using a RNEasy kit from Qiagen. RNAs were quality controlled using the Agilent 2100 Bioanalyzer to determine the presence of RNAs.

The results from the Affymetrix array analyses are summarized in the Table below. Data from the arrays corresponding to VX-944 and MPA at the two time-points—14 hours (Early) and 30 hours (Late) were analyzed using Spotfire™, microarray data analysis software. Some of the data analysis parameters used are outlined below:

a) All the arrays were normalized by using trimmed means, with a 5% exclusion limit

b) Only genes present in at least 5 of the 6 chips used for the analyses were included in the genesets

c) Only statistically significant genes (ANOVA, p<0.05) were included in the final analyses to generate gene lists.

| | Number of Genes | | |
|---|---|---|---|
| | Upregulated | Downregulated | Total |
| VX-944: treated vs. DMSO (early) | 172 | 119 | 291 |
| VX-944: treated vs. DMSO (late) | 434 | 0 | 434 |
| MPA: treated vs. DMSO (early) | 105 | 0 | 105 |
| MPA: treated vs. DMSO (late) | 602 | 560 | 1162 |
| VX-944 (early) vs. MPA (early) | 77 | 0 | 77 |
| VX-944 (late) vs. MPA (late) | 335 | 0 | 335 |
| VX-944 (early) vs. VX-944 (late) | 102 | 0 | 102 |
| MPA (early) vs. MPA (late) | 66 | 0 | 66 |
| VX-944 (Early and Late) vs MPA | 43 | 0 | 43 |
| (early and late) | | | |

A global view of datasets identified by comparing VX-944 and MPA at the early (14 h) time-point showed that there were 77 genes in common between VX-944 and MPA. At the late (30 h) time-point, 335 genes were identified as overlapping between the VX-944 and MPA datasets. A global view of datasets identified by comparing VX-944 and MPA at the two time-points, 14 h (EARLY) and 30 h (LATE) showed that with VX-944 102 genes modulated at the early time-point were also modulated at the late time-point. With MPA 66 genes are in common between the two time-points. As to a comparison between the two data sets, of the 102 genes modulated by VX-944 and the 66 genes modulated by MPA, 43 genes modulated at 14 hours were also modulated at 30 hours. To add additional value to the gene lists and provide additional information to the investigator, the genes underlying each of these expression patterns was queried against publicly available databases. These databases included Locus Link, Gene Cards, KEGG, GO annotations, OMIM, PubMed. The information gathered from such an analysis is organized below in data tables.

VX-944 (Early) vs. MPA (Early): 77 genes were found to be common between these two comparisons. These genes were further assigned to the following functional categories:

| CATEGORY | NUMBER of Genes |
|---|---|
| nucleobase, nucleoside, nucleotide | 13 |
| and nucleic acid metabolism | |
| protein metabolism | 10 |
| response to biotic stimulus | 10 |
| biosynthesis | 9 |
| signal transduction | 9 |
| cell proliferation | 8 |
| lipid metabolism | 8 |
| response to pest/pathogen/parasite | 7 |
| response to wounding | 7 |
| organic acid metabolism | 6 |
| cell adhesion | 5 |
| organogenesis | 5 |
| catabolism | 4 |
| cell-cell signaling | 4 |
| electron transport | 4 |
| carbohydrate metabolism | 3 |
| cell death | 3 |
| cell organization and biogenesis | 3 |
| energy pathways | 3 |
| phosphorus metabolism | 3 |
| programmed cell death | 3 |
| transport | 3 |
| unclassified | 30 |

Interestingly, for this dataset a large number of genes were classified into the Nucleotide metabolism functional category. Some of the other major categories include protein metabolism, response to biotic stimulus, biosynthesis, signal transduction, cell proliferation, lipid metabolism and response to pest/pathogen/parasite. However, 30/77 genes could not be readily classified using the GO functional categorization. Even with these genes, every effort has been made to provide as much information as possible.

The genes in some of these categories are shown below:

### Nucleotide Metabolism

| | LOCUS | | |
|---|---|---|---|
| AFFX_ID | LINK | GENE NAME | CLASSIFICATION |
| 205042_at | 10020 | UDP-N-acetylglucosamine-2-epimerase/N- | alcohol metabolism |
| | | acetylmannosamine kinase | |
| 201612_at | 223 | aldehyde dehydrogenase 9 family, member A1 | aldehyde metabolism |
| 205042_at | 10020 | UDP-N-acetylglucosamine-2-epimerase/N- | amine metabolism |
| | | acetylmannosamine kinase | |
| 210942_s_at | 10402 | alpha2,3-sialyltransferase | amine metabolism |
| 205042_at | 10020 | UDP-N-acetylglucosamine-2-epimerase/N- | biosynthesis |
| | | acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating protein | biosynthesis |
| 202912_at | 133 | adrenomedullin | biosynthesis |
| 204044_at | 23475 | quinolinate phosphoribosyltransferase (nicotinate- | biosynthesis |
| | | nucleotide pyrophosphorylase (carboxylating)) | |
| 208771_s_at | 4048 | leukotriene A4 hydrolase | biosynthesis |
| 205042_at | 10020 | UDP-N-acetylglucosamine-2-epimerase/N- | carbohydrate metabolism |
| | | acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2-epimerase/N- | catabolism |
| | | acetylmannosamine kinase | |
| 208771_s_at | 4048 | leukotriene A4 hydrolase | catabolism |
| 212190_at | 5270 | serine (or cysteine) proteinase inhibitor, clade E | catabolism |
| | | (nexin, plasminogen activator inhibitor type 1), | |
| | | member 2 | |
| 218875_s_at | 26271 | F-box only protein 5 | catabolism |
| 205042_at | 10020 | UDP-N-acetylglucosamine-2-epimerase/N- | cell adhesion |
| | | acetylmannosamine kinase | |
| 211506_s_at | 3576 | interleukin 8 | cell adhesion |
| 203336_s_at | 9270 | integrin cytoplasmic domain-associated protein 1 | cell adhesion |
| 216250_s_at | 9404 | leupaxin | cell adhesion |
| 219890_at | 23601 | C-type (calcium dependent, carbohydrate- | cell adhesion |
| | | recognition domain) lectin, superfamily member 5 | |
| 214727_at | 675 | breast cancer 2, early onset | cell death |
| 201710_at | 4605 | v-myb myeloblastosis viral oncogene homolog | cell death |
| | | (avian)-like 2 | |
| 204614_at | 5055 | serine (or cysteine) proteinase inhibitor, clade B | cell death |
| | | (ovalbumin), member 2 | |
| 212190_at | 5270 | serine (or cysteine) proteinase inhibitor, clade E | cell migration |
| | | (nexin, plasminogen activator inhibitor type 1), | |
| | | member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1-like 1 | cell organization and biogenesis |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene homolog | cell proliferation |
| | | (avian)-like 2 | |
| 200660_at | 6282 | S100 calcium binding protein A11 (calgizzarin) | cell proliferation |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase (nicotinate- | cell-cell signaling |
| | | nucleotide pyrophosphorylase (carboxylating)) | |
| 204044_at | 23475 | quinolinate phosphoribosyltransferase (nicotinate- | coenzymes and prosthetic group |
| | | nucleotide pyrophosphorylase (carboxylating)) | metabolism |
| 203923_s_at | 1536 | cytochrome b-245, beta polypeptide (chronic | electron transport |
| | | granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2-epimerase/N- | energy pathways |
| | | acetylmannosamine kinase | |
| 203923_s_at | 1536 | cytochrome b-245, beta polypeptide (chronic | energy pathways |
| | | granulomatous disease) | |
| 204044_at | 23475 | quinolinate phosphoribosyltransferase (nicotinate- | energy pathways |
| | | nucleotide pyrophosphorylase (carboxylating)) | |
| 202912_at | 133 | adrenomedullin | hormone metabolism |
| 205042_at | 10020 | UDP-N-acetylglucosamine-2-epimerase/N- | lipid metabolism |
| | | acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating protein | lipid metabolism |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, nucleotide |
| | | | and nucleic acid metabolism |
| 205042_at | 10020 | UDP-N-acetylglucosamine-2-epimerase/N- | nucleobase, nucleoside, nucleotide |
| | | acetylmannosamine kinase | and nucleic acid metabolism |
| 218258_at | 51082 | polymerase (RNA) I polypeptide D, 16 kDa | nucleobase, nucleoside, nucleotide |
| | | | and nucleic acid metabolism |
| 205053_at | 5557 | primase, polypeptide 1, 49 kDa | nucleobase, nucleoside, nucleotide |
| | | | and nucleic acid metabolism |
| 203409_at | 1643 | damage-specific DNA binding protein 2, 48 kDa | nucleobase, nucleoside, nucleotide |
| | | | and nucleic acid metabolism |
| 214727_at | 675 | breast cancer 2, early onset | nucleobase, nucleoside, nucleotide |
| | | | and nucleic acid metabolism |
| 209773_s_at | 6241 | ribonucleotide reductase M2 polypeptide | nucleobase, nucleoside, nucleotide |
| | | | and nucleic acid metabolism |
| 202678_at | 2958 | general transcription factor IIA, 2, 12 kDa | nucleobase, nucleoside, nucleotide |
| | | | and nucleic acid metabolism |
| 201710_at | 4605 | v-myb myeloblastosis viral oncogene homolog | nucleobase, nucleoside, nucleotide |
| | | (avian)-like 2 | and nucleic acid metabolism |
| 202912_at | 133 | adrenomedullin | nucleobase, nucleoside, nucleotide |
| | | | and nucleic acid metabolism |
| 200660_at | 6282 | S100 calcium binding protein A11 (calgizzarin) | nucleobase, nucleoside, nucleotide |
| | | | and nucleic acid metabolism |
| 209930_s_at | 4778 | nuclear factor (erythroid-derived 2), 45 kDa | nucleobase, nucleoside, nucleotide |
| | | | and nucleic acid metabolism |
| 201989_s_at | 1389 | cAMP responsive element binding protein-like 2 | nucleobase, nucleoside, nucleotide |
| | | | and nucleic acid metabolism |

### Protein Metabolism

| | LOCUS | | |
|---|---|---|---|
| AFFX_ID | LINK | GENE NAME | CLASSIFICATION |
| 205042_at | 10020 | UDP-N-acetylglucosamine-2-epimerase/N- | alcohol metabolism |
| | | acetylmannosamine kinase | |
| 201612_at | 223 | aldehyde dehydrogenase 9 family, member | aldehyde metabolism |
| | | A1 | |
| 205042_at | 10020 | UDP-N-acetylglucosamine-2-epimerase/N- | amine metabolism |
| | | acetylmannosamine kinase | |
| 210942_s_at | 10402 | alpha2,3-sialyltransferase | amine metabolism |
| 205042_at | 10020 | UDP-N-acetylglucosamine-2-epimerase/N- | biosynthesis |
| | | acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |
| 205042_at | 10020 | UDP-N-acetylglucosamine-2-epimerase/N- | carbohydrate metabolism |
| | | acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2-epimerase/N- | catabolism |
| | | acetylmannosamine kinase | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 218875_s_at | 26271 | F-box only protein 5 | catabolism |
| 205042_at | 10020 | UDP-N-acetylglucosamine-2-epimerase/N- | cell adhesion |
| | | acetylmannosamine kinase | |
| 211506_s_at | 3576 | interleukin 8 | cell adhesion |
| 203336_s_at | 9270 | integrin cytoplasmic domain-associated | cell adhesion |
| | | protein 1 | |
| 216250_s_at | 9404 | leupaxin | cell adhesion |
| 219890_at | 23601 | C-type (calcium dependent, carbohydrate- | cell adhesion |
| | | recognition domain) lectin, superfamily | |
| | | member 5 | |
| 214727_at | 675 | breast cancer 2, early onset | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1- | cell organization and biogenesis |
| | | like 1 | |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase | cell-cell signaling |
| | | (nicotinate-nucleotide pyrophosphorylase | |
| | | (carboxylating)) | |
| 204044_at | 23475 | quinolinate phosphoribosyltransferase | coenzymes and prosthetic |
| | | (nicotinate-nucleotide pyrophosphorylase | group metabolism |
| | | (carboxylating)) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2-epimerase/N- | energy pathways |
| | | acetylmannosamine kinase | |
| 203923_s_at | 1536 | cytochrome b-245, beta polypeptide | energy pathways |
| | | (chronic granulomatous disease) | |
| 204044_at | 23475 | quinolinate phosphoribosyltransferase | energy pathways |
| | | (nicotinate-nucleotide pyrophosphorylase | |
| | | (carboxylating)) | |
| 202912_at | 133 | adrenomedullin | hormone metabolism |
| 205042_at | 10020 | UDP-N-acetylglucosamine-2-epimerase/N- | lipid metabolism |
| | | acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, |
| | | | nucleotide and nucleic acid |
| | | | metabolism |
| 205042_at | 10020 | UDP-N-acetylglucosamine-2-epimerase/N- | nucleobase, nucleoside, |
| | | acetylmannosamine kinase | nucleotide and nucleic acid |
| | | | metabolism |
| 218258_at | 51082 | polymerase (RNA) I polypeptide D, 16 kDa | nucleobase, nucleoside, |
| | | | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 202678_at | 2958 | general transcription factor IIA, 2, 12 kDa | nucleobase, nucleoside, |
| | | | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 202912_at | 133 | adrenomedullin | nucleobase, nucleoside, |
| | | | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), 45 kDa | nucleobase, nucleoside, |
| | | | nucleotide and nucleic acid |
| | | | metabolism |
| 201989_s_at | 1389 | cAMP responsive element binding protein- | nucleobase, nucleoside, |
| | | like 2 | nucleotide and nucleic acid |
| | | | metabolism |
| 205042_at | 10020 | UDP-N-acetylglucosamine-2-epimerase/N- | organic acid metabolism |
| | | acetylmannosamine kinase | |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 205807_s_at | 7286 | tuftelin 1 | ossification |
| 202437_s_at | 1545 | cytochrome P450, family 1, subfamily B, | perception of external stimulus |
| | | polypeptide 1 | |
| 202933_s_at | 7525 | v-yes-1 Yamaguchi sarcoma viral | phosphorus metabolism |
| | | oncogene homolog 1 | |
| 209457_at | 1847 | dual specificity phosphatase 5 | phosphorus metabolism |
| 209585_s_at | 9562 | multiple inositol polyphosphate histidine | phosphorus metabolism |
| | | phosphatase, 1 | |
| 214727_at | 675 | breast cancer 2, early onset | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 205042_at | 10020 | UDP-N-acetylglucosamine-2-epimerase/N- | protein metabolism |
| | | acetylmannosamine kinase | |
| 202933_s_at | 7525 | v-yes-1 Yamaguchi sarcoma viral oncogene | protein metabolism |
| | | homolog 1 | |
| 209457_at | 1847 | dual specificity phosphatase 5 | protein metabolism |
| 210942_s_at | 10402 | alpha2,3-sialyltransferase | protein metabolism |
| 219649_at | 29929 | dolichyl-P-Glc:Man9GlcNAc2-PP- | protein metabolism |
| | | dolichylglucosyltransferase | |
| 200962_at | 6160 | ribosomal protein L31 | protein metabolism |
| 216250_s_at | 9404 | leupaxin | protein metabolism |
| 208771_s_at | 4048 | leukotriene A4 hydrolase | protein metabolism |
| 212190_at | 5270 | serine (or cysteine) proteinase inhibitor, clade E | protein metabolism |
| | | (nexin, plasminogen activator inhibitor type 1), | |
| | | member 2 | |
| 218875_s_at | 26271 | F-box only protein 5 | protein metabolism |

### Response to Biotic Stimulus

| | LOCUS | | |
|---|---|---|---|
| AFFX_ID | LINK | GENE NAME | CLASSIFICATION |
| 205042_at | 10020 | UDP-N-acetylglucosamine-2-epimerase/N- | alcohol metabolism |
| | | acetylmannosamine kinase | |
| 201612_at | 223 | aldehyde dehydrogenase 9 family, member | aldehyde metabolism |
| | | A1 | |
| 205042_at | 10020 | UDP-N-acetylglucosamine-2-epimerase/N- | amine metabolism |
| | | acetylmannosamine kinase | |
| 210942_s_at | 10402 | alpha2,3-sialyltransferase | amine metabolism |
| 205042_at | 10020 | UDP-N-acetylglucosamine-2-epimerase/N- | biosynthesis |
| | | acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |
| 205042_at | 10020 | UDP-N-acetylglucosamine-2-epimerase/N- | carbohydrate metabolism |
| | | acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2-epimerase/N- | catabolism |
| | | acetylmannosamine kinase | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 218875_s_at | 26271 | F-box only protein 5 | catabolism |
| 205042_at | 10020 | UDP-N-acetylglucosamine-2-epimerase/N- | cell adhesion |
| | | acetylmannosamine kinase | |
| 211506_s_at | 3576 | interleukin 8 | cell adhesion |
| 203336_s_at | 9270 | integrin cytoplasmic domain-associated | cell adhesion |
| | | protein 1 | |
| 216250_s_at | 9404 | leupaxin | cell adhesion |
| 219890_at | 23601 | C-type (calcium dependent, carbohydrate- | cell adhesion |
| | | recognition domain) lectin, superfamily | |
| | | member 5 | |
| 214727_at | 675 | breast cancer 2, early onset | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1- | cell organization and biogenesis |
| | | like 1 | |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase | cell-cell signaling |
| | | (nicotinate-nucleotide pyrophosphorylase | |
| | | (carboxylating)) | |
| 204044_at | 23475 | quinolinate phosphoribosyltransferase | coenzymes and prosthetic |
| | | (nicotinate-nucleotide pyrophosphorylase | group metabolism |
| | | (carboxylating)) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2-epimerase/N- | energy pathways |
| | | acetylmannosamine kinase | |
| 203923_s_at | 1536 | cytochrome b-245, beta polypeptide | energy pathways |
| | | (chronic granulomatous disease) | |
| 204044_at | 23475 | quinolinate phosphoribosyltransferase | energy pathways |
| | | (nicotinate-nucleotide pyrophosphorylase | |
| | | (carboxylating)) | |
| 202912_at | 133 | adrenomedullin | hormone metabolism |
| 205042_at | 10020 | UDP-N-acetylglucosamine-2-epimerase/N- | lipid metabolism |
| | | acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, |
| | | | nucleotide and nucleic acid |
| | | | metabolism |
| 205042_at | 10020 | UDP-N-acetylglucosamine-2-epimerase/N- | nucleobase, nucleoside, |
| | | acetylmannosamine kinase | nucleotide and nucleic acid |
| | | | metabolism |
| 218258_at | 51082 | polymerase (RNA) I polypeptide D, 16 kDa | nucleobase, nucleoside, |
| | | | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 202678_at | 2958 | general transcription factor IIA, 2, 12 kDa | nucleobase, nucleoside, |
| | | | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 202912_at | 133 | adrenomedullin | nucleobase, nucleoside, |
| | | | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), 45 kDa | nucleobase, nucleoside, |
| | | | nucleotide and nucleic acid |
| | | | metabolism |
| 201989_s_at | 1389 | cAMP responsive element binding protein- | nucleobase, nucleoside, |
| | | like 2 | nucleotide and nucleic acid |
| | | | metabolism |
| 205042_at | 10020 | UDP-N-acetylglucosamine-2-epimerase/N- | organic acid metabolism |
| | | acetylmannosamine kinase | |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 205807_s_at | 7286 | tuftelin 1 | ossification |
| 202437_s_at | 1545 | cytochrome P450, family 1, subfamily B, | perception of external stimulus |
| | | polypeptide 1 | |
| 202933_s_at | 7525 | v-yes-1 Yamaguchi sarcoma viral | phosphorus metabolism |
| | | oncogene homolog 1 | |
| 209457_at | 1847 | dual specificity phosphatase 5 | phosphorus metabolism |
| 209585_s_at | 9562 | multiple inositol polyphosphate histidine | phosphorus metabolism |
| | | phosphatase, 1 | |
| 214727_at | 675 | breast cancer 2, early onset | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 205042_at | 10020 | UDP-N-acetylglucosamine-2-epimerase/N- | protein metabolism |
| | | acetylmannosamine kinase | |
| 202933_s_at | 7525 | v-yes-1 Yamaguchi sarcoma viral | protein metabolism |
| | | oncogene homolog 1 | |
| 209457_at | 1847 | dual specificity phosphatase 5 | protein metabolism |
| 210942_s_at | 10402 | alpha2,3-sialyltransferase | protein metabolism |
| 219649_at | 29929 | dolichyl-P-Glc:Man9GlcNAc2-PP- | protein metabolism |
| | | dolichylglucosyltransferase | |
| 200962_at | 6160 | ribosomal protein L31 | protein metabolism |
| 216250_s_at | 9404 | leupaxin | protein metabolism |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 218875_s_at | 26271 | F-box only protein 5 | protein metabolism |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing protein | response to abiotic stimulus |
| 202437_s_at | 1545 | cytochrome P450, family 1, subfamily B, | response to abiotic stimulus |
| | | polypeptide 1 | |
| 211506_s_at | 3576 | interleukin 8 | response to biotic stimulus |
| 205557_at | 671 | bactericidal/permeability-increasing protein | response to biotic stimulus |
| 203923_s_at | 1536 | cytochrome b-245, beta polypeptide (chronic | response to biotic stimulus |
| | | granulomatous disease) | |
| 204446_s_at | 240 | arachidonate 5-lipoxygenase | response to biotic stimulus |
| 204174_at | 241 | arachidonate 5-lipoxygenase-activating protein | response to biotic stimulus |
| 202912_at | 133 | adrenomedullin | response to biotic stimulus |
| 201641_at | 684 | bone marrow stromal cell antigen 2 | response to biotic stimulus |
| 208771_s_at | 4048 | leukotriene A4 hydrolase | response to biotic stimulus |
| 209575_at | 3588 | interleukin 10 receptor, beta | response to biotic stimulus |
| 216950_s_at | 2209 | Fc fragment of IgG, high affinity Ia, receptor for | response to biotic stimulus |
| | | (CD64) | |

### Biosynthesis

| | LOCUS | | |
|---|---|---|---|
| AFFX_ID | LINK | GENE NAME | CLASSIFICATION |
| 205042_at | 10020 | UDP-N-acetylglucosamine-2-epimerase/N- | alcohol metabolism |
| | | acetylmannosamine kinase | |
| 201612_at | 223 | aldehyde dehydrogenase 9 family, member | aldehyde metabolism |
| | | A1 | |
| 205042_at | 10020 | UDP-N-acetylglucosamine-2-epimerase/N- | amine metabolism |
| | | acetylmannosamine kinase | |
| 210942_s_at | 10402 | alpha2,3-sialyltransferase | amine metabolism |
| 205042_at | 10020 | UDP-N-acetylglucosamine-2-epimerase/N- | biosynthesis |
| | | acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating protein | biosynthesis |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |

### Signal Transduction

| | LOCUS | | |
|---|---|---|---|
| AFFX_ID | LINK | GENE NAME | CLASSIFICATION |
| 205042_at | 10020 | UDP-N-acetylglucosamine-2- | alcohol metabolism |
| | | epimerase/N-acetylmannosamine kinase | |
| 201612_at | 223 | aldehyde dehydrogenase 9 family, | aldehyde metabolism |
| | | member A1 | |
| 205042_at | 10020 | UDP-N-acetylglucosamine-2- | amine metabolism |
| | | epimerase/N-acetylmannosamine kinase | |
| 210942_s_at | 10402 | alpha2,3-sialyltransferase | amine metabolism |
| 205042_at | 10020 | UDP-N-acetylglucosamine-2- | biosynthesis |
| | | epimerase/N-acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |
| 205042_at | 10020 | UDP-N-acetylglucosamine-2- | carbohydrate metabolism |
| | | epimerase/N-acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2- | catabolism |
| | | epimerase/N-acetylmannosamine kinase | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 218875_s_at | 26271 | F-box only protein 5 | catabolism |
| 205042_at | 10020 | UDP-N-acetylglucosamine-2- | cell adhesion |
| | | epimerase/N-acetylmannosamine kinase | |
| 211506_s_at | 3576 | interleukin 8 | cell adhesion |
| 203336_s_at | 9270 | integrin cytoplasmic domain-associated | cell adhesion |
| | | protein 1 | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 214727_at | 675 | breast cancer 2, early onset | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1- | cell organization and biogenesis |
| | | like 1 | |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase | cell-cell signaling |
| | | (nicotinate-nucleotide pyrophosphorylase | |
| | | (carboxylating)) | |
| 204044_at | 23475 | quinolinate phosphoribosyltransferase | coenzymes and prosthetic |
| | | (nicotinate-nucleotide pyrophosphorylase | group metabolism |
| | | (carboxylating)) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2- | energy pathways |
| | | epimerase/N-acetylmannosamine kinase | |
| 203923_s_at | 1536 | cytochrome b-245, beta polypeptide | energy pathways |
| | | (chronic granulomatous disease) | |
| 204044_at | 23475 | quinolinate phosphoribosyltransferase | energy pathways |
| | | (nicotinate-nucleotide pyrophosphorylase | |
| | | (carboxylating)) | |
| 202912_at | 133 | adrenomedullin | hormone metabolism |
| 205042_at | 10020 | UDP-N-acetylglucosamine-2- | lipid metabolism |
| | | epimerase/N-acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, |
| | | | nucleotide and nucleic acid |
| | | | metabolism |
| 205042_at | 10020 | UDP-N-acetylglucosamine-2- | nucleobase, nucleoside, |
| | | epimerase/N-acetylmannosamine kinase | nucleotide and nucleic acid |
| | | | metabolism |
| 218258_at | 51082 | polymerase (RNA) I polypeptide D, | nucleobase, nucleoside, |
| | | 16 kDa | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 202678_at | 2958 | general transcription factor IIA, 2, 12 kDa | nucleobase, nucleoside, |
| | | | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 202912_at | 133 | adrenomedullin | nucleobase, nucleoside, |
| | | | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), | nucleobase, nucleoside, |
| | | 45 kDa | nucleotide and nucleic acid |
| | | | metabolism |
| 201989_s_at | 1389 | cAMP responsive element binding | nucleobase, nucleoside, |
| | | protein-like 2 | nucleotide and nucleic acid |
| | | | metabolism |
| 205042_at | 10020 | UDP-N-acetylglucosamine-2- | organic acid metabolism |
| | | epimerase/N-acetylmannosamine kinase | |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 205807_s_at | 7286 | tuftelin 1 | ossification |
| 202437_s_at | 1545 | cytochrome P450, family 1, subfamily B, | perception of external stimulus |
| | | polypeptide 1 | |
| 202933_s_at | 7525 | v-yes-1 Yamaguchi sarcoma viral | phosphorus metabolism |
| | | oncogene homolog 1 | |
| 209457_at | 1847 | dual specificity phosphatase 5 | phosphorus metabolism |
| 209585_s_at | 9562 | multiple inositol polyphosphate histidine | phosphorus metabolism |
| | | phosphatase, 1 | |
| 214727_at | 675 | breast cancer 2, early onset | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 205042_at | 10020 | UDP-N-acetylglucosamine-2- | protein metabolism |
| | | epimerase/N-acetylmannosamine kinase | |
| 202933_s_at | 7525 | v-yes-1 Yamaguchi sarcoma viral | protein metabolism |
| | | oncogene homolog 1 | |
| 209457_at | 1847 | dual specificity phosphatase 5 | protein metabolism |
| 210942_s_at | 10402 | alpha2,3-sialyltransferase | protein metabolism |
| 219649_at | 29929 | dolichyl-P-Glc:Man9GlcNAc2-PP- | protein metabolism |
| | | dolichylglucosyltransferase | |
| 200962_at | 6160 | ribosomal protein L31 | protein metabolism |
| 216250_s_at | 9404 | leupaxin | protein metabolism |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 218875_s_at | 26271 | F-box only protein 5 | protein metabolism |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing | response to abiotic stimulus |
| | | protein | |
| 202437_s_at | 1545 | cytochrome P450, family 1, subfamily B, | response to abiotic stimulus |
| | | polypeptide 1 | |
| 211506_s_at | 3576 | interleukin 8 | response to biotic stimulus |
| 205557_at | 671 | bactericidal/permeability-increasing | response to biotic stimulus |
| | | protein | |
| 203923_s_at | 1536 | cytochrome b-245, beta polypeptide | response to biotic stimulus |
| | | (chronic granulomatous disease) | |
| 204446_s_at | 240 | arachidonate 5-lipoxygenase | response to biotic stimulus |
| 204174_at | 241 | arachidonate 5-lipoxygenase-activating | response to biotic stimulus |
| | | protein | |
| 202912_at | 133 | adrenomedullin | response to biotic stimulus |
| 201641_at | 684 | bone marrow stromal cell antigen 2 | response to biotic stimulus |
| 208771_s_at | 4048 | leukotriene A4 hydrolase | response to biotic stimulus |
| 209575_at | 3588 | interleukin 10 receptor, beta | response to biotic stimulus |
| 216950_s_at | 2209 | Fc fragment of IgG, high affinity Ia, | response to biotic stimulus |
| | | receptor for (CD64) | |
| 211506_s_at | 3576 | interleukin 8 | response to |
| | | | pest/pathogen/parasite |
| 203923_s_at | 1536 | cytochrome b-245, beta polypeptide | response to |
| | | (chronic granulomatous disease) | pest/pathogen/parasite |
| 204446_s_at | 240 | arachidonate 5-lipoxygenase | response to |
| | | | pest/pathogen/parasite |
| 204174_at | 241 | arachidonate 5-lipoxygenase-activating | response to |
| | | protein | pest/pathogen/parasite |
| 201641_at | 684 | bone marrow stromal cell antigen 2 | response to |
| | | | pest/pathogen/parasite |
| 208771_s_at | 4048 | leukotriene A4 hydrolase | response to |
| | | | pest/pathogen/parasite |
| 209575_at | 3588 | interleukin 10 receptor, beta | response to |
| | | | pest/pathogen/parasite |
| 211506_s_at | 3576 | interleukin 8 | response to wounding |
| 203923_s_at | 1536 | cytochrome b-245, beta polypeptide | response to wounding |
| | | (chronic granulomatous disease) | |
| 204446_s_at | 240 | arachidonate 5-lipoxygenase | response to wounding |
| 204174_at | 241 | arachidonate 5-lipoxygenase-activating | response to wounding |
| | | protein | |
| 202912_at | 133 | adrenomedullin | response to wounding |
| 208771_s_at | 4048 | leukotriene A4 hydrolase | response to wounding |
| 209575_at | 3588 | interleukin 10 receptor, beta | response to wounding |
| 218224_at | 9240 | paraneoplastic antigen MA1 | sexual reproduction |
| 202933_s_at | 7525 | v-yes-1 Yamaguchi sarcoma viral oncogene | signal transduction |
| | | homolog 1 | |
| 211506_s_at | 3576 | interleukin 8 | signal transduction |
| 202912_at | 133 | adrenomedullin | signal transduction |
| 203336_s_at | 9270 | integrin cytoplasmic domain-associated | signal transduction |
| | | protein 1 | |
| 216250_s_at | 9404 | leupaxin | signal transduction |
| 209154_at | 30851 | Tax interaction protein 1 | signal transduction |
| 209575_at | 3588 | interleukin 10 receptor, beta | signal transduction |
| 216950_s_at | 2209 | Fc fragment of IgG, high affinity Ia, receptor | signal transduction |
| | | for (CD64) | |
| 201989_s_at | 1389 | cAMP responsive element binding protein-like 2 | signal transduction |

### Programmed Cell Death

| | LOCUS | | |
|---|---|---|---|
| AFFX_ID | LINK | GENE NAME | CLASSIFICATION |
| 205042_at | 10020 | UDP-N-acetylglucosamine-2- | alcohol metabolism |
| | | epimerase/N-acetylmannosamine kinase | |
| 201612_at | 223 | aldehyde dehydrogenase 9 family, | aldehyde metabolism |
| | | member A1 | |
| 205042_at | 10020 | UDP-N-acetylglucosamine-2- | amine metabolism |
| | | epimerase/N-acetylmannosamine kinase | |
| 210942_s_at | 10402 | alpha2,3-sialyltransferase | amine metabolism |
| 205042_at | 10020 | UDP-N-acetylglucosamine-2- | biosynthesis |
| | | epimerase/N-acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |
| 205042_at | 10020 | UDP-N-acetylglucosamine-2- | carbohydrate metabolism |
| | | epimerase/N-acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2- | catabolism |
| | | epimerase/N-acetylmannosamine kinase | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 218875_s_at | 26271 | F-box only protein 5 | catabolism |
| 205042_at | 10020 | UDP-N-acetylglucosamine-2- | cell adhesion |
| | | epimerase/N-acetylmannosamine kinase | |
| 211506_s_at | 3576 | interleukin 8 | cell adhesion |
| 203336_s_at | 9270 | integrin cytoplasmic domain-associated | cell adhesion |
| | | protein 1 | |
| 216250_s_at | 9404 | leupaxin | cell adhesion |
| 219890_at | 23601 | C-type (calcium dependent, carbohydrate- | cell adhesion |
| | | recognition domain) lectin, superfamily | |
| | | member 5 | |
| 214727_at | 675 | breast cancer 2, early onset | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1- | cell organization and biogenesis |
| | | like 1 | |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase | cell-cell signaling |
| | | (nicotinate-nucleotide pyrophosphorylase | |
| | | (carboxylating)) | |
| 204044_at | 23475 | quinolinate phosphoribosyltransferase | coenzymes and prosthetic |
| | | (nicotinate-nucleotide pyrophosphorylase | group metabolism |
| | | (carboxylating)) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2- | energy pathways |
| | | epimerase/N-acetylmannosamine kinase | |
| 203923_s_at | 1536 | cytochrome b-245, beta polypeptide | energy pathways |
| | | (chronic granulomatous disease) | |
| 204044_at | 23475 | quinolinate phosphoribosyltransferase | energy pathways |
| | | (nicotinate-nucleotide pyrophosphorylase | |
| | | (carboxylating)) | |
| 202912_at | 133 | adrenomedullin | hormone metabolism |
| 205042_at | 10020 | UDP-N-acetylglucosamine-2- | lipid metabolism |
| | | epimerase/N-acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, |
| | | | nucleotide and nucleic acid |
| | | | metabolism |
| 205042_at | 10020 | UDP-N-acetylglucosamine-2- | nucleobase, nucleoside, |
| | | epimerase/N-acetylmannosamine kinase | nucleotide and nucleic acid |
| | | | metabolism |
| 218258_at | 51082 | polymerase (RNA) I polypeptide D, 16 kDa | nucleobase, nucleoside, |
| | | | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 202678_at | 2958 | general transcription factor IIA, 2, 12 kDa | nucleobase, nucleoside, |
| | | | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 202912_at | 133 | adrenomedullin | nucleobase, nucleoside, |
| | | | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), | nucleobase, nucleoside, |
| | | 45 kDa | nucleotide and nucleic acid |
| | | | metabolism |
| 201989_s_at | 1389 | cAMP responsive element binding | nucleobase, nucleoside, |
| | | protein-like 2 | nucleotide and nucleic acid |
| | | | metabolism |
| 205042_at | 10020 | UDP-N-acetylglucosamine-2- | organic acid metabolism |
| | | epimerase/N-acetylmannosamine kinase | |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 205807_s_at | 7286 | tuftelin 1 | ossification |
| 202437_s_at | 1545 | cytochrome P450, family 1, subfamily B, | perception of external stimulus |
| | | polypeptide 1 | |
| 202933_s_at | 7525 | v-yes-1 Yamaguchi sarcoma viral | phosphorus metabolism |
| | | oncogene homolog 1 | |
| 209457_at | 1847 | dual specificity phosphatase 5 | phosphorus metabolism |
| 209585_s_at | 9562 | multiple inositol polyphosphate histidine | phosphorus metabolism |
| | | phosphatase, 1 | |
| 214727_at | 675 | breast cancer 2, early onset | programmed cell death |
| 201710_at | 4605 | v-myb myeloblastosis viral oncogene homolog | programmed cell death |
| | | (avian)-like 2 | |
| 204614_at | 5055 | serine (or cysteine) proteinase inhibitor, clade | programmed cell death |
| | | B (ovalbumin), member 2 | |

### Pathogen Response

| | LOCUS | | |
|---|---|---|---|
| AFFX_ID | LINK | GENE NAME | CLASSIFICATION |
| 205042_at | 10020 | UDP-N-acetylglucosamine-2- | alcohol metabolism |
| | | epimerase/N-acetylmannosamine kinase | |
| 201612_at | 223 | aldehyde dehydrogenase 9 family, | aldehyde metabolism |
| | | member A1 | |
| 205042_at | 10020 | UDP-N-acetylglucosamine-2- | amine metabolism |
| | | epimerase/N-acetylmannosamine kinase | |
| 210942_s_at | 10402 | alpha2,3-sialyltransferase | amine metabolism |
| 205042_at | 10020 | UDP-N-acetylglucosamine-2- | biosynthesis |
| | | epimerase/N-acetylmannosamine kinase | |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202912_at | 133 | adrenomedullin | biosynthesis |
| 204044_at | 23475 | quinolinate phosphoribosyltransferase | biosynthesis |
| | | (nicotinate-nucleotide pyrophosphorylase | |
| | | (carboxylating)) | |
| 208771_s_at | 4048 | leukotriene A4 hydrolase | biosynthesis |
| 205042_at | 10020 | UDP-N-acetylglucosamine-2- | carbohydrate metabolism |
| | | epimerase/N-acetylmannosamine kinase | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 205042_at | 10020 | UDP-N-acetylglucosamine-2- | catabolism |
| | | epimerase/N-acetylmannosamine kinase | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 218875_s_at | 26271 | F-box only protein 5 | catabolism |
| 205042_at | 10020 | UDP-N-acetylglucosamine-2- | cell adhesion |
| | | epimerase/N-acetylmannosamine kinase | |
| 211506_s_at | 3576 | interleukin 8 | cell adhesion |
| 203336_s_at | 9270 | integrin cytoplasmic domain-associated | cell adhesion |
| | | protein 1 | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 214727_at | 675 | breast cancer 2, early onset | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201714_at | 7283 | tubulin, gamma 1 | cell organization and biogenesis |
| 201999_s_at | 6993 | t-complex-associated-testis-expressed 1- | cell organization and biogenesis |
| | | like 1 | |
| 1053_at | 5982 | replication factor C (activator 1) 2, 40 kDa | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 201641_at | 684 | bone marrow stromal cell antigen 2 | cell proliferation |
| 211506_s_at | 3576 | interleukin 8 | cell-cell signaling |
| 202912_at | 133 | adrenomedullin | cell-cell signaling |
| 201641_at | 684 | bone marrow stromal cell antigen 2 | cell-cell signaling |
| 204044_at | 23475 | quinolinate phosphoribosyltransferase | cell-cell signaling |
| | | (nicotinate-nucleotide pyrophosphorylase | |
| | | (carboxylating)) | |
| 204044_at | 23475 | quinolinate phosphoribosyltransferase | coenzymes and prosthetic |
| | | (nicotinate-nucleotide pyrophosphorylase | group metabolism |
| | | (carboxylating)) | |
| 203923_s_at | 1536 | cytochrome b-245, beta polypeptide | electron transport |
| | | (chronic granulomatous disease) | |
| 204446_s_a | 240 | arachidonate 5-lipoxygenase | electron transport |
| 202437_s_at | 1545 | cytochrome P450, family 1, subfamily B, | electron transport |
| | | polypeptide 1 | |
| 206662_at | 2745 | glutaredoxin (thioltransferase) | electron transport |
| 205042_at | 10020 | UDP-N-acetylglucosamine-2- | energy pathways |
| | | epimerase/N-acetylmannosamine kinase | |
| 203923_s_at | 1536 | cytochrome b-245, beta polypeptide | energy pathways |
| | | (chronic granulomatous disease) | |
| 204044_at | 23475 | quinolinate phosphoribosyltransferase | energy pathways |
| | | (nicotinate-nucleotide pyrophosphorylase | |
| | | (carboxylating)) | |
| 202912_at | 133 | adrenomedullin | hormone metabolism |
| 205042_at | 10020 | UDP-N-acetylglucosamine-2- | lipid metabolism |
| | | epimerase/N-acetylmannosamine kinase | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 202912_at | 133 | adrenomedullin | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 1053_at | 5982 | replication factor C (activator 1) 2, 40 kDa | nucleobase, nucleoside, |
| | | | nucleotide and nucleic acid |
| | | | metabolism |
| 205042_at | 10020 | UDP-N-acetylglucosamine-2- | nucleobase, nucleoside, |
| | | epimerase/N-acetylmannosamine kinase | nucleotide and nucleic acid |
| | | | metabolism |
| 218258_at | 51082 | polymerase (RNA) I polypeptide D, | nucleobase, nucleoside, |
| | | 16 kDa | nucleotide and nucleic acid |
| | | | metabolism |
| 205053_at | 5557 | primase, polypeptide 1, 49 kDa | nucleobase, nucleoside,. |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside,. |
| | | | nucleotide and nucleic acid |
| | | | metabolism |
| 202678_at | 2958 | general transcription factor IIA, 2, 12 kDa | nucleobase, nucleoside, |
| | | | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 202912_at | 133 | adrenomedullin | nucleobase, nucleoside, |
| | | | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), | nucleobase, nucleoside,. |
| | | 45 kDa | nucleotide and nucleic acid |
| | | | metabolism |
| 201989_s_at | 1389 | cAMP responsive element binding | nucleobase, nucleoside, |
| | | protein-like 2 | nucleotide and nucleic acid |
| | | | metabolism |
| 205042_at | 10020 | UDP-N-acetylglucosamine-2- | organic acid metabolism |
| | | epimerase/N-acetylmannosamine kinase | |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 205807_s_at | 7286 | tuftelin 1 | ossification |
| 202437_s_at | 1545 | cytochrome P450, family 1, subfamily B, | perception of external stimulus |
| | | polypeptide 1 | |
| 202933_s_at | 7525 | v-yes-1 Yamaguchi sarcoma viral | phosphorus metabolism |
| | | oncogene homolog 1 | |
| 209457_at | 1847 | dual specificity phosphatase 5 | phosphorus metabolism |
| 209585_s_at | 9562 | multiple inositol polyphosphate histidine | phosphorus metabolism |
| | | phosphatase, 1 | |
| 214727_at | 675 | breast cancer 2, early onset | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 205042_at | 10020 | UDP-N-acetylglucosamine-2- | protein metabolism |
| | | epimerase/N-acetylmannosamine kinase | |
| 202933_s_at | 7525 | v-yes-1 Yamaguchi sarcoma viral | protein metabolism |
| | | oncogene homolog 1 | |
| 209457_at | 1847 | dual specificity phosphatase 5 | protein metabolism |
| 210942_s_at | 10402 | alpha2,3-sialyltransferase | protein metabolism |
| 219649_at | 29929 | dolichyl-P-Glc:Man9GlcNAc2-PP- | protein metabolism |
| | | dolichylglucosyltransferase | |
| 200962_at | 6160 | ribosomal protein L31 | protein metabolism |
| 216250_s_at | 9404 | leupaxin | protein metabolism |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 218875_s_at | 26271 | F-box only protein 5 | protein metabolism |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing | response to abiotic stimulus |
| | | protein | |
| 202437_s_at | 1545 | cytochrome P450, family 1, subfamily B, | response to abiotic stimulus |
| | | polypeptide 1 | |
| 211506_s_at | 3576 | interleukin 8 | response to biotic stimulus |
| 205557_at | 671 | bactericidal/permeability-increasing | response to biotic stimulus |
| | | protein | |
| 203923_s_at | 1536 | cytochrome b-245, beta polypeptide | response to biotic stimulus |
| | | (chronic granulomatous disease) | |
| 204446_s_at | 240 | arachidonate 5-lipoxygenase | response to biotic stimulus |
| 204174_at | 241 | arachidonate 5-lipoxygenase-activating | response to biotic stimulus |
| | | protein | |
| 202912_at | 133 | adrenomedullin | response to biotic stimulus |
| 201641_at | 684 | bone marrow stromal cell antigen 2 | response to biotic stimulus |
| 208771_s_at | 4048 | leukotriene A4 hydrolase | response to biotic stimulus |
| 209575_at | 3588 | interleukin 10 receptor, beta | response to biotic stimulus |
| 216950_s_at | 2209 | Fc fragment of IgG, high affinity Ia, | response to biotic stimulus |
| | | receptor for (CD64) | |
| 211506_s_at | 3576 | interleukin 8 | response to pest/pathogen/parasite |
| 203923_s_at | 1536 | cytochrome b-245, beta polypeptide (chronic | response to pest/pathogen/parasite |
| | | granulomatous disease) | |
| 204446_s_at | 240 | arachidonate 5-lipoxygenase | response to pest/pathogen/parasite |
| 204174_at | 241 | arachidonate 5-lipoxygenase-activating | response to pest/pathogen/parasite |
| | | protein | |
| 201641_at | 684 | bone marrow stromal cell antigen 2 | response to pest/pathogen/parasite |
| 208771_s_at | 4048 | leukotriene A4 hydrolase | response to pest/pathogen/parasite |
| 209575_at | 3588 | interleukin 10 receptor, beta | response to pest/pathogen/parasite |

VX-944 (Late) vs. MPA (Late): 335 genes were found to be common between these two comparisons. These genes were further assigned to the following functional categories:

| | NUMBER |
|---|---|
| | of Genes |
| Phosphatidylinositol signaling system | 15 |
| Cell cycle | 7 |
| Apoptosis | 6 |
| Inositol phosphatemetabolism | 4 |
| Integrin-mediated celladhesion | 4 |
| Arginine and proline metabolism | 3 |
| Fatty acid metabolism | 3 |
| Purine metabolism | 3 |
| Oxidative phosphorylation | 2 |
| Parkinson's disease | 2 |
| Prostaglandin andleukotriene metabolism | 2 |
| Pyrimidine metabolism | 2 |
| Pyruvate metabolism | 2 |
| Ribosome | 2 |
| Starch and sucrosemetabolism | 2 |
| Urea cycle and metabolism ofamino groups | 2 |
| Glycosylphosphatidylinositol(GPI)-anchor | 2 |
| biosynthesis | |
| ATP synthesis | 1 |
| Androgen and estrogen metabolism | 1 |
| Benzoate degradation viaCoA ligation | 1 |
| Carbon fixation | 1 |
| DNApolymerase | 1 |
| Glutathione metabolism | 1 |
| Glycerolipid metabolism | 1 |
| Glycolysis/Gluconeogenesis | 1 |
| Histidine metabolism | 1 |
| N-Glycandegradation | 1 |
| Nitrogen metabolism | 1 |
| Proteasome | 1 |
| Selenoamino acid metabolism | 1 |
| Sphingoglycolipidmetabolism | 1 |
| Sulfur metabolism | 1 |
| Transcription factors | 1 |
| Tryptophan metabolism | 1 |
| Ubiquinonebiosynthesis | 1 |
| gamma-Hexachlorocyclohexane degradation | 1 |
| unclassified | 277 |

### Phosphatidyl Inositol Signaling System:

| | LOCUS | | |
|---|---|---|---|
| AFFYID | LINK | GENE NAME | PATHWAY |
| 208745_at | 10632 | ATP synthase, H+ transporting, | ATP synthesis |
| | | mitochondrial F0 complex, subunit g | |
| 201413_at | 3295 | hydroxysteroid (17-beta) | Androgen and estrogen |
| | | dehydrogenase 4 | metabolism |
| 200797_s_at | 4170 | myeloid cell leukemia sequence 1 | Apoptosis |
| | | (BCL2-related) | |
| 201502_s_at | 4792 | nuclear factor of kappa light | Apoptosis |
| | | polypeptide gene enhancer in B-cells | |
| | | inhibitor, alpha | |
| 205681_at | 597 | BCL2-related protein A1 | Apoptosis |
| 207113_s_at | 7124 | tumor necrosis factor (TNF | Apoptosis |
| | | superfamily, member 2) | |
| 203508_at | 7133 | tumor necrosis factor receptor | Apoptosis |
| | | superfamily, member 1B | |
| 203984_s_at | 842 | caspase 9, apoptosis-related | Apoptosis |
| | | cysteine protease | |
| 200884_at | 1152 | creatine kinase, brain | Arginine and proline metabolism |
| 206177_s_at | 383 | arginase, liver | Arginine and proline metabolism |
| 203455_s_at | 6303 | spermidine/spermine N1- | Arginine and proline metabolism |
| | | acetyltransferase | |
| 205260_s_at | 97 | acylphosphatase 1, erythrocyte | Benzoate degradation viaCoA |
| | | (common) type | ligation |
| 204059_s_at | 4199 | malic enzyme 1, NADP(+)- | Carbon fixation |
| | | dependent, cytosolic | |
| 205899_at | 8900 | cyclin A1 | Cell cycle |
| 201700_at | 896 | cyclin D3 | Cell cycle |
| 205034_at | 9134 | cyclin E2 | Cell cycle |
| 38158_at | 9700 | extra spindle poles like 1 (S. cerevisiae) | Cell cycle |
| 204695_at | 993 | cell division cycle 25A | Cell cycle |
| 201853_s_at | 994 | cell division cycle 25B | Cell cycle |
| 205167_s_at | 995 | cell division cycle 25C | Cell cycle |
| 205053_at | 5557 | primase, polypeptide 1, 49 kDa | DNApolymerase |
| 202437_s_at | 1545 | cytochrome P450, family 1, | Fatty acid metabolism |
| | | subfamily B, polypeptide 1 | |
| 207275_s_at | 2179 | fatty-acid-Coenzyme A ligase, long- | Fatty acid metabolism |
| | | chain 1 | |
| 201963_at | 2180 | fatty-acid-Coenzyme A ligase, long- | Fatty acid metabolism |
| | | chain 2 | |
| 202554_s_at | 2947 | glutathione S-transferase M3 (brain) | Glutathione metabolism |
| 203385_at | 1606 | diacylglycerol kinase, alpha 80 kDa | Glycerolipid metabolism |
| 205260_s_at | 97 | acylphosphatase 1, erythrocyte | Glycolysis/Gluconeogenesis |
| | | (common) type | |
| 206643_at | 3034 | histidine ammonia-lyase | Histidine metabolism |
| 202794_at | 3628 | inositol polyphosphate-1- | Inositol phosphatemetabolism |
| | | phosphatase | |
| 204552_at | 3631 | inositol polyphosphate-4- | Inositol phosphatemetabolism |
| | | phosphatase, type I, 107 kDa | |
| 201598_s_at | 3636 | inositol polyphosphate phosphatase- | Inositol phosphatemetabolism |
| | | like 1 | |
| 212990_at | 8867 | synaptojanin 1 | Inositol phosphatemetabolism |
| 218807_at | 10451 | vav 3 oncogene | Integrin-mediated celladhesion |
| 207121_s_at | 5597 | mitogen-activated protein kinase 6 | Integrin-mediated celladhesion |
| 200931_s_at | 7414 | vinculin | Integrin-mediated celladhesion |
| 208683_at | 824 | calpain 2, (m/II) large subunit | Integrin-mediated celladhesion |
| 208926_at | 4758 | sialidase 1 (lysosomal sialidase) | N-Glycandegradation |
| 206643_at | 3034 | histidine ammonia-lyase | Nitrogen metabolism |
| 208745_at | 10632 | ATP synthase, H+ transporting, | Oxidative phosphorylation |
| | | mitochondrial F0 complex, subunit g | |
| 206790_s_at | 4707 | NADH dehydrogenase (ubiquinone) | Oxidative phosphorylation |
| | | 1 beta subcomplex, 1, 7 kDa | |
| 202530_at | 1432 | mitogen-activated protein kinase 14 | Parkinson's disease |
| 203984_s_at | 842 | caspase 9, apoptosis-related | Parkinson's disease |
| | | cysteine protease | |
| 204446_s_at | 240 | arachidonate 5-lipoxygenase | Prostaglandin andleukotriene |
| | | | metabolism |
| 208771_s_at | 4048 | leukotriene A4 hydrolase | Prostaglandin andleukotriene |
| | | | metabolism |
| 200882_s_at | 5710 | proteasome (prosome, macropain) | Proteasome |
| | | 26S subunit, non-ATPase, 4 | |
| 209773_s_at | 6241 | ribonucleotide reductase M2 | Purine metabolism |
| | | polypeptide | |
| 209043_at | 9061 | 3′-phosphoadenosine 5′- | Purine metabolism |
| | | phosphosulfate synthase 1 | |
| 209473_at | 953 | ectonucleoside triphosphate | Purine metabolism |
| | | diphosphohydrolase 1 | |
| 209773_s_at | 6241 | ribonucleotide reductase M2 | Pyrimidine metabolism |
| | | polypeptide | |
| 209473_at | 953 | ectonucleoside triphosphate | Pyrimidine metabolism |
| | | diphosphohydrolase 1 | |
| 204059_s_at | 4199 | malic enzyme 1, NADP(+)- | Pyruvate metabolism |
| | | dependent, cytosolic | |
| 205260_s_at | 97 | acylphosphatase 1, erythrocyte | Pyruvate metabolism |
| | | (common) type | |
| 221989_at | 6134 | ribosomal protein L10 | Ribosome |
| 200962_at | 6160 | ribosomal protein L31 | Ribosome |
| 209043_at | 9061 | 3′-phosphoadenosine 5′- | Selenoamino acidmetabolism |
| | | phosphosulfate synthase 1 | |
| 208926_at | 4758 | sialidase 1 (lysosomal sialidase) | Sphingoglycolipidmetabolism |
| 208498_s_at | 276 | amylase, alpha 1A; salivary | Starch and sucrosemetabolism |
| 202990_at | 5836 | phosphorylase, glycogen; liver (Hers | Starch and sucrosemetabolism |
| | | disease, glycogen storage disease | |
| | | type VI) | |
| 209043_at | 9061 | 3′-phosphoadenosine 5′- | Sulfur metabolism |
| | | phosphosulfate synthase 1 | |
| 210053_at | 6877 | TAF5 RNA polymerase II, TATA box | Transcription factors |
| | | binding protein (TBP)-associated | |
| | | factor, 100 kDa | |
| 202437_s_at | 1545 | cytochrome P450, family 1, | Tryptophan metabolism |
| | | subfamily B, polypeptide 1 | |
| 206790_s_at | 4707 | NADH dehydrogenase (ubiquinone) | Ubiquinonebiosynthesis |
| | | 1 beta subcomplex, 1, 7 kDa | |
| 200884_at | 1152 | creatine kinase, brain | Urea cycle and metabolism |
| | | | ofamino groups |
| 206177_s_at | 383 | arginase, liver | Urea cycle and metabolism |
| | | | ofamino groups |
| 205281_s_at | 5277 | phosphatidylinositol glycan, class A | Glycosylphosphatidylinositol(GPI)- |
| | | (paroxysmal nocturnal | anchor biosynthesis |
| | | hemoglobinuria) | |
| 205452_at | 9488 | phosphatidylinositol glycan, class B | Glycosylphosphatidylinositol(GPI)- |
| | | | anchor biosynthesis |
| 209714_s_at | 1033 | cyclin-dependent kinase inhibitor 3 | Phosphatidylinositol signaling |
| | | (CDK2-associated dual specificity | system |
| | | phosphatase) | |
| 221563_at | 11221 | dual specificity phosphatase 10 | Phosphatidylinositol signaling |
| | | | system |
| 203385_at | 1606 | diacylglycerol kinase, alpha 80 kDa | Phosphatidylinositol signaling |
| | | | system |
| 209457_at | 1847 | dual specificity phosphatase 5 | Phosphatidylinositol signaling |
| | | | system |
| 208892_s_at | 1848 | dual specificity phosphatase 6 | Phosphatidylinositol signaling |
| | | | system |
| 202794_at | 3628 | inositol polyphosphate-1- | Phosphatidylinositol signaling |
| | | phosphatase | system |
| 204552_at | 3631 | inositol polyphosphate-4- | Phosphatidylinositol signaling |
| | | phosphatase, type I, 107 kDa | system |
| 201598_s_at | 3636 | inositol polyphosphate phosphatase- | Phosphatidylinositol signaling |
| | | like 1 | system |
| 204852_s_at | 5778 | protein tyrosine phosphatase, non- | Phosphatidylinositol signaling |
| | | receptor type 7 | system |
| 202006_at | 5782 | protein tyrosine phosphatase, non- | Phosphatidylinositol signaling |
| | | receptor type 12 | system |
| 221840_at | 5791 | protein tyrosine phosphatase, | Phosphatidylinositol signaling |
| | | receptor type, E | system |
| 212990_at | 8867 | synaptojanin 1 | Phosphatidylinositol signaling |
| | | | system |
| 204695_at | 993 | cell division cycle 25A | Phosphatidylinositol signaling |
| | | | system |
| 201853_s_at | 994 | cell division cycle 25B | Phosphatidylinositol signaling |
| | | | system |
| 205167_s_at | 995 | cell division cycle 25C | Phosphatidylinositol signaling |
| | | | system |

### Cell Cycle

| | LOCUS | | |
|---|---|---|---|
| AFFYID | LINK | GENE NAME | PATHWAY |
| 208745_at | 10632 | ATP synthase, H+ transporting, | ATP synthesis |
| | | mitochondrial F0 complex, subunit g | |
| 201413_at | 3295 | hydroxysteroid (17-beta) | Androgen and estrogen |
| | | dehydrogenase 4 | metabolism |
| 200797_s_at | 4170 | myeloid cell leukemia sequence 1 | Apoptosis |
| | | (BCL2-related) | |
| 201502_s_at | 4792 | nuclear factor of kappa light | Apoptosis |
| | | polypeptide gene enhancer in B-cells | |
| | | inhibitor, alpha | |
| 205681_at | 597 | BCL2-related protein A1 | Apoptosis |
| 207113_s_at | 7124 | tumor necrosis factor (TNF | Apoptosis |
| | | superfamily, member 2) | |
| 203508_at | 7133 | tumor necrosis factor receptor | Apoptosis |
| | | superfamily, member 1B | |
| 203984_s_at | 842 | caspase 9, apoptosis-related | Apoptosis |
| | | cysteine protease | |
| 200884_at | 1152 | creatine kinase, brain | Arginine and proline metabolism |
| 206177_s_at | 383 | arginase, liver | Arginine and proline metabolism |
| 203455_s_at | 6303 | spermidine/spermine N1- | Arginine and proline metabolism |
| | | acetyltransferase | |
| 205260_s_at | 97 | acylphosphatase 1, erythrocyte | Benzoate degradation viaCoA |
| | | (common) type | ligation |
| 204059_s_at | 4199 | malic enzyme 1, NADP(+)- | Carbon fixation |
| | | dependent, cytosolic | |
| 205899_at | 8900 | cyclin A1 | Cell cycle |
| 201700_at | 896 | cyclin D3 | Cell cycle |
| 205034_at | 9134 | cyclin E2 | Cell cycle |
| 38158_at | 9700 | extra spindle poles like 1 (S. cerevisiae) | Cell cycle |
| 204695_at | 993 | cell division cycle 25A | Cell cycle |
| 201853_s_at | 994 | cell division cycle 25B | Cell cycle |
| 205167_s_at | 995 | cell division cycle 25C | Cell cycle |

### Apoptosis

| | LOCUS | | |
|---|---|---|---|
| AFFYID | LINK | GENE NAME | PATHWAY |
| 208745_at | 10632 | ATP synthase, H+ transporting, | ATP synthesis |
| | | mitochondrial F0 complex, subunit g | |
| 201413_at | 3295 | hydroxysteroid (17-beta) | Androgen and estrogen |
| | | dehydrogenase 4 | metabolism |
| 200797_s_at | 4170 | myeloid cell leukemia sequence 1 | Apoptosis |
| | | (BCL2-related) | |
| 201502_s_at | 4792 | nuclear factor of kappa light polypeptide | Apoptosis |
| | | gene enhancer in B-cells inhibitor, alpha | |
| 205681_at | 597 | BCL2-related protein A1 | Apoptosis |
| 207113_s_at | 7124 | tumor necrosis factor (TNF superfamily, | Apoptosis |
| | | member 2) | |
| 203508_at | 7133 | tumor necrosis factor receptor | Apoptosis |
| | | superfamily, member 1B | |
| 203984_s_at | 842 | caspase 9, apoptosis-related cysteine | Apoptosis |
| | | protease | |

### Inositol Phosphate Metabolism

| | LOCUS | | |
|---|---|---|---|
| AFFYID | LINK | GENE NAME | PATHWAY |
| 208745_at | 10632 | ATP synthase, H+ transporting, | ATP synthesis |
| | | mitochondrial F0 complex, subunit g | |
| 201413_at | 3295 | hydroxysteroid (17-beta) | Androgen and estrogen |
| | | dehydrogenase 4 | metabolism |
| 200797_s_at | 4170 | myeloid cell leukemia sequence 1 | Apoptosis |
| | | (BCL2-related) | |
| 201502_s_at | 4792 | nuclear factor of kappa light | Apoptosis |
| | | polypeptide gene enhancer in B-cells | |
| | | inhibitor, alpha | |
| 205681_at | 597 | BCL2-related protein A1 | Apoptosis |
| 207113_s_at | 7124 | tumor necrosis factor (TNF | Apoptosis |
| | | superfamily, member 2) | |
| 203508_at | 7133 | tumor necrosis factor receptor | Apoptosis |
| | | superfamily, member 1B | |
| 203984_s_at | 842 | caspase 9, apoptosis-related | Apoptosis |
| | | cysteine protease | |
| 200884_at | 1152 | creatine kinase, brain | Arginine and proline metabolism |
| 206177_s_at | 383 | arginase, liver | Arginine and proline metabolism |
| 203455_s_at | 6303 | spermidine/spermine N1- | Arginine and proline metabolism |
| | | acetyltransferase | |
| 205260_s_at | 97 | acylphosphatase 1, erythrocyte | Benzoate degradation viaCoA |
| | | (common) type | ligation |
| 204059_s_at | 4199 | malic enzyme 1, NADP(+)- | Carbon fixation |
| | | dependent, cytosolic | |
| 205899_at | 8900 | cyclin A1 | Cell cycle |
| 201700_at | 896 | cyclin D3 | Cell cycle |
| 205034_at | 9134 | cyclin E2 | Cell cycle |
| 38158_at | 9700 | extra spindle poles like 1 (S. cerevisiae) | Cell cycle |
| 204695_at | 993 | cell division cycle 25A | Cell cycle |
| 201853_s_at | 994 | cell division cycle 25B | Cell cycle |
| 205167_s_at | 995 | cell division cycle 25C | Cell cycle |
| 205053_at | 5557 | primase, polypeptide 1, 49 kDa | DNApolymerase |
| 202437_s_at | 1545 | cytochrome P450, family 1, | Fatty acid metabolism |
| | | subfamily B, polypeptide 1 | |
| 207275_s_at | 2179 | fatty-acid-Coenzyme A ligase, long- | Fatty acid metabolism |
| | | chain 1 | |
| 201963_at | 2180 | fatty-acid-Coenzyme A ligase, long- | Fatty acid metabolism |
| | | chain 2 | |
| 202554_s_at | 2947 | glutathione S-transferase M3 (brain) | Glutathione metabolism |
| 203385_at | 1606 | diacylglycerol kinase, alpha 80 kDa | Glycerolipid metabolism |
| 205260_s_at | 97 | acylphosphatase 1, erythrocyte | Glycolysis/Gluconeogenesis |
| | | (common) type | |
| 206643_at | 3034 | histidine ammonia-lyase | Histidine metabolism |
| 202794_at | 3628 | inositol polyphosphate-1-phosphatase | Inositol phosphatemetabolism |
| 204552_at | 3631 | inositol polyphosphate-4-phosphatase, | Inositol phosphatemetabolism |
| | | type I, 107 kDa | |
| 201598_s_at | 3636 | inositol polyphosphate phosphatase-like 1 | Inositol phosphatemetabolism |
| 212990_at | 8867 | synaptojanin 1 | Inositol phosphatemetabolism |

### Fatty Acid Metabolism

| | LOCUS | | |
|---|---|---|---|
| AFFYID | LINK | GENE NAME | PATHWAY |
| 208745_at | 10632 | ATP synthase, H+ transporting, | ATP synthesis |
| | | mitochondrial F0 complex, subunit g | |
| 201413_at | 3295 | hydroxysteroid (17-beta) | Androgen and estrogen |
| | | dehydrogenase 4 | metabolism |
| 200797_s_at | 4170 | myeloid cell leukemia sequence 1 | Apoptosis |
| | | (BCL2-related) | |
| 201502_s_at | 4792 | nuclear factor of kappa light | Apoptosis |
| | | polypeptide gene enhancer in B-cells | |
| | | inhibitor, alpha | |
| 205681_at | 597 | BCL2-related protein A1 | Apoptosis |
| 207113_s_at | 7124 | tumor necrosis factor (TNF | Apoptosis |
| | | superfamily, member 2) | |
| 203508_at | 7133 | tumor necrosis factor receptor | Apoptosis |
| | | superfamily, member 1B | |
| 203984_s_at | 842 | caspase 9, apoptosis-related | Apoptosis |
| | | cysteine protease | |
| 200884_at | 1152 | creatine kinase, brain | Arginine and proline metabolism |
| 206177_s_at | 383 | arginase, liver | Arginine and proline metabolism |
| 203455_s_at | 6303 | spermidine/spermine N1- | Arginine and proline metabolism |
| | | acetyltransferase | |
| 205260_s_at | 97 | acylphosphatase 1, erythrocyte | Benzoate degradation viaCoA |
| | | (common) type | ligation |
| 204059_s_at | 4199 | malic enzyme 1, NADP(+)- | Carbon fixation |
| | | dependent, cytosolic | |
| 205899_at | 8900 | cyclin A1 | Cell cycle |
| 201700_at | 896 | cyclin D3 | Cell cycle |
| 205034_at | 9134 | cyclin E2 | Cell cycle |
| 38158_at | 9700 | extra spindle poles like 1 (S. cerevisiae) | Cell cycle |
| 204695_at | 993 | cell division cycle 25A | Cell cycle |
| 201853_s_at | 994 | cell division cycle 25B | Cell cycle |
| 205167_s_at | 995 | cell division cycle 25C | Cell cycle |
| 205053_at | 5557 | primase, polypeptide 1, 49 kDa | DNApolymerase |
| 202437_s_at | 1545 | cytochrome P450, family 1, subfamily B, | Fatty acid metabolism |
| | | polypeptide 1 | |
| 207275_s_at | 2179 | fatty-acid-Coenzyme A ligase, long-chain 1 | Fatty acid metabolism |
| 201963_at | 2180 | fatty-acid-Coenzyme A ligase, long-chain 2 | Fatty acid metabolism |

VX-944 (Early) vs. VX-944 (Late): 102 genes found common between these two comparisons were binned into the following functional categories.

### Signal Transduction:

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase receptor | blood coagulation |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, carbohydrate- | cell adhesion |
| | | recognition domain) lectin, superfamily | |
| | | member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 (Drosophila) | cell organization and biogenesis |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 domain | cell proliferation |
| | | of STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |
| 211506_s_at | 3576 | interleukin 8 | cell-cell signaling |
| 209473_at | 953 | ectonucleoside triphosphate | cell-cell signaling |
| | | diphosphohydrolase 1 | |
| 204655_at | 6352 | chemokine (C-C motif) ligand 5 | cell-cell signaling |
| 207339_s_at | 4050 | lymphotoxin beta (TNF superfamily, | cell-cell signaling |
| | | member 3) | |
| 205204_at | 4828 | neuromedin B | cell-cell signaling |
| 218212_s_at | 4338 | molybdenum cofactor synthesis 2 | coenzymes and prosthetic |
| | | | group metabolism |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 202942_at | 2109 | electron-transfer-flavoprotein, beta | electron transport |
| | | polypeptide | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201012_at | 301 | annexin A1 | lipid metabolism |
| 210946_at | 8611 | phosphatidic acid phosphatase type 2A | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 206066_s_at | 5889 | RAD51 homolog C (S. cerevisiae) | nucleobase, nucleoside, |
| | | | nucleotide and nucleic acid |
| | | | metabolism |
| 203358_s_at | 2146 | enhancer of zeste homolog 2 (Drosophila) | nucleobase, nucleoside, |
| | | | nucleotide and nucleic acid |
| | | | metabolism. |
| 205053_at | 5557 | primase, polypeptide 1, 49 kDa | nucleobase, nucleoside, |
| | | | nucleotide and nucleic acid |
| | | | metabolism |
| 203564_at | 2189 | Fanconi anemia, complementation group G | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 206111_at | 6036 | ribonuclease, RNase A family, 2 (liver, | nucleobase, nucleoside, |
| | | eosinophil-derived neurotoxin) | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 202532_s_at | 1719 | dihydrofolate reductase | nucleobase, nucleoside, |
| | | | nucleotide and nucleic acid |
| | | | metabolism |
| 202532_s_at | 1719 | dihydrofolate reductase | one-carbon compound |
| | | | metabolism |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 202532_s_at | 1719 | dihydrofolate reductase | organic acid metabolism |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 202191_s_at | 8522 | growth arrest-specific 7 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 201908_at | 1857 | dishevelled, dsh homolog 3 (Drosophila) | organogenesis |
| 202554_s_at | 2947 | glutathione S-transferase M3 (brain) | organogenesis |
| 205807_s_at | 7286 | tuftelin 1 | ossification |
| 204655_at | 6352 | chemokine (C-C motif) ligand 5 | oxygen and reactive oxygen |
| | | | species metabolism |
| 202554_s_at | 2947 | glutathione S-transferase M3 (brain) | oxygen and reactive oxygen |
| | | | species metabolism |
| 202437_s_at | 1545 | cytochrome P450, family 1, subfamily B, | perception of external stimulus |
| | | polypeptide 1 | |
| 208892_s_at | 1848 | dual specificity phosphatase 6 | phosphorus metabolism |
| 209457_at | 1847 | dual specificity phosphatase 5 | phosphorus metabolism |
| 221563_at | 11221 | dual specificity phosphatase 10 | phosphorus metabolism |
| 205260_s_at | 97 | acylphosphatase 1, erythrocyte (common) | phosphorus metabolism |
| | | type | |
| 221840_at | 5791 | protein tyrosine phosphatase, receptor | phosphorus metabolism |
| | | type, E | |
| 208892_s_at | 1848 | dual specificity phosphatase 6 | programmed cell death |
| 202644_s_at | 7128 | tumor necrosis factor, alpha-induced | programmed cell death |
| | | protein 3 | |
| 214727_at | 675 | breast cancer 2, early onset | programmed cell death |
| 214467_at | 8477 | G protein-coupled receptor 65 | programmed cell death |
| 205681_at | 597 | BCL2-related protein A1 | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 208892_s_at | 1848 | dual specificity phosphatase 6 | protein metabolism |
| 209457_at | 1847 | dual specificity phosphatase 5 | protein metabolism |
| 210942_s_at | 10402 | alpha2,3-sialyltransferase | protein metabolism |
| 221563_at | 11221 | dual specificity phosphatase 10 | protein metabolism |
| 207563_s_at | 8473 | O-linked N-acetylglucosamine (GlcNAc) | protein metabolism |
| | | transferase (UDP-N- | |
| | | acetylglucosamine:polypeptide-N- | |
| | | acetylglucosaminyl transferase) | |
| 200962_at | 6160 | ribosomal protein L31 | protein metabolism |
| 216250_s_at | 9404 | leupaxin | protein metabolism |
| 201069_at | 4313 | matrix metalloproteinase 2 (gelatinase A, | protein metabolism |
| | | 72 kDa gelatinase, 72 kDa type IV | |
| | | collagenase) | |
| 221840_at | 5791 | protein tyrosine phosphatase, receptor | protein metabolism |
| | | type, E | |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing protein | response to abiotic stimulus |
| 207563_s_at | 8473 | O-linked N-acetylglucosamine (GlcNAc) | response to abiotic stimulus |
| | | transferase (UDP-N- | |
| | | acetylglucosamine:polypeptide-N- | |
| | | acetylglucosaminyl transferase) | |
| 202437_s_at | 1545 | cytochrome P450, family 1, subfamily B, | response to abiotic stimulus |
| | | polypeptide 1 | |
| 211506_s_at | 3576 | interleukin 8 | response to biotic stimulus |
| 214467_at | 8477 | G protein-coupled receptor 65 | response to biotic stimulus |
| 205419_at | 1880 | Epstein-Barr virus induced gene 2 | response to biotic stimulus |
| | | (lymphocyte-specific G protein-coupled | |
| | | receptor) | |
| 205557_at | 671 | bactericidal/permeability-increasing protein | response to biotic stimulus |
| 209473_at | 953 | ectonucleoside triphosphate | response to biotic stimulus |
| | | diphosphohydrolase 1 | |
| 203923_s_at | 1536 | cytochrome b-245, beta polypeptide | response to biotic stimulus |
| | | (chronic granulomatous disease) | |
| 204655_at | 6352 | chemokine (C-C motif) ligand 5 | response to biotic stimulus |
| 204446_s_at | 240 | arachidonate 5-lipoxygenase | response to biotic stimulus |
| 204174_at | 241 | arachidonate 5-lipoxygenase-activating | response to biotic stimulus |
| | | protein | |
| 207339_s_at | 4050 | lymphotoxin beta (TNF superfamily, | response to biotic stimulus |
| | | member 3) | |
| 201012_at | 301 | annexin A1 | response to biotic stimulus |
| 208771_s_at | 4048 | leukotriene A4 hydrolase | response to biotic stimulus |
| 209575_at | 3588 | interleukin 10 receptor, beta | response to biotic stimulus |
| 216950_s_at | 2209 | Fc fragment of IgG, high affinity Ia, receptor | response to biotic stimulus |
| | | for (CD64) | |
| 205269_at | 3937 | lymphocyte cytosolic protein 2 (SH2 | response to biotic stimulus |
| | | domain containing leukocyte protein of | |
| | | 76 kDa) | |
| 207563_s_at | 8473 | O-linked N-acetylglucosamine (GlcNAc) | response to extracellular |
| | | transferase (UDP-N- | stimulus |
| | | acetylglucosamine:polypeptide-N- | |
| | | acetylglucosaminyl transferase) | |
| 204655_at | 6352 | chemokine (C-C motif) ligand 5 | response to oxidative stress |
| 211506_s_at | 3576 | interleukin 8 | response to |
| | | | pest/pathogen/parasite |
| 209473_at | 953 | ectonucleoside triphosphate | response to |
| | | diphosphohydrolase 1 | pest/pathogen/parasite |
| 203923_s_at | 1536 | cytochrome b-245, beta polypeptide | response to |
| | | (chronic granulomatous disease) | pest/pathogen/parasite |
| 204655_at | 6352 | chemokine (C-C motif) ligand 5 | response to |
| | | | pest/pathogen/parasite |
| 204446_s_at | 240 | arachidonate 5-lipoxygenase | response to |
| | | | pest/pathogen/parasite |
| 204174_at | 241 | arachidonate 5-lipoxygenase-activating | response to |
| | | protein | pest/pathogen/parasite |
| 201012_at | 301 | annexin A1 | response to |
| | | | pest/pathogen/parasite |
| 208771_s_at | 4048 | leukotriene A4 hydrolase | response to |
| | | | pest/pathogen/parasite |
| 209575_at | 3588 | interleukin 10 receptor, beta | response to |
| | | | pest/pathogen/parasite |
| 211506_s_at | 3576 | interleukin 8 | response to wounding |
| 203923_s_at | 1536 | cytochrome b-245, beta polypeptide | response to wounding |
| | | (chronic granulomatous disease) | |
| 204655_at | 6352 | chemokine (C-C motif) ligand 5 | response to wounding |
| 204446_s_at | 240 | arachidonate 5-lipoxygenase | response to wounding |
| 204174_at | 241 | arachidonate 5-lipoxygenase-activating | response to wounding |
| | | protein | |
| 201012_at | 301 | annexin A1 | response to wounding |
| 208771_s_at | 4048 | leukotriene A4 hydrolase | response to wounding |
| 209575_at | 3588 | interleukin 10 receptor, beta | response to wounding |
| 210946_at | 8611 | phosphatidic acid phosphatase type 2A | sexual reproduction |
| 218224_at | 9240 | paraneoplastic antigen MA1 | sexual reproduction |
| 208892_s_at | 1848 | dual specificity phosphatase 6 | signal transduction |
| 211506_s_at | 3576 | interleukin 8 | signal transduction |
| 214467_at | 8477 | G protein-coupled receptor 65 | signal transduction |
| 205419_at | 1880 | Epstein-Barr virus induced gene 2 (lymphocyte- | signal transduction |
| | | specific G protein-coupled receptor) | |
| 210845_s_at | 5329 | plasminogen activator, urokinase receptor | signal transduction |
| 202207_at | 10123 | ADP-ribosylation factor-like 7 | signal transduction |
| 210840_s_at | 8826 | IQ motif containing GTPase activating protein 1 | signal transduction |
| 202811_at | 10617 | associated molecule with the SH3 domain of | signal transduction |
| | | STAM | |
| 221563_at | 11221 | dual specificity phosphatase 10 | signal transduction |
| 207563_s_at | 8473 | O-linked N-acetylglucosamine (GlcNAc) | signal transduction |
| | | transferase (UDP-N- | |
| | | acetylglucosamine:polypeptide-N- | |
| | | acetylglucosaminyl transferase) | |
| 221830_at | 5911 | RAP2A, member of RAS oncogene family | signal transduction |
| 204655_at | 6352 | chemokine (C-C motif) ligand 5 | signal transduction |
| 217941_s_at | 55914 | erbb2 interacting protein | signal transduction |
| 207339_s_at | 4050 | lymphotoxin beta (TNF superfamily, member 3) | signal transduction |
| 201012_at | 301 | annexin A1 | signal transduction |
| 205176_s_at | 23421 | integrin beta 3 binding protein (beta3-endonexin) | signal transduction |
| 216250_s_at | 9404 | leupaxin | signal transduction |
| 209288_s_at | 10602 | CDC42 effector protein (Rho GTPase binding) 3 | signal transduction |
| 205204_at | 4828 | neuromedin B | signal transduction |
| 201908_at | 1857 | dishevelled, dsh homolog 3 (Drosophila) | signal transduction |
| 209575_at | 3588 | interleukin 10 receptor, beta | signal transduction |
| 216950_s_at | 2209 | Fc fragment of IgG, high affinity Ia, receptor for | signal transduction |
| | | (CD64) | |
| 205269_at | 3937 | lymphocyte cytosolic protein 2 (SH2 domain | signal transduction |
| | | containing leukocyte protein of 76 kDa) | |

### Response to Biotic Stimulus

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase receptor | blood coagulation |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, carbohydrate- | cell adhesion |
| | | recognition domain) lectin, superfamily | |
| | | member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 (Drosophila) | cell organization and biogenesis |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 domain | cell proliferation |
| | | of STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |
| 211506_s_at | 3576 | interleukin 8 | cell-cell signaling |
| 209473_at | 953 | ectonucleoside triphosphate | cell-cell signaling |
| | | diphosphohydrolase 1 | |
| 204655_at | 6352 | chemokine (C-C motif) ligand 5 | cell-cell signaling |
| 207339_s_at | 4050 | lymphotoxin beta (TNF superfamily, | cell-cell signaling |
| | | member 3) | |
| 205204_at | 4828 | neuromedin B | cell-cell signaling |
| 218212_s_at | 4338 | molybdenum cofactor synthesis 2 | coenzymes and prosthetic |
| | | | group metabolism |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 202942_at | 2109 | electron-transfer-flavoprotein, beta | electron transport |
| | | polypeptide | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201012_at | 301 | annexin A1 | lipid metabolism |
| 210946_at | 8611 | phosphatidic acid phosphatase type 2A | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 206066_s_at | 5889 | RAD51 homolog C (S. cerevisiae) | nucleobase, nucleoside, |
| | | | nucleotide and nucleic acid |
| | | | metabolism |
| 203358_s_at | 2146 | enhancer of zeste homolog 2 (Drosophila) | nucleobase, nucleoside, |
| | | | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203564_at | 2189 | Fanconi anemia, complementation group G | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 206111_at | 6036 | ribonuclease, RNase A family, 2 (liver, | nucleobase, nucleoside, |
| | | eosinophil-derived neurotoxin) | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 202532_s_at | 1719 | dihydrofolate reductase | nucleobase, nucleoside, |
| | | | nucleotide and nucleic acid |
| | | | metabolism |
| 202532_s_at | 1719 | dihydrofolate reductase | one-carbon compound |
| | | | metabolism |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 202532_s_at | 1719 | dihydrofolate reductase | organic acid metabolism |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 202191_s_at | 8522 | growth arrest-specific 7 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 201908_at | 1857 | dishevelled, dsh homolog 3 (Drosophila) | organogenesis |
| 202554_s_at | 2947 | glutathione S-transferase M3 (brain) | organogenesis |
| 205807_s_at | 7286 | tuftelin 1 | ossification |
| 204655_at | 6352 | chemokine (C-C motif) ligand 5 | oxygen and reactive oxygen |
| | | | species metabolism |
| 202554_s_at | 2947 | glutathione S-transferase M3 (brain) | oxygen and reactive oxygen |
| | | | species metabolism |
| 202437_s_at | 1545 | cytochrome P450, family 1, subfamily B, | perception of external stimulus |
| | | polypeptide 1 | |
| 208892_s_at | 1848 | dual specificity phosphatase 6 | phosphorus metabolism |
| 209457_at | 1847 | dual specificity phosphatase 5 | phosphorus metabolism |
| 221563_at | 11221 | dual specificity phosphatase 10 | phosphorus metabolism |
| 205260_s_at | 97 | acylphosphatase 1, erythrocyte (common) | phosphorus metabolism |
| | | type | |
| 221840_at | 5791 | protein tyrosine phosphatase, receptor | phosphorus metabolism |
| | | type, E | |
| 208892_s_at | 1848 | dual specificity phosphatase 6 | programmed cell death |
| 202644_s_at | 7128 | tumor necrosis factor, alpha-induced | programmed cell death |
| | | protein 3 | |
| 214727_at | 675 | breast cancer 2, early onset | programmed cell death |
| 214467_at | 8477 | G protein-coupled receptor 65 | programmed cell death |
| 205681_at | 597 | BCL2-related protein A1 | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, | programmed cell death |
| | | clade B (ovalbumin), member 2 | |
| 208892_s_at | 1848 | dual specificity phosphatase 6 | protein metabolism |
| 209457_at | 1847 | dual specificity phosphatase 5 | protein metabolism |
| 210942_s_at | 10402 | alpha2,3-sialyltransferase | protein metabolism |
| 221563_at | 11221 | dual specificity phosphatase 10 | protein metabolism |
| 207563_s_at | 8473 | O-linked N-acetylglucosamine (GlcNAc) | protein metabolism |
| | | transferase (UDP-N- | |
| | | acetylglucosamine:polypeptide-N- | |
| | | acetylglucosaminyl transferase) | |
| 200962_at | 6160 | ribosomal protein L31 | protein metabolism |
| 216250_s_at | 9404 | leupaxin | protein metabolism |
| 201069_at | 4313 | matrix metalloproteinase 2 (gelatinase A, | protein metabolism |
| | | 72 kDa gelatinase, 72 kDa type IV | |
| | | collagenase) | |
| 221840_at | 5791 | protein tyrosine phosphatase, receptor | protein metabolism |
| | | type, E | |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing protein | response to abiotic stimulus |
| 207563_s_at | 8473 | O-linked N-acetylglucosamine (GlcNAc) | response to abiotic stimulus |
| | | transferase (UDP-N- | |
| | | acetylglucosamine:polypeptide-N- | |
| | | acetylglucosaminyl transferase) | |
| 202437_s_at | 1545 | cytochrome P450, family 1, subfamily B, | response to abiotic stimulus |
| | | polypeptide 1 | |
| 211506_s_at | 3576 | interleukin 8 | response to biotic stimulus |
| 214467_at | 8477 | G protein-coupled receptor 65 | response to biotic stimulus |
| 205419_at | 1880 | Epstein-Barr virus induced gene 2 | response to biotic stimulus |
| | | (lymphocyte-specific G protein-coupled | |
| | | receptor) | |
| 205557_at | 671 | bactericidal/permeability-increasing protein | response to biotic stimulus |
| 209473_at | 953 | ectonucleoside triphosphate | response to biotic stimulus |
| | | diphosphohydrolase 1 | |
| 203923_s_at | 1536 | cytochrome b-245, beta polypeptide (chronic | response to biotic stimulus |
| | | granulomatous disease) | |
| 204655_at | 6352 | chemokine (C-C motif) ligand 5 | response to biotic stimulus |
| 204446_s_at | 240 | arachidonate 5-lipoxygenase | response to biotic stimulus |
| 204174_at | 241 | arachidonate 5-lipoxygenase-activating | response to biotic stimulus |
| | | protein | |
| 207339_s_at | 4050 | lymphotoxin beta (TNF superfamily, member | response to biotic stimulus |
| | | 3) | |
| 201012_at | 301 | annexin A1 | response to biotic stimulus |
| 208771_s_at | 4048 | leukotriene A4 hydrolase | response to biotic stimulus |
| 209575_at | 3588 | interleukin 10 receptor, beta | response to biotic stimulus |
| 216950_s_at | 2209 | Fc fragment of IgG, high affinity Ia, receptor | response to biotic stimulus |
| | | for (CD64) | |
| 205269_at | 3937 | lymphocyte cytosolic protein 2 (SH2 domain | response to biotic stimulus |
| | | containing leukocyte protein of 76 kDa) | |

### Cell Proliferation

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase | blood coagulation |
| | | receptor | |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 | cell organization and biogenesis |
| | | (Drosophila) | |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 domain of | cell proliferation |
| | | STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |

### Nucleotide Metabolism

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase | blood coagulation |
| | | receptor | |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 | cell organization and biogenesis |
| | | (Drosophila) | |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 | cell proliferation |
| | | domain of STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |
| 211506_s_at | 3576 | interleukin 8 | cell-cell signaling |
| 209473_at | 953 | ectonucleoside triphosphate | cell-cell signaling |
| | | diphosphohydrolase 1 | |
| 204655_at | 6352 | chemokine (C-C motif) ligand 5 | cell-cell signaling |
| 207339_s_at | 4050 | lymphotoxin beta (TNF superfamily, | cell-cell signaling |
| | | member 3) | |
| 205204_at | 4828 | neuromedin B | cell-cell signaling |
| 218212_s_at | 4338 | molybdenum cofactor synthesis 2 | coenzymes and prosthetic |
| | | | group metabolism |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 202942_at | 2109 | electron-transfer-flavoprotein, beta | electron transport |
| | | polypeptide | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201012_at | 301 | annexin A1 | lipid metabolism |
| 210946_at | 8611 | phosphatidic acid phosphatase type 2A | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 206066_s_at | 5889 | RAD51 homolog C (S. cerevisiae) | nucleobase, nucleoside, nucleotide |
| | | | and nucleic acid metabolism |
| 203358_s_at | 2146 | enhancer of zeste homolog 2 (Drosophila) | nucleobase, nucleoside, nucleotide |
| | | | and nucleic acid metabolism |
| 205053_at | 5557 | primase, polypeptide 1, 49 kDa | nucleobase, nucleoside, nucleotide |
| | | | and nucleic acid metabolism |
| 203564_at | 2189 | Fanconi anemia, complementation group G | nucleobase, nucleoside, nucleotide |
| | | | and nucleic acid metabolism |
| 203409_at | 1643 | damage-specific DNA binding protein 2, | nucleobase, nucleoside, nucleotide |
| | | 48 kDa | and nucleic acid metabolism |
| 214727_at | 675 | breast cancer 2, early onset | nucleobase, nucleoside, nucleotide |
| | | | and nucleic acid metabolism |
| 209773_s_at | 6241 | ribonucleotide reductase M2 polypeptide | nucleobase, nucleoside, nucleotide |
| | | | and nucleic acid metabolism |
| 206111_at | 6036 | ribonuclease, RNase A family, 2 (liver, | nucleobase, nucleoside, nucleotide |
| | | eosinophil-derived neurotoxin) | and nucleic acid metabolism |
| 201710_at | 4605 | v-myb myeloblastosis viral oncogene | nucleobase, nucleoside, nucleotide |
| | | homolog (avian)-like 2 | and nucleic acid metabolism |
| 200660_at | 6282 | S100 calcium binding protein A11 | nucleobase, nucleoside, nucleotide |
| | | (calgizzarin) | and nucleic acid metabolism |
| 202532_s_at | 1719 | dihydrofolate reductase | nucleobase, nucleoside, nucleotide |
| | | | and nucleic acid metabolism |

### Programmed Cell Death

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase | blood coagulation |
| | | receptor | |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 | cell organization and biogenesis |
| | | (Drosophila) | |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 | cell proliferation |
| | | domain of STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |
| 211506_s_at | 3576 | interleukin 8 | cell-cell signaling |
| 209473_at | 953 | ectonucleoside triphosphate | cell-cell signaling |
| | | diphosphohydrolase 1 | |
| 204655_at | 6352 | chemokine (C-C motif) ligand 5 | cell-cell signaling |
| 207339_s_at | 4050 | lymphotoxin beta (TNF superfamily, | cell-cell signaling |
| | | member 3) | |
| 205204_at | 4828 | neuromedin B | cell-cell signaling |
| 218212_s_at | 4338 | molybdenum cofactor synthesis 2 | coenzymes and prosthetic |
| | | | group metabolism |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 202942_at | 2109 | electron-transfer-flavoprotein, beta | electron transport |
| | | polypeptide | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201012_at | 301 | annexin A1 | lipid metabolism |
| 210946_at | 8611 | phosphatidic acid phosphatase type 2A | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |
| 206066_s_at | 5889 | RAD51 homolog C (S. cerevisiae) | nucleobase, nucleoside, |
| | | | nucleotide and nucleic acid |
| | | | metabolism |
| 203358_s_at | 2146 | enhancer of zeste homolog 2 | nucleobase, nucleoside, |
| | | (Drosophila) | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203564_at | 2189 | Fanconi anemia, complementation group G | nucleobase, nucleoside, |
| | | | nucleotide and nucleic acid |
| | | | metabolism |
| 203409_at | 1643 | damage-specific DNA binding protein 2, | nucleobase, nucleoside, |
| | | 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 polypeptide | nucleobase, nucleoside, |
| | | | nucleotide and nucleic acid |
| | | | metabolism |
| 206111_at | 6036 | ribonuclease, RNase A family, 2 (liver, | nucleobase, nucleoside, |
| | | eosinophil-derived neurotoxin) | nucleotide and nucleic acid |
| | | | metabolism |
| 201710_at | 4605 | v-myb myeloblastosis viral oncogene | nucleobase, nucleoside, |
| | | homolog (avian)-like 2 | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 202532_s_at | 1719 | dihydrofolate reductase | nucleobase, nucleoside, |
| | | | nucleotide and nucleic acid |
| | | | metabolism |
| 202532_s_at | 1719 | dihydrofolate reductase | one-carbon compound |
| | | | metabolism |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 202532_s_at | 1719 | dihydrofolate reductase | organic acid metabolism |
| 201963_at | 2180 | fatty-acid-Coenzyme A ligase, long-chain 2 | organic acid metabolism |
| 208771_s_at | 4048 | leukotriene A4 hydrolase | organic acid metabolism |
| 207275_s_at | 2179 | fatty-acid-Coenzyme A ligase, long-chain 1 | organic acid metabolism |
| 206734_at | 8690 | jerky homolog-like (mouse) | organogenesis |
| 211506_s_at | 3576 | interleukin 8 | organogenesis |
| 205807_s_at | 7286 | tuftelin 1 | organogenesis |
| 202191_s_at | 8522 | growth arrest-specific 7 | organogenesis |
| 218224_at | 9240 | paraneoplastic antigen MA1 | organogenesis |
| 202437_s_at | 1545 | cytochrome P450, family 1, subfamily B, | organogenesis |
| | | polypeptide 1 | |
| 201908_at | 1857 | dishevelled, dsh homolog 3 (Drosophila) | organogenesis |
| 202554_s_at | 2947 | glutathione S-transferase M3 (brain) | organogenesis |
| 205807_s_at | 7286 | tuftelin 1 | ossification |
| 204655_at | 6352 | chemokine (C-C motif) ligand 5 | oxygen and reactive oxygen |
| | | | species metabolism |
| 202554_s_at | 2947 | glutathione S-transferase M3 (brain) | oxygen and reactive oxygen |
| | | | species metabolism |
| 202437_s_at | 1545 | cytochrome P450, family 1, subfamily B, | perception of external stimulus |
| | | polypeptide 1 | |
| 208892_s_at | 1848 | dual specificity phosphatase 6 | phosphorus metabolism |
| 209457_at | 1847 | dual specificity phosphatase 5 | phosphorus metabolism |
| 221563_at | 11221 | dual specificity phosphatase 10 | phosphorus metabolism |
| 205260_s_at | 97 | acylphosphatase 1, erythrocyte | phosphorus metabolism |
| | | (common) type | |
| 221840_at | 5791 | protein tyrosine phosphatase, receptor | phosphorus metabolism |
| | | type, E | |
| 208892_s_at | 1848 | dual specificity phosphatase 6 | programmed cell death |
| 202644_s_at | 7128 | tumor necrosis factor, alpha-induced protein 3 | programmed cell death |
| 214727_at | 675 | breast cancer 2, early onset | programmed cell death |
| 214467_at | 8477 | G protein-coupled receptor 65 | programmed cell death |
| 205681_at | 597 | BCL2-related protein A1 | programmed cell death |
| 201710_at | 4605 | v-myb myeloblastosis viral oncogene | programmed cell death |
| | | homolog (avian)-like 2 | |
| 204614_at | 5055 | serine (or cysteine) proteinase inhibitor, clade | programmed cell death |
| | | B (ovalbumin), member 2 | |

### Lipid Metabolism

| | LOCUS | | |
|---|---|---|---|
| AFFX_ID | LINK | GENE NAME | CLASSIFICATION |
| 210942_s_at | 10402 | alpha2,3-sialyltransferase | amine metabolism |
| 202532_s_at | 1719 | dihydrofolate reductase | amine metabolism |
| 202532_s_at | 1719 | dihydrofolate reductase | amino acid and derivative |
| | | | metabolism |
| 218212_s_at | 4338 | molybdenum cofactor synthesis 2 | aromatic compound metabolism |
| 210942_s_at | 10402 | alpha2,3-sialyltransferase | biosynthesis |
| 218212_s_at | 4338 | molybdenum cofactor synthesis 2 | biosynthesis |
| 207563_s_at | 8473 | O-linked N-acetylglucosamine (GlcNAc) | biosynthesis |
| | | transferase (UDP-N- | |
| | | acetylglucosamine:polypeptide-N- | |
| | | acetylglucosaminyl transferase) | |
| 200962_at | 6160 | ribosomal protein L31 | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 202532_s_at | 1719 | dihydrofolate reductase | biosynthesis |
| 208771_s_at | 4048 | leukotriene A4 hydrolase | biosynthesis |
| 210845_s_at | 5329 | plasminogen activator, urokinase | blood coagulation |
| | | receptor | |
| 209473_at | 953 | ectonucleoside triphosphate | blood coagulation |
| | | diphosphohydrolase 1 | |
| 210942_s_at | 10402 | alpha2,3-sialyltransferase | carbohydrate metabolism |
| 202499_s_at | 6515 | solute carrier family 2 (facilitated glucose | carbohydrate metabolism |
| | | transporter), member 3 | |
| 219373_at | 54344 | dolichyl-phosphate mannosyltransferase | carbohydrate metabolism |
| | | polypeptide 3 | |
| 206111_at | 6036 | ribonuclease, RNase A family, 2 (liver, | catabolism |
| | | eosinophil-derived neurotoxin) | |
| 201069_at | 4313 | matrix metalloproteinase 2 (gelatinase A, | catabolism |
| | | 72 kDa gelatinase, 72 kDa type IV | |
| | | collagenase) | |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 209473_at | 953 | ectonucleoside triphosphate | cell adhesion |
| | | diphosphohydrolase 1 | |
| 204655_at | 6352 | chemokine (C-C motif) ligand 5 | cell adhesion |
| 205176_s_at | 23421 | integrin beta 3 binding protein (beta3- | cell adhesion |
| | | endonexin) | |
| 216250_s_at | 9404 | leupaxin | cell adhesion |
| 219890_at | 23601 | C-type (calcium dependent, | cell adhesion |
| | | carbohydrate-recognition domain) lectin, | |
| | | superfamily member 5 | |
| 208892_s_at | 1848 | dual specificity phosphatase 6 | cell death |
| 202644_s_at | 7128 | tumor necrosis factor, alpha-induced | cell death |
| | | protein 3 | |
| 214727_at | 675 | breast cancer 2, early onset | cell death |
| 214467_at | 8477 | G protein-coupled receptor 65 | cell death |
| 205681_at | 597 | BCL2-related protein A1 | cell death |
| 201710_at | 4605 | v-myb myeloblastosis viral oncogene | cell death |
| | | homolog (avian)-like 2 | |
| 204614_at | 5055 | serine (or cysteine) proteinase inhibitor, | cell death |
| | | clade B (ovalbumin), member 2 | |
| 204655_at | 6352 | chemokine (C-C motif) ligand 5 | cell homeostasis |
| 210946_at | 8611 | phosphatidic acid phosphatase type 2A | cell migration |
| 212190_at | 5270 | serine (or cysteine) proteinase inhibitor, | cell migration |
| | | clade E (nexin, plasminogen activator | |
| | | inhibitor type 1), member 2 | |
| 203358_s_at | 2146 | enhancer of zeste homolog 2 | cell organization and biogenesis |
| | | (Drosophila) | |
| 214727_at | 675 | breast cancer 2, early onset | cell organization and biogenesis |
| 201999_s_at | 6993 | t-complex-associated-testis-expressed 1- | cell organization and biogenesis |
| | | like 1 | |
| 205046_at | 1062 | centromere protein E, 312 kDa | cell proliferation |
| 208892_s_at | 1848 | dual specificity phosphatase 6 | cell proliferation |
| 205053_at | 5557 | primase, polypeptide 1, 49 kDa | cell proliferation |
| 203564_at | 2189 | Fanconi anemia, complementation group G | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 202811_at | 10617 | associated molecule with the SH3 | cell proliferation |
| | | domain of STAM | |
| 201710_at | 4605 | v-myb myeloblastosis viral oncogene | cell proliferation |
| | | homolog (avian)-like 2 | |
| 200660_at | 6282 | S100 calcium binding protein A11 | cell proliferation |
| | | (calgizzarin) | |
| 202191_s_at | 8522 | growth arrest-specific 7 | cell proliferation |
| 211506_s_at | 3576 | interleukin 8 | cell-cell signaling |
| 209473_at | 953 | ectonucleoside triphosphate | cell-cell signaling |
| | | diphosphohydrolase 1 | |
| 204655_at | 6352 | chemokine (C-C motif) ligand 5 | cell-cell signaling |
| 207339_s_at | 4050 | lymphotoxin beta (TNF superfamily, | cell-cell signaling |
| | | member 3) | |
| 205204_at | 4828 | neuromedin B | cell-cell signaling |
| 218212_s_at | 4338 | molybdenum cofactor synthesis 2 | coenzymes and prosthetic |
| | | | group metabolism |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily B, | electron transport |
| | | polypeptide 1 | |
| 202942_at | 2109 | electron-transfer-flavoprotein, beta | electron transport |
| | | polypeptide | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201012_at | 301 | annexin A1 | lipid metabolism |
| 210946_at | 8611 | phosphatidic acid phosphatase type 2A | lipid metabolism |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |

MPA (Early) vs. MPA (Late): 66 genes were binned into this category. These genes were further binned into the following functional categories.

### Nucleotide Metabolism

| | LOCUS | | |
|---|---|---|---|
| AFFX_ID | LINK | GENE NAME | CLASSIFICATION |
| 201612_at | 223 | aldehyde dehydrogenase 9 family, | aldehyde metabolism |
| | | member A1 | |
| 210942_s_at | 10402 | alpha2,3-sialyltransferase | amine metabolism |
| 206643_at | 3034 | histidine ammonia-lyase | amine metabolism |
| 206643_at | 3034 | histidine ammonia-lyase | amino acid and derivative |
| | | | metabolism |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 206643_at | 3034 | histidine ammonia-lyase | biosynthesis |
| 204446_s_at | 240 | arachidonate 5-lipoxygenase | biosynthesis |
| 204174_at | 241 | arachidonate 5-lipoxygenase- | biosynthesis |
| | | activating protein | |
| 208771_s_at | 4048 | leukotriene A4 hydrolase | biosynthesis |
| 210942_s_at | 10402 | alpha2,3-sialyltransferase | carbohydrate metabolism |
| 202499_s_at | 6515 | solute carrier family 2 (facilitated | carbohydrate metabolism |
| | | glucose transporter), member 3 | |
| 206643_at | 3034 | histidine ammonia-lyase | catabolism |
| 202551_s_at | 51232 | cysteine-rich motor neuron 1 | catabolism |
| 208771_s_at | 4048 | leukotriene A4 hydrolase | catabolism |
| 212190_at | 5270 | serine (or cysteine) proteinase | catabolism |
| | | inhibitor, clade E (nexin, | |
| | | plasminogen activator inhibitor type | |
| | | 1), member 2 | |
| 211506_s_at | 3576 | interleukin 8 | cell adhesion |
| 207113_s_at | 7124 | tumor necrosis factor (TNF | cell adhesion |
| | | superfamily, member 2) | |
| 201105_at | 3956 | lectin, galactoside-binding, soluble, 1 | cell adhesion |
| | | (galectin 1) | |
| 216250_s_at | 9404 | leupaxin | cell adhesion |
| 219890_at | 23601 | C-type (calcium dependent, | cell adhesion |
| | | carbohydrate-recognition domain) | |
| | | lectin, superfamily member 5 | |
| 214727_at | 675 | breast cancer 2, early onset | cell death |
| 207113_s_at | 7124 | tumor necrosis factor (TNF | cell death |
| | | superfamily, member 2) | |
| 204614_at | 5055 | serine (or cysteine) proteinase | cell death |
| | | inhibitor, clade B (ovalbumin), | |
| | | member 2 | |
| 201105_at | 3956 | lectin, galactoside-binding, soluble, 1 | cell death |
| | | (galectin 1) | |
| 202551_s_at | 51232 | cysteine-rich motor neuron 1 | cell growth |
| 212190_at | 5270 | serine (or cysteine) proteinase | cell migration |
| | | inhibitor, clade E (nexin, | |
| | | plasminogen activator inhibitor type | |
| | | 1), member 2 | |
| 214727_at | 675 | breast cancer 2, early onset | cell organization and biogenesis |
| 201999_s_at | 6993 | t-complex-associated-testis- | cell organization and biogenesis |
| | | expressed 1-like 1 | |
| 204603_at | 9156 | exonuclease 1 | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 | cell proliferation |
| | | polypeptide | |
| 211506_s_at | 3576 | interleukin 8 | cell proliferation |
| 200660_at | 6282 | S100 calcium binding protein A11 | cell proliferation |
| | | (calgizzarin) | |
| 202769_at | 901 | cyclin G2 | cell proliferation |
| 201700_at | 896 | cyclin D3 | cell proliferation |
| 211506_s_at | 3576 | interleukin 8 | cell-cell signaling |
| 207113_s_at | 7124 | tumor necrosis factor (TNF | cell-cell signaling |
| | | superfamily, member 2) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, | electron transport |
| | | subfamily B, polypeptide 1 | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase- | lipid metabolism |
| | | activating protein | |
| 201963_at | 2180 | fatty-acid-Coenzyme A ligase, long- | lipid metabolism |
| | | chain 2 | |
| 208771_s_at | 4048 | leukotriene A4 hydrolase | lipid metabolism |
| 207275_s_at | 2179 | fatty-acid-Coenzyme A ligase, long- | lipid metabolism |
| | | chain 1 | |
| 209906_at | 719 | complement component 3a receptor 1 | muscle contraction |
| 204603_at | 9156 | exonuclease 1 | nucleobase, nucleoside, nucleotide |
| | | | and nucleic acid metabolism |
| 202338_at | 7083 | thymidine kinase 1, soluble | nucleobase, nucleoside, nucleotide |
| | | | and nucleic acid metabolism |
| 218384_at | 23589 | calcium regulated heat stable protein 1, | nucleobase, nucleoside, nucleotide |
| | | 24 kDa | and nucleic acid metabolism |
| 205053_at | 5557 | primase, polypeptide 1, 49 kDa | nucleobase, nucleoside, nucleotide |
| | | | and nucleic acid metabolism |
| 203409_at | 1643 | damage-specific DNA binding protein 2, | nucleobase, nucleoside, nucleotide |
| | | 48 kDa | and nucleic acid metabolism |
| 214727_at | 675 | breast cancer 2, early onset | nucleobase, nucleoside, nucleotide |
| | | | and nucleic acid metabolism |
| 209773_s_at | 6241 | ribonucleotide reductase M2 polypeptide | nucleobase, nucleoside, nucleotide |
| | | | and nucleic acid metabolism |
| 207113_s_at | 7124 | tumor necrosis factor (TNF superfamily, | nucleobase, nucleoside, nucleotide |
| | | member 2) | and nucleic acid metabolism |
| 200660_at | 6282 | S100 calcium binding protein A11 | nucleobase, nucleoside, nucleotide |
| | | (calgizzarin) | and nucleic acid metabolism |
| 209930_s_at | 4778 | nuclear factor (erythroid-derived 2), | nucleobase, nucleoside, nucleotide |
| | | 45 kDa | and nucleic acid metabolism |
| 201989_s_at | 1389 | cAMP responsive element binding | nucleobase, nucleoside, nucleotide |
| | | protein-like 2 | and nucleic acid metabolism |

### Response to Biotic Stimuli

| | LOCUS | | |
|---|---|---|---|
| AFFX_ID | LINK | GENE NAME | CLASSIFICATION |
| 201612_at | 223 | aldehyde dehydrogenase 9 family, | aldehyde metabolism |
| | | member A1 | |
| 210942_s_at | 10402 | alpha2,3-sialyltransferase | amine metabolism |
| 206643_at | 3034 | histidine ammonia-lyase | amine metabolism |
| 206643_at | 3034 | histidine ammonia-lyase | amino acid and derivative |
| | | | metabolism |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 206643_at | 3034 | histidine ammonia-lyase | biosynthesis |
| 204446_s_at | 240 | arachidonate 5-lipoxygenase | biosynthesis |
| 204174_at | 241 | arachidonate 5-lipoxygenase-activating | biosynthesis |
| | | protein | |
| 208771_s_at | 4048 | leukotriene A4 hydrolase | biosynthesis |
| 210942_s_at | 10402 | alpha2,3-sialyltransferase | carbohydrate metabolism |
| 202499_s_at | 6515 | solute carrier family 2 (facilitated | carbohydrate metabolism |
| | | glucose transporter), member 3 | |
| 206643_at | 3034 | histidine ammonia-lyase | catabolism |
| 202551_s_at | 51232 | cysteine-rich motor neuron 1 | catabolism |
| 208771_s_at | 4048 | leukotriene A4 hydrolase | catabolism |
| 212190_at | 5270 | serine (or cysteine) proteinase inhibitor, | catabolism |
| | | clade E (nexin, plasminogen activator | |
| | | inhibitor type 1), member 2 | |
| 211506_s_at | 3576 | interleukin 8 | cell adhesion |
| 207113_s_at | 7124 | tumor necrosis factor (TNF superfamily, | cell adhesion |
| | | member 2) | |
| 201105_at | 3956 | lectin, galactoside-binding, soluble, 1 | cell adhesion |
| | | (galectin 1) | |
| 216250_s_at | 9404 | leupaxin | cell adhesion |
| 219890_at | 23601 | C-type (calcium dependent, | cell adhesion |
| | | carbohydrate-recognition domain) | |
| | | lectin, superfamily member 5 | |
| 214727_at | 675 | breast cancer 2, early onset | cell death |
| 207113_s_at | 7124 | tumor necrosis factor (TNF superfamily, | cell death |
| | | member 2) | |
| 204614_at | 5055 | serine (or cysteine) proteinase inhibitor, | cell death |
| | | clade B (ovalbumin), member 2 | |
| 201105_at | 3956 | lectin, galactoside-binding, soluble, 1 | cell death |
| | | (galectin 1) | |
| 202551_s_at | 51232 | cysteine-rich motor neuron 1 | cell growth |
| 212190_at | 5270 | serine (or cysteine) proteinase inhibitor, | cell migration |
| | | clade E (nexin, plasminogen activator | |
| | | inhibitor type 1), member 2 | |
| 214727_at | 675 | breast cancer 2, early onset | cell organization and biogenesis |
| 201999_s_at | 6993 | t-complex-associated-testis-expressed | cell organization and biogenesis |
| | | 1-like 1 | |
| 204603_at | 9156 | exonuclease 1 | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 | cell proliferation |
| | | polypeptide | |
| 211506_s_at | 3576 | interleukin 8 | cell proliferation |
| 200660_at | 6282 | S100 calcium binding protein A11 | cell proliferation |
| | | (calgizzarin) | |
| 202769_at | 901 | cyclin G2 | cell proliferation |
| 201700_at | 896 | cyclin D3 | cell proliferation |
| 211506_s_at | 3576 | interleukin 8 | cell-cell signaling |
| 207113_s_at | 7124 | tumor necrosis factor (TNF superfamily, | cell-cell signaling |
| | | member 2) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, subfamily | electron transport |
| | | B, polypeptide 1 | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201963_at | 2180 | fatty-acid-Coenzyme A ligase, long- | lipid metabolism |
| | | chain 2 | |
| 208771_s_at | 4048 | leukotriene A4 hydrolase | lipid metabolism |
| 207275_s_at | 2179 | fatty-acid-Coenzyme A ligase, long- | lipid metabolism |
| | | chain 1 | |
| 209906_at | 719 | complement component 3a receptor 1 | muscle contraction |
| 204603_at | 9156 | exonuclease 1 | nucleobase, nucleoside, |
| | | | nucleotide and nucleic acid |
| | | | metabolism |
| 202338_at | 7083 | thymidine kinase 1, soluble | nucleobase, nucleoside, |
| | | | nucleotide and nucleic acid |
| | | | metabolism |
| 218384_at | 23589 | calcium regulated heat stable protein 1, | nucleobase, nucleoside, |
| | | 24 kDa | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding protein | nucleobase, nucleoside, |
| | | 2, 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 | nucleobase, nucleoside, |
| | | polypeptide | nucleotide and nucleic acid |
| | | | metabolism |
| 207113_s_at | 7124 | tumor necrosis factor (TNF superfamily, | nucleobase, nucleoside, |
| | | member 2) | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), | nucleobase, nucleoside, |
| | | 45 kDa | nucleotide and nucleic acid |
| | | | metabolism |
| 201989_s_at | 1389 | cAMP responsive element binding | nucleobase, nucleoside, |
| | | protein-like 2 | nucleotide and nucleic acid |
| | | | metabolism |
| 209301_at | 760 | carbonic anhydrase II | one-carbon compound |
| | | | metabolism |
| 206643_at | 3034 | histidine ammonia-lyase | organic acid metabolism |
| 204446_s_at | 240 | arachidonate 5-lipoxygenase | organic acid metabolism |
| 204174_at | 241 | arachidonate 5-lipoxygenase-activating | organic acid metabolism |
| | | protein | |
| 201963_at | 2180 | fatty-acid-Coenzyme A ligase, long- | organic acid metabolism |
| | | chain 2 | |
| 208771_s_at | 4048 | leukotriene A4 hydrolase | organic acid metabolism |
| 207275_s_at | 2179 | fatty-acid-Coenzyme A ligase, long- | organic acid metabolism |
| | | chain 1 | |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202551_s_at | 51232 | cysteine-rich motor neuron 1 | organogenesis |
| 202437_s_at | 1545 | cytochrome P450, family 1, subfamily | organogenesis |
| | | B, polypeptide 1 | |
| 205807_s_at | 7286 | tuftelin 1 | ossification |
| 202437_s_at | 1545 | cytochrome P450, family 1, subfamily | perception of external stimulus |
| | | B, polypeptide 1 | |
| 209457_at | 1847 | dual specificity phosphatase 5 | phosphorus metabolism |
| 214727_at | 675 | breast cancer 2, early onset | programmed cell death |
| 207113_s_at | 7124 | tumor necrosis factor (TNF superfamily, | programmed cell death |
| | | member 2) | |
| 204614_at | 5055 | serine (or cysteine) proteinase inhibitor, | programmed cell death |
| | | clade B (ovalbumin), member 2 | |
| 201105_at | 3956 | lectin, galactoside-binding, soluble, 1 | programmed cell death |
| | | (galectin 1) | |
| 209457_at | 1847 | dual specificity phosphatase 5 | protein metabolism |
| 210942_s_at | 10402 | alpha2,3-sialyltransferase | protein metabolism |
| 219649_at | 29929 | dolichyl-P-Glc:Man9GlcNAc2-PP- | protein metabolism |
| | | dolichylglucosyltransferase | |
| 200962_at | 6160 | ribosomal protein L31 | protein metabolism |
| 216250_s_at | 9404 | leupaxin | protein metabolism |
| 202551_s_at | 51232 | cysteine-rich motor neuron 1 | protein metabolism |
| 208771_s_at | 4048 | leukotriene A4 hydrolase | protein metabolism |
| 212190_at | 5270 | serine (or cysteine) proteinase inhibitor, | protein metabolism |
| | | clade E (nexin, plasminogen activator | |
| | | inhibitor type 1), member 2 | |
| 212190_at | 5270 | serine (or cysteine) proteinase inhibitor, | regulation of metabolism |
| | | clade E (nexin, plasminogen activator | |
| | | inhibitor type 1), member 2 | |
| 205557_at | 671 | bactericidal/permeability-increasing | response to abiotic stimulus |
| | | protein | |
| 202437_s_at | 1545 | cytochrome P450, family 1, subfamily | response to abiotic stimulus |
| | | B, polypeptide 1 | |
| 209906_at | 719 | complement component 3a receptor 1 | response to biotic stimulus |
| 211506_s_at | 3576 | interleukin 8 | response to biotic stimulus |
| 205557_at | 671 | bactericidal/permeability-increasing protein | response to biotic stimulus |
| 207113_s_at | 7124 | tumor necrosis factor (TNF superfamily, | response to biotic stimulus |
| | | member 2) | |
| 203923_s_at | 1536 | cytochrome b-245, beta polypeptide | response to biotic stimulus |
| | | (chronic granulomatous disease) | |
| 204446_s_at | 240 | arachidonate 5-lipoxygenase | response to biotic stimulus |
| 204174_at | 241 | arachidonate 5-lipoxygenase-activating | response to biotic stimulus |
| | | protein | |
| 204118_at | 962 | CD48 antigen (B-cell membrane protein) | response to biotic stimulus |
| 208771_s_at | 4048 | leukotriene A4 hydrolase | response to biotic stimulus |
| 216950_s_at | 2209 | Fc fragment of IgG, high affinity Ia, receptor | response to biotic stimulus |
| | | for (CD64) | |

### Cell Proliferation

| | LOCUS | | |
|---|---|---|---|
| AFFX_ID | LINK | GENE NAME | CLASSIFICATION |
| 201612_at | 223 | aldehyde dehydrogenase 9 family, | aldehyde metabolism |
| | | member A1 | |
| 210942_s_at | 10402 | alpha2,3-sialyltransferase | amine metabolism |
| 206643_at | 3034 | histidine ammonia-lyase | amine metabolism |
| 206643_at | 3034 | histidine ammonia-lyase | amino acid and derivative |
| | | | metabolism |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 206643_at | 3034 | histidine ammonia-lyase | biosynthesis |
| 204446_s_at | 240 | arachidonate 5-lipoxygenase | biosynthesis |
| 204174_at | 241 | arachidonate 5-lipoxygenase- | biosynthesis |
| | | activating protein | biosynthesis |
| 208771_s_at | 4048 | leukotriene A4 hydrolase | biosynthesis |
| 210942_s_at | 10402 | alpha2,3-sialyltransferase | carbohydrate metabolism |
| 202499_s_at | 6515 | solute carrier family 2 (facilitated | carbohydrate metabolism |
| | | glucose transporter), member 3 | |
| 206643_at | 3034 | histidine ammonia-lyase | catabolism |
| 202551_s_at | 51232 | cysteine-rich motor neuron 1 | catabolism |
| 208771_s_at | 4048 | leukotriene A4 hydrolase | catabolism |
| 212190_at | 5270 | serine (or cysteine) proteinase | catabolism |
| | | inhibitor, clade E (nexin, | |
| | | plasminogen activator inhibitor type | |
| | | 1), member 2 | |
| 211506_s_at | 3576 | interleukin 8 | cell adhesion |
| 207113_s_at | 7124 | tumor necrosis factor (TNF | cell adhesion |
| | | superfamily, member 2) | |
| 201105_at | 3956 | lectin, galactoside-binding, soluble, 1 | cell adhesion |
| | | (galectin 1) | |
| 216250_s_at | 9404 | leupaxin | cell adhesion |
| 219890_at | 23601 | C-type (calcium dependent, | cell adhesion |
| | | carbohydrate-recognition domain) | |
| | | lectin, superfamily member 5 | |
| 214727_at | 675 | breast cancer 2, early onset | cell death |
| 207113_s_at | 7124 | tumor necrosis factor (TNF | cell death |
| | | superfamily, member 2) | |
| 204614_at | 5055 | serine (or cysteine) proteinase | cell death |
| | | inhibitor, clade B (ovalbumin), | |
| | | member 2 | |
| 201105_at | 3956 | lectin, galactoside-binding, soluble, 1 | cell death |
| | | (galectin 1) | |
| 202551_s_at | 51232 | cysteine-rich motor neuron 1 | cell growth |
| 212190_at | 5270 | serine (or cysteine) proteinase | cell migration |
| | | inhibitor, clade E (nexin, | |
| | | plasminogen activator inhibitor type | |
| | | 1), member 2 | |
| 214727_at | 675 | breast cancer 2, early onset | cell organization and biogenesis |
| 201999_s_at | 6993 | t-complex-associated-testis- | cell organization and biogenesis |
| | | expressed 1-like 1 | |
| 204603_at | 9156 | exonuclease 1 | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 polypeptide | cell proliferation |
| 211506_s_at | 3576 | interleukin 8 | cell proliferation |
| 200660_at | 6282 | S100 calcium binding protein A11 | cell proliferation |
| | | (calgizzarin) | |
| 202769_at | 901 | cyclin G2 | cell proliferation |
| 201700_at | 896 | cyclin D3 | cell proliferation |

### Lipid Metabolism

| | LOCUS | | |
|---|---|---|---|
| AFFX_ID | LINK | GENE NAME | CLASSIFICATION |
| 201612_at | 223 | aldehyde dehydrogenase 9 family, | aldehyde metabolism |
| | | member A1 | |
| 210942_s_at | 10402 | alpha2,3-sialyltransferase | amine metabolism |
| 206643_at | 3034 | histidine ammonia-lyase | amine metabolism |
| 206643_at | 3034 | histidine ammonia-lyase | amino acid and derivative |
| | | | metabolism |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 206643_at | 3034 | histidine ammonia-lyase | biosynthesis |
| 204446_s_at | 240 | arachidonate 5-lipoxygenase | biosynthesis |
| 204174_at | 241 | arachidonate 5-lipoxygenase- | biosynthesis |
| | | activating protein | |
| 208771_s_at | 4048 | leukotriene A4 hydrolase | biosynthesis |
| 210942_s_at | 10402 | alpha2,3-sialyltransferase | carbohydrate metabolism |
| 202499_s_at | 6515 | solute carrier family 2 (facilitated | carbohydrate metabolism |
| | | glucose transporter), member 3 | |
| 206643_at | 3034 | histidine ammonia-lyase | catabolism |
| 202551_s_at | 51232 | cysteine-rich motor neuron 1 | catabolism |
| 208771_s_at | 4048 | leukotriene A4 hydrolase | catabolism |
| 212190_at | 5270 | serine (or cysteine) proteinase | catabolism |
| | | inhibitor, clade E (nexin, | |
| | | plasminogen activator inhibitor type | |
| | | 1), member 2 | |
| 211506_s_at | 3576 | interleukin 8 | cell adhesion |
| 207113_s_at | 7124 | tumor necrosis factor (TNF | cell adhesion |
| | | superfamily, member 2) | |
| 201105_at | 3956 | lectin, galactoside-binding, soluble, 1 | cell adhesion |
| | | (galectin 1) | |
| 216250_s_at | 9404 | leupaxin | cell adhesion |
| 219890_at | 23601 | C-type (calcium dependent, | cell adhesion |
| | | carbohydrate-recognition domain) | |
| | | lectin, superfamily member 5 | |
| 214727_at | 675 | breast cancer 2, early onset | cell death |
| 207113_s_at | 7124 | tumor necrosis factor (TNF | cell death |
| | | superfamily, member 2) | |
| 204614_at | 5055 | serine (or cysteine) proteinase | cell death |
| | | inhibitor, clade B (ovalbumin), | |
| | | member 2 | |
| 201105_at | 3956 | lectin, galactoside-binding, soluble, 1 | cell death |
| | | (galectin 1) | |
| 202551_s_at | 51232 | cysteine-rich motor neuron 1 | cell growth |
| 212190_at | 5270 | serine (or cysteine) proteinase | cell migration |
| | | inhibitor, clade E (nexin, | |
| | | plasminogen activator inhibitor type | |
| | | 1), member 2 | |
| 214727_at | 675 | breast cancer 2, early onset | cell organization and biogenesis |
| 201999_s_at | 6993 | t-complex-associated-testis- | cell organization and biogenesis |
| | | expressed 1-like 1 | |
| 204603_at | 9156 | exonuclease 1 | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 | cell proliferation |
| | | polypeptide | |
| 211506_s_at | 3576 | interleukin 8 | cell proliferation |
| 200660_at | 6282 | S100 calcium binding protein A11 | cell proliferation |
| | | (calgizzarin) | |
| 202769_at | 901 | cyclin G2 | cell proliferation |
| 201700_at | 896 | cyclin D3 | cell proliferation |
| 211506_s_at | 3576 | interleukin 8 | cell-cell signaling |
| 207113_s_at | 7124 | tumor necrosis factor (TNF | cell-cell signaling |
| | | superfamily, member 2) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, | electron transport |
| | | subfamily B, polypeptide 1 | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase-activating | lipid metabolism |
| | | protein | |
| 201963_at | 2180 | fatty-acid-Coenzyme A ligase, long-chain 2 | lipid metabolism |
| 208771_s_at | 4048 | leukotriene A4 hydrolase | lipid metabolism |
| 207275_s_at | 2179 | fatty-acid-Coenzyme A ligase, long-chain 1 | lipid metabolism |

### Programmed Cell Death

| | LOCUS | | |
|---|---|---|---|
| AFFX_ID | LINK | GENE NAME | CLASSIFICATION |
| 201612_at | 223 | aldehyde dehydrogenase 9 family, | aldehyde metabolism |
| | | member A1 | |
| 210942_s_at | 10402 | alpha2,3-sialyltransferase | amine metabolism |
| 206643_at | 3034 | histidine ammonia-lyase | amine metabolism |
| 206643_at | 3034 | histidine ammonia-lyase | amino acid and derivative |
| | | | metabolism |
| 210942_s_at | 10402 | alpha2,3-sialyltransferase | biosynthesis |
| 219649_at | 29929 | dolichyl-P-Glc:Man9GlcNAc2-PP- | biosynthesis |
| | | dolichylglucosyltransferase | |
| 200962_at | 6160 | ribosomal protein L31 | biosynthesis |
| 206643_at | 3034 | histidine ammonia-lyase | biosynthesis |
| 204446_s_at | 240 | arachidonate 5-lipoxygenase | biosynthesis |
| 204174_at | 241 | arachidonate 5-lipoxygenase- | biosynthesis |
| | | activating protein | |
| 208771_s_at | 4048 | leukotriene A4 hydrolase | biosynthesis |
| 210942_s_at | 10402 | alpha2,3-sialyltransferase | carbohydrate metabolism |
| 202499_s_at | 6515 | solute carrier family 2 (facilitated | carbohydrate metabolism |
| | | glucose transporter), member 3 | |
| 206643_at | 3034 | histidine ammonia-lyase | catabolism |
| 202551_s_at | 51232 | cysteine-rich motor neuron 1 | catabolism |
| 208771_s_at | 4048 | leukotriene A4 hydrolase | catabolism |
| 212190_at | 5270 | serine (or cysteine) proteinase | catabolism |
| | | inhibitor, clade E (nexin, | |
| | | plasminogen activator inhibitor type | |
| | | 1), member 2 | |
| 211506_s_at | 3576 | interleukin 8 | cell adhesion |
| 207113_s_at | 7124 | tumor necrosis factor (TNF | cell adhesion |
| | | superfamily, member 2) | |
| 201105_at | 3956 | lectin, galactoside-binding, soluble, 1 | cell adhesion |
| | | (galectin 1) | |
| 216250_s_at | 9404 | leupaxin | cell adhesion |
| 219890_at | 23601 | C-type (calcium dependent, | cell adhesion |
| | | carbohydrate-recognition domain) | |
| | | lectin, superfamily member 5 | |
| 214727_at | 675 | breast cancer 2, early onset | cell death |
| 207113_s_at | 7124 | tumor necrosis factor (TNF | cell death |
| | | superfamily, member 2) | |
| 204614_at | 5055 | serine (or cysteine) proteinase | cell death |
| | | inhibitor, clade B (ovalbumin), | |
| | | member 2 | |
| 201105_at | 3956 | lectin, galactoside-binding, soluble, 1 | cell death |
| | | (galectin 1) | |
| 202551_s_at | 51232 | cysteine-rich motor neuron 1 | cell growth |
| 212190_at | 5270 | serine (or cysteine) proteinase | cell migration |
| | | inhibitor, clade E (nexin, | |
| | | plasminogen activator inhibitor type | |
| | | 1), member 2 | |
| 214727_at | 675 | breast cancer 2, early onset | cell organization and biogenesis |
| 201999_s_at | 6993 | t-complex-associated-testis- | cell organization and biogenesis |
| | | expressed 1-like 1 | |
| 204603_at | 9156 | exonuclease 1 | cell proliferation |
| 205053_at | 5557 | primase, polypeptide 1, 49 kDa | cell proliferation |
| 214727_at | 675 | breast cancer 2, early onset | cell proliferation |
| 209773_s_at | 6241 | ribonucleotide reductase M2 | cell proliferation |
| | | polypeptide | |
| 211506_s_at | 3576 | interleukin 8 | cell proliferation |
| 200660_at | 6282 | S100 calcium binding protein A11 | cell proliferation |
| | | (calgizzarin) | |
| 202769_at | 901 | cyclin G2 | cell proliferation |
| 201700_at | 896 | cyclin D3 | cell proliferation |
| 211506_s_at | 3576 | interleukin 8 | cell-cell signaling |
| 207113_s_at | 7124 | tumor necrosis factor (TNF | cell-cell signaling |
| | | superfamily, member 2) | |
| 203923_s_at | 1536 | cytochrome b-245, beta polypeptide | electron transport |
| | | (chronic granulomatous disease) | |
| 204446_s_at | 240 | arachidonate 5-lipoxygenase | electron transport |
| 202437_s_at | 1545 | cytochrome P450, family 1, | electron transport |
| | | subfamily B, polypeptide 1 | |
| 203923_s_at | 1536 | cytochrome b-245, beta polypeptide | energy pathways |
| | | (chronic granulomatous disease) | |
| 210942_s_at | 10402 | alpha2,3-sialyltransferase | lipid metabolism |
| 204446_s_at | 240 | arachidonate 5-lipoxygenase | lipid metabolism |
| 204174_at | 241 | arachidonate 5-lipoxygenase- | lipid metabolism |
| | | activating protein | |
| 201963_at | 2180 | fatty-acid-Coenzyme A ligase, long- | lipid metabolism |
| | | chain 2 | |
| 208771_s_at | 4048 | leukotriene A4 hydrolase | lipid metabolism |
| 207275_s_at | 2179 | fatty-acid-Coenzyme A ligase, long- | lipid metabolism |
| | | chain 1 | |
| 209906_at | 719 | complement component 3a receptor 1 | muscle contraction |
| 204603_at | 9156 | exonuclease 1 | nucleobase, nucleoside, |
| | | | nucleotide and nucleic acid |
| | | | metabolism |
| 202338_at | 7083 | thymidine kinase 1, soluble | nucleobase, nucleoside, |
| | | | nucleotide and nucleic acid |
| | | | metabolism |
| 218384_at | 23589 | calcium regulated heat stable protein | nucleobase, nucleoside, |
| | | 1, 24 kDa | nucleotide and nucleic acid |
| | | | metabolism |
| 205053_at | 5557 | primase, polypeptide 1, 49 kDa | nucleobase, nucleoside, |
| | | | nucleotide and nucleic acid |
| | | | metabolism |
| 203409_at | 1643 | damage-specific DNA binding | nucleobase, nucleoside, |
| | | protein 2, 48 kDa | nucleotide and nucleic acid |
| | | | metabolism |
| 214727_at | 675 | breast cancer 2, early onset | nucleobase, nucleoside, |
| | | | nucleotide and nucleic acid |
| | | | metabolism |
| 209773_s_at | 6241 | ribonucleotide reductase M2 | nucleobase, nucleoside, |
| | | polypeptide | nucleotide and nucleic acid |
| | | | metabolism |
| 207113_s_at | 7124 | tumor necrosis factor (TNF | nucleobase, nucleoside, |
| | | superfamily, member 2) | nucleotide and nucleic acid |
| | | | metabolism |
| 200660_at | 6282 | S100 calcium binding protein A11 | nucleobase, nucleoside, |
| | | (calgizzarin) | nucleotide and nucleic acid |
| | | | metabolism |
| 209930_s_at | 4778 | nuclear factor (erythroid-derived 2), | nucleobase, nucleoside, |
| | | 45 kDa | nucleotide and nucleic acid |
| | | | metabolism |
| 201989_s_at | 1389 | cAMP responsive element binding | nucleobase, nucleoside, |
| | | protein-like 2 | nucleotide and nucleic acid |
| | | | metabolism |
| 209301_at | 760 | carbonic anhydrase II | one-carbon compound |
| | | | metabolism |
| 206643_at | 3034 | histidine ammonia-lyase | organic acid metabolism |
| 204446_s_at | 240 | arachidonate 5-lipoxygenase | organic acid metabolism |
| 204174_at | 241 | arachidonate 5-lipoxygenase- | organic acid metabolism |
| | | activating protein | |
| 201963_at | 2180 | fatty-acid-Coenzyme A ligase, long- | organic acid metabolism |
| | | chain 2 | |
| 208771_s_at | 4048 | leukotriene A4 hydrolase | organic acid metabolism |
| 207275_s_at | 2179 | fatty-acid-Coenzyme A ligase, long- | organic acid metabolism |
| | | chain 1 | |
| 211506_s_at | 3576 | interleukin 8 | organogenesis |
| 205807_s_at | 7286 | tuftelin 1 | organogenesis |
| 218224_at | 9240 | paraneoplastic antigen MA1 | organogenesis |
| 202551_s_at | 51232 | cysteine-rich motor neuron 1 | organogenesis |
| 202437_s_at | 1545 | cytochrome P450, family 1, | organogenesis |
| | | subfamily B, polypeptide 1 | |
| 205807_s_at | 7286 | tuftelin 1 | ossification |
| 202437_s_at | 1545 | cytochrome P450, family 1, | perception of external stimulus |
| | | subfamily B, polypeptide 1 | |
| 209457_at | 1847 | dual specificity phosphatase 5 | phosphorus metabolism |
| 214727_at | 675 | breast cancer 2, early onset | programmed cell death |
| 207113_s_at | 7124 | tumor necrosis factor (TNF superfamily, | programmed cell death |
| | | member 2) | |
| 204614_at | 5055 | serine (or cysteine) proteinase inhibitor, | programmed cell death |
| | | clade B (ovalbumin), member 2 | |
| 201105_at | 3956 | lectin, galactoside-binding, soluble, 1 | programmed cell death |
| | | (galectin 1) | |

VX-944 (Early n Late) vs. MPA (Early ∩ Late): 43 genes were found to be in common for this stringent comparison. These genes were binned into the following functional

### Fatty Acid Metabolism

| | LOCUS | | |
|---|---|---|---|
| AFFYID | LINK | GENE NAME | CLASSIFICATION |
| 203455_s_at | 6303 | spermidine/spermine N1- | Arginine and proline |
| | | acetyltransferase | metabolism |
| 205053_at | 5557 | primase, polypeptide 1, | DNApolymerase |
| | | 49 kDa | |
| 202437_s_at | 1545 | cytochrome P450, | Fatty acid |
| | | family 1, subfamily B, | metabolism |
| | | polypeptide 1 | |
| 207275_s_at | 2179 | fatty-acid-Coenzyme A | Fatty acid |
| | | ligase, long-chain 1 | metabolism |
| 201963_at | 2180 | fatty-acid-Coenzyme A | Fatty acid |
| | | ligase, long-chain 2 | metabolism |

### Prostaglandin and Leukotriene Metabolism

| | LOCUS | | |
|---|---|---|---|
| AFFYID | LINK | GENE NAME | CLASSIFICATION |
| 203455_s_at | 6303 | spermidine/spermine N1- | Arginine and proline metabolism |
| | | acetyltransferase | |
| 205053_at | 5557 | primase, polypeptide 1, 49 kDa | DNApolymerase |
| 202437_s_at | 1545 | cytochrome P450, family 1, | Fatty acid metabolism |
| | | subfamily B, polypeptide 1 | |
| 207275_s_at | 2179 | fatty-acid-Coenzyme A ligase, long- | Fatty acid metabolism |
| | | chain 1 | |
| 201963_at | 2180 | fatty-acid-Coenzyme A ligase, long- | Fatty acid metabolism |
| | | chain 2 | |
| 204446_s_at | 240 | arachidonate 5-lipoxygenase | Prostaglandin and leukotriene |
| | | | metabolism |
| 208771_s_at | 4048 | leukotriene A4 hydrolase | Prostaglandin and leukotriene |
| | | | metabolism |

### Others

| | LOCUS | | |
|---|---|---|---|
| AFFYID | LINK | GENE NAME | CLASSIFICATION |
| 203455_s_at | 6303 | spermidine/spermine N1-acetyltransferase | Arginine and proline metabolism |
| 205053_at | 5557 | primase, polypeptide 1, 49 kDa | DNApolymerase |
| 204446_s_at | 240 | arachidonate 5-lipoxygenase | Prostaglandin andleukotriene |
| | | | metabolism |
| 208771_s_at | 4048 | leukotriene A4 hydrolase | Prostaglandin andleukotriene |
| | | | metabolism |
| 209773_s_at | 6241 | ribonucleotide reductase M2 polypeptide | Purine metabolism |
| 209773_s_at | 6241 | ribonucleotide reductase M2 polypeptide | Pyrimidine metabolism |
| 200962_at | 6160 | ribosomal protein L31 | Ribosome |
| 202437_s_at | 1545 | cytochrome P450, family 1, subfamily B, | Tryptophan metabolism |
| | | polypeptide 1 | |
| 209457_at | 1847 | dual specificity phosphatase 5 | Phosphatidylinositol signaling |
| | | | system |
| 202437_s_at | 1545 | cytochrome P450, family 1, subfamily B, | gamma-Hexachlorocyclohexane |
| | | polypeptide 1 | degradation |
| 214086_s_at | 10038 | ADP-ribosyltransferase (NAD+; poly(ADP- | |
| | | ribose) polymerase)-like 2 | |
| 210942_s_at | 10402 | alpha2,3-sialyltransferase | |
| 202814_s_at | 10614 | HMBA-inducible | |
| 203923_s_at | 1536 | cytochrome b-245, beta polypeptide (chronic | |
| | | granulomatous disease) | |
| 203409_at | 1643 | damage-specific DNA binding protein 2, 48 kDa | |
| 205229_s_at | 1690 | coagulation factor C homolog, cochlin (Limulus | |
| | | polyphemus) | |
| 213572_s_at | 1992 | serine (or cysteine) proteinase inhibitor, clade B | |
| | | (ovalbumin), member 1 | |
| 216950_s_at | 2209 | Fc fragment of IgG, high affinity Ia, receptor for | |
| | | (CD64) | |
| 219890_at | 23601 | C-type (calcium dependent, carbohydrate- | |
| | | recognition domain) lectin, superfamily member 5 | |
| 204174_at | 241 | arachidonate 5-lipoxygenase-activating protein | |
| 211506_s_at | 3576 | interleukin 8 | |
| 200618_at | 3927 | LIM and SH3 protein 1 | |
| 204614_at | 5055 | serine (or cysteine) proteinase inhibitor, clade B | |
| | | (ovalbumin), member 2 | |
| 203960_s_at | 51668 | HSPCO34 protein | |
| 212190_at | 5270 | serine (or cysteine) proteinase inhibitor, clade E | |
| | | (nexin, plasminogen activator inhibitor type 1), | |
| | | member 2 | |
| 219296_at | 54503 | HIP14-related protein | |
| 219148_at | 55872 | T-LAK cell-originated protein kinase | |
| 221882_s_at | 58986 | transmembrane protein 8 (five membrane- | |
| | | spanning domains) | |
| 203344_s_at | 5932 | retinoblastoma binding protein 8 | |
| 200660_at | 6282 | S100 calcium binding protein A11 (calgizzarin) | |
| 204351_at | 6286 | S100 calcium binding protein P | |
| 202499_s_at | 6515 | solute carrier family 2 (facilitated glucose | |
| | | transporter), member 3 | |
| 205557_at | 671 | bactericidal/permeability-increasing protein | |
| 214727_at | 675 | breast cancer 2, early onset | |
| 203303_at | 6990 | t-complex-associated-testis-expressed 1-like | |
| 201999_s_at | 6993 | t-complex-associated-testis-expressed 1-like 1 | |
| 205807_s_at | 7286 | tuftelin 1 | |
| 201426_s_at | 7431 | vimentin | |
| 203046_s_at | 8914 | timeless homolog (Drosophila) | |
| 218224_at | 9240 | paraneoplastic antigen MA1 | |
| 216250_s_at | 9404 | leupaxin | |
| 200704_at | 9516 | lipopolysaccharide-induced TNF factor | |
| 211429_s_at | | | |

### Processed Data—Gene Lists

For each of the comparative analyses set up for this project, the processed data in the form of gene lists with hyperlinks to various databases has been prepared and is represented in Appendix III.

A snapshot of one of these gene lists is shown below. Each of the columns referring to an external database (GENE CARDS at the Weizmann Institute, Locus Link and OMIM at the NIH) has a hyperlink, which when accessed will pull in data corresponding to that gene.

| | | FOLD | FOLD | | | | | | |
|---|---|---|---|---|---|---|---|---|---|
| | | CHANGE | CHANGE | | | | | | Path- |
| Gene | | (944, | (MPA, | Functional | GENE | LOCUS | | Pathways | ways |
| Symbol | TITLE | EARLY) | EARLY) | Annotation | CARDS | LINK | OMIM | GenMAPP | KEGG |
| C6orf18 | chromosome 6 open reading | 2.0 | 1.5 | | C6orf18 | 54535 | 605310 | — | — |
| | frame 18 | | | | | | | | |
| TMEM8 | transmembrane protein 8 (five | 1.5 | 1.7 | biological_proce [symbol] | TMEM8 | 58986 | | — | — |
| | membrane-spanning domains) | | | | | | | | |
| MGC13024 | hypothetical protein | 1.6 | 1.5 | | MGC13024 | 93129 | | — | — |
| | MGC13024 | | | | | | | | |
| C8orf2 | chromosome 8 open reading | 1.7 | 1.5 | biological_proce [symbol] | C8orf2 | 11160 | | — | — |
| | frame 2 | | | | | | | | |
| RAGD | Rag D protein | 1.7 | 1.5 | | RAGD | 58528 | | — | — |
| MLC-B | myosin regulatory light chain | 1.9 | 1.8 | calcium ion bind [symbol] | MRLC2 | 103910 | | — | — |
| | MRLC2 | | | | | | | | |
| CLECSF5 | C-type (calcium dependent, | 2.1 | 1.5 | heterophilic cell | CLECSF5 | 23601 | 604987 | — | — |
| | carbohydrate-recognition | | | | | | | | |
| | domain) lectin, superfamily | | | | | | | | |
| | member 5 | | | | | | | | |
| ALG6 | dolichyl-P- | 1.6 | 1.5 | N-linked glycos [symbol] | ALG6 | 29929 | 604566 | GPCRs_Other | — |
| | Glc:Man9GlcNAc2-PP- | | | | | | | | |
| | dolichylglucosyltransferase | | | | | | | | |
| C21orf4 | chromosome 21 open reading | 1.5 | 1.5 | biological_proce [symbol] | C21orf4 | 757 | | — | — |
| | frame 4 | | | | | | | | |
| FLJ10852 | HIP14-related protein | 1.7 | 1.5 | | HIP14L | 54503 | | — | — |
| KLHL2 | kelch-like 2, Mayven | 2.0 | 1.6 | actin binding ac [symbol] | KLHL2 | 11275 | 605774 | — | — |
| | (Drosophila) | | | | | | | | |
| TOPK | T-LAK cell-originated protein | 1.7 | 1.5 | | TOPK | 55872 | | — | — |
| | kinase | | | | | | | | |
| FBXO5 | F-box only protein 5 | 1.7 | 1.6 | proteolysis and | FBXO5 | 26271 | 606013 | — | — |
| SNX10 | sorting nexin 10 | 1.8 | 1.5 | | SNX10 | 29887 | | — | — |
| RPAC2 | polymerase (RNA) I | 1.5 | 1.5 | DNA binding act [symbol] | POLR1D | 51082 | | — | — |
| | polypeptide D, 16 kDa | | | | | | | | |

[symbol]

indicates data missing or illegible when filed

In addition, a subset of the data was mapped onto relevant pathways, using the KEGG database. The following gene sets 2 and 3 also may be used in the arrays of the invention and are useful predictors of IMPDH inhibition. Particularly preferred exemplary markers of IMPDH inhibition are ARF4 (SEQ ID NO:______); CCNE2 (SEQ ID NO:______); CGR19 (SEQ ID NO:______); CPR8 (SEQ ID NO:______); EDF1 (SEQ ID NO:______); GTF2F1 (______); LGALS1 (SEQ ID NO:______); PDGFC (SEQ ID NO:______); PSMC5 (SEQ ID NO:______); PTK9 (SEQ ID NO:______); RABGGTA (SEQ ID NO:______); SCAP2 (SEQ ID NO:______); SERPINB2 (SEQ ID NO:______); TBPL1 (SEQ ID NO:______).

### Gene Set 2

| Affymetrix_ID | Title | Gene Symbol |
|---|---|---|
| 200618_at | LIM and SH3 protein 1 | LASP1 |
| 200644_at | MARCKS-like protein | MLP |
| 200660_at | S100 calcium binding protein A11 (calgizzarin) | S100A11 |
| 200704_at | LPS-induced TNF-alpha factor | PIG7 |
| 200788_s_at | phosphoprotein enriched in astrocytes 15 | PEA15 |
| 200904_at | major histocompatibility complex, class I, E | HLA-E |
| 200911_s_at | transforming, acidic coiled-coil containing protein 1 | TACC1 |
| 200931_s_at | vinculin | VCL |
| 200962_at | Homo sapiens cDNA FLJ36224 fis, clone | — |
| | THYMU2000990, mRNA sequence | |
| 201012_at | annexin A1 | ANXA1 |
| 201034_at | adducin 3 (gamma) | ADD3 |
| 201069_at | matrix metalloproteinase 2 (gelatinase A, 72 kDa | MMP2 |
| | gelatinase, 72 kDa type IV collagenase) | |
| 201097_s_at | ADP-ribosylation factor 4 | ARF4 |
| 201136_at | proteolipid protein 2 (colonic epithelium-enriched) | PLP2 |
| 201170_s_at | basic helix-loop-helix domain containing, class B, 2 | BHLHB2 |
| 201300_s_at | prion protein (p27-30) (Creutzfeld-Jakob disease, | PRNP |
| | Gerstmann-Strausler-Scheinker syndrome, fatal | |
| | familial insomnia) | |
| 201329_s_at | v-ets erythroblastosis virus E26 oncogene homolog 2 | ETS2 |
| | (avian) | |
| 201362_at | NS1-binding protein | NS1-BP |
| 201422_at | interferon, gamma-inducible protein 30 | IFI30 |
| 201426_s_at | vimentin | VIM |
| 201468_s_at | NAD(P)H dehydrogenase, quinone 1 | NQO1 |
| 201471_s_at | sequestosome 1 | SQSTM1 |
| 201502_s_at | nuclear factor of kappa light polypeptide gene | NFKBIA |
| | enhancer in B-cells inhibitor, alpha | |
| 201590_x_at | annexin A2 | ANXA2 |
| 201612_at | aldehyde dehydrogenase 9 family, member A1 | ALDH9A1 |
| 201628_s_at | Ras-related GTP-binding protein | RAGA |
| 201641_at | bone marrow stromal cell antigen 2 | BST2 |
| 201656_at | integrin, alpha 6 | ITGA6 |
| 201666_at | tissue inhibitor of metalloproteinase 1 (erythroid | TIMP1 |
| | potentiating activity, collagenase inhibitor) | |
| 201710_at | v-myb myeloblastosis viral oncogene homolog | MYBL2 |
| | (avian)-like 2 | |
| 201714_at | tubulin, gamma 1 | TUBG1 |
| 201745_at | PTK9 protein tyrosine kinase 9 /// PTK9 protein | PTK9 |
| | tyrosine kinase 9 | |
| 201772_at | ornithine decarboxylase antizyme inhibitor | OAZIN |
| 201850_at | capping protein (actin filament), gelsolin-like | CAPG |
| 201908_at | dishevelled, dsh homolog 3 (Drosophila) | DVL3 |
| 201920_at | solute carrier family 20 (phosphate transporter), | SLC20A1 |
| | member 1 | |
| 201921_at | guanine nucleotide binding protein (G protein), | GNG10 |
| | gamma 10 | |
| 201925_s_at | decay accelerating factor for complement (CD55, | DAF |
| | Cromer blood group system) | |
| 201963_at | fatty-acid-Coenzyme A ligase, long-chain 2 | FACL2 |
| 201969_at | nuclear autoantigenic sperm protein (histone-binding) | NASP |
| 201999_s_at | t-complex-associated-testis-expressed 1-like 1 | TCTEL1 |
| 202145_at | lymphocyte antigen 6 complex, locus E | LY6E |
| 202191_s_at | growth arrest-specific 7 | GAS7 |
| 202197_at | myotubularin related protein 3 | MTMR3 |
| 202207_at | ADP-ribosylation factor-like 7 | ARL7 |
| 202241_at | phosphoprotein regulated by mitogenic pathways | C8FW |
| 202256_at | CD2 antigen (cytoplasmic tail) binding protein 2 | CD2BP2 |
| 202276_at | Deleted in split-hand/split-foot 1 region | DSS1 |
| 202355_s_at | general transcription factor IIF, polypeptide 1, 74 kDa | GTF2F1 |
| 202384_s_at | Treacher Collins-Franceschetti syndrome 1 | TCOF1 |
| 202393_s_at | TGFB inducible early growth response | TIEG |
| 202405_at | Homo sapiens cDNA FLJ43274 fis, clone | — |
| | KIDNE2008914 | |
| 202437_s_at | cytochrome P450, subfamily I (dioxin-inducible), | CYP1B1 |
| | polypeptide 1 (glaucoma 3, primary infantile) | |
| 202447_at | 2,4-dienoyl CoA reductase 1, mitochondrial | DECR1 |
| 202506_at | sperm specific antigen 2 | SSFA2 |
| 202528_at | galactose-4-epimerase, UDP- | GALE |
| 202530_at | mitogen-activated protein kinase 14 | MAPK14 |
| 202532_s_at | dihydrofolate reductase | DHFR |
| 202551_s_at | cysteine-rich motor neuron 1 | CRIM1 |
| 202554_s_at | glutathione S-transferase M3 (brain) | GSTM3 |
| 202569_s_at | MAP/microtubule affinity-regulating kinase 3 | MARK3 |
| 202589_at | thymidylate synthetase | TYMS |
| 202611_s_at | cofactor required for Sp1 transcriptional activation, | CRSP2 |
| | subunit 2, 150 kDa | |
| 202611_s_at | cofactor required for Sp1 transcriptional activation, | CRSP2 |
| | subunit 2, 150 kDa | |
| 202644_s_at | tumor necrosis factor, alpha-induced protein 3 | TNFAIP3 |
| 202679_at | Niemann-Pick disease, type C1 | NPC1 |
| 202745_at | ubiquitin specific protease 8 | USP8 |
| 202769_at | Homo sapiens mRNA; cDNA DKFZp434B142 (from | — |
| | clone DKFZp434B142), mRNA sequence | |
| 202794_at | inositol polyphosphate-1-phosphatase | INPP1 |
| 202811_at | associated molecule with the SH3 domain of STAM | AMSH |
| 202814_s_at | HMBA-inducible | HIS1 |
| 202842_s_at | DnaJ (Hsp40) homolog, subfamily B, member 9 | DNAJB9 |
| 202917_s_at | S100 calcium binding protein A8 (calgranulin A) | S100A8 |
| 202933_s_at | v-yes-1 Yamaguchi sarcoma viral oncogene homolog 1 | YES1 |
| 202942_at | electron-transfer-flavoprotein, beta polypeptide | ETFB |
| 202951_at | serine/threonine kinase 38 | STK38 |
| 202957_at | hematopoietic cell-specific Lyn substrate 1 | HCLS1 |
| 202990_at | phosphorylase, glycogen; liver (Hers disease, | PYGL |
| | glycogen storage disease type VI) | |
| 203006_at | inositol polyphosphate-5-phosphatase, 40 kDa | INPP5A |
| 203046_s_at | timeless homolog (Drosophila) | TIMELESS |
| 203185_at | Ras association (RalGDS/AF-6) domain family 2 | RASSF2 |
| 203201_at | phosphomannomutase 2 | PMM2 |
| 203274_at | coagulation factor VIII-associated (intronic transcript) | F8A |
| 203275_at | interferon regulatory factor 2 | IRF2 |
| 203301_s_at | cyclin D binding myb-like transcription factor 1 | DMTF1 |
| 203303_at | H1 histone family, member X | H1FX |
| 203314_at | pseudoautosomal GTP-binding protein-like | PGPL |
| 203320_at | lymphocyte adaptor protein | LNK |
| 203335_at | phytanoyl-CoA hydroxylase (Refsum disease) | PHYH |
| 203344_s_at | retinoblastoma binding protein 8 | RBBP8 |
| 203349_s_at | ets variant gene 5 (ets-related molecule) | ETV5 |
| 203358_s_at | enhancer of zeste homolog 2 (Drosophila) | EZH2 |
| 203370_s_at | enigma (LIM domain protein) | ENIGMA |
| 203388_at | arrestin, beta 2 | ARRB2 |
| 203401_at | phosphoribosyl pyrophosphate synthetase 2 | PRPS2 |
| 203409_at | damage-specific DNA binding protein 2, 48 kDa | DDB2 |
| 203414_at | monocyte to macrophage differentiation-associated | MMD |
| 203455_s_at | spermidine/spermine N1-acetyltransferase | SAT |
| 203471_s_at | pleckstrin | PLEK |
| 203521_s_at | endocrine regulator | ZFP318 |
| 203535_at | S100 calcium binding protein A9 (calgranulin B) | S100A9 |
| 203559_s_at | amiloride binding protein 1 (amine oxidase (copper- | ABP1 |
| | containing)) | |
| 203560_at | gamma-glutamyl hydrolase (conjugase, | GGH |
| | folylpolygammaglutamyl hydrolase) | |
| 203564_at | Fanconi anemia, complementation group G | FANCG |
| 203573_s_at | Rab geranylgeranyltransferase, alpha subunit | RABGGTA |
| 203574_at | nuclear factor, interleukin 3 regulated | NFIL3 |
| 203611_at | telomeric repeat binding factor 2 | TERF2 |
| 203659_s_at | ret finger protein 2 | RFP2 |
| 203706_s_at | frizzled homolog 7 (Drosophila) | FZD7 |
| 203752_s_at | jun D proto-oncogene | JUND |
| 203757_s_at | carcinoembryonic antigen-related cell adhesion | CEACAM6 |
| | molecule 6 (non-specific cross reacting antigen) | |
| 203761_at | Src-like-adaptor | SLA |
| 203880_at | popeye domain containing 2 | POPDC2 |
| 203923_s_at | cytochrome b-245, beta polypeptide (chronic | CYBB |
| | granulomatous disease) | |
| 203943_at | kinesin family member 3B | KIF3B |
| 203960_s_at | HSPCO34 protein | LOC51668 |
| 204023_at | replication factor C (activator 1) 4, 37 kDa | RFC4 |
| 204026_s_at | ZW10 interactor | ZWINT |
| 204116_at | interleukin 2 receptor, gamma (severe combined | IL2RG |
| | immunodeficiency) | |
| 204143_s_at | rTS beta protein | HSRTSBETA |
| 204146_at | RAD51-interacting protein | PIR51 |
| 204162_at | highly expressed in cancer, rich in leucine heptad | HEC |
| | repeats | |
| 204174_at | arachidonate 5-lipoxygenase-activating protein | ALOX5AP |
| 204203_at | CCAAT/enhancer binding protein (C/EBP), gamma | CEBPG |
| 204220_at | glia maturation factor, gamma | GMFG |
| 204232_at | Fc fragment of IgE, high affinity I, receptor for; | FCER1G |
| | gamma polypeptide | |
| 204275_at | small optic lobes homolog (Drosophila) | SOLH |
| 204286_s_at | phorbol-12-myristate-13-acetate-induced protein 1 | PMAIP1 |
| 204351_at | S100 calcium binding protein P | S100P |
| 204362_at | src family associated phosphoprotein 2 | SCAP2 |
| 204417_at | galactosylceramidase (Krabbe disease) | GALC |
| 204440_at | CD83 antigen (activated B lymphocytes, | CD83 |
| | immunoglobulin superfamily) | |
| 204444_at | kinesin-like 1 | KNSL1 |
| 204446_s_at | arachidonate 5-lipoxygenase | ALOX5 |
| 204490_s_at | CD44 antigen (homing function and Indian blood | CD44 |
| | group system) | |
| 204493_at | BH3 interacting domain death agonist | BID |
| 204531_s_at | breast cancer 1, early onset | BRCA1 |
| 204603_at | exonuclease 1 | EXO1 |
| 204605_at | cell growth regulator with ring finger domain 1 | CGRRF1 |
| 204614_at | serine (or cysteine) proteinase inhibitor, clade B | SERPINB2 |
| | (ovalbumin), member 2 | |
| 204635_at | ribosomal protein S6 kinase, 90 kDa, polypeptide 5 | RPS6KA5 |
| 204655_at | chemokine (C-C motif) ligand 5 | CCL5 |
| 204690_at | syntaxin 8 | STX8 |
| 204695_at | cell division cycle 25A | CDC25A |
| 204742_s_at | androgen-induced proliferation inhibitor | APRIN |
| 204759_at | chromosome condensation 1-like | CHC1L |
| 204774_at | ecotropic viral integration site 2A | EVI2A |
| 204786_s_at | interferon (alpha, beta and omega) receptor 2 | IFNAR2 |
| 204857_at | MAD1 mitotic arrest deficient-like 1 (yeast) | MAD1L1 |
| 204887_s_at | serine/threonine kinase 18 | STK18 |
| 204961_s_at | neutrophil cytosolic factor 1 (47 kDa, chronic | NCF1 |
| | granulomatous disease, autosomal 1) | |
| 204971_at | cystatin A (stefin A) | CSTA |
| 205034_at | cyclin E2 | CCNE2 |
| 205041_s_at | orosomucoid 1 | ORM1 |
| 205053_at | primase, polypeptide 1, 49 kDa | PRIM1 |
| 205063_at | survival of motor neuron protein interacting protein 1 | SIP1 |
| 205070_at | inhibitor of growth family, member 3 | ING3 |
| 205098_at | chemokine (C-C motif) receptor 1 | CCR1 |
| 205142_x_at | ATP-binding cassette, sub-family D (ALD), member 1 | ABCD1 |
| 205176_s_at | integrin beta 3 binding protein (beta3-endonexin) | ITGB3BP |
| 205204_at | neuromedin B | NMB |
| 205214_at | serine/threonine kinase 17b (apoptosis-inducing) | STK17B |
| 205229_s_at | coagulation factor C homolog, cochlin (Limulus | COCH |
| | polyphemus) | |
| 205260_s_at | acylphosphatase 1, erythrocyte (common) type | ACYP1 |
| 205269_at | lymphocyte cytosolic protein 2 (SH2 domain | LCP2 |
| | containing leukocyte protein of 76 kDa) | |
| 205282_at | low density lipoprotein receptor-related protein 8, | LRP8 |
| | apolipoprotein e receptor | |
| 205345_at | BRCA1 associated RING domain 1 | BARD1 |
| 205361_s_at | prefoldin 4 | PFDN4 |
| 205419_at | Epstein-Barr virus induced gene 2 (lymphocyte- | EBI2 |
| | specific G protein-coupled receptor) | |
| 205546_s_at | tyrosine kinase 2 | TYK2 |
| 205548_s_at | BTG family, member 3 | BTG3 |
| 205557_at | bactericidal/permeability-increasing protein | BPI |
| 205658_s_at | small nuclear RNA activating complex, polypeptide 4, | SNAPC4 |
| | 190 kDa | |
| 205681_at | BCL2-related protein A1 | BCL2A1 |
| 205726_at | diaphanous homolog 2 (Drosophila) | DIAPH2 |
| 205756_s_at | coagulation factor VIII, procoagulant component | F8 |
| | (hemophilia A) | |
| 205756_s_at | coagulation factor VIII, procoagulant component | F8 |
| | (hemophilia A) | |
| 205769_at | solute carrier family 27 (fatty acid transporter), | SLC27A2 |
| | member 2 | |
| 205807_s_at | tuftelin 1 | TUFT1 |
| 206060_s_at | protein tyrosine phosphatase, non-receptor type 22 | PTPN22 |
| | (lymphoid) | |
| 206066_s_at | RAD51 homolog C (S. cerevisiae) | RAD51C |
| 206111_at | ribonuclease, RNase A family, 2 (liver, eosinophil- | RNASE2 |
| | derived neurotoxin) | |
| 206157_at | pentaxin-related gene, rapidly induced by IL-1 beta | PTX3 |
| 206332_s_at | interferon, gamma-inducible protein 16 | IFI16 |
| 206332_s_at | interferon, gamma-inducible protein 16 | IFI16 |
| 206550_s_at | nucleoporin 155 kDa | NUP155 |
| 206571_s_at | mitogen-activated protein kinase kinase kinase kinase 4 | MAP4K4 |
| 206572_x_at | zinc finger protein 85 (HPF4, HTF1) | ZNF85 |
| 206589_at | growth factor independent 1 | GFI1 |
| 206643_at | histidine ammonia-lyase | HAL |
| 206676_at | carcinoembryonic antigen-related cell adhesion | CEACAM8 |
| | molecule 8 | |
| 206695_x_at | zinc finger protein 43 (HTF6) | ZNF43 |
| 206734_at | jerky homolog-like (mouse) | JRKL |
| 206790_s_at | NADH dehydrogenase (ubiquinone) 1 beta | NDUFB1 |
| | subcomplex, 1, 7 kDa | |
| 207121_s_at | mitogen-activated protein kinase 6 | MAPK6 |
| 207153_s_at | FKBP-associated protein | FAP48 |
| 207198_s_at | LIM and senescent cell antigen-like domains 1 | LIMS1 |
| 207275_s_at | fatty-acid-Coenzyme A ligase, long-chain 1 | FACL1 |
| 207338_s_at | zinc finger protein 200 | ZNF200 |
| 207339_s_at | lymphotoxin beta (TNF superfamily, member 3) | LTB |
| 207522_s_at | ATPase, Ca++ transporting, ubiquitous | ATP2A3 |
| 207563_s_at | O-linked N-acetylglucosamine (GlcNAc) transferase | OGT |
| | (UDP-N-acetylglucosamine:polypeptide-N- | |
| | acetylglucosaminyl transferase) | |
| 208082_x_at | makorin, ring finger protein, 4 | MKRN4 |
| 208107_s_at | exonuclease NEF-sp | LOC81691 |
| 208151_x_at | DEAD/H (Asp-Glu-Ala-Asp/His) box polypeptide 17, | DDX17 |
| | 72 kDa | |
| 208369_s_at | glutaryl-Coenzyme A dehydrogenase | GCDH |
| 208398_s_at | TBP-like 1 | TBPL1 |
| 208540_x_at | S100 calcium binding protein A11 pseudogene | S100A11P |
| 208611_s_at | spectrin, alpha, non-erythrocytic 1 (alpha-fodrin) | SPTAN1 |
| 208624_s_at | eukaryotic translation initiation factor 4 gamma, 1 | EIF4G1 |
| 208683_at | calpain 2, (m/II) large subunit | CAPN2 |
| 208703_s_at | amyloid beta (A4) precursor-like protein 2 | APLP2 |
| 208771_s_at | leukotriene A4 hydrolase | LTA4H |
| 208892_s_at | dual specificity phosphatase 6 | DUSP6 |
| 208925_at | chromosome 3 open reading frame 4 | C3orf4 |
| 208949_s_at | lectin, galactoside-binding, soluble, 3 (galectin 3) | LGALS3 |
| 209043_at | 3′-phosphoadenosine 5′-phosphosulfate synthase 1 | PAPSS1 |
| 209058_at | endothelial differentiation-related factor 1 | EDF1 |
| 209094_at | dimethylarginine dimethylaminohydrolase 1 | DDAH1 |
| 209154_at | Tax interaction protein 1 | TIP-1 |
| 209179_s_at | leukocyte receptor cluster (LRC) member 4 | LENG4 |
| 209185_s_at | insulin receptor substrate 2 | IRS2 |
| 209250_at | degenerative spermatocyte homolog, lipid desaturase | DEGS |
| | (Drosophila) | |
| 209272_at | NGFI-A binding protein 1 (EGR1 binding protein 1) | NAB1 |
| 209288_s_at | CDC42 effector protein (Rho GTPase binding) 3 | CDC42EP3 |
| 209337_at | PC4 and SFRS1 interacting protein 2 | PSIP2 |
| 209396_s_at | chitinase 3-like 1 (cartilage glycoprotein-39) | CHI3L1 |
| 209407_s_at | deformed epidermal autoregulatory factor 1 | DEAF1 |
| | (Drosophila) | |
| 209413_at | UDP-Gal:betaGlcNAc beta 1,4-galactosyltransferase, | B4GALT2 |
| | polypeptide 2 | |
| 209457_at | dual specificity phosphatase 5 | DUSP5 |
| 209473_at | ectonucleoside triphosphate diphosphohydrolase 1 | ENTPD1 |
| 209503_s_at | proteasome (prosome, macropain) 26S subunit, | PSMC5 |
| | ATPase, 5 /// proteasome (prosome, macropain) 26S | |
| | subunit, ATPase, 5 | |
| 209575_at | interleukin 10 receptor, beta | IL10RB |
| 209585_s_at | multiple inositol polyphosphate histidine phosphatase, 1 | MINPP1 |
| 209585_s_at | multiple inositol polyphosphate histidine phosphatase, 1 | MINPP1 |
| 209666_s_at | conserved helix-loop-helix ubiquitous kinase | CHUK |
| 209714_s_at | cyclin-dependent kinase inhibitor 3 (CDK2-associated | CDKN3 |
| | dual specificity phosphatase) | |
| 209773_s_at | ribonucleotide reductase M2 polypeptide | RRM2 |
| 209798_at | nuclear protein, ataxia-telangiectasia locus | NPAT |
| 209906_at | complement component 3a receptor 1 | C3AR1 |
| 209930_s_at | nuclear factor (erythroid-derived 2), 45 kDa | NFE2 |
| 210555_s_at | nuclear factor of activated T-cells, cytoplasmic, | NFATC3 |
| | calcineurin-dependent 3 | |
| 210840_s_at | IQ motif containing GTPase activating protein 1 | IQGAP1 |
| 210845_s_at | plasminogen activator, urokinase receptor | PLAUR |
| 210942_s_at | alpha2,3-sialyltransferase | ST3GALVI |
| 210946_at | phosphatidic acid phosphatase type 2A | PPAP2A |
| 211004_s_at | aldehyde dehydrogenase 3 family, member B1 | ALDH3B1 |
| 211317_s_at | CASP8 and FADD-like apoptosis regulator | CFLAR |
| 211429_s_at | serine (or cysteine) proteinase inhibitor, clade A | SERPINA1 |
| | (alpha-1 antiproteinase, antitrypsin), member 1 | |
| 211506_s_at | interleukin 8 | IL8 |
| 212063_at | CD44 antigen (homing function and Indian blood | CD44 |
| | group system) | |
| 212223_at | iduronate 2-sulfatase (Hunter syndrome) | IDS |
| 212410_at | hypothetical protein FLJ34588 | FLJ34588 |
| 212481_s_at | tropomyosin 4 | TPM4 |
| 212588_at | protein tyrosine phosphatase, receptor type, C | PTPRC |
| 212657_s_at | interleukin 1 receptor antagonist | IL1RN |
| 212681_at | erythrocyte membrane protein band 4.1-like 3 | EPB41L3 |
| 212753_at | hypothetical protein MGC40413 | MGC40413 |
| 212990_at | synaptojanin 1 | SYNJ1 |
| 213164_at | mitochondrial ribosomal protein S6 | MRPS6 |
| 213253_at | SMC2 structural maintenance of chromosomes 2-like | SMC2L1 |
| | 1 (yeast) | |
| 213326_at | vesicle-associated membrane protein 1 | VAMP1 |
| | (synaptobrevin 1) | |
| 213373_s_at | caspase 8, apoptosis-related cysteine protease | CASP8 |
| 213457_at | malignant fibrous histiocytoma amplified sequence 1 | MFHAS1 |
| 213572_s_at | serine (or cysteine) proteinase inhibitor, clade B | SERPINB1 |
| | (ovalbumin), member 1 | |
| 213587_s_at | vacuolar proton-ATPase subunit | LOC155066 |
| 213650_at | golgin-67 | GOLGIN-67 |
| 213698_at | hypothetical protein MGC14276 | MGC14276 |
| 213791_at | proenkephalin | PENK |
| 213915_at | natural killer cell group 7 sequence | NKG7 |
| 213951_s_at | GT198, complete ORF | HUMGT198A |
| 214075_at | secreted protein of unknown function | SPUF |
| 214086_s_at | ADP-ribosyltransferase (NAD+; poly(ADP-ribose) | ADPRTL2 |
| | polymerase)-like 2 | |
| 214086_s_at | ADP-ribosyltransferase (NAD+; poly(ADP-ribose) | ADPRTL2 |
| | polymerase)-like 2 | |
| 214157_at | GNAS complex locus | GNAS |
| 214182_at | ADP-ribosylation factor 6 | ARF6 |
| 214467_at | G protein-coupled receptor 65 | GPR65 |
| 214684_at | MADS box transcription enhancer factor 2, | MEF2A |
| | polypeptide A (myocyte enhancer factor 2A) | |
| 214727_at | breast cancer 2, early onset | BRCA2 |
| 216250_s_at | leupaxin | LPXN |
| 216268_s_at | jagged 1 (Alagille syndrome) | JAG1 |
| 216521_s_at | — | — |
| 216598_s_at | chemokine (C-C motif) ligand 2 | CCL2 |
| 216950_s_at | FcRI b form (AA 1-344) [Homo sapiens], mRNA | — |
| | sequence | |
| 217028_at | chemokine (C—X—C motif) receptor 4 | CXCR4 |
| 217478_s_at | major histocompatibility complex, class II, DM alpha | HLA-DMA |
| 217738_at | pre-B-cell colony-enhancing factor | PBEF |
| 217762_s_at | RAB31, member RAS oncogene family | RAB31 |
| 217782_s_at | G protein pathway suppressor 1 | GPS1 |
| 217931_at | trinucleotide repeat containing 5 | TNRC5 |
| 217941_s_at | erbb2 interacting protein | ERBB2IP |
| 217967_s_at | chromosome 1 open reading frame 24 | C1orf24 |
| 218039_at | nucleolar protein ANKT | ANKT |
| 218061_at | male-enhanced antigen | MEA |
| 218084_x_at | FXYD domain containing ion transport regulator 5 | FXYD5 |
| 218111_s_at | cytidine monophosphate N-acetylneuraminic acid | CMAS |
| | synthetase | |
| 218111_s_at | cytidine monophosphate N-acetylneuraminic acid | CMAS |
| | synthetase | |
| 218212_s_at | molybdenum cofactor synthesis 2 | MOCS2 |
| 218224_at | paraneoplastic antigen MA1 | PNMA1 |
| 218224_at | paraneoplastic antigen MA1 | PNMA1 |
| 218263_s_at | transposon-derived Buster1 transposase-like protein | LOC58486 |
| 218276_s_at | WW45 protein | WW45 |
| 218276_s_at | salvador homolog 1 (Drosophila) | SAV1 |
| 218285_s_at | oxidoreductase UCPA | LOC56898 |
| 218327_s_at | synaptosomal-associated protein, 29 kDa | SNAP29 |
| 218384_at | calcium regulated heat stable protein 1, 24 kDa | CARHSP1 |
| 218662_s_at | chromosome condensation protein G | HCAP-G |
| 218718_at | platelet derived growth factor C | PDGFC |
| 218718_at | platelet derived growth factor C | PDGFC |
| 218767_at | XPMC2 prevents mitotic catastrophe 2 homolog | XPMC2H |
| | (Xenopus laevis) | |
| 218966_at | myosin 5C | MYO5C |
| 218983_at | complement C1r-like proteinase precursor, | LOC51279 |
| 219020_at | hypothetical protein FLJ14249 similar to HS1 binding | FLJ14249 |
| | protein 3 | |
| 219098_at | MYB binding protein (P160) 1a | MYBBP1A |
| 219148_at | T-LAK cell-originated protein kinase | TOPK |
| 219157_at | kelch-like 2, Mayven (Drosophila) | KLHL2 |
| 219161_s_at | chemokine-like factor 1 | CKLF1 |
| 219228_at | zinc finger protein 331; zinc finger protein 463 | ZNF361 |
| 219296_at | hypothetical protein similar to ankyrin repeat- | FLJ10852 |
| | containing priotein AKR1 | |
| 219373_at | dolichyl-phosphate mannosyltransferase polypeptide 3 | DPM3 |
| 219649_at | dolichyl-P-Glc:Man9GlcNAc2-PP- | ALG6 |
| | dolichylglucosyltransferase | |
| 219767_s_at | crystallin, zeta (quinone reductase)-like 1 | CRYZL1 |
| 219863_at | cyclin-E binding protein 1 | CEB1 |
| 219890_at | C-type (calcium dependent, carbohydrate-recognition | CLECSF5 |
| | domain) lectin, superfamily member 5 | |
| 220046_s_at | cyclin L ania-6a | LOC57018 |
| 220059_at | BCR downstream signaling 1 | BRDG1 |
| 220162_s_at | caspase recruitment domain family, member 9 | CARD9 |
| 220330_s_at | SAM domain, SH3 domain and nuclear localisation | SAMSN1 |
| | signals, 1 | |
| 220397_at | nuclear protein double minute 1 | MDM1 |
| 220740_s_at | solute carrier family 12 (potassium/chloride | SLC12A6 |
| | transporters), member 6 | |
| 221060_s_at | toll-like receptor 4 | TLR4 |
| 221156_x_at | cell cycle progression 8 protein | CPR8 |
| 221156_x_at | cell cycle progression 8 protein | CPR8 |
| 221158_at | chromosome 21 open reading frame 66 | C21orf66 |
| 221190_s_at | colon cancer-associated protein Mic1 | MIC1 |
| 221191_at | DKFZp434A0131 protein | DKFZP434A0131 |
| 221381_s_at | mortality factor 4 | MORF4 |
| 221563_at | dual specificity phosphatase 10 | DUSP10 |
| 221571_at | TNF receptor-associated factor 3 | TRAF3 |
| 221645_s_at | zinc finger protein 83 (HPF1) | ZNF83 |
| 221649_s_at | peter pan homolog (Drosophila) | PPAN |
| 221676_s_at | coronin, actin binding protein, 1C | CORO1C |
| 221830_at | RAP2A, member of RAS oncogene family | RAP2A |
| 221840_at | protein tyrosine phosphatase, receptor type, E | PTPRE |
| 221882_s_at | transmembrane protein 8 (five membrane-spanning | TMEM8 |
| | domains) | |
| 221989_at | ribosomal protein L10 | RPL10 |
| 222201_s_at | CASP8 associated protein 2 | CASP8AP2 |
| 37424_at | chromosome 6 open reading frame 18 | C6orf18 |
| 204655_at | chemokine (C-C motif) ligand 5 | CCL5 |
| 216598_s_at | chemokine (C-C motif) ligand 2 | CCL2 |
| 210946_at | phosphatidic acid phosphatase type 2A | PPAP2A |
| 211429_s_at | serine (or cysteine) proteinase inhibitor, clade A | SERPINA1 |
| | (alpha-1 antiproteinase, antitrypsin), member 1 | |
| 203140_at | B-cell CLL/lymphoma 6 (zinc finger protein 51) | BCL6 |
| 206157_at | pentaxin-related gene, rapidly induced by IL-1 beta | PTX3 |
| 204351_at | S100 calcium binding protein P | S100P |
| 205034_at | cyclin E2 | CCNE2 |
| 205681_at | BCL2-related protein A1 | BCL2A1 |
| 203455_s_at | spermidine/spermine N1-acetyltransferase | SAT |
| 211506_s_at | interleukin 8 | IL8 |
| 204961_s_at | neutrophil cytosolic factor 1 (47 kDa, chronic | NCF1 |
| | granulomatous disease, autosomal 1) | |
| 201426_s_at | vimentin | VIM |
| 209457_at | dual specificity phosphatase 5 | DUSP5 |
| 209906_at | complement component 3a receptor 1 | C3AR1 |
| 203388_at | arrestin, beta 2 | ARRB2 |
| 213915_at | natural killer cell group 7 sequence | NKG7 |
| 200660_at | S100 calcium binding protein A11 (calgizzarin) | S100A11 |
| 219890_at | C-type (calcium dependent, carbohydrate-recognition | CLECSF5 |
| | domain) lectin, superfamily member 5 | |
| 203303_at | H1 histone family, member X | H1FX |
| 202551_s_at | cysteine-rich motor neuron 1 | CRIM1 |
| 210942_s_at | alpha2,3-sialyltransferase | ST3GALVI |
| 202814_s_at | HMBA-inducible | HIS1 |
| 200904_at | major histocompatibility complex, class I, E | HLA-E |
| 205807_s_at | tuftelin 1 | TUFT1 |
| 207275_s_at | fatty-acid-Coenzyme A ligase, long-chain 1 | FACL1 |
| 204286_s_at | phorbol-12-myristate-13-acetate-induced protein 1 | PMAIP1 |
| 206643_at | histidine ammonia-lyase | HAL |
| 208107_s_at | exonuclease NEF-sp | LOC81691 |
| 217762_s_at | RAB31, member RAS oncogene family | RAB31 |
| 204174_at | arachidonate 5-lipoxygenase-activating protein | ALOX5AP |
| 214086_s_at | ADP-ribosyltransferase (NAD+; poly(ADP-ribose) | ADPRTL2 |
| | polymerase)-like 2 | |
| 201925_s_at | decay accelerating factor for complement (CD55, | DAF |
| | Cromer blood group system) | |
| 212223_at | Homo sapiens mRNA; cDNA DKFZp434G012 (from | — |
| | clone DKFZp434G012), mRNA sequence | |
| 203923_s_at | cytochrome b-245, beta polypeptide (chronic | CYBB |
| | granulomatous disease) | |
| 203409_at | damage-specific DNA binding protein 2, 48 kDa | DDB2 |
| 221830_at | RAP2A, member of RAS oncogene family | RAP2A |
| 205557_at | bactericidal/permeability-increasing protein | BPI |
| 209773_s_at | ribonucleotide reductase M2 polypeptide | RRM2 |
| 205098_at | chemokine (C-C motif) receptor 1 | CCR1 |
| 218224_at | paraneoplastic antigen MA1 | PNMA1 |
| 201710_at | v-myb myeloblastosis viral oncogene homolog | MYBL2 |
| | (avian)-like 2 | |
| 204614_at | serine (or cysteine) proteinase inhibitor, clade B | SERPINB2 |
| | (ovalbumin), member 2 | |
| 200618_at | LIM and SH3 protein 1 | LASP1 |
| 204116_at | interleukin 2 receptor, gamma (severe combined | IL2RG |
| | immunodeficiency) | |
| 204446_s_at | arachidonate 5-lipoxygenase | ALOX5 |
| 207610_s_at | egf-like module containing, mucin-like, hormone | EMR2 |
| | receptor-like sequence 2 | |
| 216950_s_at | FcRI b form (AA 1-344) [Homo sapiens], mRNA | — |
| | sequence | |
| 209473_at | ectonucleoside triphosphate diphosphohydrolase 1 | ENTPD1 |
| 207339_s_at | lymphotoxin beta (TNF superfamily, member 3) | LTB |
| 213622_at | collagen, type IX, alpha 2 | COL9A2 |
| 202393_s_at | TGFB inducible early growth response | TIEG |
| 209094_at | dimethylarginine dimethylaminohydrolase 1 | DDAH1 |
| 201963_at | fatty-acid-Coenzyme A ligase, long-chain 2 | FACL2 |
| 201850_at | capping protein (actin filament), gelsolin-like | CAPG |
| 206111_at | ribonuclease, RNase A family, 2 (liver, eosinophil- | RNASE2 |
| | derived neurotoxin) | |
| 204786_s_at | interferon (alpha, beta and omega) receptor 2 | IFNAR2 |
| 218384_at | calcium regulated heat stable protein 1, 24 kDa | CARHSP1 |
| 201012_at | annexin A1 | ANXA1 |
| 203535_at | S100 calcium binding protein A9 (calgranulin B) | S100A9 |
| 214727_at | breast cancer 2, early onset | BRCA2 |
| 202437_s_at | cytochrome P450, subfamily I (dioxin-inducible), | CYP1B1 |
| | polypeptide 1 (glaucoma 3, primary infantile) | |
| 210845_s_at | plasminogen activator, urokinase receptor | PLAUR |
| 213572_s_at | serine (or cysteine) proteinase inhibitor, clade B | SERPINB1 |
| | (ovalbumin), member 1 | |
| 201989_s_at | cAMP responsive element binding protein-like 2 | CREBL2 |
| 218662_s_at | chromosome condensation protein G | HCAP-G |
| 221882_s_at | transmembrane protein 8 (five membrane-spanning | TMEM8 |
| | domains) | |
| 206676_at | carcinoembryonic antigen-related cell adhesion | CEACAM8 |
| | molecule 8 | |
| 205229_s_at | coagulation factor C homolog, cochlin (Limulus | COCH |
| | polyphemus) | |
| 204971_at | cystatin A (stefin A) | CSTA |
| 216250_s_at | leupaxin | LPXN |
| 221156_x_at | cell cycle progression 8 protein | CPR8 |
| 203960_s_at | HSPCO34 protein | LOC51668 |
| 219157_at | kelch-like 2, Mayven (Drosophila) | KLHL2 |
| 202207_at | ADP-ribosylation factor-like 7 | ARL7 |
| 209396_s_at | chitinase 3-like 1 (cartilage glycoprotein-39) | CHI3L1 |
| 213164_at | Homo sapiens G21VN02 mRNA, mRNA sequence | — |
| 200962_at | Homo sapiens cDNA FLJ36224 fis, clone | — |
| | THYMU2000990, mRNA sequence | |
| 203046_s_at | timeless homolog (Drosophila) | TIMELESS |
| 205041_s_at | orosomucoid 1 | ORM1 |
| 203974_at | DNA segment, numerous copies, expressed probes | DXF68S1E |
| | (GS1 gene) | |
| 202811_at | associated molecule with the SH3 domain of STAM | AMSH |
| 202554_s_at | glutathione S-transferase M3 (brain) | GSTM3 |
| 207121_s_at | mitogen-activated protein kinase 6 | MAPK6 |
| 201999_s_at | t-complex-associated-testis-expressed 1-like 1 | TCTEL1 |
| 209301_at | carbonic anhydrase II | CA2 |
| 201034_at | adducin 3 (gamma) | ADD3 |
| 201471_s_at | sequestosome 1 | SQSTM1 |
| 203757_s_at | carcinoembryonic antigen-related cell adhesion | CEACAM6 |
| | molecule 6 (non-specific cross reacting antigen) | |
| 218145_at | chromosome 20 open reading frame 97 | C20orf97 |
| 204490_s_at | CD44 antigen (homing function and Indian blood | CD44 |
| | group system) | |
| 219494_at | RAD54B homolog | RAD54B |
| 218966_at | myosin 5C | MYO5C |
| 203761_at | Src-like-adaptor | SLA |
| 204561_x_at | apolipoprotein C-II | APOC2 |
| 217967_s_at | chromosome 1 open reading frame 24 | C1orf24 |
| 203344_s_at | retinoblastoma binding protein 8 | RBBP8 |
| 209154_at | Tax interaction protein 1 | TIP-1 |
| 205053_at | primase, polypeptide 1, 49 kDa | PRIM1 |
| 213698_at | Unknown (protein for MGC: 32701) [Homo sapiens], | — |
| | mRNA sequence | |
| 205260_s_at | acylphosphatase 1, erythrocyte (common) type | ACYP1 |
| 203370_s_at | enigma (LIM domain protein) | ENIGMA |
| 204220_at | glia maturation factor, gamma | GMFG |
| 203574_at | nuclear factor, interleukin 3 regulated | NFIL3 |
| 205070_at | inhibitor of growth family, member 3 | ING3 |
| 200788_s_at | phosphoprotein enriched in astrocytes 15 | PEA15 |
| 213587_s_at | vacuolar proton-ATPase subunit | LOC155066 |
| 204603_at | exonuclease 1 | EXO1 |
| 218061_at | male-enhanced antigen | MEA |
| 212501_at | CCAAT/enhancer binding protein (C/EBP), beta | CEBPB |
| 201628_s_at | Ras-related GTP-binding protein | RAGA |
| 212481_s_at | tropomyosin 4 | TPM4 |
| 217976_s_at | dynein, cytoplasmic, light intermediate polypeptide 1 | DNCLI1 |
| 203358_s_at | enhancer of zeste homolog 2 (Drosophila) | EZH2 |
| 207543_s_at | procollagen-proline, 2-oxoglutarate 4-dioxygenase | P4HA1 |
| | (proline 4-hydroxylase), alpha polypeptide I | |
| 205345_at | BRCA1 associated RING domain 1 | BARD1 |
| 206572_x_at | zinc finger protein 85 (HPF4, HTF1) | ZNF85 |
| 201422_at | interferon, gamma-inducible protein 30 | IFI30 |
| 200704_at | LPS-induced TNF-alpha factor | PIG7 |
| 209798_at | nuclear protein, ataxia-telangiectasia locus | NPAT |
| 209272_at | NGFI-A binding protein 1 (EGR1 binding protein 1) | NAB1 |
| 218212_s_at | molybdenum cofactor synthesis 2 | MOCS2 |
| 206734_at | jerky homolog-like (mouse) | JRKL |
| 213951_s_at | GT198, complete ORF | HUMGT198A |
| 208771_s_at | leukotriene A4 hydrolase | LTA4H |
| 202917_s_at | S100 calcium binding protein A8 (calgranulin A) | S100A8 |
| 202644_s_at | tumor necrosis factor, alpha-induced protein 3 | TNFAIP3 |
| 204887_s_at | serine/threonine kinase 18 | STK18 |
| 219296_at | hypothetical protein similar to ankyrin repeat- | FLJ10852 |
| | containing priotein AKR1 | |
| 202111_at | solute carrier family 4, anion exchanger, member 2 | SLC4A2 |
| | (erythrocyte membrane protein band 3-like 1) | |
| 205204_at | neuromedin B | NMB |
| 209930_s_at | nuclear factor (erythroid-derived 2), 45 kDa | NFE2 |
| 200931_s_at | vinculin | VCL |
| 208398_s_at | TBP-like 1 | TBPL1 |
| 204143_s_at | rTS beta protein | HSRTSBETA |
| 219649_at | dolichyl-P-Glc:Man9GlcNAc2-PP- | ALG6 |
| | dolichylglucosyltransferase | |
| 211004_s_at | aldehyde dehydrogenase 3 family, member B1 | ALDH3B1 |
| 219119_at | U6 snRNA-associated Sm-like protein LSm8 | LSM8 |
| 207008_at | interleukin 8 receptor, beta | IL8RB |
| 201612_at | aldehyde dehydrogenase 9 family, member A1 | ALDH9A1 |
| 210555_s_at | nuclear factor of activated T-cells, cytoplasmic, | NFATC3 |
| | calcineurin-dependent 3 | |
| 202679_at | Niemann-Pick disease, type C1 | NPC1 |
| 212410_at | hypothetical protein FLJ34588 | FLJ34588 |
| 220202_s_at | membrane-associated nucleic acid binding protein | MNAB |
| 37424_at | chromosome 6 open reading frame 18 | C6orf18 |
| 201097_s_at | ADP-ribosylation factor 4 | ARF4 |

### Gene Set 3

| Affymetrix_ID | Title | Gene Symbol |
|---|---|---|
| 204655_at | chemokine (C-C motif) ligand 5 | CCL5 |
| 206157_at | pentaxin-related gene, rapidly induced by IL-1 beta | PTX3 |
| 210946_at | phosphatidic acid phosphatase type 2A | PPAP2A |
| 212657_s_at | interleukin 1 receptor antagonist | IL1RN |
| 216598_s_at | chemokine (C-C motif) ligand 2 | CCL2 |
| 205681_at | BCL2-related protein A1 | BCL2A1 |
| 208949_s_at | lectin, galactoside-binding, soluble, 3 (galectin 3) | LGALS3 |
| 202769_at | Homo sapiens mRNA; cDNA DKFZp434B142 (from | — |
| | clone DKFZp434B142), mRNA sequence | |
| 211429_s_at | serine (or cysteine) proteinase inhibitor, clade A (alpha-1 | SERPINA1 |
| | antiproteinase, antitrypsin), member 1 | |
| 220397_at | nuclear protein double minute 1 | MDM1 |
| 201666_at | tissue inhibitor of metalloproteinase 1 (erythroid | TIMP1 |
| | potentiating activity, collagenase inhibitor) | |
| 205034_at | cyclin E2 | CCNE2 |
| 204614_at | serine (or cysteine) proteinase inhibitor, clade B | SERPINB2 |
| | (ovalbumin), member 2 | |
| 204351_at | S100 calcium binding protein P | S100P |
| 202551_s_at | cysteine-rich motor neuron 1 | CRIM1 |
| 212223_at | iduronate 2-sulfatase (Hunter syndrome) | IDS |
| 201426_s_at | vimentin | VIM |
| 209457_at | dual specificity phosphatase 5 | DUSP5 |
| 211506_s_at | interleukin 8 | IL8 |
| 219890_at | C-type (calcium dependent, carbohydrate-recognition | CLECSF5 |
| | domain) lectin, superfamily member 5 | |
| 203455_s_at | spermidine/spermine N1-acetyltransferase | SAT |
| 201925_s_at | decay accelerating factor for complement (CD55, | DAF |
| | Cromer blood group system) | |
| 207339_s_at | lymphotoxin beta (TNF superfamily, member 3) | LTB |
| 200660_at | S100 calcium binding protein A11 (calgizzarin) | S100A11 |
| 213915_at | natural killer cell group 7 sequence | NKG7 |
| 203303_at | H1 histone family, member X | H1FX |
| 205260_s_at | acylphosphatase 1, erythrocyte (common) type | ACYP1 |
| 204961_s_at | neutrophil cytosolic factor 1 (47 kDa, chronic | NCF1 |
| | granulomatous disease, autosomal 1) | |
| 207275_s_at | fatty-acid-Coenzyme A ligase, long-chain 1 | FACL1 |
| 205807_s_at | tuftelin 1 | TUFT1 |
| 202814_s_at | HMBA-inducible | HIS1 |
| 204759_at | chromosome condensation 1-like | CHC1L |
| 221840_at | protein tyrosine phosphatase, receptor type, E | PTPRE |
| 221156_x_at | cell cycle progression 8 protein | CPR8 |
| 204174_at | arachidonate 5-lipoxygenase-activating protein | ALOX5AP |
| 210942_s_at | alpha2,3-sialyltransferase | ST3GALVI |
| 217762_s_at | RAB31, member RAS oncogene family | RAB31 |
| 203923_s_at | cytochrome b-245, beta polypeptide (chronic | CYBB |
| | granulomatous disease) | |
| 204286_s_at | phorbol-12-myristate-13-acetate-induced protein 1 | PMAIP1 |
| 201502_s_at | nuclear factor of kappa light polypeptide gene enhancer | NFKBIA |
| | in B-cells inhibitor, alpha | |
| 209906_at | complement component 3a receptor 1 | C3AR1 |
| 209473_at | ectonucleoside triphosphate diphosphohydrolase 1 | ENTPD1 |
| 202191_s_at | growth arrest-specific 7 | GAS7 |
| 203535_at | S100 calcium binding protein A9 (calgranulin B) | S100A9 |
| 218983_at | complement C1r-like proteinase precursor, | LOC51279 |
| 206643_at | histidine ammonia-lyase | HAL |
| 201963_at | fatty-acid-Coenzyme A ligase, long-chain 2 | FACL2 |
| 200904_at | major histocompatibility complex, class I, E | HLA-E |
| 204162_at | highly expressed in cancer, rich in leucine heptad | HEC |
| | repeats | |
| 205098_at | chemokine (C-C motif) receptor 1 | CCR1 |
| 205557_at | bactericidal/permeability-increasing protein | BPI |
| 203388_at | arrestin, beta 2 | ARRB2 |
| 201590_x_at | annexin A2 | ANXA2 |
| 202207_at | ADP-ribosylation factor-like 7 | ARL7 |
| 214086_s_at | ADP-ribosyltransferase (NAD+; poly(ADP-ribose) | ADPRTL2 |
| | polymerase)-like 2 | |
| 213326_at | vesicle-associated membrane protein 1 (synaptobrevin | VAMP1 |
| | 1) | |
| 207563_s_at | O-linked N-acetylglucosamine (GlcNAc) transferase | OGT |
| | (UDP-N-acetylglucosamine:polypeptide-N- | |
| | acetylglucosaminyl transferase) | |
| 201034_at | adducin 3 (gamma) | ADD3 |
| 201999_s_at | t-complex-associated-testis-expressed 1-like 1 | TCTEL1 |
| 200962_at | Homo sapiens cDNA FLJ36224 fis, clone | — |
| | THYMU2000990, mRNA sequence | |
| 203706_s_at | frizzled homolog 7 (Drosophila) | FZD7 |
| 221830_at | RAP2A, member of RAS oncogene family | RAP2A |
| 216250_s_at | leupaxin | LPXN |
| 210845_s_at | plasminogen activator, urokinase receptor | PLAUR |
| 202437_s_at | cytochrome P450, subfamily I (dioxin-inducible), | CYP1B1 |
| | polypeptide 1 (glaucoma 3, primary infantile) | |
| 213951_s_at | GT198, complete ORF | HUMGT198A |
| 214727_at | breast cancer 2, early onset | BRCA2 |
| 213457_at | malignant fibrous histiocytoma amplified sequence 1 | MFHAS1 |
| 203409_at | damage-specific DNA binding protein 2, 48 kDa | DDB2 |
| 214467_at | G protein-coupled receptor 65 | GPR65 |
| 209396_s_at | chitinase 3-like 1 (cartilage glycoprotein-39) | CHI3L1 |
| 218224_at | paraneoplastic antigen MA1 | PNMA1 |
| 203574_at | nuclear factor, interleukin 3 regulated | NFIL3 |
| 201012_at | annexin A1 | ANXA1 |
| 202644_s_at | tumor necrosis factor, alpha-induced protein 3 | TNFAIP3 |
| 203757_s_at | carcinoembryonic antigen-related cell adhesion molecule | CEACAM6 |
| | 6 (non-specific cross reacting antigen) | |
| 218212_s_at | molybdenum cofactor synthesis 2 | MOCS2 |
| 206332_s_at | interferon, gamma-inducible protein 16 | IFI16 |
| 209585_s_at | multiple inositol polyphosphate histidine phosphatase, 1 | MINPP1 |
| 206676_at | carcinoembryonic antigen-related cell adhesion molecule 8 | CEACAM8 |
| 206734_at | jerky homolog-like (mouse) | JRKL |
| 219373_at | dolichyl-phosphate mannosyltransferase polypeptide 3 | DPM3 |
| 216950_s_at | FcRI b form (AA 1-344) [Homo sapiens], mRNA | — |
| | sequence | |
| 204446_s_at | arachidonate 5-lipoxygenase | ALOX5 |
| 205229_s_at | coagulation factor C homolog, cochlin (Limulus | COCH |
| | polyphemus) | |
| 200644_at | MARCKS-like protein | MLP |
| 203140_at | B-cell CLL/lymphoma 6 (zinc finger protein 51) | BCL6 |
| 219767_s_at | crystallin, zeta (quinone reductase)-like 1 | CRYZL1 |
| 218718_at | platelet derived growth factor C | PDGFC |
| 218276_s_at | WW45 protein | WW45 |
| 218111_s_at | cytidine monophosphate N-acetylneuraminic acid | CMAS |
| | synthetase | |
| 213253_at | SMC2 structural maintenance of chromosomes 2-like 1 | SMC2L1 |
| | (yeast) | |
| 209666_s_at | conserved helix-loop-helix ubiquitous kinase | CHUK |
| 206695_x_at | zinc finger protein 43 (HTF6) | ZNF43 |
| 205756_s_at | coagulation factor VIII, procoagulant component | F8 |
| | (hemophilia A) | |
| 205063_at | survival of motor neuron protein interacting protein 1 | SIP1 |
| 204742_s_at | androgen-induced proliferation inhibitor | APRIN |
| 204605_at | cell growth regulator with ring finger domain 1 | CGRRF1 |
| 202611_s_at | cofactor required for Sp1 transcriptional activation, | CRSP2 |
| | subunit 2, 150 kDa | |
| 202405_at | Homo sapiens cDNA FLJ43274 fis, clone | — |
| | KIDNE2008914 | |
| 201745_at | PTK9 protein tyrosine kinase 9 /// PTK9 protein tyrosine | PTK9 |
| | kinase 9 | |
| 201656_at | integrin, alpha 6 | ITGA6 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. Each of the tables, pages and figures of priority application No. 60/575,076 filed on May 27, 2004 are specifically incorporated by reference in their entirety. The following references are exemplary of the state of the art of the development and use of IMPDH inhibitors and are incorporated herein by reference in their entirety:

### REFERENCES

Albrecht W. Storck M, Pfetch E Martin W, Abendroth D (2000) Development and application of a high-performance liquid chromatography-based assay for determination of the activity of inosine 5′-monophosphate dehydrogenase in whole blood and isolated mononuclear cells. Ther Drug Monitoring 22: 283-294.
Allison, A C, Eugui, E M (2000) Mycophenolate mofetil and its mechanisms of action. Immunopharmacology 47: 85-118.
Budde K, Glander P, Bauer S, Braun K, Waiser J, Fritsche L, Mai I, Roots I, Neumayer H-H (2000) Pharmacodynamic monitoring of Mycophenolate Mofetil. Clin Chem Lab Med 38: 1213-1216.
Davis, G L, Esteban-Mur, R, Rustgi, V, Hoefs, J, Gordon, S C, Trepo, C, Shiffman, M L, Zeuzem, S, Craxi, A, Ling, M H, Albrecht, J (1998) Interferon alfa-2b alone or in combination with ribavirin for the treatment of relapse of chronic hepatitis C. International Hepatitis Interventional Therapy Group. N. Engl. J. Med 339: 1493-1499.
Daxecker H, Raab M, Cichna M, Markl P, Muller M M (2001) Determination of the effects of mycophenolic acid on the nucleotide pool of human peripheral blood mononuclear cells in vitro by high-performance liquid chromatography. Clin Chim Acta 310: 81-87.
Dayton J S, Lindsten T, Thompson C B, and Mitchell B S (1994) Effects of human T lymphocyte activation in inosine monophosphate dehyrogensase expression. J Immunol 152: 984-991.
Decker C J, Jain J, Almquist S J, Heiser A D, Nimmesgern E, Lyons S M, Shlyakhter D (2001) In vitro and in vivo characterization of novel non-nucleoside uncompetitive inhibitors of IMPDH. 11^{th} International Congress of Immunology, Stockholm, 22-27 Jul. 2001. Published as a supplement to the Scandinavian J of Immunol 54: suppl 1: July/August 2001.
Dhar T G M, Shen Z, Guo J, Liu C, Watterson S H, Gu H H, Pitts W J, Fleener C A, Rouleau K A, Sherbina N Z, McIntyre K W, Witmer M R, Tredup J A, Chen B-C, Zhao R, Bednarz M S, Cheney D L, MacMaster J F, Miller L M, Berry K K, Harper T W, Barrish J C, Hollenbaugh D L, Iwanowicz E J (2002) Discovery of N-[2-[2-[[3-Methoxy-4-(5-0xazolyl)phenyl]-amino]-5-oxazolyl]phenyl]-N-methyl-4-morpholineacetamide as a novel and potent inhibitor of inosine monophosphate dehydrogenase with excellent in vivo activity. J Med Chem 45: 2127-2130.
Eugui, E M, Almquist, S J, Muller C D, Allison, A C (1991a) Lymphocyte-selective cytostatic and immunosuppressive effects of mycophenolic acid in vitro: role of deoxyguanosine nucleotide depletion. Scan J Immunol 33: 161-173.
Eugui, E M, Mirkovich, A, Allison, A C (1991b) Lymphocyte-selective antiproliferative and immunosuppressive effect of mycophenolic acid in mice. Scand. J. Immunol 33: 175-183.
Fairbanks L D, Bofill M, Ruckemann K and Simmonds A H. 1995. Importance of ribonucleotide availability to proliferating T-lymphocytes from healthy humans. J Biol Chem 270: 29682-29689.
Franchetti P, and Grifantini M (1999) Nucleoside and non-nucleoside IMP dehydrogenase inhibitors as antitumor and antiviral agents. Curr Med Chem 6: 599-614.
Griesmacher A, Weigel G, Seebacher G, Muller M M (1997) IMP-dehydrogenase inhibition in human lymphocytes and lymphoblasts by mycophenolic acid and mycophenolic acid glucuronide. Clin Chem 43: 2312-2317.
Griesmacher A, Weigel G, Seebacher G, Mallinger R, Laufer G, Muller M W (1998). Inhibition of inosine monophosphate dehyrogensase activity by the plasma of heart transplant recipients receiving mycophenolate mofetil. Adv Exp Med Biol 431: 537-541
Gummert J F, Barten M J, Sherwood S W, Gelder T V, Morris R E (1999). Pharmacodynamics of immunosuppression by mycophenolic acid: inhibition of both lymphocyte proliferation and activation correlates with pharmacokinetics. J Pharmacol and Exp Ther 291: 1100-1112.
Heiser A, Bridson G, Leon E, Jones P, Badia M, Saunders J, Armistead D, Decker C, Chaturvedi P. 1996. Single dose mycophenolate mofetil and mycophenolic acid metabolism and disposition in bile duct-cannulated and intact rats. ISSX Proceedings, San Diego, Calif.: October, 1996.
Hirohata S and Yanagida T (1995). Inhibition of expression of cyclin A in human B cells by an immunosuppressant mizoribine. J Immunol 155: 5175-5183.
Ishikawa, H (1999) Mizoribine and mycophenolate mofetil. Curr Med Chem 6: 575-597.
Jain J, Almquist S J, Shlyakhter D, and Harding M W (2001) VX-497: a novel, selective IMPDH inhibitor and immunosuppressive agent. J Pharm Sci 90: 625-637.
Jain J, Almquist S J, Heiser A D, Shlyakhter D, Leon E, Memmott C, Moody C S, Nimmesgem E and Decker C (2002) Characterization of pharmacological efficacy of VX-148, a new, potent immunosuppressive inosine 5′-monophosphate dehydrogenase inhibitor. J Pharmacol Exp Ther 302: 1272-1277.
Jayaram H N, Grusch M, Cooney D A and Krupitza G. 1999. Consequences of IMP dehydrogenase inhibition, and its relationship to cancer and apoptosis. Curr Med Chem 6: 561-574.
Laliberte J, Yee A, Xiong Y and Mitchell B S (1998). Effects of guanine nucleotide depletion on cell cycle progression in human T lymphocytes. Blood 91: 2896-2904.:
Langman L J, LeGatt D F, Yatscoff R W 1995. Pharmacodynamic assessment of mycophenolic acid induced immunosuppression. Clin Chem 41: 295-299.
Langman L J, LeGatt D F, Halloran P F, Yatskoff R W 1996. Pharmacodynamic assessment of mycophenolic acid induced immunosuppression in renal transplant recipients. Transplantation 62: 666-672.
Markland, W, McQuaid, T J, Jain, J, Kwong, A D (2000) Broad-spectrum antiviral activity of the IMP dehydrogenase inhibitor VX-497: a comparison with ribavirin and demonstration of antiviral additivity with alpha interferon. Antimicrobial Agents Chemother 44: 859-866.
McHutchison, J G, Gordon, S C, Schiff, E R, Shiffman, M L, Lee, W M, Rustgi, V K, Goodman, Z D, Ling, M H, Cort, S, Albrecht, J K (1998) Interferon alfa-2b alone or in combination with ribavirin as initial treatment for chronic hepatitis C. Hepatitis Interventional Therapy Group. N Engl J Med 339: 1485-1492.
Mele T S and Halloran P F (2000) The use of mycophenolate mofetil in transplant recipients. Immunopharmacology 47: 215-245.
Millan O, Oppenheimer F, Brunet M, Vilardell J, Rojo I, Vives J, Martorell J (2000). Assessment of mycophenolic acid-induced immunosuppression: a new approach. Clin Chem 46: 1376-1383.
Montero C, Duley J A, Fairbanks L D, McBride M B, Micheli V, Cant A J, Morgan G. 1995. Demonstration of induction of erythrocyte inosine monophosphate dehydrogenase activity in ribavirin-treated patients using a high performance liquid chromatography linked method. Clin Chim Acta 238: 169-178.
Poynard, T, Marcellin, P, Lee, S S, Niederau, C, Minuk, G S, Ideo, G, Bain, V, Heathcote, J, Zeuzem, S, Trepo, C, Albrecht, J K (1998) Randomised trial of interferon alpha2b plus ribavirin for 48 weeks or for 24 weeks versus interferon alpha2b plus placebo for 48 weeks for treatment of chronic infection with hepatitis C virus. International Hepatitis Interventional Therapy Group. Lancet 352: 1426-1432.
Qiu Y, Fairbanks L D, Ruckemann K, Hawrylowicz C M, Richards D F, Kirschbaum B, Simmonds H A. 2000. Mycophenolic acid-induced GTP depletion also affects ATP and pyrimidine synthesis in mitogen-stimulated primary human T-lymphocytes. Transplantation 69: 890-897.
Sanquer S, Breil M, Baron C, Dhamane D, Astier A, Lang P (1999) Induction of inosine monophosphate dehydrogenase activity after long-term treatment with mycophenolate mofetil. Clin Pharmacol Ther 65: 640-664.
Saunders J O and Raybuck S A. 2000. Inosine monophosphate dehydrogenase: consideration of structure, kinetics, and therapeutic potential. Ann Reports in Med Chem 35: 201-210.
Sintchak, M D, Fleming, M A, Futer, O, Raybuck, S A, Chambers, S P, Caron, P R, Murcko, M A, Wilson, K P (1996) Structure and mechanism of inosine monophosphate dehydrogenase in complex with the immunosuppressant Mycophenolic acid. Cell 85: 921-930.
Sintchak M D and Nimmesgem E (2000) The structure of inosine 5′-monophosphate dehydrogenase and the design of novel inhibitors. Immunopharmacology 47: 163-184.
Thomson A W, Woo J, Yao G Z, Todo S, Starzl T E, Zeevi A. 1993 Effect of combined administration of FK506 and the purine biosynthesis inhibitors mizoribine or mycophenolic acid on lymphocyte DNA synthesis and T cell activation molecule expression in human mixed lymphocyte cultures. Transplant Immunology 1: 146-150.
Weaver J L, Pine P S and Aszalos A. 1991 Comparison of the in vitro and biophysical effects of cyclosporin A, FK-506, and mycophenolic acid on human peripheral blood lymphocytes. Immunopharmacology and Immunotoxicology 13: 563-576.

TABLE IX

| Gene Name | Gene Description | Target Description | Gene Symbol | Change_24 h | Change_48 h | Change_72 h |
|---|---|---|---|---|---|---|
| 201110_s_at | thrombospondin 1 | gb: NM_003246.1 /DEF = Homo sapiens | THBS1 | 1.9 | 86.1 | 61.4 |
| | | thrombospondin 1 (THBS1), mRNA. /FEA = mRNA | | | | |
| | | /GEN = THBS1 /PROD = thrombospondin 1 | | | | |
| | | /DB_XREF = gi: 4507484 /UG = Hs.87409 | | | | |
| | | thrombospondin 1 /FL = gb: NM_003246.1 | | | | |
| 206618_at | interleukin 18 receptor 1 | gb: NM_003855.1 /DEF = Homo sapiens interleukin | IL18R1 | 1.5 | 1.3 | 1.0 |
| | | 18 receptor 1 (IL18R1), mRNA. /FEA = mRNA | | | | |
| | | /GEN = IL18R1 /PROD = interleukin 18 receptor 1 | | | | |
| | | /DB_XREF = gi: 4504654 /UG = Hs.159301 | | | | |
| | | interleukin 18 receptor 1 /FL = gb: U43672.1 | | | | |
| | | gb: NM_003855.1 | | | | |
| 212224_at | aldehyde dehydrogenase 1 family, m [symbol] | Consensus includes gb: NM_000689.1 | ALDH1A1 | 1.0 | −1.8 | −1.8 |
| | | /DEF = Homo sapiens aldehyde dehydrogenase 1, | | | | |
| | | soluble (ALDH1), mRNA. /FEA = CDS | | | | |
| | | /GEN = ALDH1 /PROD = aldehyde dehydrogenase | | | | |
| | | 1, soluble /DB_XREF = gi: 4502030 /UG = Hs.76392 | | | | |
| | | aldehyde dehydrogenase 1 family, member A1 | | | | |
| | | /FL = gb: AF003341.1 gb: NM_000689.1 | | | | |
| 202953_at | complement component 1, q subcom [symbol] | gb: NM_000491.2 /DEF = Homo sapiens | C1QB | 1.0 | −1.4 | −1.5 |
| | | complement component 1, q subcomponent, beta | | | | |
| | | polypeptide (C1QB), mRNA. /FEA = mRNA | | | | |
| | | /GEN = C1QB /PROD = complement component 1, | | | | |
| | | q subcomponent, betapolypeptide precursor | | | | |
| | | /DB_XREF = gi: 11038661 /UG = Hs.8986 | | | | |
| | | complement component 1, q subcomponent, beta | | | | |
| | | polypeptide /FL = gb: NM_000491.2 | | | | |
| 209396_s_at | chitinase 3-like 1 (cartilage glycoprot [symbol] | gb: M80927.1 /DEF = Human glycoprotein mRNA, | CHI3L1 | 1.0 | −1.4 | −1.7 |
| | | complete cds. /FEA = mRNA /PROD = glycoprotein | | | | |
| | | /DB_XREF = gi: 348911 /UG = Hs.75184 chitinase 3-like 1 (cartilage | | | | |
| | | glycoprotein-39) /FL = gb: M80927.1 | | | | |
| | | gb: NM_001276.1 | | | | |
| 220059_at | BCR downstream signaling 1 | gb: NM_012108.1 /DEF = Homo sapiens BCR | BRDG1 | 123.9 | −1.5 | −1.1 |
| | | downstream signaling 1 (BRDG1), mRNA. | | | | |
| | | /FEA = mRNA /GEN = BRDG1 /PROD = BCR | | | | |
| | | downstream signaling 1 /DB_XREF = gi: 6912271 | | | | |
| | | /UG = Hs.121128 BCR downstream signaling 1 | | | | |
| | | /FL = gb: AB023483.1 gb: NM_012108.1 | | | | |
| 206133_at | XIAP associated factor 1.0 | gb: NM_017523.1 /DEF = Homo sapiens XIAP | HSXIAPAF1 | 1.4 | −2.4 | 1.4 |
| | | associated factor1.0 (HSXIAPAF1), mRNA. | | | | |
| | | /FEA = mRNA /GEN = HSXIAPAF1 /PROD = XIAP | | | | |
| | | associated factor1.0 /DB_XREF = gi: 8923794 | | | | |
| | | /UG = Hs.139262 XIAP associated factor1.0 | | | | |
| | | /FL = gb: NM_017523.1 | | | | |
| 212628_at | protein kinase N2 | Consensus includes gb: BG292065 /FEA = EST | PKN2 | 1.6 | −1.6 | −1.3 |
| | | /DB_XREF = gi: 13050507 | | | | |
| | | /DB_XREF = est: 602386350F1 | | | | |
| | | /CLONE = IMAGE: 4515036 /UG = Hs.69171 protein | | | | |
| | | kinase C-like 2 | | | | |
| 203595_s_at | interferon-induced protein with tetratr [symbol] | Consensus includes gb: N47725 /FEA = EST | IFIT5 | 1.5 | −2.2 | 1.0 |
| | | /DB_XREF = gi: 1188891 | | | | |
| | | /DB_XREF = est: yy92h11.s1 | | | | |
| | | /CLONE = IMAGE: 281061 /UG = Hs.27610 retinoic | | | | |
| | | acid- and interferon-inducible protein (58 kD) | | | | |
| | | /FL = gb: U34605.1 gb: NM_012420.1 | | | | |
| 205692_s_at | CD38 antigen (p45) | gb: NM_001775.1 /DEF = Homo sapiens CD38 | CD38 | 1.7 | −2.1 | −1.2 |
| | | antigen (p45) (CD38), mRNA. /FEA = mRNA | | | | |
| | | /GEN = CD38 /PROD = CD38 antigen (p45) | | | | |
| | | /DB_XREF = gi: 4502664 /UG = Hs.66052 CD38 | | | | |
| | | antigen (p45) /FL = gb: D84276.1 gb: NM_001775.1 | | | | |
| | | gb: M34461.1 | | | | |
| 201707_at | peroxisomal biogenesis factor 19 | gb: NM_002857.1 /DEF = Homo sapiens | PEX19 | 90.1 | −1.5 | −1.5 |
| | | peroxisomal famesylated protein (PXF), mRNA. | | | | |
| | | /FEA = mRNA /GEN = PXF /PROD = peroxisomal | | | | |
| | | famesylated protein /DB_XREF = gi: 4506338 | | | | |
| | | /UG = Hs.168670 peroxisomal famesylated protein | | | | |
| | | /FL = gb: BC000496.1 gb: NM_002857.1 | | | | |
| | | gb: AB018541.1 | | | | |
| 205033_s_at | defensin, alpha 1, myeloid-related se [symbol] | gb: NM_004084.2 /DEF = Homo sapiens defensin, | DEFA1 | 1.2 | 1.6 | −1.8 |
| | | alpha 1, myeloid-related sequence (DEFA1), | | | | |
| | | mRNA. /FEA = mRNA /GEN = DEFA1 | | | | |
| | | /PROD = defensin, alpha 1, preproprotein | | | | |
| | | /DB_XREF = gi: 12621915 /UG = Hs.274463 | | | | |
| | | defensin, alpha 1, myeloid-related sequence | | | | |
| | | /FL = gb: NM_004084.2 gb: M21130.1 gb: M26602.1 | | | | |
| 33132_at | cleavage and polyadenylation specifi [symbol] | Cluster Incl. U37012: Human cleavage and | CPSF1 | 1.0 | 1.8 | 1.2 |
| | | polyadenylation specificity factor mRNA, complete | | | | |
| | | cds /cds = (51,4379) /gb = U37012 /gi = 1045573 | | | | |
| | | /ug = Hs.83727 /len = 4463 | | | | |
| 211330_s_at | hemochromatosis | gb: AF144242.1 /DEF = Homo sapiens | HFE | 1.1 | −1.1 | 82.1 |
| | | hemochromatosis splice variant delE3 mRNA. | | | | |
| | | complete cds. /FEA = mRNA /GEN = HFE | | | | |
| | | /PROD = hemochromatosis splice variant delE3 | | | | |
| | | /DB_XREF = gi: 11094324 /UG = Hs.20019 | | | | |
| | | hemochromatosis /FL = gb: AF144242.1 | | | | |
| 204439_at | chromosome 1 open reading frame 2 | gb: NM_006820.1 /DEF = Homo sapiens | C1orf29 | 1.0 | −894.0 | 2.2 |
| | | hypothetical protein, expressed in osteoblast | | | | |
| | | (GS3686), mRNA. /FEA = mRNA /GEN = GS3686 | | | | |
| | | /PROD = hypothetical protein, expressed in | | | | |
| | | osteoblast /DB_XREF = gi: 5803026 /UG = Hs.75470 hypothetical | | | | |
| | | protein, expressed in osteoblast | | | | |
| | | /FL = gb: AB000115.1 gb: NM_006820.1 | | | | |
| 206632_s_at | apolipoprotein B mRNA editing enzy [symbol] | gb: NM_004900.1 /DEF = Homo sapiens phorbolin | APOBEC3B | 1.0 | −113.2 | 1.2 |
| | | (similar to apolipoprotein B mRNA editing protein) | | | | |
| | | (DJ742C19.2), mRNA. /FEA = mRNA | | | | |
| | | /GEN = DJ742C19.2 /PROD = phorbolin (similar to | | | | |
| | | apolipoprotein B mRNAediting protein) | | | | |
| | | /DB_XREF = gi: 4758159 /UG = Hs.226307 phorbolin (similar to | | | | |
| | | apolipoprotein B mRNA editing protein) | | | | |
| | | /FL = gb: U61083.1 gb: NM_004900.1 | | | | |
| 202411_at | interferon, alpha-inducible protein 27 | gb: NM_005532.1 /DEF = Homo sapiens interferon, | IFI27 | 1.0 | −120.6 | 1.2 |
| | | alpha-inducible protein 27 (IFI27), mRNA. | | | | |
| | | /FEA = mRNA /GEN = IFI27 /PROD = interferon, | | | | |
| | | alpha-inducible protein 27 /DB_XREF = gi: 5031780 | | | | |
| | | /UG = Hs.278613 interferon, alpha-inducible protein | | | | |
| | | 27 /FL = gb: NM_005532.1 | | | | |
| 219211_at | ubiquitin specific protease 18 | gb: NM_017414.1 /DEF = Homo sapiens ubiquitin | USP18 | 1.0 | −183.7 | 1.0 |
| | | specific protease 18 (USP18), mRNA. | | | | |
| | | /FEA = mRNA /GEN = USP18 /PROD = ubiquitin | | | | |
| | | specific protease 18 /DB_XREF = gi: 8394518 | | | | |
| | | /UG = Hs.38260 ubiquitin specific protease 18 | | | | |
| | | /FL = gb: AL136690.1 gb: AF176642.2 | | | | |
| | | gb: NM_017414.1 | | | | |
| 203153_at | interferon-induced protein with tetratr [symbol] | gb: NM_001548.1 /DEF = Homo sapiens interferon- | | 1.0 | −1084.5 | 1.0 |
| | | induced protein with tetratricopeptide repeats 1 | | | | |
| | | (IFIT1), mRNA. /FEA = mRNA /GEN = IFIT1 | | | | |
| | | /PROD = interferon-induced protein | | | | |
| | | withtetratricopeptide repeats 1 | | | | |
| | | /DB_XREF = gi: 4504584 /UG = Hs.20315 interferon-induced protein with | | | | |
| | | tetratricopeptide repeats 1 /FL = gb: M24594.1 gb: NM_001548.1 | | | | |
| 203167_at | tissue inhibitor of metalloproteinase 2 [symbol] | gb: NM_003255.2 /DEF = Homo sapiens tissue | TIMP2 | 1.2 | −1.9 | 1.1 |
| | | inhibitor of metalloproteinase 2 (TIMP2), mRNA. | | | | |
| | | /FEA = mRNA /GEN = TIMP2 /PROD = tissue | | | | |
| | | inhibitor of metalloproteinase 2precursor | | | | |
| | | /DB_XREF = gi: 9257247 /UG = Hs.325495 tissue | | | | |
| | | inhibitor of metalloproteinase 2 /FL = gb: M32304.1 | | | | |
| | | gb: J05593.1 gb: NM_003255.2 | | | | |
| 202086_at | myxovirus (influenza virus) resistance [symbol] | gb: NM_002462.1 /DEF = Homo sapiens myxovirus | MX1 | 1.1 | −7.3 | −1.2 |
| | | (influenza) resistance 1, homolog of murine | | | | |
| | | (interferon-inducible protein p78) (MX1), mRNA. | | | | |
| | | /FEA = mRNA /GEN = MX1 /PROD = myxovirus | | | | |
| | | (influenza) resistance 1, homolog ofmurine | | | | |
| | | (interferon-inducible protein p78) | | | | |
| | | /DB_XREF = gi: 4505290 /UG = Hs.76391 myxovirus | | | | |
| | | (influenza) resistance 1, homolog of murine | | | | |
| | | (interferon-inducible protein p78) | | | | |
| | | /FL = gb: M30817.1 gb: M33882.1 gb: NM_002462.1 | | | | |
| 214038_at | chemokine (C-C motif) ligand 8 | Consensus includes gb: AI984980 /FEA = EST | CCL8 | 1.1 | −8.4 | −1.2 |
| | | /DB_XREF = gi: 5812257 | | | | |
| | | /DB_XREF = est: wr88g11.x1 | | | | |
| | | /CLONE = IMAGE: 2494820 /UG = Hs.271387 small | | | | |
| | | inducible cytokine subfamily A (Cys-Cys), member | | | | |
| | | 8 (monocyte chemotactic protein 2) | | | | |
| | | /FL = gb: NM_005623.1 | | | | |
| 208436_s_at | interferon regulatory factor 7 | gb: NM_004030.1 /DEF = Homo sapiens interferon | IRF7 | 1.0 | −2.8 | 1.1 |
| | | regulatory factor 7 (IRF7), transcript variant c, | | | | |
| | | mRNA. /FEA = mRNA /GEN = IRF7 | | | | |
| | | /PROD = interferon regulatory factor 7, | | | | |
| | | transcriptvariant c /DB_XREF = gi: 4809285 | | | | |
| | | /UG = Hs.166120 interferon regulatory factor 7 | | | | |
| | | /FL = gb: NM_004030.1 | | | | |
| 218400_at | 2′-5′-oligoadenylate synthetase 3, 10 [symbol] | gb: NM_006187.1 /DEF = Homo sapiens 2- | OAS3 | −1.1 | −3.1 | −1.2 |
| | | 5oligoadenylate synthetase 3 (OAS3), mRNA. | | | | |
| | | /FEA = mRNA /GEN = OAS3 /PROD = 2- | | | | |
| | | 5oligoadenylate synthetase 3 | | | | |
| | | /DB_XREF = gi: 5453623 /UG = Hs.56009 2- | | | | |
| | | 5oligoadenylate synthetase 3 /FL = gb: AB044545.1 | | | | |
| | | gb: AF063613.1 gb: NM_006187.1 | | | | |
| 44673_at | sialoadhesin | Cluster Incl. N53555: yv43e09.s1 Homo sapiens | SN | −1.1 | −3.0 | 1.0 |
| | | cDNA, 3 end /clone = IMAGE-245512 | | | | |
| | | /clone_end = 3′ /gb = N53555 /gi = 1194721 | | | | |
| | | /ug = Hs.31869 /len = 680 | | | | |
| 214453_s_at | interferon-induced protein 44 | Consensus includes gb: NM_006417.1 | IFI44 | 1.4 | −4.9 | 1.1 |
| | | /DEF = Homo sapiens interferon-induced, hepatitis | | | | |
| | | C-associated microtubular aggregate protein | | | | |
| | | (44 kD) (MTAP44), mRNA. /FEA = CDS | | | | |
| | | /GEN = MTAP44 /PROD = interferon-induced, | | | | |
| | | hepatitis C-associatedmicrotubular aggregate | | | | |
| | | protein (44 kD) /DB_XREF = gi: 5453743 | | | | |
| | | /UG = Hs.82316 interferon-induced, hepatitis C- | | | | |
| | | associated microtubular aggregate protein (44 kD) | | | | |
| | | /FL = gb: NM_006417.1 | | | | |
| 204994_at | myxovirus (influenza virus) resistance [symbol] | gb: NM_002463.1 /DEF = Homo sapiens myxovirus | MX2 | 1.0 | −2.8 | 1.1 |
| | | (influenza) resistance 2, homolog of murine | | | | |
| | | (MX2), mRNA. /FEA = mRNA /GEN = MX2 | | | | |
| | | /PROD = myxovirus (influenza) resistance 2, | | | | |
| | | homolog ofmurine /DB_XREF = gi: 11342663 | | | | |
| | | /UG = Hs.926 myxovirus (influenza) resistance 2, | | | | |
| | | homolog of murine /FL = gb: NM_002463.1 | | | | |
| | | gb: M30818.1 gb: M33883.1 | | | | |
| 202446_s_at | phospholipid scramblase 1 | Consensus includes gb: AI825926 /FEA = EST | PLSCR1 | 1.3 | −2.5 | −1.1 |
| | | /DB_XREF = gi: 5446597 | | | | |
| | | /DB_XREF = est: to92d05.x1 | | | | |
| | | /CLONE = IMAGE: 2185737 /UG = Hs.198282 | | | | |
| | | phospholipid scramblase 1 /FL = gb: NM_021105.1 | | | | |
| | | gb: AB006746.1 gb: AF098642.1 | | | | |
| 219863_at | hect domain and RLD 5 | gb: NM_016323.1 /DEF = Homo sapiens cyclin-E | HERC5 | 1.2 | −3.6 | −1.2 |
| | | binding protein 1 (LOC51191), mRNA. | | | | |
| | | /FEA = mRNA /GEN = LOC51191 /PROD = cyclin-E | | | | |
| | | binding protein 1 /DB_XREF = gi: 7705930 | | | | |
| | | /UG = Hs.26663 cyclin-E binding protein 1 | | | | |
| | | /FL = gb: AB027289.1 gb: NM_016323.1 | | | | |
| 204415_at | interferon, alpha-inducible protein (cl [symbol] | gb: NM_022873.1 /DEF = Homo sapiens interferon, | G1P3 | −1.1 | −3.5 | 1.4 |
| | | alpha-inducible protein (clone IFI-61.06) (G1P3), | | | | |
| | | transcript variant 3, mRNA. /FEA = mRNA | | | | |
| | | /GEN = G1P3 /PROD = interferon induced 61.06 | | | | |
| | | protein, isoform, c /DB_XREF = gi: 13259549 | | | | |
| | | /UG = Hs.265827 interferon, alpha-inducible protein | | | | |
| | | (clone IFI-61.06) /FL = gb: NM_022872.1 | | | | |
| | | gb: NM_022873.1 gb: NM_002038.2 | | | | |
| 205483_s_at | interferon, alpha-inducible protein (cl [symbol] | gb: NM_005101.1 /DEF = Homo sapiens interferon- | G1P2 | 1.0 | −9.9 | −1.1 |
| | | stimulated protein, 15 kDa (ISG15), mRNA. | | | | |
| | | /FEA = mRNA /GEN = ISG15 /PROD = interferon- | | | | |
| | | stimulated protein, 15 kDa/DB_XREF = gi: 4826773 | | | | |
| | | /UG = Hs.833 interferon-stimulated protein, 15 kDa | | | | |
| | | /FL = gb: M13755.1 gb: NM_005101.1 | | | | |
| 210001_s_at | suppressor of cytokine signaling 1 | gb: AB005043.1 /DEF = Homo sapiens mRNA for | SOCS1 | 1.1 | −1.7 | −2.7 |
| | | STAT induced STAT inhibitor1.0, complete cds. | | | | |
| | | /FEA = mRNA /PROD = STAT induced STAT | | | | |
| | | inhibitor1.0 /DB_XREF = gi: 2443364 | | | | |
| | | /UG = Hs.50640 JAK binding protein | | | | |
| | | /FL = gb: U88326.1 gb: AB005043.1 gb: AB000734.1 | | | | |
| | | gb: NM_003745.1 | | | | |
| 209196_at | chromosome 6 open reading frame 1 | gb: BC000388.1 /DEF = Homo sapiens, BING4, | C6orf11 | 1.3 | −1.3 | −65.8 |
| | | clone MGC: 8435, mRNA, complete cds. | | | | |
| | | /FEA = mRNA /PROD = BING4 | | | | |
| | | /DB_XREF = gi: 12653238 /UG = Hs.17930 | | | | |
| | | chromosome 6 open reading frame 11 | | | | |
| | | /FL = gb: BC000388.1 gb: NM_005452.2 | | | | |
| 206407_s_at | chemokine (C-C motif) ligand 13 | gb: NM_005408.1 /DEF = Homo sapiens small | CCL13 | 1.0 | −2.9 | −2.1 |
| | | inducible cytokine subfamily A (Cys-Cys), member | | | | |
| | | 13 (SCYA13), mRNA. /FEA = mRNA | | | | |
| | | /GEN = SCYA13 /PROD = small inducible cytokine | | | | |
| | | subfamily A (Cys-Cys), member 13 | | | | |
| | | /DB_XREF = gi: 4885586 /UG = Hs.11383 small | | | | |
| | | inducible cytokine subfamily A (Cys-Cys), member | | | | |
| | | 13 /FL = gb: U46767.1 gb: U59808.1 | | | | |
| | | gb: NM_005408.1 | | | | |
| 216598_s_at | chemokine (C-C motif) ligand 2 | Consensus includes gb: S69738.1 | CCL2 | −1.3 | −3.1 | −2.4 |
| | | /DEF = MCP1.0 = monocyte chemotactic protein | | | | |
| | | human, aortic endothelial cells, mRNA, 661 nt. | | | | |
| | | /FEA = mRNA /GEN = MCP1.0 /PROD = MCP1.0 | | | | |
| | | /DB_XREF = gi: 545464 /UG = Hs.303649 small | | | | |
| | | inducible cytokine A2 (monocyte chemotactic | | | | |
| | | protein 1, homologous to mouse Sig-je) | | | | |
| 212657_s_at | interleukin 1 receptor antagonist | Consensus includes gb: U65590 /DEF = Homo | IL1RN | −1.2 | −2.6 | −3.0 |
| | | sapiens IL1.0 receptor antagonist IL1.0Ra | | | | |
| | | (IL1.0RN) gene, alternatively spliced forms, | | | | |
| | | complete cds /FEA = mRNA_2 | | | | |
| | | /DB_XREF = gi: 2707374 /UG = Hs.81134 interleukin | | | | |
| | | 1 receptor antagonist | | | | |
| 204533_at | chemokine (C—X—C motif) ligand 10 | gb: NM_001565.1 /DEF = Homo sapiens small | CXCL10 | −1.2 | −4.9 | −3.9 |
| | | inducible cytokine subfamily B (Cys-X-Cys), | | | | |
| | | member 10 (SCYB10), mRNA. /FEA = mRNA | | | | |
| | | /GEN = SCYB10 /PROD = interferon gamma- | | | | |
| | | induced precursor /DB_XREF = gi: 4504700 | | | | |
| | | /UG = Hs.2248 small inducible cytokine subfamily B | | | | |
| | | (Cys-X-Cys), member 10 /FL = gb: NM_001565.1 | | | | |
| 203915_at | chemokine (C—X—C motif) ligand 9 | gb: NM_002416.1 /DEF = Homo sapiens monokine | CXCL9 | −1.2 | −1.7 | −3.2 |
| | | induced by gamma interferon (MIG), mRNA. | | | | |
| | | /FEA = mRNA /GEN = MIG /PROD = monokine | | | | |
| | | induced by gamma interferon | | | | |
| | | /DB_XREF = gi: 4505186 /UG = Hs.77367 monokine induced by gamma interferon | | | | |
| | | /FL = gb: NM_002416.1 | | | | |
| 209396_s_at | chitinase 3-like 1 (cartilage glycoprotei [symbol] | gb: M80927.1 /DEF = Human glycoprotein | CHI3L1 | 1.0 | −1.4 | −1.7 |
| | | mRNA, complete cds. /FEA = mRNA | | | | |
| | | /PROD = glycoprotein | | | | |
| | | /DB_XREF = gi: 348911 /UG = Hs.75184 | | | | |
| | | chitinase 3-like 1 (cartilage glycoprotein- | | | | |
| | | 39) /FL = gb: M80927.1 gb: NM_001276.1 | | | | |
| 212657_s_at | interleukin 1 receptor antagonist | Consensus includes gb: U65590 | IL1RN | −1.2 | −2.6 | −3.0 |
| | | /DEF = Homo sapiens IL-1 receptor | | | | |
| | | antagonist IL-1Ra (IL-1RN) gene, | | | | |
| | | alternatively spliced forms, complete cds | | | | |
| | | /FEA = mRNA_2 /DB_XREF = gi: 2707374 | | | | |
| | | /UG = Hs.81134 interleukin 1 receptor | | | | |
| | | antagonist | | | | |
| 209396_s_at | chitinase 3-like 1 (c [symbol] | gb: M80927.1 /DEF = Human | CHI3L1 | 1.0 | −1.4 | −1.7 |
| | | glycoprotein mRNA, complete cds. | | | | |
| | | /FEA = mRNA /PROD = glycoprotein | | | | |
| | | /DB_XREF = gi: 348911 /UG = Hs.75184 | | | | |
| | | chitinase 3-like 1 (cartilage | | | | |
| | | glycoprotein-39) /FL = gb: M80927.1 | | | | |
| | | gb: NM_001276.1 | | | | |
| 212657_s_at | interleukin 1 recept [symbol] | Consensus includes gb: U65590 | IL1RN | −1.2 | −2.6 | −3.0 |
| | | /DEF = Homo sapiens IL-1 receptor | | | | |
| | | antagonist IL-1Ra (IL-1RN) gene, | | | | |
| | | alternatively spliced forms, complete | | | | |
| | | cds /FEA = mRNA_2 | | | | |
| | | /DB_XREF = gi: 2707374 /UG = Hs.81134 | | | | |
| | | interleukin 1 receptor antagonist | | | | |
| 219494_at | RAD54 homolog B | gb: NM_012415.1 /DEF = Homo sapiens | RAD54B | 1.0 | 1.0 | 1.0 |
| | (S. cerevisiae) | RAD54, S. cerevisiae, homolog of, B | | | | |
| | | (RAD54B), mRNA. /FEA = mRNA | | | | |
| | | /GEN = RAD54B /PROD = RAD54, S. cerevisiae, | | | | |
| | | homolog of, B | | | | |
| | | /DB_XREF = gi: 6912621 /UG = Hs.128501 | | | | |
| | | RAD54, S. cerevisiae, homolog of, B | | | | |
| | | /FL = gb: BC001965.1 gb: AF112481.1 | | | | |
| | | gb: NM_012415.1 | | | | |
| 216598_s_at | chemokine (C-C | Consensus includes gb: S69738.1 | CCL2 | −1.3 | −3.1 | −2.4 |
| | motif) ligand 2 | /DEF = MCP-1 = monocyte chemotactic | | | | |
| | | protein human, aortic endothelial cells, | | | | |
| | | mRNA, 661 nt. /FEA = mRNA /GEN = MCP- | | | | |
| | | 1 /PROD = MCP-1 /DB_XREF = gi: 545464 | | | | |
| | | /UG = Hs.303649 small inducible cytokine | | | | |
| | | A2 (monocyte chemotactic protein 1, | | | | |
| | | homologous to mouse Sig-je) | | | | |
| 218355_at | kinesin family | gb: NM_012310.2 /DEF = Homo sapiens | KIF4A | −1.0 | −1.2 | −1.0 |
| | member 4A | kinesin family member 4A (KIF4A), | | | | |
| | | mRNA. /FEA = mRNA /GEN = KIF4A | | | | |
| | | /PROD = kinesin family member 4 | | | | |
| | | /DB_XREF = gi: 7305204 /UG = Hs.279766 kinesin family member | | | | |
| | | 4A/FL = gb: AF179308.1 gb: AF071592.2 | | | | |
| | | gb: NM_012310.2 | | | | |
| 205312_at | spleen focus | gb: NM_003120.1 /DEF = Homo sapiens | SPI1 | 1.0 | 1.0 | −1.2 |
| | forming virus | spleen focus forming virus (SFFV) proviral | | | | |
| | (SFFV) proviral | integration oncogene spi1 (SPI1), mRNA. | | | | |
| | integration | /FEA = mRNA /GEN = SPI1 /PROD = spleen | | | | |
| | oncogene spi1 | focus forming virus (SFFV) | | | | |
| | | proviralintegration oncogene spi1 | | | | |
| | | /DB_XREF = gi: 4507174 /UG = Hs.157441 | | | | |
| | | spleen focus forming virus (SFFV) proviral | | | | |
| | | integration oncogene spi1 | | | | |
| | | /FL = gb: NM_003120.1 | | | | |
| 206499_s_at | chromosome | gb: NM_001269.1 /DEF = Homo sapiens | CHC1 | 1.3 | 1.2 | −1.5 |
| | condensation 1 | chromosome condensation 1 (CHC1), | | | | |
| | | mRNA. /FEA = mRNA /GEN = CHC1 | | | | |
| | | /PROD = chromosome condensation 1 | | | | |
| | | /DB_XREF = gi: 4502800 /UG = Hs.84746 | | | | |
| | | chromosome condensation 1 | | | | |
| | | /FL = gb: NM_001269.1 | | | | |
| 216950_s_at | Fc fragment of IgG, | Consensus includes gb: X14355.1 | FCGR1A | 1.0 | −1.1 | −1.2 |
| | high affinity Ia, receptor for (CD64) | /DEF = Human mRNA for high affinity Fc | | | | |
| | | receptor (FcRI) b form. /FEA = mRNA | | | | |
| | | /DB_XREF = gi: 31333 /UG = Hs.77424 Fc | | | | |
| | | fragment of IgG, high affinity Ia, receptor | | | | |
| | | for (CD64) | | | | |
| 206130_s_at | asialoglycoprotein | gb: NM_001181.1 /DEF = Homo sapiens | ASGR2 | 1.4 | 1.0 | −1.3 |
| | receptor 2 | asialoglycoprotein receptor 2 (ASGR2), | | | | |
| | | mRNA. /FEA = mRNA /GEN = ASGR2 | | | | |
| | | /PROD = asialoglycoprotein receptor 2 | | | | |
| | | /DB_XREF = gi: 4502252 /UG = Hs.1259 | | | | |
| | | asialoglycoprotein receptor 2 | | | | |
| | | /FL = gb: M11025.1 gb: NM_001181.1 | | | | |
| 209464_at | aurora kinase B | gb: AB011446.1 /DEF = Homo sapiens | AURKB | −1.0 | −1.0 | −1.1 |
| | | mRNA for Aik2, complete cds. | | | | |
| | | /FEA = mRNA /GEN = aik2 /PROD = Aik2 | | | | |
| | | /DB_XREF = gi: 5688865 /UG = Hs.180655 | | | | |
| | | serinethreonine kinase 12 | | | | |
| | | /FL = gb: BC000442.1 gb: AF004022.1 | | | | |
| | | gb: AF008552.1 gb: AB011450.1 | | | | |
| | | gb: AF015254.1 gb: NM_004217.1 | | | | |
| | | gb: AB011446.1 | | | | |
| 205997_at | a disintegrin and | gb: NM_021778.1 /DEF = Homo sapiens a | ADAM28 | 1.0 | −1.3 | −1.2 |
| | metalloproteinase domain 28 | disintegrin and metalloproteinase domain | | | | |
| | | 28 (ADAM28), transcript variant 2, mRNA. | | | | |
| | | /FEA = mRNA /GEN = ADAM28 /PROD = a | | | | |
| | | disintegrin and metalloproteinase domain | | | | |
| | | 28, isoform 2 preproprotein | | | | |
| | | /DB_XREF = gi: 11496995 /UG = Hs.174030 | | | | |
| | | a disintegrin and metalloproteinase | | | | |
| | | domain 28 /FL = gb: NM_021778.1 | | | | |
| | | gb: AF137334.1 gb: NM_014265.1 | | | | |
| 202191_s_at | growth arrest- | Consensus includes gb: BE439987 | GAS7 | 1.2 | −1.4 | −1.1 |
| | specific 7 | /FEA = EST /DB_XREF = gi: 9439470 | | | | |
| | | /DB_XREF = est:HTM1-745F | | | | |
| | | /UG = Hs.226133 growth arrest-specific 7 | | | | |
| | | /FL = gb: AB007854.1 gb: NM_005890.1 | | | | |
| 219952_s_at | mucolipin 1 | gb: NM_020533.1 /DEF = Homo sapiens | MCOLN1 | 1.8 | −1.6 | 1.4 |
| | | mucolipin 1 (MCOLN1), mRNA. | | | | |
| | | /FEA = mRNA /GEN = MCOLN1 | | | | |
| | | /PROD = mucolipin 1 | | | | |
| | | /DB_XREF = gi: 10092596 /UG = Hs.12909 | | | | |
| | | mucolipin 1 /FL = gb: NM_020533.1 | | | | |
| | | gb: BC005149.1 gb: AF287269.1 | | | | |
| | | gb: AF249319.1 | | | | |
| 204244_s_at | activator of S | gb: NM_006716.1 /DEF = Homo sapiens | ASK | 1.1 | 1.0 | 1.1 |
| | phase kinase | activator of S phase kinase (ASK), mRNA. | | | | |
| | | /FEA = mRNA /GEN = ASK /PROD = activator | | | | |
| | | of S phase kinase /DB_XREF = gi: 5729733 | | | | |
| | | /UG = Hs.152759 activator of S phase | | | | |
| | | kinase /FL = gb: AB028069.1 | | | | |
| | | gb: AF160249.1 gb: AF160876.1 | | | | |
| | | gb: NM_006716.1 | | | | |
| 205936_s_at | hexokinase 3 | gb: NM_002115.1 /DEF = Homo sapiens | HK3 | 2.4 | −2.5 | −1.8 |
| | (white cell) | hexokinase 3 (white cell) (HK3), mRNA. | | | | |
| | | /FEA = mRNA /GEN = HK3 | | | | |
| | | /PROD = hexokinase 3 (white cell) | | | | |
| | | /DB_XREF = gi: 4504394 /UG = Hs.159237 | | | | |
| | | hexokinase 3 (white cell) /FL = gb: U51333.1 | | | | |
| | | gb: NM_002115.1 | | | | |
| 204766_s_at | nudix (nucleoside | gb: NM_002452.1 /DEF = Homo sapiens | NUDT1 | 67.7 | −1.2 | 1.0 |
| | diphosphate linked moiety X)-type | nudix (nucleoside diphosphate linked | | | | |
| | motif 1 | moiety X)-type motif 1 (NUDT1), mRNA. | | | | |
| | | /FEA = mRNA /GEN = NUDT1 /PROD = nudix | | | | |
| | | (nucleoside diphosphate linked moietyX)- | | | | |
| | | type motif 1 /DB_XREF = gi: 4505274 | | | | |
| | | /UG = Hs.388 nudix (nucleoside | | | | |
| | | diphosphate linked moiety X)-type motif 1 | | | | |
| | | /FL = gb: D16581.1 gb: NM_002452.1 | | | | |
| | | gb: AB025233.1 gb: AB025235.1 | | | | |
| | | gb: AB025237.1 gb: AB025239.1 | | | | |
| | | gb: AB025240.1 gb: AB025241.1 | | | | |
| | | gb: AB025242.1 | | | | |
| 206760_s_at | Fc fragment of IgE, | gb: NM_002002.1 /DEF = Homo sapiens Fc | FCER2 | −1.0 | −1.0 | −1.0 |
| | low affinity II, | fragment of IgE, low affinity II, receptor for | | | | |
| | receptor for | (CD23A) (FCER2), mRNA. /FEA = mRNA | | | | |
| | (CD23A) | /GEN = FCER2 /PROD = Fc fragment of IgE, | | | | |
| | | low affinity II, receptorfor (CD23A) | | | | |
| | | /DB_XREF = gi: 4503678 /UG = Hs.1416 Fc | | | | |
| | | fragment of IgE, low affinity II, receptor for | | | | |
| | | (CD23A) /FL = gb: M15059.1 gb: M14766.1 | | | | |
| | | gb: NM_002002.1 | | | | |
| 208018_s_at | hemopoietic cell | gb: NM_002110.1 /DEF = Homo sapiens | HCK | 1.0 | −1.4 | −1.5 |
| | kinase | hemopoietic cell kinase (HCK), mRNA. | | | | |
| | | /FEA = mRNA /GEN = HCK | | | | |
| | | /PROD = hemopoietic cell kinase | | | | |
| | | /DB_XREF = gi: 4504356 /UG = Hs.89555 | | | | |
| | | hemopoietic cell kinase /FL = gb: M16591.1 | | | | |
| | | gb: NM_002110.1 | | | | |
| 204444_at | kinesin family | gb: NM_004523.2 /DEF = Homo sapiens | KIF11 | −1.0 | −1.0 | −1.0 |
| | member 11 | kinesin-like 1 (KNSL1), mRNA. | | | | |
| | | /FEA = mRNA /GEN = KNSL1 | | | | |
| | | /PROD = kinesin-like 1 | | | | |
| | | /DB_XREF = gi: 13699823 /UG = Hs.8878 | | | | |
| | | kinesin-like 1 /FL = gb: U37426.1 | | | | |
| | | gb: NM_004523.2 | | | | |
| 217785_s_at | SNARE protein | gb: NM_006555.1 /DEF = Homo sapiens | YKT6 | −1.0 | −84.4 | 58.3 |
| | Ykt6 | SNARE protein (YKT6), mRNA. | | | | |
| | | /FEA = mRNA /GEN = YKT6 | | | | |
| | | /PROD = SNARE protein | | | | |
| | | /DB_XREF = gi: 5730119 /UG = Hs.296244 | | | | |
| | | SNARE protein /FL = gb: U95735.1 | | | | |
| | | gb: NM_006555.1 | | | | |
| 203711_s_at | 3-hydroxyisobutyryl- | gb: NM_014362.1 /DEF = Homo sapiens 3- | HIBCH | 50.6 | −1.0 | −1.0 |
| | Coenzyme A | hydroxyisobutyryl-Coenzyme A hydrolase | | | | |
| | hydrolase | (HIBCH), mRNA. /FEA = mRNA | | | | |
| | | /GEN = HIBCH /PROD = 3-hydroxyisobutyryl- | | | | |
| | | Coenzyme A hydrolase | | | | |
| | | /DB_XREF = gi: 7657159 /UG = Hs.236642 3- | | | | |
| | | hydroxyisobutyryl-Coenzyme A hydrolase | | | | |
| | | /FL = gb: BC005190.1 gb: U66669.1 | | | | |
| | | gb: NM_014362.1 | | | | |
| 202437_s_at | cytochrome P450, | gb: NM_000104.2 /DEF = Homo sapiens | CYP1B1 | 1.3 | 1.1 | −1.2 |
| | family 1, subfamily | cytochrome P450, subfamily I (dioxin- | | | | |
| | B, polypeptide 1 | inducible), polypeptide 1 (glaucoma 3, | | | | |
| | | primary infantile) (CYP1B1), mRNA. | | | | |
| | | /FEA = mRNA /GEN = CYP1B1 | | | | |
| | | /PROD = cytochrome P450, subfamily I | | | | |
| | | (dioxin-inducible), polypeptide 1 | | | | |
| | | /DB_XREF = gi: 13325059 /UG = Hs.154654 | | | | |
| | | cytochrome P450, subfamily I (dioxin- | | | | |
| | | inducible), polypeptide 1 (glaucoma 3, | | | | |
| | | primary infantile) /FL = gb: NM_000104.2 | | | | |
| | | gb: U03688.1 | | | | |
| 214567_s_at | chemokine (C | Consensus includes gb: NM_003175.1 | XCL1 /// | 1.0 | 1.0 | −1.2 |
| | motif) ligand 1 | /DEF = Homo sapiens small inducible | XCL2 | | | |
| | | cytokine subfamily C, member 2 (SCYC2), | | | | |
| | | mRNA. /FEA = CDS /GEN = SCYC2 | | | | |
| | | /PROD = small inducible cytokine subfamily | | | | |
| | | C, member 2 /DB_XREF = gi: 4506854 | | | | |
| | | /UG = Hs.174228 small inducible cytokine | | | | |
| | | subfamily C, member 2 | | | | |
| | | /FL = gb: NM_003175.1 | | | | |
| 204826_at | cyclin F | gb: NM_001761.1 /DEF = Homo sapiens | CCNF | −72.4 | −1.0 | −1.0 |
| | | cyclin F (CCNF), mRNA. /FEA = mRNA | | | | |
| | | /GEN = CCNF /PROD = cyclin F | | | | |
| | | /DB_XREF = gi: 4502620 /UG = Hs.1973 | | | | |
| | | cyclin F /FL = gb: NM_001761.1 | | | | |
| | | gb: U17105.1 | | | | |
| 205628_at | primase, | gb: NM_000947.1 /DEF = Homo sapiens | PRIM2A | −1.0 | −1.0 | −1.0 |
| | polypeptide 2A, | primase, polypeptide 2A (58 kD) (PRIM2A), | | | | |
| | 58 kDa | mRNA. /FEA = mRNA /GEN = PRIM2A | | | | |
| | | /PROD = primase, polypeptide 2A (58 kD) | | | | |
| | | /DB_XREF = gi: 4506052 /UG = Hs.74519 | | | | |
| | | primase, polypeptide 2A (58 kD) | | | | |
| | | /FL = gb: NM_000947.1 | | | | |
| 206207_at | Charot-Leyden | gb: NM_001828.3 /DEF = Homo sapiens | CLC | 1.2 | 1.2 | −2.1 |
| | crystal protein | Charot-Leyden crystal protein (CLC), | | | | |
| | | mRNA. /FEA = mRNA /GEN = CLC | | | | |
| | | /PROD = Charot-Leyden crystal protein | | | | |
| | | /DB_XREF = gi: 6325464 /UG = Hs.889 | | | | |
| | | Charot-Leyden crystal protein | | | | |
| | | /FL = gb: L01664.1 gb: NM_001828.3 | | | | |
| 217757_at | alpha-2- | gb: NM_000014.3 /DEF = Homo sapiens | A2M | 1.0 | 1.0 | 1.3 |
| | macroglobulin | alpha-2-macroglobulin (A2M), mRNA. | | | | |
| | | /FEA = mRNA /GEN = A2M /PROD = alpha 2 | | | | |
| | | macroglobulin precursor | | | | |
| | | /DB_XREF = gi: 6226959 /UG = Hs.74561 | | | | |
| | | alpha-2-macroglobulin /FL = gb: M11313.1 | | | | |
| | | gb: NM_000014.3 | | | | |
| 204603_at | exonuclease 1 | gb: NM_003686.1 /DEF = Homo sapiens | EXO1 | −1.0 | −1.0 | −1.0 |
| | | exonuclease 1 (EXO1), mRNA. | | | | |
| | | /FEA = mRNA /GEN = EXO1 /PROD = Rad2 | | | | |
| | | nuclease family member, homolog of | | | | |
| | | S. cerevisiae exonuclease 1 | | | | |
| | | /DB_XREF = gi: 4504368 /UG = Hs.47504 | | | | |
| | | exonuclease 1 /FL = gb: NM_006027.1 | | | | |
| | | gb: AF042282.1 gb: AF060479.1 | | | | |
| | | gb: AF091740.1 gb: AF084974.1 | | | | |
| | | gb: NM_003686.1 | | | | |
| 203968_s_at | CDC6 cell division | gb: NM_001254.1 /DEF = Homo sapiens | CDC6 | −1.0 | −1.0 | 1.1 |
| | cycle 6 homolog | CDC6 (cell division cycle 6, S. cerevisiae) | | | | |
| | (S. cerevisiae) | homolog (CDC6), mRNA. /FEA = mRNA | | | | |
| | | /GEN = CDC6 /PROD = CDC6 (cell division | | | | |
| | | cycle 6, S. cerevisiae)homolog | | | | |
| | | /DB_XREF = gi: 4502702 /UG = Hs.69563 | | | | |
| | | CDC6 (cell division cycle 6, S. cerevisiae) | | | | |
| | | homolog /FL = gb: U77949.1 gb: AF022109.1 | | | | |
| | | gb: NM_001254.1 | | | | |
| 204475_at | matrix | gb: NM_002421.2 /DEF = Homo sapiens | MMP1 | −1.0 | −1.0 | −1.0 |
| | metalloproteinase | matrix metalloproteinase 1 (interstitial | | | | |
| | 1 (interstitial | collagenase) (MMP1), mRNA. | | | | |
| | collagenase) | /FEA = mRNA /GEN = MMP1 /PROD = matrix | | | | |
| | | metalloproteinase 1 preproprotein | | | | |
| | | /DB_XREF = gi: 13027798 /UG = Hs.83169 | | | | |
| | | matrix metalloproteinase 1 (interstitial | | | | |
| | | collagenase) /FL = gb: NM_002421.2 | | | | |
| | | gb: M13509.1 | | | | |
| 218009_s_at | protein regulator of | gb: NM_003981.1 /DEF = Homo sapiens | PRC1 | 1.1 | 1.0 | −61.2 |
| | cytokinesis 1 | protein regulator of cytokinesis 1 (PRC1), | | | | |
| | | mRNA. /FEA = mRNA /GEN = PRC1 | | | | |
| | | /PROD = protein regulator of cytokinesis 1 | | | | |
| | | /DB_XREF = gi: 4506038 /UG = Hs.5101 | | | | |
| | | protein regulator of cytokinesis 1 | | | | |
| | | /FL = gb: BC003138.1 gb: AF044588.1 | | | | |
| | | gb: NM_003981.1 | | | | |
| 207857_at | leukocyte | gb: NM_006866.1 /DEF = Homo sapiens | LILRA2 | 1.2 | 1.1 | −1.3 |
| | immunoglobulin- | leukocyte immunoglobulin-like receptor, | | | | |
| | like receptor, | subfamily A (with TM domain), member 2 | | | | |
| | subfamily B (with | (LILRA2), mRNA. /FEA = mRNA | | | | |
| | TM and ITIM | /GEN = LILRA2 /PROD = leukocyte | | | | |
| | domains), member 1 | immunoglobulin-like receptor, subfamily A | | | | |
| | | (with TM domain), member 2 | | | | |
| | | /DB_XREF = gi: 5803067 /UG = Hs.94498 | | | | |
| | | leukocyte immunoglobulin-like receptor, | | | | |
| | | subfamily A (with TM domain), member 2 | | | | |
| | | /FL = gb: U82275.1 gb: NM_006866.1 | | | | |
| 203973_s_at | KIAA0146 protein | gb: NM_005195.1 /DEF = Homo sapiens | CEBPD | 1.6 | 1.2 | 1.2 |
| | | CCAATenhancer binding protein (CEBP), | | | | |
| | | delta (CEBPD), mRNA. /FEA = mRNA | | | | |
| | | /GEN = CEBPD /PROD = CCAATenhancer | | | | |
| | | binding protein (CEBP), delta | | | | |
| | | /DB_XREF = gi: 4885130 /UG = Hs.76722 | | | | |
| | | CCAATenhancer binding protein (CEBP), | | | | |
| | | delta /FL = gb: M83667.1 gb: NM_005195.1 | | | | |
| 201897_s_at | CDC28 protein | gb: NM_001826.1 /DEF = Homo sapiens | CKS1B | 1.1 | 1.0 | −1.3 |
| | kinase regulatory | CDC28 protein kinase 1 (CKS1), mRNA. | | | | |
| | subunit 1B | /FEA = mRNA /GEN = CKS1 /PROD = CDC28 | | | | |
| | | protein kinase 1 /DB_XREF = gi: 4502856 | | | | |
| | | /UG = Hs.77550 CDC28 protein kinase 1 | | | | |
| | | /FL = gb: BC001425.1 gb: AF274941.1 | | | | |
| | | gb: AF279897.1 gb: NM_001826.1 | | | | |
| 202095_s_at | baculoviral IAP | gb: NM_001168.1 /DEF = Homo sapiens | BIRC5 | −1.0 | −1.0 | −1.0 |
| | repeat-containing 5 | baculoviral IAP repeat-containing 5 | | | | |
| | (survivin) | (survivin) (BIRC5), mRNA. /FEA = mRNA | | | | |
| | | /GEN = BIRC5 /PROD = baculoviral IAP | | | | |
| | | repeat-containing protein 5 | | | | |
| | | /DB_XREF = gi: 4502144 /UG = Hs.1578 | | | | |
| | | baculoviral IAP repeat-containing 5 | | | | |
| | | (survivin) /FL = gb: NM_001168.1 | | | | |
| | | gb: AF077350.1 | | | | |
| 222077_s_at | Rac GTPase | Consensus includes gb: AU153848 | RACGAP1 | 2.0 | −1.2 | −1.2 |
| | activating protein 1 | /FEA = EST /DB_XREF = gi: 11015369 | | | | |
| | | /DB_XREF = est: AU153848 | | | | |
| | | /CLONE = NT2RP3004175 /UG = Hs.23900 | | | | |
| | | GTPase activating protein | | | | |
| 203603_s_at | zinc finger | gb: NM_014795.1 /DEF = Homo sapiens | ZFHX1B | 1.6 | 1.0 | −1.3 |
| | homeobox 1b | zinc finger homeobox 1B (ZFHX1B), | | | | |
| | | mRNA. /FEA = mRNA /GEN = ZFHX1B | | | | |
| | | /PROD = zinc finger homeobox 1B | | | | |
| | | /DB_XREF = gi: 7662183 /UG = Hs.34871 | | | | |
| | | zinc finger homeobox 1B | | | | |
| | | /FL = gb: AB011141.1 gb: NM_014795.1 | | | | |
| 201141_at | glycoprotein | gb: NM_002510.1 /DEF = Homo sapiens | GPNMB | 1.4 | 1.1 | 1.1 |
| | (transmembrane) | glycoprotein (transmembrane) nmb | | | | |
| | nmb | (GPNMB), mRNA. /FEA = mRNA | | | | |
| | | /GEN = GPNMB /PROD = glycoprotein | | | | |
| | | (transmembrane) nmb | | | | |
| | | /DB_XREF = gi: 4505404 /UG = Hs.82226 | | | | |
| | | glycoprotein (transmembrane) nmb | | | | |
| | | /FL = gb: AF322909.1 gb: NM_002510.1 | | | | |
| 218847_at | IGF-II mRNA- | gb: NM_006548.1 /DEF = Homo sapiens | IMP-2 | 1.5 | −1.2 | 1.1 |
| | binding protein 2 | IGF-II mRNA-binding protein 2 (IMP-2), | | | | |
| | | mRNA. /FEA = mRNA /GEN = IMP-2 | | | | |
| | | /PROD = IGF-II mRNA-binding protein 2 | | | | |
| | | /DB_XREF = gi: 5729883 /UG = Hs.30299 | | | | |
| | | IGF-II mRNA-binding protein 2 | | | | |
| | | /FL = gb: AF057352.1 gb: NM_006548.1 | | | | |
| 205769_at | solute carrier | gb: NM_003645.1 /DEF = Homo sapiens | SLC27A2 | −1.0 | −1.0 | −1.0 |
| | family 27 (fatty acid | fatty-acid-Coenzyme A ligase, very long- | | | | |
| | transporter), | chain 1 (FACVL1), mRNA. /FEA = mRNA | | | | |
| | member 2 | /GEN = FACVL1 /PROD = very long-chain | | | | |
| | | fatty-acid-Coenzyme A ligase 1 | | | | |
| | | /DB_XREF = gi: 4503652 /UG = Hs.11729 | | | | |
| | | fatty-acid-Coenzyme A ligase, very long- | | | | |
| | | chain 1 /FL = gb: D88308.1 gb: AF096290.1 | | | | |
| | | gb: NM_003645.1 | | | | |
| 209714_s_at | cyclin-dependent | gb: AF213033.1 /DEF = Homo sapiens | CDKN3 | −1.3 | 1.0 | −1.2 |
| | kinase inhibitor 3 | isolate BX-01 cyclin-dependent kinase | | | | |
| | (CDK2-associated | associated protein phosphatase mRNA, | | | | |
| | dual specificity | complete cds. /FEA = mRNA | | | | |
| | phosphatase) | /PROD = cyclin-dependent kinase | | | | |
| | | associated proteinphosphatase | | | | |
| | | /DB_XREF = gi: 12734643 /UG = Hs.84113 | | | | |
| | | cyclin-dependent kinase inhibitor 3 (CDK2- | | | | |
| | | associated dual specificity phosphatase) | | | | |
| | | /FL = gb: AF213033.1 gb: AF213034.1 | | | | |
| | | gb: AF213035.1 gb: AF213036.1 | | | | |
| | | gb: AF213037.1 gb: AF213038.1 | | | | |
| | | gb: AF213039.1 gb: AF213040.1 | | | | |
| | | gb: AF213041.1 gb: AF213042.1 | | | | |
| | | gb: AF213044.1 gb: AF213046.1 | | | | |
| | | gb: AF213047.1 gb: AF213048.1 | | | | |
| | | gb: AF213049.1 gb: AF213050.1 | | | | |
| | | gb: AF213051.1 gb: AF213052.1 | | | | |
| | | gb: AF213053.1 gb: U02681.1 gb: L25876.1 | | | | |
| | | gb: NM_005192.1 gb: L27711.1 | | | | |
| 205898_at | chemokine (C—X3—C | gb: U20350.1 /DEF = Human G protein- | CX3CR1 | 1.1 | 1.0 | −1.2 |
| | motif) receptor 1 | coupled receptor V28 mRNA, complete | | | | |
| | | cds. /FEA = mRNA /PROD = V28 | | | | |
| | | /DB_XREF = gi: 665580 /UG = Hs.78913 | | | | |
| | | chemokine (C—X3—C) receptor 1 | | | | |
| | | /FL = gb: NM_001337.1 gb: U20350.1 | | | | |
| | | gb: U28934.1 | | | | |
| 203145_at | sperm associated | gb: NM_006461.1 /DEF = Homo sapiens | SPAG5 | 1.1 | 1.3 | 94.7 |
| | antigen 5 | mitotic spindle coiled-coil related protein | | | | |
| | | (DEEPEST), mRNA. /FEA = mRNA | | | | |
| | | /GEN = DEEPEST /PROD = mitotic spindle | | | | |
| | | coiled-coil related protein | | | | |
| | | /DB_XREF = gi: 5453631 /UG = Hs.16244 | | | | |
| | | mitotic spindle coiled-coil related protein | | | | |
| | | /FL = gb: BC000322.1 gb: AF063308.1 | | | | |
| | | gb: NM_006461.1 | | | | |
| 205119_s_at | formyl peptide | gb: NM_002029.1 /DEF = Homo sapiens | | 1.3 | 1.0 | −1.4 |
| | receptor 1 | formyl peptide receptor 1 (FPR1), mRNA. | | | | |
| | | /FEA = mRNA /GEN = FPR1 /PROD = formyl | | | | |
| | | peptide receptor 1 /DB_XREF = gi: 4503778 | | | | |
| | | /UG = Hs.753 formyl peptide receptor 1 | | | | |
| | | /FL = gb: BC005315.1 gb: M60626.1 | | | | |
| | | gb: M60627.1 gb: NM_002029.1 | | | | |
| 203256_at | cadherin 3, type 1, | gb: NM_001793.1 /DEF = Homo sapiens | CDH3 | −1.0 | −1.0 | −1.0 |
| | P-cadherin | cadherin 3, type 1, P-cadherin (placental) | | | | |
| | (placental) | (CDH3), mRNA. /FEA = mRNA | | | | |
| | | /GEN = CDH3 /PROD = cadherin 3, type 1 P- | | | | |
| | | cadherin (placental) | | | | |
| | | /DB_XREF = gi: 4502722 /UG = Hs.2877 | | | | |
| | | cadherin 3, type 1, P-cadherin (placental) | | | | |
| | | /FL = gb: NM_001793.1 | | | | |
| 205085_at | origin recognition | gb: NM_004153.1 /DEF = Homo sapiens | ORC1L | −1.0 | −1.0 | −1.0 |
| | complex, subunit 1- | origin recognition complex, subunit 1 | | | | |
| | like (yeast) | (yeast homolog)-like (ORC1L), mRNA. | | | | |
| | | /FEA = mRNA /GEN = ORC1L /PROD = origin | | | | |
| | | recognition complex, subunit 1 | | | | |
| | | (yeasthomolog)-like | | | | |
| | | /DB_XREF = gi: 4758849 /UG = Hs.17908 | | | | |
| | | origin recognition complex, subunit 1 | | | | |
| | | (yeast homolog)-like /FL = gb: U40152.1 | | | | |
| | | gb: U43416.1 gb: NM_004153.1 | | | | |
| 204759_at | chromosome | gb: NM_001268.1 /DEF = Homo sapiens | CHC1L | 1.6 | 1.0 | 1.2 |
| | condensation 1-like | chromosome condensation 1-like | | | | |
| | | (CHC1L), mRNA. /FEA = mRNA | | | | |
| | | /GEN = CHC1L /PROD = RCC1-like G | | | | |
| | | exchanging factor RLG | | | | |
| | | /DB_XREF = gi: 4557444 /UG = Hs.27007 | | | | |
| | | chromosome condensation 1-like | | | | |
| | | /FL = gb: AF060219.1 gb: NM_001268.1 | | | | |
| 202006_at | protein tyrosine | gb: NM_002835.1 /DEF = Homo sapiens | PTPN12 | 1.1 | −1.2 | −1.7 |
| | phosphatase, non- | protein tyrosine phosphatase, non- | | | | |
| | receptor type 12 | receptor type 12 (PTPN12), mRNA. | | | | |
| | | /FEA = mRNA /GEN = PTPN12 | | | | |
| | | /PROD = protein tyrosine phosphatase, non- | | | | |
| | | receptor type12 /DB_XREF = gi: 4506286 | | | | |
| | | /UG = Hs.62 protein tyrosine phosphatase, | | | | |
| | | non-receptor type 12 /FL = gb: D13380.1 | | | | |
| | | gb: M93425.1 gb: NM_002835.1 | | | | |
| 202086_at | myxovirus | gb: NM_002462.1 /DEF = Homo sapiens | MX1 | 1.1 | −7.3 | −1.2 |
| | (influenza virus) | myxovirus (influenza) resistance 1, | | | | |
| | resistance 1, | homolog of murine (interferon-inducible | | | | |
| | interferon-inducible | protein p78) (MX1), mRNA. /FEA = mRNA | | | | |
| | protein p78 | /GEN = MX1 /PROD = myxovirus (influenza) | | | | |
| | (mouse) | resistance 1, homolog ofmurine (interferon- | | | | |
| | | inducible protein p78) | | | | |
| | | /DB_XREF = gi: 4505290 /UG = Hs.76391 | | | | |
| | | myxovirus (influenza) resistance 1, | | | | |
| | | homolog of murine (interferon-inducible | | | | |
| | | protein p78) /FL = gb: M30817.1 | | | | |
| | | gb: M33882.1 gb: NM_002462.1 | | | | |
| 210423_s_at | solute carrier | gb: L32185.1 /DEF = Homo sapiens integral | SLC11A1 | 1.1 | 1.1 | 1.1 |
| | family 11 (proton- | membrane protein (NRAMP1) mRNA, | | | | |
| | coupled divalent | complete cds. /FEA = mRNA | | | | |
| | metal ion | /GEN = NRAMP1 /PROD = integral | | | | |
| | transporters), | membrane protein /DB_XREF = gi: 600219 | | | | |
| | member 1 | /UG = Hs.182611 solute carrier family 11 | | | | |
| | | (proton-coupled divalent metal ion | | | | |
| | | transporters), member 1 /FL = gb: D50402.1 | | | | |
| | | gb: D50403.1 gb: NM_000578.1 | | | | |
| | | gb: L32185.1 | | | | |
| 204588_s_at | solute carrier | gb: NM_003982.1 /DEF = Homo sapiens | SLC7A7 | 1.0 | −1.1 | 1.0 |
| | family 7 (cationic | solute carrier family 7 (cationic amino acid | | | | |
| | amino acid | transporter, y+ system), member 7 | | | | |
| | transporter, y+ | (SLC7A7), mRNA. /FEA = mRNA | | | | |
| | system), member 7 | /GEN = SLC7A7 /PROD = solute carrier | | | | |
| | | family 7 (cationic amino acidtransporter, | | | | |
| | | y+ system), member 7 | | | | |
| | | /DB_XREF = gi: 4507054 /UG = Hs.194693 | | | | |
| | | solute carrier family 7 (cationic amino acid | | | | |
| | | transporter, y+ system), member 7 | | | | |
| | | /FL = gb: BC003062.1 gb: AF092032.1 | | | | |
| | | gb: NM_003982.1 gb: AB011263.1 | | | | |
| 214437_s_at | serine | Consensus includes gb: NM_005412.1 | SHMT2 | 1.3 | −1.3 | −1.6 |
| | hydroxymethyltransferase 2 | /DEF = Homo sapiens serine | | | | |
| | (mitochondrial) | hydroxymethyltransferase 2 | | | | |
| | | (mitochondrial) (SHMT2), mRNA. | | | | |
| | | /FEA = CDS /GEN = SHMT2 /PROD = serine | | | | |
| | | hydroxymethyltransferase 2(mitochondrial) | | | | |
| | | /DB_XREF = gi: 4885594 /UG = Hs.75069 | | | | |
| | | serine hydroxymethyltransferase 2 | | | | |
| | | (mitochondrial) /FL = gb: NM_005412.1 | | | | |
| 216248_s_at | nuclear receptor | Consensus includes gb: S77154.1 | NR4A2 | 1.2 | 1.0 | −1.1 |
| | subfamily 4, group | /DEF = TINUR = NGFI-Bnur77 beta-type | | | | |
| | A, member 2 | transcription factor homolog human, T | | | | |
| | | lymphoid cell line, PEER, mRNA, 2469 nt. | | | | |
| | | /FEA = mRNA /GEN = TINUR | | | | |
| | | /DB_XREF = gi: 913966 /UG = Hs.82120 | | | | |
| | | nuclear receptor subfamily 4, group A, | | | | |
| | | member 2 | | | | |
| 204170_s_at | CDC28 protein | gb: NM_001827.1 /DEF = Homo sapiens | CKS2 | 1.1 | −1.7 | −1.9 |
| | kinase regulatory | CDC28 protein kinase 2 (CKS2), mRNA. | | | | |
| | subunit 2 | /FEA = mRNA /GEN = CKS2 /PROD = CDC28 | | | | |
| | | protein kinase 2 /DB_XREF = gi: 4502858 | | | | |
| | | /UG = Hs.83758 CDC28 protein kinase 2 | | | | |
| | | /FL = gb: NM_001827.1 | | | | |
| 201425_at | aldehyde | gb: NM_000690.1 /DEF = Homo sapiens | ALDH2 | 1.1 | 1.1 | −1.3 |
| | dehydrogenase 2 | aldehyde dehydrogenase 2, mitochondrial | | | | |
| | family | (ALDH2), mRNA. /FEA = mRNA | | | | |
| | (mitochondrial) | /GEN = ALDH2 /PROD = aldehyde | | | | |
| | | dehydrogenase 2, mitochondrial | | | | |
| | | /DB_XREF = gi: 4502032 /UG = Hs.195432 | | | | |
| | | aldehyde dehydrogenase 2 family | | | | |
| | | (mitochondrial) /FL = gb: BC002967.1 | | | | |
| | | gb: NM_000690.1 | | | | |
| 204575_s_at | matrix | gb: U38321.1 /DEF = Homo sapiens clone | MMP19 | −1.4 | −69.0 | 51.1 |
| | metalloproteinase | rasi-11 matrix metalloproteinase RASI-1 | | | | |
| | 19 | mRNA, complete cds. /FEA = mRNA | | | | |
| | | /PROD = matrix metalloproteinase RASI-1 | | | | |
| | | /DB_XREF = gi: 2228243 /UG = Hs.154057 | | | | |
| | | matrix metalloproteinase 19 | | | | |
| | | /FL = gb: NM_002429.2 gb: U38321.1 | | | | |
| | | gb: U37791.1 | | | | |
| 206414_s_at | development and | gb: NM_003887.1 /DEF = Homo sapiens | DDEF2 | −1.0 | −1.0 | −1.0 |
| | differentiation | development and differentiation enhancing | | | | |
| | enhancing factor 2 | factor 2 (DDEF2), mRNA. /FEA = mRNA | | | | |
| | | /GEN = DDEF2 /PROD = ADP-ribosylation | | | | |
| | | factorarf-directed GTPaseactivating | | | | |
| | | protein /DB_XREF = gi: 4502248 | | | | |
| | | /UG = Hs.12802 development and | | | | |
| | | differentiation enhancing factor 2 | | | | |
| | | /FL = gb: AB007860.1 gb: NM_003887.1 | | | | |
| 218567_x_at | dipeptidylpeptidase 3 | gb: NM_005700.1 /DEF = Homo sapiens | DPP3 | −1.2 | −1.7 | −1.6 |
| | | dipeptidylpeptidase III (DPP3), mRNA. | | | | |
| | | /FEA = mRNA /GEN = DPP3 | | | | |
| | | /PROD = dipeptidylpeptidase III | | | | |
| | | /DB_XREF = gi: 11342681 /UG = Hs.22880 | | | | |
| | | dipeptidylpeptidase III | | | | |
| | | /FL = gb: NM_005700.1 gb: BC001446.1 | | | | |
| 202820_at | aryl hydrocarbon | gb: NM_001621.2 /DEF = Homo sapiens | AHR | 1.2 | 1.1 | 1.2 |
| | receptor | aryl hydrocarbon receptor (AHR), mRNA. | | | | |
| | | /FEA = mRNA /GEN = AHR /PROD = aryl | | | | |
| | | hydrocarbon receptor | | | | |
| | | /DB_XREF = gi: 5016091 /UG = Hs.170087 | | | | |
| | | aryl hydrocarbon receptor | | | | |
| | | /FL = gb: L19872.1 gb: D16354.1 | | | | |
| | | gb: NM_001621.2 | | | | |
| 214038_at | chemokine (C-C | Consensus includes gb: AI984980 | CCL8 | 1.1 | −8.4 | −1.2 |
| | motif) ligand 8 | /FEA = EST /DB_XREF = gi: 5812257 | | | | |
| | | /DB_XREF = est: wr88g11.x1 | | | | |
| | | /CLONE = IMAGE: 2494820 | | | | |
| | | /UG = Hs.271387 small inducible cytokine | | | | |
| | | subfamily A (Cys-Cys), member 8 | | | | |
| | | (monocyte chemotactic protein 2) | | | | |
| | | /FL = gb: NM_005623.1 | | | | |
| 204439_at | chromosome 1 | gb: NM_006820.1 /DEF = Homo sapiens | C1orf29 | −1.0 | −894.0 | 2.2 |
| | open reading | hypothetical protein, expressed in | | | | |
| | frame 29 | osteoblast (GS3686), mRNA. | | | | |
| | | /FEA = mRNA /GEN = GS3686 | | | | |
| | | /PROD = hypothetical protein, expressed in | | | | |
| | | osteoblast /DB_XREF = gi: 5803026 | | | | |
| | | /UG = Hs.75470 hypothetical protein, | | | | |
| | | expressed in osteoblast | | | | |
| | | /FL = gb: AB000115.1 gb: NM_006820.1 | | | | |
| 217853_at | tensin-like SH2 | gb: NM_022748.1 /DEF = Homo sapiens | TENS1 | 1.2 | −1.4 | −1.9 |
| | domain containing 1 | hypothetical protein FLJ13732 similar to | | | | |
| | | tensin (FLJ13732), mRNA. /FEA = mRNA | | | | |
| | | /GEN = FLJ13732 /PROD = hypothetical | | | | |
| | | protein FLJ13732 similar to tensin | | | | |
| | | /DB_XREF = gi: 12232408 /UG = Hs.12210 | | | | |
| | | hypothetical protein FLJ13732 similar to | | | | |
| | | tensin /FL = gb: NM_022748.1 | | | | |
| 220646_s_at | killer cell lectin-like | gb: NM_016523.1 /DEF = Homo sapiens | KLRF1 | 1.7 | 1.1 | 1.1 |
| | receptor subfamily | killer cell lectin-like receptor F1 (KLRF1), | | | | |
| | F, member 1 | mRNA. /FEA = mRNA /GEN = KLRF1 | | | | |
| | | /PROD = killer cell lectin-like receptor F1 | | | | |
| | | /DB_XREF = gi: 7705573 /UG = Hs.183125 | | | | |
| | | killer cell lectin-like receptor F1 | | | | |
| | | /FL = gb: AF175206.1 gb: NM_016523.1 | | | | |
| 201666_at | tissue inhibitor of | gb: NM_003254.1 /DEF = Homo sapiens | TIMP1 | 1.0 | −1.3 | 1.0 |
| | metalloproteinase | tissue inhibitor of metalloproteinase 1 | | | | |
| | 1 (erythroid | (erythroid potentiating activity, collagenase | | | | |
| | potentiating | inhibitor) (TIMP1), mRNA. /FEA = mRNA | | | | |
| | activity, | /GEN = TIMP1 /PROD = tissue inhibitor of | | | | |
| | collagenase | metalloproteinase 1precursor | | | | |
| | inhibitor) | /DB_XREF = gi: 4507508 /UG = Hs.5831 | | | | |
| | | tissue inhibitor of metalloproteinase 1 | | | | |
| | | (erythroid potentiating activity, collagenase | | | | |
| | | inhibitor) /FL = gb: BC000866.1 | | | | |
| | | gb: M12670.1 gb: M59906.1 | | | | |
| | | gb: NM_003254.1 | | | | |
| 219505_at | cat eye syndrome | gb: NM_017424.1 /DEF = Homo sapiens cat | CECR1 | −1.4 | −1.2 | 1.1 |
| | chromosome | eye syndrome chromosome region, | | | | |
| | region, candidate 1 | candidate 1 (CECR1), mRNA. | | | | |
| | | /FEA = mRNA /GEN = CECR1 /PROD = cat | | | | |
| | | eye syndrome critical region protein 1 | | | | |
| | | /DB_XREF = gi: 8393092 /UG = Hs.170310 | | | | |
| | | cat eye syndrome chromosome region, | | | | |
| | | candidate 1 /FL = gb: AF190746.1 | | | | |
| | | gb: NM_017424.1 | | | | |
| 204936_at | splicing factor 1 | gb: NM_004579.1 /DEF = Homo sapiens | MAP4K2 | −1.0 | −1.0 | −1.0 |
| | [BLAST] | mitogen-activating protein kinase kinase | | | | |
| | | kinase kinase 2 (MAP4K2), mRNA. | | | | |
| | | /FEA = mRNA /GEN = MAP4K2 | | | | |
| | | /PROD = mitogen-activating protein kinase | | | | |
| | | kinase kinasekinase 2 | | | | |
| | | /DB_XREF = gi: 4759009 /UG = Hs.82979 | | | | |
| | | mitogen-activating protein kinase kinase | | | | |
| | | kinase kinase 2 /FL = gb: NM_004579.1 | | | | |
| | | gb: U07349.1 | | | | |
| 209189_at | v-fos FBJ murine | gb: BC004490.1 /DEF = Homo sapiens, v- | FOS | 1.1 | 1.0 | 1.5 |
| | osteosarcoma viral | fos FBJ murine osteosarcoma viral | | | | |
| | oncogene homolog | oncogene homolog, clone MGC: 11074, | | | | |
| | | mRNA, complete cds. /FEA = mRNA | | | | |
| | | /PROD = v-fos FBJ murine osteosarcoma | | | | |
| | | viral oncogenehomolog | | | | |
| | | /DB_XREF = gi: 13325363 /UG = Hs.25647 v- | | | | |
| | | fos FBJ murine osteosarcoma viral | | | | |
| | | oncogene homolog /FL = gb: BC004490.1 | | | | |
| | | gb: NM_005252.2 | | | | |
| 217996_at | pleckstrin | Consensus includes gb: AA576961 | PHLDA1 | 1.2 | 63.7 | −66.0 |
| | homology-like | /FEA = EST /DB_XREF = gi: 2354435 | | | | |
| | domain, family A, | /DB_XREF = est: nm82d08.s1 | | | | |
| | member 1 | /CLONE = IMAGE: 1074735 /UG = Hs.82101 | | | | |
| | | pleckstrin homology-like domain, family A, | | | | |
| | | member 1 /FL = gb: NM_007350.1 | | | | |
| 218350_s_at | geminin, DNA | gb: NM_015895.1 /DEF = Homo sapiens | GMNN | 1.2 | −1.3 | −1.3 |
| | replication inhibitor | geminin (LOC51053), mRNA. | | | | |
| | | /FEA = mRNA /GEN = LOC51053 | | | | |
| | | /PROD = geminin /DB_XREF = gi: 7705681 | | | | |
| | | /UG = Hs.234896 geminin | | | | |
| | | /FL = gb: AF067855.1 gb: NM_015895.1 | | | | |
| 209709_s_at | hyaluronan- | gb: U29343.1 /DEF = Homo sapiens | HMMR | −71.2 | −1.0 | −74.3 |
| | mediated motility | hyaluronan receptor (RHAMM) mRNA, | | | | |
| | receptor (RHAMM) | complete cds. /FEA = mRNA | | | | |
| | | /GEN = RHAMM /PROD = hyaluronan | | | | |
| | | receptor /DB_XREF = gi: 2959555 | | | | |
| | | /UG = Hs.72550 hyaluronan-mediated | | | | |
| | | motility receptor (RHAMM) | | | | |
| | | /FL = gb: U29343.1 gb: AF032862.1 | | | | |
| | | gb: NM_012484.1 | | | | |
| 220688_s_at | chromosome 1 | gb: NM_016183.1 /DEF = Homo sapiens | C1orf33 | 1.3 | −1.2 | −1.3 |
| | open reading | 60S acidic ribosomal protein PO | | | | |
| | frame 33 | (LOC51154), mRNA. /FEA = mRNA | | | | |
| | | /GEN = LOC51154 /PROD = 60S acidic | | | | |
| | | ribosomal protein PO | | | | |
| | | /DB_XREF = gi: 7705874 /UG = Hs.274201 | | | | |
| | | 60S acidic ribosomal protein PO | | | | |
| | | /FL = gb: AF173378.1 gb: NM_016183.1 | | | | |
| 202388_at | regulator of G- | gb: NM_002923.1 /DEF = Homo sapiens | RGS2 | 1.4 | 1.2 | 1.3 |
| | protein signalling 2, | regulator of G-protein signalling 2, 24 kD | | | | |
| | 24 kDa | (RGS2), mRNA. /FEA = mRNA | | | | |
| | | /GEN = RGS2 /PROD = regulator of G- | | | | |
| | | protein signalling 2, 24 kD | | | | |
| | | /DB_XREF = gi: 4506516 /UG = Hs.78944 | | | | |
| | | regulator of G-protein signalling 2, 24 kD | | | | |
| | | /FL = gb: L13463.1 gb: NM_002923.1 | | | | |
| 201506_at | transforming | gb: NM_000358.1 /DEF = Homo sapiens | TGFBI | 1.0 | −1.2 | 1.0 |
| | growth factor, beta- | transforming growth factor, beta-induced, | | | | |
| | induced, 68 kDa | 68 kD (TGFBI), mRNA. /FEA = mRNA | | | | |
| | | /GEN = TGFBI /PROD = transforming growth | | | | |
| | | factor, beta-induced, 68 kD | | | | |
| | | /DB_XREF = gi: 4507466 /UG = Hs.118787 | | | | |
| | | transforming growth factor, beta-induced, | | | | |
| | | 68 kD /FL = gb: BC000097.1 gb: BC004972.1 | | | | |
| | | gb: M77349.1 gb: NM_000358.1 | | | | |
| 204092_s_at | serine/threonine | gb: NM_003600.1 /DEF = Homo sapiens | STK6 | −1.0 | −60.9 | 55.8 |
| | kinase 6 | serinethreonine kinase 15 (STK15), | | | | |
| | | mRNA. /FEA = mRNA /GEN = STK15 | | | | |
| | | /PROD = serinethreonine kinase 15 | | | | |
| | | /DB_XREF = gi: 4507274 /UG = Hs.48915 | | | | |
| | | serinethreonine kinase 15 | | | | |
| | | /FL = gb: AF008551.1 gb: AF011468.1 | | | | |
| | | gb: NM_003600.1 | | | | |
| 203561_at | Fc fragment of IgG, | gb: NM_021642.1 /DEF = Homo sapiens Fc | FCGR2A | 1.1 | −1.2 | 1.2 |
| | low affinity IIa, | fragment of IgG, low affinity IIa, receptor | | | | |
| | receptor for (CD32) | for (CD32) (FCGR2A), mRNA. | | | | |
| | | /FEA = mRNA /GEN = FCGR2A /PROD = Fc | | | | |
| | | fragment of IgG, low affinity IIa, | | | | |
| | | receptorfor (CD32) | | | | |
| | | /DB_XREF = gi: 11056051 /UG = Hs.78864 | | | | |
| | | Fc fragment of IgG, low affinity IIa, | | | | |
| | | receptor for (CD32) /FL = gb: NM_021642.1 | | | | |
| | | gb: M31932.1 gb: J03619.1 gb: M28697.1 | | | | |
| 217983_s_at | ribonuclease T2 | gb: NM_003730.2 /DEF = Homo sapiens | RNASET2 | 1.4 | 1.1 | 1.1 |
| | | ribonuclease 6 precursor (RNASE6PL), | | | | |
| | | mRNA. /FEA = mRNA /GEN = RNASE6PL | | | | |
| | | /PROD = ribonuclease 6 precursor | | | | |
| | | /DB_XREF = gi: 5231227 /UG = Hs.8297 | | | | |
| | | ribonuclease 6 precursor | | | | |
| | | /FL = gb: BC001660.1 gb: BC001819.1 | | | | |
| | | gb: U85625.2 gb: NM_003730.2 | | | | |
| 204446_s_at | arachidonate 5- | gb: NM_000698.1 /DEF = Homo sapiens | ALOX5 | 1.2 | −1.3 | −1.3 |
| | lipoxygenase | arachidonate 5-lipoxygenase (ALOX5), | | | | |
| | | mRNA. /FEA = mRNA /GEN = ALOX5 | | | | |
| | | /PROD = arachidonate 5-lipoxygenase | | | | |
| | | /DB_XREF = gi: 4502056 /UG = Hs.89499 | | | | |
| | | arachidonate 5-lipoxygenase | | | | |
| | | /FL = gb: J03600.1 gb: J03571.1 | | | | |
| | | gb: NM_000698.1 | | | | |
| 208303_s_at | cytokine receptor- | gb: NM_022148.1 /DEF = Homo sapiens | CRLF2 | −65.7 | 67.0 | 56.7 |
| | like factor 2 | cytokine receptor-like factor 2 (CRLF2), | | | | |
| | | mRNA. /FEA = mRNA /GEN = CRLF2 | | | | |
| | | /PROD = cytokine receptor-like factor 2 | | | | |
| | | /DB_XREF = gi: 13375623 /UG = Hs.287729 | | | | |
| | | cytokine receptor-like factor 2 | | | | |
| | | /FL = gb: NM_022148.1 | | | | |
| 218589_at | purinergic receptor | gb: NM_005767.1 /DEF = Homo sapiens | P2RY5 | 1.4 | −1.1 | 1.1 |
| | P2Y, G-protein | purinergic receptor (family A group 5) | | | | |
| | coupled, 5 | (P2Y5), mRNA. /FEA = mRNA /GEN = P2Y5 | | | | |
| | | /PROD = purinergic receptor (family A | | | | |
| | | group 5) /DB_XREF = gi: 5031968 | | | | |
| | | /UG = Hs.189999 purinergic receptor | | | | |
| | | (family A group 5) /FL = gb: AF000546.1 | | | | |
| | | gb: NM_005767.1 | | | | |
| 209715_at | chromobox | gb: L07515.1 /DEF = Human | CBX5 | −62.7 | −1.0 | −1.0 |
| | homolog 5 (HP1 | heterochromatin protein homologue (HP1) | | | | |
| | alpha homolog, | mRNA, complete cds. /FEA = mRNA | | | | |
| | Drosophila) | /DB_XREF = gi: 184310 /UG = Hs.89232 | | | | |
| | | chromobox homolog 5 (Drosophila HP1 | | | | |
| | | alpha) /FL = gb: L07515.1 gb: NM_012117.1 | | | | |
| 203126_at | inositol(myo)-1(or | gb: NM_014214.1 /DEF = Homo sapiens | IMPA2 | 1.0 | −1.7 | 1.0 |
| | 4)- | inositol(myo)-1(or 4)-monophosphatase 2 | | | | |
| | monophosphatase 2 | (IMPA2), mRNA. /FEA = mRNA | | | | |
| | | /GEN = IMPA2 /PROD = inositol(myo)-1(or | | | | |
| | | 4)-monophosphatase 2 | | | | |
| | | /DB_XREF = gi: 7657235 /UG = Hs.5753 | | | | |
| | | inositol(myo)-1(or 4)-monophosphatase 2 | | | | |
| | | /FL = gb: AF200432.1 gb: NM_014214.1 | | | | |
| | | gb: AF014398.2 | | | | |
| 218400_at | 2′-5′-oligoadenylate | gb: NM_006187.1 /DEF = Homo sapiens 2- | OAS3 | −1.1 | −3.1 | −1.2 |
| | synthetase 3, | 5oligoadenylate synthetase 3 (OAS3), | | | | |
| | 100 kDa | mRNA. /FEA = mRNA /GEN = OAS3 | | | | |
| | | /PROD = 2-5oligoadenylate synthetase 3 | | | | |
| | | /DB_XREF = gi: 5453823 /UG = Hs.56009 2- | | | | |
| | | 5oligoadenylate synthetase 3 | | | | |
| | | /FL = gb: AB044545.1 gb: AF063613.1 | | | | |
| | | gb: NM_006187.1 | | | | |
| 206715_at | transcription factor | gb: NM_012252.1 /DEF = Homo sapiens | TFEC | 1.1 | −1.3 | −1.6 |
| | EC | transcription factor EC (TFEC), mRNA. | | | | |
| | | /FEA = mRNA /GEN = TFEC | | | | |
| | | /PROD = transcription factor EC | | | | |
| | | /DB_XREF = gi: 6912701 /UG = Hs.113274 | | | | |
| | | transcription factor EC /FL = gb: D43945.1 | | | | |
| | | gb: NM_012252.1 | | | | |
| 204620_s_at | chondroitin sulfate | gb: NM_004385.1 /DEF = Homo sapiens | CSPG2 | 1.5 | 1.0 | −1.1 |
| | proteoglycan 2 | chondroitin sulfate proteoglycan 2 | | | | |
| | (versican) | (versican) (CSPG2), mRNA. /FEA = mRNA | | | | |
| | | /GEN = CSPG2 /PROD = chondroitin sulfate | | | | |
| | | proteoglycan 2 (versican) | | | | |
| | | /DB_XREF = gi: 4758081 /UG = Hs.81800 | | | | |
| | | chondroitin sulfate proteoglycan 2 | | | | |
| | | (versican) /FL = gb: NM_004385.1 | | | | |
| 215071_s_at | histone 1, H2ac | Consensus includes gb: AL353759 | HIST1H2AC | 1.5 | 1.4 | 65.8 |
| | | /DEF = Human DNA sequence from clone | | | | |
| | | RP1-221C16 on chromosome 6. Contains | | | | |
| | | two genes for novel histone 4 family | | | | |
| | | members, two genes for novel histone 1 | | | | |
| | | family members, three genes for novel | | | | |
| | | histone 2B family members, a gene for a | | | | |
| | | novel histone 2A family me . . . | | | | |
| | | /FEA = mRNA_18 /DB_XREF = gi: 8745068 | | | | |
| | | /UG = Hs.28777 H2A histone family, | | | | |
| | | member L | | | | |
| 208581_x_at | metallothionein 1X | gb: NM_005952.1 /DEF = Homo sapiens | MT1X | 1.2 | −1.4 | −1.5 |
| | | metallothionein 1X (MT1X), mRNA. | | | | |
| | | /FEA = CDS /GEN = MT1X | | | | |
| | | /PROD = metallothionein 1X | | | | |
| | | /DB_XREF = gi: 10835231 /UG = Hs.278462 | | | | |
| | | metallothionein 1X /FL = gb: NM_005952.1 | | | | |
| 39248_at | aquaporin 3 | Cluster Incl. N74607: za55a01.s1 Homo | AQP3 | 1.5 | 1.0 | 1.1 |
| | | sapiens cDNA, 3 end /clone = IMAGE- | | | | |
| | | 296424 /clone_end = 3′ /gb = N74607 | | | | |
| | | /gi = 1231892 /ug = Hs.234642 /len = 487 | | | | |
| 219466_s_at | apolipoprotein A-II | gb: NM_001643.1 /DEF = Homo sapiens | APOA2 | 54.0 | 1.0 | −1.0 |
| | | apolipoprotein A-II (APOA2), mRNA. | | | | |
| | | /FEA = mRNA /GEN = APOA2 | | | | |
| | | /PROD = apolipoprotein A-II precursor | | | | |
| | | /DB_XREF = gi: 4502148 /UG = Hs.237658 | | | | |
| | | apolipoprotein A-II /FL = gb: BC005282.1 | | | | |
| | | gb: M29882.1 gb: NM_001643.1 | | | | |
| 206365_at | chemokine (C | gb: NM_002995.1 /DEF = Homo sapiens | XCL1 | −1.0 | −1.0 | 60.5 |
| | motif) ligand 1 | small inducible cytokine subfamily C, | | | | |
| | | member 1 (lymphotactin) (SCYC1), | | | | |
| | | mRNA. /FEA = mRNA /GEN = SCYC1 | | | | |
| | | /PROD = small inducible cytokine subfamily | | | | |
| | | C, member 1(lymphotactin) | | | | |
| | | /DB_XREF = gi: 4506852 /UG = Hs.3195 | | | | |
| | | small inducible cytokine subfamily C, | | | | |
| | | member 1 (lymphotactin) | | | | |
| | | /FL = gb: NM_002995.1 gb: U23772.1 | | | | |
| | | gb: D43768.1 | | | | |
| 205024_s_at | RAD51 homolog | gb: NM_002875.1 /DEF = Homo sapiens | RAD51 | −51.2 | −1.3 | 1.1 |
| | (RecA homolog, E. coli) | RAD51 (S. cerevisiae) homolog (E coli | | | | |
| | (S. cerevisiae) | RecA homolog) (RAD51), mRNA. | | | | |
| | | /FEA = mRNA /GEN = RAD51 | | | | |
| | | /PROD = RAD51 (S. cerevisiae) homolog | | | | |
| | | (E coli RecAhomolog) | | | | |
| | | /DB_XREF = gi: 4506388 /UG = Hs.23044 | | | | |
| | | RAD51 (S. cerevisiae) homolog (E coli | | | | |
| | | RecA homolog) /FL = gb: D14134.1 | | | | |
| | | gb: D13804.1 gb: NM_002875.1 | | | | |
| 219890_at | C-type (calcium | gb: NM_013252.1 /DEF = Homo sapiens C- | CLECSF5 | 1.5 | 1.0 | −1.2 |
| | dependent, | type (calcium dependent, carbohydrate- | | | | |
| | carbohydrate- | recognition domain) lectin, superfamily | | | | |
| | recognition | member 5 (CLECSF5), mRNA. | | | | |
| | domain) lectin, | /FEA = mRNA /GEN = CLECSF5 /PROD = C- | | | | |
| | superfamily | type (calcium dependent, carbohydrate- | | | | |
| | member 5 | recognition domain) lectin, | | | | |
| | | superfamilymember 5 | | | | |
| | | /DB_XREF = gi: 10281668 /UG = Hs.126355 | | | | |
| | | C-type (calcium dependent, carbohydrate- | | | | |
| | | recognition domain) lectin, superfamily | | | | |
| | | member 5 /FL = gb: NM_013252.1 | | | | |
| | | gb: AF139768.1 | | | | |
| 204122_at | TYRO protein | gb: NM_003332.1 /DEF = Homo sapiens | TYROBP | 1.0 | −1.2 | 1.0 |
| | tyrosine kinase | TYRO protein tyrosine kinase binding | | | | |
| | binding protein | protein (TYROBP), mRNA. /FEA = mRNA | | | | |
| | | /GEN = TYROBP /PROD = TYRO protein | | | | |
| | | tyrosine kinase binding protein | | | | |
| | | /DB_XREF = gi: 4507754 /UG = Hs.9963 | | | | |
| | | TYRO protein tyrosine kinase binding | | | | |
| | | protein /FL = gb: AF019562.1 | | | | |
| | | gb: NM_003332.1 | | | | |
| 205909_at | polymerase (DNA | gb: NM_002692.1 /DEF = Homo sapiens | POLE2 | 1.0 | −1.1 | 1.0 |
| | directed), epsilon 2 | polymerase (DNA directed), epsilon 2 | | | | |
| | (p59 subunit) | (POLE2), mRNA. /FEA = mRNA | | | | |
| | | /GEN = POLE2 /PROD = polymerase (DNA | | | | |
| | | directed), epsilon 2 | | | | |
| | | /DB_XREF = gi: 4505934 /UG = Hs.99185 | | | | |
| | | polymerase (DNA directed), epsilon 2 | | | | |
| | | /FL = gb: AF025840.1 gb: AF036899.1 | | | | |
| | | gb: NM_002692.1 | | | | |
| 202768_at | FBJ murine | gb: NM_006732.1 /DEF = Homo sapiens | FOSB | 1.4 | −1.5 | 53.4 |
| | osteosarcoma viral | FBJ murine osteosarcoma viral oncogene | | | | |
| | oncogene homolog B | homolog B (FOSB), mRNA. /FEA = mRNA | | | | |
| | | /GEN = FOSB /PROD = FBJ murine | | | | |
| | | osteosarcoma viral oncogene homologB | | | | |
| | | /DB_XREF = gi: 5803016 /UG = Hs.75678 | | | | |
| | | FBJ murine osteosarcoma viral oncogene | | | | |
| | | homolog B /FL = gb: L49169.1 | | | | |
| | | gb: NM_006732.1 | | | | |
| 203988_s_at | fucosyltransferase | gb: NM_004480.1 /DEF = Homo sapiens | FUT8 | 51.5 | −1.0 | −1.0 |
| | 8 (alpha (1,6) | fucosyltransferase 8 (alpha (1,6) | | | | |
| | fucosyltransferase) | fucosyltransferase) (FUT8), mRNA. | | | | |
| | | /FEA = mRNA /GEN = FUT8 | | | | |
| | | /PROD = fucosyltransferase 8 (alpha | | | | |
| | | (1,6)fucosyltransferase) | | | | |
| | | /DB_XREF = gi: 4758407 /UG = Hs.118722 | | | | |
| | | fucosyltransferase 8 (alpha (1,6) | | | | |
| | | fucosyltransferase) /FL = gb: D89289.1 | | | | |
| | | gb: NM_004480.1 | | | | |
| 201743_at | CD14 antigen | gb: NM_000591.1 /DEF = Homo sapiens | CD14 | 1.1 | −1.1 | 1.2 |
| | | CD14 antigen (CD14), mRNA. | | | | |
| | | /FEA = mRNA /GEN = CD14 /PROD = CD14 | | | | |
| | | antigen precursor /DB_XREF = gi: 4557416 | | | | |
| | | /UG = Hs.75627 CD14 antigen | | | | |
| | | /FL = gb: M86511.1 gb: AF097942.1 | | | | |
| | | gb: NM_000591.1 | | | | |
| 204222_s_at | GLI pathogenesis- | gb: NM_006851.1 /DEF = Homo sapiens | GLIPR1 | 1.0 | −1.2 | 1.1 |
| | related 1 (glioma) | glioma pathogenesis-related protein | | | | |
| | | (RTVP1), mRNA. /FEA = mRNA | | | | |
| | | /GEN = RTVP1 /PROD = glioma | | | | |
| | | pathogenesis-related protein | | | | |
| | | /DB_XREF = gi: 5803150 /UG = Hs.64639 | | | | |
| | | glioma pathogenesis-related protein | | | | |
| | | /FL = gb: U16307.1 gb: NM_006851.1 | | | | |
| 207277_at | CD209 antigen | gb: AF290886.1 /DEF = Homo sapiens DC- | CD209 | 1.4 | −1.2 | −1.5 |
| | | SIGN mRNA, complete cds. /FEA = mRNA | | | | |
| | | /PROD = DC-SIGN | | | | |
| | | /DB_XREF = gi: 13383467 /UG = Hs.278694 | | | | |
| | | CD209 antigen /FL = gb: NM_021155.1 | | | | |
| | | gb: AF290886.1 gb: M98457.1 | | | | |
| 202338_at | thymidine kinase 1, | gb: NM_003258.1 /DEF = Homo sapiens | TK1 | −1.0 | −1.0 | −1.4 |
| | soluble | thymidine kinase 1, soluble (TK1), mRNA. | | | | |
| | | /FEA = mRNA /GEN = TK1 | | | | |
| | | /PROD = thymidine kinase 1, soluble | | | | |
| | | /DB_XREF = gi: 4507518 /UG = Hs.105097 | | | | |
| | | thymidine kinase 1, soluble | | | | |
| | | /FL = gb: K02581.1 gb: NM_003258.1 | | | | |
| 208193_at | interleukin 9 | gb: NM_000590.1 /DEF = Homo sapiens | IL9 | −1.0 | −1.0 | −1.0 |
| | | interleukin 9 (IL9), mRNA. /FEA = mRNA | | | | |
| | | /GEN = IL9 /PROD = interleukin 9 | | | | |
| | | /DB_XREF = gi: 10834979 /UG = Hs.960 | | | | |
| | | interleukin 9 /FL = gb: NM_000590.1 | | | | |
| | | gb: M30134.1 | | | | |
| 204646_at | dihydropyrimidine | gb: NM_000110.2 /DEF = Homo sapiens | DPYD | 1.8 | −1.3 | −1.4 |
| | dehydrogenase | dihydropyrimidine dehydrogenase (DPYD), | | | | |
| | | mRNA. /FEA = mRNA /GEN = DPYD | | | | |
| | | /PROD = dihydropyrimidine dehydrogenase | | | | |
| | | /DB_XREF = gi: 4557874 /UG = Hs.1602 | | | | |
| | | dihydropyrimidine dehydrogenase | | | | |
| | | /FL = gb: U20938.1 gb: NM_000110.2 | | | | |
| | | gb: U09178.1 gb: AB003063.1 | | | | |
| 208450_at | lectin, galactoside- | gb: NM_006498.1 /DEF = Homo sapiens | LGALS2 | 1.3 | −1.3 | −1.7 |
| | binding, soluble, 2 | lectin, galactoside-binding, soluble, 2 | | | | |
| | (galectin 2) | (galectin 2) (LGALS2), mRNA. /FEA = CDS | | | | |
| | | /GEN = LGALS2 /PROD = lectin, galactoside- | | | | |
| | | binding, soluble, 2(galectin 2) | | | | |
| | | /DB_XREF = gi: 5729902 /UG = Hs.113987 | | | | |
| | | lectin, galactoside-binding, soluble, 2 | | | | |
| | | (galectin 2) /FL = gb: M87842.1 | | | | |
| | | gb: NM_006498.1 | | | | |
| 221841_s_at | Kruppel-like factor | Consensus includes gb: BF514079 | KLF4 | 1.4 | −1.4 | −1.2 |
| | 4 (gut) | /FEA = EST /DB_XREF = gi: 11599258 | | | | |
| | | /DB_XREF = est: UI-H-BW1-amw-b-08-0- | | | | |
| | | UI.s1 /CLONE = IMAGE: 3071198 | | | | |
| | | /UG = Hs.7934 Kruppel-like factor 4 (gut) | | | | |
| 209773_s_at | ribonucleotide | gb: BC001886.1 /DEF = Homo sapiens, | RRM2 | −1.9 | −1.5 | −1.5 |
| | reductase M2 | ribonucleotide reductase M2 polypeptide, | | | | |
| | polypeptide | clone MGC: 1551, mRNA, complete cds. | | | | |
| | | /FEA = mRNA /PROD = ribonucleotide | | | | |
| | | reductase M2 polypeptide | | | | |
| | | /DB_XREF = gi: 12804874 /UG = Hs.75319 | | | | |
| | | ribonucleotide reductase M2 polypeptide | | | | |
| | | /FL = gb: BC001886.1 | | | | |
| 201202_at | proliferating cell | gb: NM_002592.1 /DEF = Homo sapiens | PCNA | 1.1 | −1.1 | −1.5 |
| | nuclear antigen | proliferating cell nuclear antigen (PCNA), | | | | |
| | | mRNA. /FEA = mRNA /GEN = PCNA | | | | |
| | | /PROD = proliferating cell nuclear antigen | | | | |
| | | /DB_XREF = gi: 4505640 /UG = Hs.78996 | | | | |
| | | proliferating cell nuclear antigen | | | | |
| | | /FL = gb: BC000491.1 gb: M15796.1 | | | | |
| | | gb: NM_002592.1 | | | | |
| 202705_at | cyclin B2 | gb: NM_004701.2 /DEF = Homo sapiens | CCNB2 | −1.0 | −1.3 | 1.0 |
| | | cyclin B2 (CCNB2), mRNA. /FEA = mRNA | | | | |
| | | /GEN = CCNB2 /PROD = cyclin B2 | | | | |
| | | /DB_XREF = gi: 10938017 /UG = Hs.194698 | | | | |
| | | cyclin B2 /FL = gb: NM_004701.2 | | | | |
| | | gb: AF002822.1 gb: AB020981.1 | | | | |
| | | gb: AL080146.1 | | | | |
| 214470_at | killer cell lectin-like | Consensus includes gb: NM_002258.1 | KLRB1 | 1.2 | 1.4 | −1.2 |
| | receptor subfamily | /DEF = Homo sapiens killer cell lectin-like | | | | |
| | B, member 1 | receptor subfamily B, member 1 (KLRB1), | | | | |
| | | mRNA. /FEA = CDS /GEN = KLRB1 | | | | |
| | | /PROD = killer cell lectin-like receptor | | | | |
| | | subfamily B, member 1 | | | | |
| | | /DB_XREF = gi: 4504878 /UG = Hs.169824 | | | | |
| | | killer cell lectin-like receptor subfamily B, | | | | |
| | | member 1 /FL = gb: NM_002258.1 | | | | |
| | | gb: U11276.1 | | | | |
| 205098_at | chemokine (C-C | Consensus includes gb: AI421071 | | 1.1 | −1.7 | −1.1 |
| | motif) receptor 1 | /FEA = EST /DB_XREF = gi: 4267002 | | | | |
| | | /DB_XREF = est: tf24a02.x1 | | | | |
| | | /CLONE = IMAGE: 2097098 | | | | |
| | | /UG = Hs.301921 chemokine (C-C motif) | | | | |
| | | receptor 1 /FL = gb: L09230.1 gb: L10918.1 | | | | |
| | | gb: NM_001295.1 | | | | |
| 210190_at | syntaxin 11 | gb: AF071504.1 /DEF = Homo sapiens | STX11 | −1.0 | −1.0 | −1.0 |
| | | syntaxin 11 mRNA, complete cds. | | | | |
| | | /FEA = mRNA /PROD = syntaxin 11 | | | | |
| | | /DB_XREF = gi: 3243239 /UG = Hs.118958 | | | | |
| | | syntaxin 11 /FL = gb: AF071504.1 | | | | |
| | | gb: AF044309.1 gb: AF038898.1 | | | | |
| | | gb: NM_003764.1 | | | | |
| 204438_at | mannose receptor, | gb: NM_002438.1 /DEF = Homo sapiens | MRC1 | 1.1 | −1.5 | −1.6 |
| | C type 1 | mannose receptor, C type 1 (MRC1), | | | | |
| | | mRNA. /FEA = mRNA /GEN = MRC1 | | | | |
| | | /PROD = mannose receptor, C type 1 | | | | |
| | | /DB_XREF = gi: 4505244 /UG = Hs.75182 | | | | |
| | | mannose receptor, C type 1 | | | | |
| | | /FL = gb: J05550.1 gb: NM_002438.1 | | | | |
| 221898_at | lung type-I cell | Consensus includes gb: AU154455 | T1A-2 | −53.7 | −1.0 | −1.0 |
| | membrane- | /FEA = EST /DB_XREF = gi: 11015976 | | | | |
| | associated | /DB_XREF = est: AU154455 | | | | |
| | glycoprotein | /CLONE = NT2RP4001145 /UG = Hs.135150 | | | | |
| | | lung type-I cell membrane-associated | | | | |
| | | glycoprotein | | | | |
| 205354_at | guanidinoacetate N [symbol] | gb: NM_000156.3 /DEF = Homo sapiens | GAMT | −1.0 | −1.0 | −1.0 |
| | methyltransferase | guanidinoacetate N-methyltransferase | | | | |
| | | (GAMT), mRNA. /FEA = mRNA | | | | |
| | | /GEN = GAMT /PROD = guanidinoacetate N- | | | | |
| | | methyltransferase /DB_XREF = gi: 7549759 | | | | |
| | | /UG = Hs.81131 guanidinoacetate N- | | | | |
| | | methyltransferase /FL = gb: NM_000156.3 | | | | |
| 202888_s_at | alanyl (membrane) | gb: NM_001150.1 /DEF = Homo sapiens | ANPEP | −1.2 | −1.2 | 1.1 |
| | aminopeptidase | alanyl (membrane) aminopeptidase | | | | |
| | (aminopeptidase | (aminopeptidase N, aminopeptidase M, | | | | |
| | N, aminopeptidase | microsomal aminopeptidase, CD13, p150) | | | | |
| | M, microsomal | (ANPEP), mRNA. /FEA = mRNA | | | | |
| | aminopeptidase, | /GEN = ANPEP /PROD = membrane alanine | | | | |
| | CD13, p150) | aminopeptidase precursor | | | | |
| | | /DB_XREF = gi: 4502094 /UG = Hs.1239 | | | | |
| | | alanyl (membrane) aminopeptidase | | | | |
| | | (aminopeptidase N, aminopeptidase M, | | | | |
| | | microsomal aminopeptidase, CD13, p150) | | | | |
| | | /FL = gb: M22324.1 gb: NM_001150.1 | | | | |
| 211962_s_at | zinc finger protein | Consensus includes gb: BG250310 | | 1.7 | 1.1 | 1.0 |
| | 36, C3H type-like 1 | /FEA = EST /DB_XREF = gi: 12760126 | | | | |
| | | /DB_XREF = est: 602362443F1 | | | | |
| | | /CLONE = IMAGE: 4470898 /UG = Hs.85155 | | | | |
| | | butyrate response factor 1 (EGF-response | | | | |
| | | factor 1) | | | | |
| 200862_at | 24- | gb: NM_014762.1 /DEF = Homo sapiens | DHCR24 | 1.2 | −1.3 | −1.2 |
| | dehydrocholesterol | seladin-1 (KIAA0018), mRNA. | | | | |
| | reductase | /FEA = mRNA /GEN = KIAA0018 | | | | |
| | | /PROD = seladin-1 | | | | |
| | | /DB_XREF = gi: 13375617 /UG = Hs.75616 | | | | |
| | | seladin-1 /FL = gb: AF261758.1 | | | | |
| | | gb: BC004375.1 gb: NM_014762.1 | | | | |
| 217755_at | hematological and | gb: NM_016185.1 /DEF = Homo sapiens | HN1 | 1.0 | 1.1 | −1.2 |
| | neurological | hematological and neurological expressed | | | | |
| | expressed 1 | 1 (HN1), mRNA. /FEA = mRNA/GEN = HN1 | | | | |
| | | /PROD = hematological and neurological | | | | |
| | | expressed 1 /DB_XREF = gi: 7705876 | | | | |
| | | /UG = Hs.109706 hematological and | | | | |
| | | neurological expressed 1 | | | | |
| | | /FL = gb: AF060925.1 gb: BC001420.1 | | | | |
| | | gb: AF177862.1 gb: NM_016185.1 | | | | |
| 205235_s_at | M-phase | gb: NM_016195.1 /DEF = Homo sapiens M- | MPHOSPH1 | 1.6 | −55.4 | −67.1 |
| | phosphoprotein 1 | phase phosphoprotein 1 (MPHOSPH1), | | | | |
| | | mRNA. /FEA = mRNA /GEN = MPHOSPH1 | | | | |
| | | /PROD = M-phase phosphoprotein 1 | | | | |
| | | /DB_XREF = gi: 7705347 /UG = Hs.240 M- | | | | |
| | | phase phosphoprotein 1 | | | | |
| | | /FL = gb: AL117496.1 gb: NM_016195.1 | | | | |
| 219841_at | activation-induced | gb: NM_020661.1 /DEF = Homo sapiens | AICDA | −1.0 | −1.0 | −1.0 |
| | cytidine deaminase | activation-induced cytidine deaminase | | | | |
| | | (AICDA), mRNA. /FEA = mRNA | | | | |
| | | /GEN = AICDA /PROD = activation-induced | | | | |
| | | cytidine deaminase | | | | |
| | | /DB_XREF = gi: 10190699 /UG = Hs.149342 | | | | |
| | | activation-induced cytidine deaminase | | | | |
| | | /FL = gb: NM_020661.1 gb: AB040431.1 | | | | |
| 205403_at | interleukin 1 | gb: NM_004633.1 /DEF = Homo sapiens | IL1R2 | 1.8 | −1.0 | −50.5 |
| | receptor, type II | interleukin 1 receptor, type II (IL1R2), | | | | |
| | | mRNA. /FEA = mRNA /GEN = IL1R2 | | | | |
| | | /PROD = interleukin 1 receptor, type II | | | | |
| | | /DB_XREF = gi: 4758597 /UG = Hs.25333 | | | | |
| | | interleukin 1 receptor, type II | | | | |
| | | /FL = gb: U74649.1 gb: NM_004633.1 | | | | |
| 205016_at | transforming | gb: NM_003236.1 /DEF = Homo sapiens | TGFA | −1.3 | −1.1 | 1.7 |
| | growth factor, | transforming growth factor, alpha (TGFA), | | | | |
| | alpha | mRNA. /FEA = mRNA /GEN = TGFA | | | | |
| | | /PROD = transforming growth factor, alpha | | | | |
| | | /DB_XREF = gi: 4507460 /UG = Hs.170009 | | | | |
| | | transforming growth factor, alpha | | | | |
| | | /FL = gb: BC005308.1 gb: M31172.1 | | | | |
| | | gb: K03222.1 gb: NM_003236.1 | | | | |
| 201438_at | collagen, type VI, | gb: NM_004369.1 /DEF = Homo sapiens | COL6A3 | −1.0 | −1.0 | −1.0 |
| | alpha 3 | collagen, type VI, alpha 3 (COL6A3), | | | | |
| | | mRNA. /FEA = mRNA /GEN = COL6A3 | | | | |
| | | /PROD = collagen, type VI, alpha 3 | | | | |
| | | /DB_XREF = gi: 4758027 /UG = Hs.80988 | | | | |
| | | collagen, type VI, alpha 3 | | | | |
| | | /FL = gb: NM_004369.1 | | | | |
| 203270_at | deoxythymidylate | gb: NM_012145.1 /DEF = Homo sapiens | DTYMK | 60.6 | 1.1 | −73.1 |
| | kinase (thymidylate | deoxythymidylate kinase (thymidylate | | | | |
| | kinase) | kinase) (DTYMK), mRNA. /FEA = mRNA | | | | |
| | | /GEN = DTYMK /PROD = deoxythymidylate | | | | |
| | | kinase (thymidylate kinase) | | | | |
| | | /DB_XREF = gi: 6912339 /UG = Hs.79006 | | | | |
| | | deoxythymidylate kinase (thymidylate | | | | |
| | | kinase) /FL = gb: BC001827.1 gb: L16991.1 | | | | |
| | | gb: NM_012145.1 | | | | |
| 219024_at | pleckstrin | gb: NM_021622.1 /DEF = Homo sapiens | PLEKHA1 | 1.4 | 1.0 | 1.1 |
| | homology domain | pleckstrin homology domain-containing, | | | | |
| | containing, family A | family A (phosphoinositide binding | | | | |
| | (phosphoinositide | specific) member 1 (PLEKHA1), mRNA. | | | | |
| | binding specific) | /FEA = mRNA /GEN = PLEKHA1 | | | | |
| | member 1 | /PROD = pleckstrin homology domain- | | | | |
| | | containing, family A(phosphoinositide | | | | |
| | | binding specific) member 1 | | | | |
| | | /DB_XREF = gi: 11055985 /UG = Hs.17757 | | | | |
| | | pleckstrin homology domain-containing, | | | | |
| | | family A (phosphoinositide binding | | | | |
| | | specific) member 1 /FL = gb: AF286160.1 | | | | |
| | | gb: NM_021622.1 gb: BC001136.1 | | | | |
| 204822_at | TTK protein kinase | gb: NM_003318.1 /DEF = Homo sapiens | | −1.0 | 0.8 | −1.0 |
| | | TTK protein kinase (TTK), mRNA. | | | | |
| | | /FEA = mRNA /GEN = TTK /PROD = TTK | | | | |
| | | protein kinase /DB_XREF = gi: 4507718 | | | | |
| | | /UG = Hs.169840 TTK protein kinase | | | | |
| | | /FL = gb: BC000633.1 gb: M86699.1 | | | | |
| | | gb: NM_003318.1 | | | | |
| 212672_at | ataxia | Consensus includes gb: U82828 | ATM | 2.1 | 1.0 | −1.1 |
| | telangiectasia | /DEF = Homo sapiens ataxia telangiectasia | | | | |
| | mutated (includes | (ATM) gene, complete cds /FEA = mRNA_1 | | | | |
| | complementation | /DB_XREF = gi: 2304970 /UG = Hs.194382 | | | | |
| | groups A, C and D) | ataxia telangiectasia mutated (includes | | | | |
| | | complementation groups A, C and D) | | | | |
| 207067_s_at | histidine | gb: NM_002112.1 /DEF = Homo sapiens | HDC | −1.3 | 1.0 | −1.2 |
| | decarboxylase | histidine decarboxylase (HDC), mRNA. | | | | |
| | | /FEA = mRNA /GEN = HDC /PROD = histidine | | | | |
| | | decarboxylase /DB_XREF = gi: 4504364 | | | | |
| | | /UG = Hs.1481 histidine decarboxylase | | | | |
| | | /FL = gb: M60445.1 gb: NM_002112.1 | | | | |
| 205639_at | acyloxyacyl | gb: NM_001637.1 /DEF = Homo sapiens | AOAH | 1.1 | −1.3 | 1.1 |
| | hydrolase | acyloxyacyl hydrolase (neutrophil) | | | | |
| | (neutrophil) | (AOAH), mRNA. /FEA = mRNA | | | | |
| | | /GEN = AOAH /PROD = acyloxyacyl | | | | |
| | | hydrolase precursor | | | | |
| | | /DB_XREF = gi: 4502114 /UG = Hs.82542 | | | | |
| | | acyloxyacyl hydrolase (neutrophil) | | | | |
| | | /FL = gb: M62840.1 gb: NM_001637.1 | | | | |
| 220088_at | G protein-coupled | gb: NM_001736.1 /DEF = Homo sapiens | C5R1 | 1.2 | −1.1 | 1.1 |
| | receptor 77 | complement component 5 receptor 1 (C5a | | | | |
| | | ligand) (C5R1), mRNA. /FEA = mRNA | | | | |
| | | /GEN = C5R1 /PROD = complement | | | | |
| | | component 5 receptor 1 (C5a ligand) | | | | |
| | | /DB_XREF = gi: 4502508 /UG = Hs.2161 | | | | |
| | | complement component 5 receptor 1 (C5a | | | | |
| | | ligand) /FL = gb: M62505.1 | | | | |
| | | gb: NM_001736.1 | | | | |
| 204614_at | serine (or cysteine) | gb: NM_002575.1 /DEF = Homo sapiens | SERPINB2 | 1.4 | 1.2 | 1.3 |
| | proteinase | serine (or cysteine) proteinase inhibitor, | | | | |
| | inhibitor, clade B | clade B (ovalbumin), member 2 | | | | |
| | (ovalbumin), | (SERPINB2), mRNA. /FEA = mRNA | | | | |
| | member 2 | /GEN = SERPINB2 /PROD = serine (or | | | | |
| | | cysteine) proteinase inhibitor, cladeB | | | | |
| | | (ovalbumin), member 2 | | | | |
| | | /DB_XREF = gi: 4505594 /UG = Hs.75716 | | | | |
| | | serine (or cysteine) proteinase inhibitor, | | | | |
| | | clade B (ovalbumin), member 2 | | | | |
| | | /FL = gb: J02685.1 gb: J03603.1 | | | | |
| | | gb: M18082.1 gb: NM_002575.1 | | | | |
| 209921_at | solute carrier | gb: AB040875.1 /DEF = Homo sapiens | SLC7A11 | 1.2 | 1.1 | −1.2 |
| | family 7, (cationic | hxCT mRNA for cystineglutamate | | | | |
| | amino acid | exchanger, complete cds. /FEA = mRNA | | | | |
| | transporter, y+ | /GEN = hxCT /PROD = cystineglutamate | | | | |
| | system) member | exchanger /DB_XREF = gi: 13516845 | | | | |
| | 11 | /UG = Hs.6682 solute carrier family 7, | | | | |
| | | (cationic amino acid transporter, y+ | | | | |
| | | system) member 11 /FL = gb: AB040875.1 | | | | |
| 208863_s_at | splicing factor, | gb: M72709.1 /DEF = Human alternative | SFRS1 | 1.6 | 1.1 | 1.5 |
| | arginine/serine-rich | splicing factor mRNA, complete cds. | | | | |
| | 1 (splicing factor 2, | /FEA = mRNA /DB_XREF = gi: 179073 | | | | |
| | alternate splicing | /UG = Hs.73737 splicing factor, | | | | |
| | factor) | arginineserine-rich 1 (splicing factor 2, alternate splicing factor) /FL = gb: | | | | |
| | | M72709.1 | | | | |
| 205174_s_at | glutaminyl-peptide | gb: NM_012413.2 /DEF = Homo sapiens | QPCT | 1.2 | −1.6 | −1.2 |
| | cyclotransferase | glutaminyl-peptide cyclotransferase | | | | |
| | (glutaminyl | (glutaminyl cyclase) (QPCT), mRNA. | | | | |
| | cyclase) | /FEA = mRNA /GEN = QPCT | | | | |
| | | /PROD = glutaminyl-peptide | | | | |
| | | cyclotransferase precursor | | | | |
| | | /DB_XREF = gi: 9257235 /UG = Hs.79033 | | | | |
| | | glutaminyl-peptide cyclotransferase | | | | |
| | | (glutaminyl cyclase) /FL = gb: NM_012413.2 | | | | |
| 207038_at | solute carrier | gb: NM_004694.1 /DEF = Homo sapiens | SLC16A6 | 1.1 | 1.0 | 1.3 |
| | family 16 | solute carrier family 16 (monocarboxylic | | | | |
| | (monocarboxylic | acid transporters), member 6 (SLC16A6), | | | | |
| | acid transporters), | mRNA. /FEA = mRNA /GEN = SLC16A6 | | | | |
| | member 6 | /PROD = solute carrier family 16 | | | | |
| | | (monocarboxylic acidtransporters), | | | | |
| | | member 6 /DB_XREF = gi: 4759117 | | | | |
| | | /UG = Hs.114924 solute carrier family 16 | | | | |
| | | (monocarboxylic acid transporters), | | | | |
| | | member 6 /FL = gb: U79745.1 | | | | |
| | | gb: NM_004694.1 | | | | |
| 204039_at | CCAAT/enhancer | gb: NM_004364.1 /DEF = Homo sapiens | CEBPA | 1.3 | −1.1 | 1.1 |
| | binding protein | CCAATenhancer binding protein (CEBP), | | | | |
| | (C/EBP), alpha | alpha (CEBPA), mRNA. /FEA = mRNA | | | | |
| | | /GEN = CEBPA /PROD = CCAATenhancer | | | | |
| | | binding protein (CEBP), alpha | | | | |
| | | /DB_XREF = gi: 4757971 /UG = Hs.76171 | | | | |
| | | CCAATenhancer binding protein (CEBP), | | | | |
| | | alpha /FL = gb: NM_004364.1 | | | | |
| 217786_at | SKB1 homolog (S. pombe) | gb: NM_006109.1 /DEF = Homo sapiens | SKB1 | 1.5 | 1.1 | 1.0 |
| | | skb1 (S. pombe) homolog (SKB1), mRNA. | | | | |
| | | /FEA = mRNA /GEN = SKB1 /PROD = skb1 | | | | |
| | | (S. pombe) homolog | | | | |
| | | /DB_XREF = gi: 5174682 /UG = Hs.12912 | | | | |
| | | skb1 (S. pombe) homolog | | | | |
| | | /FL = gb: AF015913.1 gb: NM_006109.1 | | | | |
| | | gb: AF167572.1 | | | | |
| 201710_at | v-myb | gb: NM_002466.1 /DEF = Homo sapiens v- | MYBL2 | −1.0 | −1.0 | −1.0 |
| | myeloblastosis | myb avian myeloblastosis viral oncogene | | | | |
| | viral oncogene | homolog-like 2 (MYBL2), mRNA. | | | | |
| | homolog (avian)- | /FEA = mRNA /GEN = MYBL2 /PROD = v- | | | | |
| | like 2 | myb avian myeloblastosis viral | | | | |
| | | oncogenehomolog-like 2 | | | | |
| | | /DB_XREF = gi: 4505292 /UG = Hs.179718 v- | | | | |
| | | myb avian myeloblastosis viral oncogene | | | | |
| | | homolog-like 2 /FL = gb: NM_002466.1 | | | | |
| 204693_at | CDC42 effector | gb: NM_007061.1 /DEF = Homo sapiens | CDC42EP1 | −1.1 | −88.6 | 1.0 |
| | protein (Rho | serum constituent protein (MSE55), | | | | |
| | GTPase binding) 1 | mRNA. /FEA = mRNA /GEN = MSE55 | | | | |
| | | /PROD = serum constituent protein | | | | |
| | | /DB_XREF = gi: 5902007 /UG = Hs.148101 | | | | |
| | | serum constituent protein | | | | |
| | | /FL = gb: M88338.1 gb: NM_007061.1 | | | | |
| 205786_s_at | integrin, alpha M | gb: NM_000632.2 /DEF = Homo sapiens | ITGAM | 1.0 | −1.2 | −1.6 |
| | (complement | integrin, alpha M (complement component | | | | |
| | component | receptor 3, alpha; also known as CD11b | | | | |
| | receptor 3, alpha; | (p170), macrophage antigen alpha | | | | |
| | also known as | polypeptide) (ITGAM), mRNA. | | | | |
| | CD11b (p170), | /FEA = mRNA /GEN = ITGAM | | | | |
| | macrophage | /PROD = integrin alpha M precursor | | | | |
| | antigen alpha | /DB_XREF = gi: 6006013 /UG = Hs.172631 | | | | |
| | polypeptide) | integrin, alpha M (complement component | | | | |
| | | receptor 3, alpha; also known as CD11b | | | | |
| | | (p170), macrophage antigen alpha | | | | |
| | | polypeptide) /FL = gb: J03925.1 | | | | |
| | | gb: NM_000632.2 | | | | |
| 209172_s_at | centromere protein | gb: U30872.1 /DEF = Human mitosin | CENPF | −1.0 | −1.0 | −1.0 |
| | F, 350/400ka | mRNA, complete cds. /FEA = mRNA | | | | |
| | (mitosin) | /PROD = mitosin /DB_XREF = gi: 1000093 | | | | |
| | | /UG = Hs.77204 centromere protein F | | | | |
| | | (350400 kD, mitosin) /FL = gb: U30872.1 | | | | |
| 201360_at | cystatin C (amyloid | gb: NM_000099.1 /DEF = Homo sapiens | CST3 | 1.0 | −1.3 | −1.3 |
| | angiopathy and | cystatin C (amyloid angiopathy and | | | | |
| | cerebral | cerebral hemorrhage) (CST3), mRNA. | | | | |
| | hemorrhage) | /FEA = mRNA /GEN = CST3 | | | | |
| | | /PROD = cystatin C (amyloid angiopathy | | | | |
| | | and cerebralhemorrhage) | | | | |
| | | /DB_XREF = gi: 4503106 /UG = Hs.135084 | | | | |
| | | cystatin C (amyloid angiopathy and | | | | |
| | | cerebral hemorrhage) | | | | |
| | | /FL = gb: NM_000099.1 | | | | |
| 216537_s_at | sialic acid binding | Consensus includes gb: AJ130713.1 | SIGLEC7 | 1.1 | 1.7 | 1.0 |
| | Ig-like lectin 7 | /DEF = Homo sapiens mRNA for QA79 | | | | |
| | | membrane protein, splice product airm-1. | | | | |
| | | /FEA = mRNA /GEN = airm /PROD = QA79 | | | | |
| | | membrane protein, splice product airm-1 | | | | |
| | | /DB_XREF = gi: 5541875 /UG = Hs.274470 | | | | |
| | | sialic acid binding Ig-like lectin 7 | | | | |
| 206682_at | C-type (calcium | gb: NM_006344.1 /DEF = Homo sapiens | CLECSF14 | 1.3 | −1.2 | −1.4 |
| | dependent, | macrophage lectin 2 (calcium dependent) | | | | |
| | carbohydrate- | (HML2), mRNA. /FEA = mRNA | | | | |
| | recognition | /GEN = HML2 /PROD = macrophage lectin 2 | | | | |
| | domain) lectin, | (calcium dependent) | | | | |
| | superfamily | /DB_XREF = gi: 5453683 /UG = Hs.54403 | | | | |
| | member 14 | macrophage lectin 2 (calcium dependent) | | | | |
| | (macrophage- | /FL = gb: D50532.1 gb: NM_006344.1 | | | | |
| | derived) | | | | | |
| 201739_at | serum/glucocorticoid | gb: NM_005627.1 /DEF = Homo sapiens | SGK | 1.3 | 1.0 | 1.0 |
| | regulated kinase | serumglucocorticoid regulated kinase | | | | |
| | | (SGK), mRNA. /FEA = mRNA /GEN = SGK | | | | |
| | | /PROD = serumglucocorticoid regulated | | | | |
| | | kinase /DB_XREF = gi: 5032090 | | | | |
| | | /UG = Hs.296323 serumglucocorticoid | | | | |
| | | regulated kinase /FL = gb: BC001263.1 | | | | |
| | | gb: NM_005627.1 gb: AF153609.1 | | | | |
| 209825_s_at | uridine | gb: BC002906.1 /DEF = Homo sapiens, | UCK2 | 1.0 | 1.0 | 68.1 |
| | monophosphate | Similar to uridine monophosphate kinase, | | | | |
| | kinase | clone MGC: 10318, mRNA, complete cds. | | | | |
| | | /FEA = mRNA /PROD = Similar to uridine | | | | |
| | | monophosphate kinase | | | | |
| | | /DB_XREF = gi: 12804106 /UG = Hs.75939 | | | | |
| | | uridine monophosphate kinase | | | | |
| | | /FL = gb: BC002906.1 gb: AF236637.1 | | | | |
| 219371_s_at | Kruppel-like factor | gb: NM_016270.1 /DEF = Homo sapiens | KLF2 | 1.1 | 1.1 | 1.0 |
| | 2 (lung) | Kruppel-like factor (LOC51713), mRNA. | | | | |
| | | /FEA = mRNA /GEN = LOC51713 | | | | |
| | | /PROD = Kruppel-like factor | | | | |
| | | /DB_XREF = gi: 7706468 /UG = Hs.107740 | | | | |
| | | Kruppel-like factor 2 (lung) | | | | |
| | | /FL = gb: AF134053.1 gb: AF205849.1 | | | | |
| | | gb: NM_016270.1 | | | | |
| 209696_at | fructose-1,6- | gb: D26054.1 /DEF = Human mRNA for | FBP1 | 1.0 | −1.5 | −1.4 |
| | bisphosphatase 1 | fructose-1,6-bisphosphatase, complete | | | | |
| | | cds. /FEA = mRNA /PROD = fructose-1,6- | | | | |
| | | bisphosphatase /DB_XREF = gi: 439225 | | | | |
| | | /UG = Hs.574 fructose-1,6-bisphosphatase | | | | |
| | | 1 /FL = gb: M19922.1 gb: L10320.1 | | | | |
| | | gb: D26054.1 gb: D26055.1 gb: D26056.1 | | | | |
| | | gb: NM_000507.1 | | | | |
| 209949_at | neutrophil cytosolic | gb: BC001606.1 /DEF = Homo sapiens, | NCF2 | 1.2 | −1.4 | −1.5 |
| | factor 2 (65 kDa, | Similar to neutrophil cytosolic factor 2 | | | | |
| | chronic | (65 kD, chronic granulomatous disease, | | | | |
| | granulomatous | autosomal 2), clone MGC: 2275, mRNA, | | | | |
| | disease, autosomal | complete cds. /FEA = mRNA | | | | |
| | 2) | /PROD = Similar to neutrophil cytosolic | | | | |
| | | factor 2 (65 kD, chronic granulomatous | | | | |
| | | disease, autosomal 2) | | | | |
| | | /DB_XREF = gi: 12804408 /UG = Hs.949 | | | | |
| | | neutrophil cytosolic factor 2 (65 kD, chronic | | | | |
| | | granulomatous disease, autosomal 2) | | | | |
| | | /FL = gb: BC001606.1 gb: M32011.1 | | | | |
| | | gb: NM_000433.1 | | | | |
| 210220_at | frizzled homolog 2 | gb: L37882.1 /DEF = Human frizzled gene | FZD2 | −1.2 | 1.1 | 1.0 |
| | (Drosophila) | product mRNA, complete cds. | | | | |
| | | /FEA = mRNA /GEN = frizzled | | | | |
| | | /DB_XREF = gi: 736678 /UG = Hs.81217 | | | | |
| | | frizzled (Drosophila) homolog 2 | | | | |
| | | /FL = gb: AB017364.1 gb: NM_001466.2 | | | | |
| | | gb: L37882.1 | | | | |
| 205718_at | integrin, beta 7 | gb: NM_000889.1 /DEF = Homo sapiens | ITGB7 | 1.3 | 1.1 | −1.1 |
| | | integrin, beta 7 (ITGB7), mRNA. | | | | |
| | | /FEA = mRNA /GEN = ITGB7 | | | | |
| | | /PROD = integrin, beta 7 | | | | |
| | | /DB_XREF = gi: 4504776 /UG = Hs.1741 | | | | |
| | | integrin, beta 7 /FL = gb: M68892.1 | | | | |
| | | gb: M62880.1 gb: NM_000889.1 | | | | |
| 206999_at | interleukin 12 | gb: NM_001559.1 /DEF = Homo sapiens | IL12RB2 | 1.0 | 59.9 | −1.0 |
| | receptor, beta 2 | interleukin 12 receptor, beta 2 (IL12RB2), | | | | |
| | | mRNA. /FEA = mRNA /GEN = IL12RB2 | | | | |
| | | /PROD = interleukin 12 receptor, beta 2 | | | | |
| | | /DB_XREF = gi: 4504642 /UG = Hs.73165 | | | | |
| | | interleukin 12 receptor, beta 2 | | | | |
| | | /FL = gb: U64198.1 gb: NM_001559.1 | | | | |
| 203104_at | colony stimulating | gb: NM_005211.1 /DEF = Homo sapiens | CSF1R | 1.1 | −1.1 | 1.1 |
| | factor 1 receptor, | colony stimulating factor 1 receptor, | | | | |
| | formerly | formerly McDonough feline sarcoma viral | | | | |
| | McDonough feline | (v-fms) oncogene homolog (CSF1R), | | | | |
| | sarcoma viral (v- | mRNA. /FEA = mRNA /GEN = CSF1R | | | | |
| | fms) oncogene | /PROD = colony stimulating factor 1 | | | | |
| | homolog | receptor, formerlyMcDonough feline | | | | |
| | | sarcoma viral (v-fms) oncogene homolog | | | | |
| | | /DB_XREF = gi: 4885158 /UG = Hs.174142 | | | | |
| | | colony stimulating factor 1 receptor, | | | | |
| | | formerly McDonough feline sarcoma viral | | | | |
| | | (v-fms) oncogene homolog | | | | |
| | | /FL = gb: NM_005211.1 | | | | |
| 209661_at | kinesin family | gb: BC001211.1 /DEF = Homo sapiens, | KIFC3 | 1.1 | −1.3 | −1.4 |
| | member C3 | kinesin family member C3, clone | | | | |
| | | MGC: 3226, mRNA, complete cds. | | | | |
| | | /FEA = mRNA /PROD = kinesin family | | | | |
| | | member C3 /DB_XREF = gi: 12654738 | | | | |
| | | /UG = Hs.23131 kinesin family member C3 | | | | |
| | | /FL = gb: BC001211.1 gb: AF004426.1 | | | | |
| | | gb: NM_005550.1 | | | | |
| 221210_s_at | N- | gb: NM_030769.1 /DEF = Homo sapiens | NPL | 1.5 | −1.1 | 1.1 |
| | acetylneuraminate | hypothetical protein similar to swine | | | | |
| | pyruvate lyase | acylneuraminate lyase (C1ORF13), | | | | |
| | (dihydrodipicolinate | mRNA. /FEA = mRNA /GEN = C1ORF13 | | | | |
| | synthase) | /PROD = hypothetical protein similar to | | | | |
| | | swineacylneuraminate lyase | | | | |
| | | /DB_XREF = gi: 13540532 | | | | |
| | | /FL = gb: NM_030769.1 | | | | |
| 221748_s_at | tensin | Consensus includes gb: AL046979 | TNS | 1.2 | 1.0 | 1.8 |
| | | /FEA = EST /DB_XREF = gi: 5435035 | | | | |
| | | /DB_XREF = est: DKFZp586K0617_s1 | | | | |
| | | /CLONE = DKFZp586K0617 /UG = Hs.9973 | | | | |
| | | tensin | | | | |
| 202878_s_at | complement | gb: NM_012072.2 /DEF = Homo sapiens | C1QR1 | 1.2 | 1.2 | 1.2 |
| | component 1, q | complement component C1q receptor | | | | |
| | subcomponent, | (C1QR), mRNA. /FEA = mRNA | | | | |
| | receptor 1 | /GEN = C1QR /PROD = complement | | | | |
| | | component C1q receptor | | | | |
| | | /DB_XREF = gi: 11496985 /UG = Hs.97199 | | | | |
| | | complement component C1q receptor | | | | |
| | | /FL = gb: NM_012072.2 gb: U94333.1 | | | | |
| 210152_at | leukocyte | gb: U82979.1 /DEF = Human | LILRB4 | −1.3 | −1.3 | −1.2 |
| | immunoglobulin- | immunoglobulin-like transcript-3 mRNA, | | | | |
| | like receptor, | complete cds. /FEA = mRNA | | | | |
| | subfamily B (with | /PROD = immunoglobulin-like transcript-3 | | | | |
| | TM and ITIM | /DB_XREF = gi: 1857460 /UG = Hs.67846 | | | | |
| | domains), member 4 | leukocyte immunoglobulin-like receptor, | | | | |
| | | subfamily B (with TM and ITIM domains), | | | | |
| | | member 4 /FL = gb: U82979.1 gb: U91925.1 | | | | |
| | | gb: AF025532.1 gb: NM_006847.1 | | | | |
| 219306_at | kinesin-like 7 | gb: NM_020242.1 /DEF = Homo sapiens | KNSL7 | −1.0 | −1.0 | −1.0 |
| | | kinesin-like protein 2 (hklp2), mRNA. | | | | |
| | | /FEA = mRNA /GEN = hklp2 /PROD = kinesin- | | | | |
| | | like protein 2 /DB_XREF = gi: 9910265 | | | | |
| | | /UG = Hs.150587 kinesin-like protein 2 | | | | |
| | | /FL = gb: AB035898.1 gb: NM_020242.1 | | | | |
| 208075_s_at | chemokine (C-C | gb: NM_006273.2 /DEF = Homo sapiens | | −1.0 | −1.0 | −1.0 |
| | motif) ligand 7 | small inducible cytokine A7 (monocyte | | | | |
| | | chemotactic protein 3) (SCYA7), mRNA. | | | | |
| | | /FEA = mRNA /GEN = SCYA7 | | | | |
| | | /PROD = monocyte chemotactic protein 3 | | | | |
| | | precursor /DB_XREF = gi: 13435401 | | | | |
| | | /FL = gb: NM_006273.2 | | | | |
| 204959_at | myeloid cell | gb: NM_002432.1 /DEF = Homo sapiens | MNDA | 1.1 | −1.5 | −1.5 |
| | nuclear | myeloid cell nuclear differentiation antigen | | | | |
| | differentiation | (MNDA), mRNA. /FEA = mRNA | | | | |
| | antigen | /GEN = MNDA /PROD = myeloid cell nuclear | | | | |
| | | differentiation antigen | | | | |
| | | /DB_XREF = gi: 4505226 /UG = Hs.153837 | | | | |
| | | myeloid cell nuclear differentiation antigen | | | | |
| | | /FL = gb: NM_002432.1 gb: M81750.1 | | | | |
| 201849_at | BCL2/adenovirus | gb: NM_004052.2 /DEF = Homo sapiens | BNIP3 | 1.6 | 1.0 | −1.3 |
| | E1B 19 kDa | BCL2adenovirus E1B 19 kD-interacting | | | | |
| | interacting protein 3 | protein 3 (BNIP3), nuclear gene encoding | | | | |
| | | mitochondrial protein, mRNA. | | | | |
| | | /FEA = mRNA /GEN = BNIP3 | | | | |
| | | /PROD = BCL2adenovirus E1B 19 kD- | | | | |
| | | interacting protein 3 | | | | |
| | | /DB_XREF = gi: 7669480 /UG = Hs.79428 | | | | |
| | | BCL2adenovirus E1B 19 kD-interacting | | | | |
| | | protein 3 /FL = gb: AF002697.1 gb: U15174.1 | | | | |
| | | gb: NM_004052.2 | | | | |
| 203554_x_at | pituitary tumor- | gb: NM_004219.2 /DEF = Homo sapiens | PTTG1 | −1.5 | −1.2 | −1.2 |
| | transforming 1 | pituitary tumor-transforming 1 (PTTG1), | | | | |
| | | mRNA. /FEA = mRNA /GEN = PTTG1 | | | | |
| | | /PROD = pituitary tumor-transforming | | | | |
| | | protein 1 /DB_XREF = gi: 11038651 | | | | |
| | | /UG = Hs.252587 pituitary tumor- | | | | |
| | | transforming 1 /FL = gb: NM_004219.2 | | | | |
| | | gb: AF095287.1 gb: AF062649.1 | | | | |
| | | gb: AF075242.1 | | | | |
| 214453_s_at | interferon-induced | Consensus includes gb: NM_006417.1 | IFI44 | 1.4 | −4.9 | 1.1 |
| | protein 44 | /DEF = Homo sapiens interferon-induced, | | | | |
| | | hepatitis C-associated microtubular | | | | |
| | | aggregate protein (44 kD) (MTAP44), | | | | |
| | | mRNA. /FEA = CDS /GEN = MTAP44 | | | | |
| | | /PROD = interferon-induced, hepatitis C- | | | | |
| | | associatedmicrotubular aggregate protein | | | | |
| | | (44 kD) /DB_XREF = gi: 5453743 | | | | |
| | | /UG = Hs.82316 interferon-induced, | | | | |
| | | hepatitis C-associated microtubular | | | | |
| | | aggregate protein (44 kD) | | | | |
| | | /FL = gb: NM_006417.1 | | | | |
| 209774_x_at | chemokine (C—X—C | gb: M57731.1 /DEF = Human gro-beta | CXCL2 | 1.3 | 1.1 | 1.4 |
| | motif) ligand 2 | mRNA, complete cds. /FEA = mRNA | | | | |
| | | /GEN = gro-beta /PROD = cytokine gro-beta | | | | |
| | | /DB_XREF = gi: 183626 /UG = Hs.75765 | | | | |
| | | GRO2 oncogene /FL = gb: M57731.1 | | | | |
| | | gb: M36820.1 gb: NM_002089.1 | | | | |
| 219386_s_at | SLAM family | gb: NM_020125.1 /DEF = Homo sapiens | SLAMF8 | −1.2 | −1.3 | −2.1 |
| | member 8 | BCM-like membrane protein precursor | | | | |
| | | (SBBI42), mRNA. /FEA = mRNA | | | | |
| | | /GEN = SBBI42 /PROD = BCM-like | | | | |
| | | membrane protein precursor | | | | |
| | | /DB_XREF = gi: 9910341 /UG = Hs.20450 | | | | |
| | | BCM-like membrane protein precursor | | | | |
| | | /FL = gb: AF144235.1 gb: NM_014036.1 | | | | |
| | | gb: AF146761.1 gb: NM_020125.1 | | | | |
| 210215_at | transferrin receptor 2 | gb: AF067864.1 /DEF = Homo sapiens | TFR2 | −1.0 | −1.0 | −1.0 |
| | | transferrin receptor 2 alpha (TFR2) | | | | |
| | | mRNA, complete cds. /FEA = mRNA | | | | |
| | | /GEN = TFR2 /PROD = transferrin receptor 2 | | | | |
| | | alpha /DB_XREF = gi: 5596369 | | | | |
| | | /UG = Hs.63758 transferrin receptor 2 | | | | |
| | | /FL = gb: AF067864.1 | | | | |
| 210184_at | integrin, alpha X | gb: M81695.1 /DEF = H. sapiens leukocyte | ITGAX | 1.1 | 1.0 | −1.2 |
| | (antigen CD11C | adhesion glycoprotein p150,95 mRNA, | | | | |
| | (p150), alpha | complete cds. /FEA = mRNA /GEN = ITGAX | | | | |
| | polypeptide) | /DB_XREF = gi: 487829 /UG = Hs.51077 | | | | |
| | | integrin, alpha X (antigen CD11C (p150), | | | | |
| | | alpha polypeptide) /FL = gb: M81695.1 | | | | |
| | | gb: NM_000887.2 | | | | |
| 208079_s_at | serine/threonine | gb: NM_003158.1 /DEF = Homo sapiens | STK6 | −1.0 | −1.0 | 1.1 |
| | kinase 6 | serinethreonine kinase 6 (STK6), mRNA. | | | | |
| | | /FEA = mRNA /GEN = STK6 | | | | |
| | | /PROD = serinethreonine kinase 6 | | | | |
| | | /DB_XREF = gi: 4507278 /UG = Hs.250822 | | | | |
| | | serinethreonine kinase 6 /FL = gb: D84212.1 | | | | |
| | | gb: NM_003158.1 | | | | |
| 205237_at | ficolin | gb: NM_002003.2 /DEF = Homo sapiens | FCN1 | −1.2 | −1.2 | 1.0 |
| | (collagen/fibrinogen | ficolin (collagenfibrinogen domain- | | | | |
| | domain | containing) 1 (FCN1), mRNA. | | | | |
| | containing) 1 | /FEA = mRNA /GEN = FCN1 /PROD = ficolin | | | | |
| | | 1 precursor /DB_XREF = gi: 8051583 | | | | |
| | | /UG = Hs.252136 ficolin (collagenfibrinogen | | | | |
| | | domain-containing) 1 /FL = gb: D83920.1 | | | | |
| | | gb: NM_002003.2 | | | | |
| 207645_s_at | chromodomain | gb: NM_004284.1 /DEF = Homo sapiens | CHD1L | −1.0 | −1.0 | −1.0 |
| | helicase DNA | chromodomain helicase DNA binding | | | | |
| | binding protein 1- | protein 1-like (CHD1L), mRNA. | | | | |
| | like | /FEA = mRNA /GEN = CHD1L | | | | |
| | | /PROD = chromodomain helicase DNA | | | | |
| | | binding protein1-like | | | | |
| | | /DB_XREF = gi: 4757977 /UG = Hs.159273 | | | | |
| | | chromodomain helicase DNA binding | | | | |
| | | protein 1-like /FL = gb: AF054177.1 | | | | |
| | | gb: NM_004284.1 | | | | |
| 210889_s_at | Fc fragment of IgG, | gb: M31933.1 /DEF = Human IgG low | FCGR2B | 1.2 | −1.2 | 1.0 |
| | low affinity IIb, | affinity Fc fragment receptor (FcRIIb3) | | | | |
| | receptor for (CD32) | mRNA, complete cds. /FEA = mRNA | | | | |
| | | /DB_XREF = gi: 182598 /UG = Hs.278443 Fc | | | | |
| | | fragment of IgG, low affinity IIb, receptor | | | | |
| | | for (CD32) /FL = gb: M31933.1 | | | | |
| 209875_s_at | secreted | gb: M83248.1 /DEF = Human nephropontin | SPP1 | −1.4 | 1.2 | 1.2 |
| | phosphoprotein 1 | mRNA, complete cds. /FEA = mRNA | | | | |
| | (osteopontin, bone | /GEN = nephropontin /PROD = nephropontin | | | | |
| | sialoprotein I, early | /DB_XREF = gi: 189150 /UG = Hs.313 | | | | |
| | T-lymphocyte | secreted phosphoprotein 1 (osteopontin, | | | | |
| | activation 1) | bone sialoprotein I, early T-lymphocyte | | | | |
| | | activation 1) /FL = gb: M83248.1 | | | | |
| 205686_s_at | CD86 antigen | gb: NM_006889.1 /DEF = Homo sapines | CD86 | 1.1 | −1.1 | 1.0 |
| | (CD28 antigen | CD86 antigen (CD28 antigen ligand 2, B7- | | | | |
| | ligand 2, B7-2 | 2 antigen) (CD86), mRNA. /FEA = mRNA | | | | |
| | antigen) | /GEN = CD86 /PROD = CD86 antigen (CD28 | | | | |
| | | antigen ligand 2, B7-2antigen) | | | | |
| | | /DB_XREF = gi: 5901919 /UG = Hs.27954 | | | | |
| | | CD86 antigen (CD28 antigen ligand 2, B7- | | | | |
| | | 2 antigen) /FL = gb: U04343.1 | | | | |
| | | gb: NM_006889.1 | | | | |
| 218075_at | achalasia, | gb: NM_015665.1 /DEF = Homo sapiens | AAAS | 1.1 | −1.2 | −1.4 |
| | adrenocortical | aladin (AAAS), mRNA. /FEA = mRNA | | | | |
| | insufficiency, | /GEN = AAAS /PROD = aladin | | | | |
| | alacrimia (Allgrove, | /DB_XREF = gi: 12962936 /UG = Hs.125262 | | | | |
| | triple-A) | aladin /FL = gb: BC000659.1 | | | | |
| | | gb: NM_015665.1 gb: AF226048.1 | | | | |
| 202240_at | polo-like kinase 1 | gb: NM_005030.1 /DEF = Homo sapiens | | | | |
| | (Drosophila) | polo (Drosophia)-like kinase (PLK), | | | | |
| | | mRNA. /FEA = mRNA /GEN = PLK | | | | |
| | | /PROD = polo (Drosophia)-like kinase | | | | |
| | | /DB_XREF = gi: 4826915 /UG = Hs.77597 | | | | |
| | | polo (Drosophia)-like kinase | | | | |
| | | /FL = gb: BC002369.1 gb: BC003002.1 | | | | |
| | | gb: U01038.1 gb: L19559.1 | | | | |
| | | gb: NM_005030.1 | | | | |
| 209392_at | ectonucleotide | gb: L35594.1 /DEF = Human autotaxin | ENPP2 | 1.0 | −2.1 | −1.7 |
| | pyrophosphatase/phosphodiesterase | mRNA, complete cds. /FEA = mRNA | | | | |
| | 2 (autotaxin) | /PROD = autotaxin /DB_XREF = gi: 537905 | | | | |
| | | /UG = Hs.174185 ectonucleotide | | | | |
| | | pyrophosphatasephosphodiesterase 2 | | | | |
| | | (autotaxin) /FL = gb: L35594.1 | | | | |
| 202600_s_at | nuclear receptor | Consensus includes gb: AI824012 | NRIP1 | 61.3 | −1.3 | −1.0 |
| | interacting protein 1 | /FEA = EST /DB_XREF = gi: 5444683 | | | | |
| | | /DB_XREF = est: wj29e06.x1 | | | | |
| | | /CLONE = IMAGE: 2404258 | | | | |
| | | /UG = Hs.155017 nuclear receptor | | | | |
| | | interacting protein 1 /FL = gb: NM_003489.1 | | | | |
| 202589_at | thymidylate | gb: NM_001071.1 /DEF = Homo sapiens | TYMS | 1.0 | −1.2 | −1.7 |
| | synthetase | thymidylate synthetase (TYMS), mRNA. | | | | |
| | | /FEA = mRNA /GEN = TYMS | | | | |
| | | /PROD = thymidylate synthetase | | | | |
| | | /DB_XREF = gi: 4507750 /UG = Hs.82962 | | | | |
| | | thymidylate synthetase | | | | |
| | | /FL = gb: BC002567.1 gb: NM_001071.1 | | | | |
| 221521_s_at | DNA replication | gb: BC003186.1 /DEF = Homo sapiens, | Pfs2 | −1.0 | −1.0 | −1.0 |
| | complex GINS | HSPC037 protein, clone MGC: 673, | | | | |
| | protein PSF2 | mRNA, complete cds. /FEA = mRNA | | | | |
| | | /PROD = HSPC037 protein | | | | |
| | | /DB_XREF = gi: 13112024 /UG = Hs.108196 | | | | |
| | | HSPC037 protein /FL = gb: BC003186.1 | | | | |
| | | gb: AF201939.1 | | | | |
| 201005_at | CD9 antigen (p24) | gb: NM_001769.1 /DEF = Homo sapiens | CD9 | 1.3 | 1.1 | 1.2 |
| | | CD9 antigen (p24) (CD9), mRNA. | | | | |
| | | /FEA = mRNA /GEN = CD9 /PROD = CD9 | | | | |
| | | antigen (p24) /DB_XREF = gi: 4502692 | | | | |
| | | /UG = Hs.1244 CD9 antigen (p24) | | | | |
| | | /FL = gb: M38690.1 gb: NM_001769.1 | | | | |
| | | gb: L34068.1 | | | | |
| 219148_at | T-LAK cell- | gb: NM_018492.1 /DEF = Homo sapiens | TOPK | −1.0 | −1.0 | −1.0 |
| | originated protein | PDZ-binding kinase; T-cell originated | | | | |
| | kinase | protein kinase (TOPK), mRNA. | | | | |
| | | /FEA = mRNA /GEN = TOPK /PROD = PDZ- | | | | |
| | | binding kinase /DB_XREF = gi: 8923876 | | | | |
| | | /UG = Hs.104741 PDZ-binding kinase; T- | | | | |
| | | cell originated protein kinase | | | | |
| | | /FL = gb: AF189722.1 gb: AF237709.1 | | | | |
| | | gb: NM_018492.1 gb: AB027249.1 | | | | |
| | | gb: AB027250.1 | | | | |
| 217762_s_at | RAB31, member | Consensus includes gb: BE789881 | RAB31 | 1.3 | −1.3 | −1.1 |
| | RAS oncogene | /FEA = EST /DB_XREF = gi: 10211079 | | | | |
| | family | /DB_XREF = est: 601476750F1 | | | | |
| | | /CLONE = IMAGE: 3879493 | | | | |
| | | /UG = Hs.223025 RAB31, member RAS | | | | |
| | | oncogene family /FL = gb: AF234995.1 | | | | |
| | | gb: BC001148.1 gb: U59877.1 gb: U57091.1 | | | | |
| | | gb: NM_006868.1 gb: AF183421.1 | | | | |
| 205046_at | centromere protein | gb: NM_001813.1 /DEF = Homo sapiens | CENPE | −1.0 | −1.0 | −1.0 |
| | E, 312 kDa | centromere protein E (312 kD) (CENPE), | | | | |
| | | mRNA. /FEA = mRNA /GEN = CENPE | | | | |
| | | /PROD = centromere protein E | | | | |
| | | /DB_XREF = gi: 4502780 /UG = Hs.75573 | | | | |
| | | centromere protein E (312 kD) | | | | |
| | | /FL = gb: NM_001813.1 | | | | |
| 204026_s_at | ZW10 interactor | gb: NM_007057.1 /DEF = Homo sapiens | ZWINT | 1.2 | 1.1 | −1.4 |
| | | ZW10 interactor (ZWINT), mRNA. | | | | |
| | | /FEA = mRNA /GEN = ZWINT | | | | |
| | | /PROD = ZW10 interactor | | | | |
| | | /DB_XREF = gi: 6857828 /UG = Hs.42650 | | | | |
| | | ZW10 interactor /FL = gb: AF067656.1 | | | | |
| | | gb: NM_007057.1 | | | | |
| 204834_at | fibrinogen-like 2 | gb: NM_006682.1 /DEF = Homo sapiens | FGL2 | 1.0 | −1.5 | −1.3 |
| | | fibrinogen-like 2 (FGL2), mRNA. | | | | |
| | | /FEA = mRNA /GEN = FGL2 | | | | |
| | | /PROD = fibrinogen-like 2 | | | | |
| | | /DB_XREF = gi: 5730074 /UG = Hs.2659 | | | | |
| | | fibrinogen-like 2 /FL = gb: NM_006682.1 | | | | |
| 207369_at | bombesin-like | Consensus includes gb: Z97632 | BRS3 | −66.2 | 74.8 | 1.4 |
| | receptor 3 | /DEF = Human DNA sequence from PAC | | | | |
| | | 196E23 on chromosome Xq26.1-27.2. | | | | |
| | | Contains the TAT-SF1 (HIV-1 | | | | |
| | | transcriptional elongation factor TAT | | | | |
| | | cofactor TAT-SF1) gene, the BRS3 | | | | |
| | | (Bombesin Receptor subtype-3 (Uterine | | | | |
| | | Bombesin Receptor, BRS-3) gene, an | | | | |
| | | unknown gene . . . /FEA = mRNA_2 | | | | |
| | | /DB_XREF = gi: 2808417 /UG = Hs.121484 | | | | |
| | | bombesin-like receptor 3 /FL = gb: L08893.1 | | | | |
| | | gb: NM_001727.1 | | | | |
| 204580_at | matrix | gb: NM_002426.1 /DEF = Homo sapiens | MMP12 | −56.1 | −1.0 | −50.9 |
| | metalloproteinase | matrix metalloproteinase 12 (macrophage | | | | |
| | 12 (macrophage | elastase) (MMP12), mRNA. /FEA = mRNA | | | | |
| | elastase) | /GEN = MMP12 /PROD = matrix | | | | |
| | | metalloproteinase 12 preproprotein | | | | |
| | | /DB_XREF = gi: 4505206 /UG = Hs.1695 | | | | |
| | | matrix metalloproteinase 12 (macrophage | | | | |
| | | elastase) /FL = gb: L23808.1 | | | | |
| | | gb: NM_002426.1 | | | | |
| 208892_s_at | dual specificity | gb: BC003143.1 /DEF = Homo sapiens, dual | DUSP6 | 1.3 | −1.4 | −1.2 |
| | phosphatase 6 | specificity phosphatase 6, clone | | | | |
| | | MGC: 3789, mRNA, complete cds. | | | | |
| | | /FEA = mRNA /PROD = dual specificity | | | | |
| | | phosphatase 6 /DB_XREF = gi: 13111942 | | | | |
| | | /UG = Hs.180383 dual specificity | | | | |
| | | phosphatase 6 /FL = gb: BC003562.1 | | | | |
| | | gb: BC003143.1 gb: BC005047.1 | | | | |
| | | gb: AB013382.1 gb: NM_001946.1 | | | | |
| 207850_at | chemokine (C—X—C | gb: NM_002090.1 /DEF = Homo sapiens | CXCL3 | 1.1 | 1.0 | 1.0 |
| | motif) ligand 3 | GRO3 oncogene (GRO3), mRNA. | | | | |
| | | /FEA = mRNA /GEN = GRO3 /PROD = GRO3 | | | | |
| | | oncogene /DB_XREF = gi: 4504156 | | | | |
| | | /UG = Hs.89690 GRO3 oncogene | | | | |
| | | /FL = gb: M36821.1 gb: NM_002090.1 | | | | |
| 203362_s_at | MAD2 mitotic | gb: NM_002358.2 /DEF = Homo sapiens | MAD2L1 | −1.0 | −1.0 | −1.1 |
| | arrest deficient-like | MAD2 (mitotic arrest deficient, yeast, | | | | |
| | 1 (yeast) | homolog)-like 1 (MAD2L1), mRNA. | | | | |
| | | /FEA = mRNA /GEN = MAD2L1 | | | | |
| | | /PROD = MAD2-like 1 | | | | |
| | | /DB_XREF = gi: 6466452 /UG = Hs.79078 | | | | |
| | | MAD2 (mitotic arrest deficient, yeast, | | | | |
| | | homolog)-like 1 /FL = gb: BC000356.1 | | | | |
| | | gb: U65410.1 gb: NM_002358.2 | | | | |
| | | gb: U31278.1 | | | | |
| 210146_x_at | leukocyte | gb: AF004231.1 /DEF = Homo sapiens | LILRB2 | 1.2 | −1.7 | −1.5 |
| | immunoglobulin- | monocytemacrophage Ig-related receptor | | | | |
| | like receptor, | MIR-10 (MIR cl-10) mRNA, complete cds. | | | | |
| | subfamily B (with | /FEA = mRNA /GEN = MIR cl-10 | | | | |
| | TM and ITIM | /PROD = MIR-10 /DB_XREF = gi: 2343110 | | | | |
| | domains), member 2 | /UG = Hs.22405 leukocyte immunoglobulin- | | | | |
| | | like receptor, subfamily B (with TM and | | | | |
| | | ITIM domains), member 2 | | | | |
| | | /FL = gb: AF000574.1 gb: AF004231.1 | | | | |
| | | gb: AF011565.1 | | | | |
| 215925_s_at | CD72 antigen | Consensus includes gb: AF283777.2 | CD72 | 69.7 | −1.4 | 1.4 |
| | | /DEF = Homo sapiens clone TCBAP0702 | | | | |
| | | mRNA sequence. /FEA = mRNA | | | | |
| | | /DB_XREF = gi: 10281735 /UG = Hs.116481 | | | | |
| | | CD72 antigen | | | | |
| 201200_at | cellular repressor | gb: NM_003851.1 /DEF = Homo sapiens | CREG1 | 1.4 | −1.7 | 1.0 |
| | of E1A-stimulated | cellular repressor of E1A-stimulated genes | | | | |
| | genes | (CREG), mRNA. /FEA = mRNA | | | | |
| | | /GEN = CREG /PROD = cellular repressor of | | | | |
| | | E1A-stimulated genes | | | | |
| | | /DB_XREF = gi: 4503036 /UG = Hs.5710 | | | | |
| | | cellular repressor of E1A-stimulated genes | | | | |
| | | /FL = gb: AF084523.1 gb: NM_003851.1 | | | | |
| 205393_s_at | CHK1 checkpoint | gb: NM_001274.1 /DEF = Homo sapiens | CHEK1 | −1.0 | −1.0 | −1.0 |
| | homolog (S. pombe) | CHK1 (checkpoint, S. pombe) homolog | | | | |
| | | (CHEK1), mRNA. /FEA = mRNA | | | | |
| | | /GEN = CHEK1 /PROD = CHK1 (checkpoint, | | | | |
| | | S. pombe) homolog | | | | |
| | | /DB_XREF = gi: 4502802 /UG = Hs.20295 | | | | |
| | | CHK1 (checkpoint, S. pombe) homolog | | | | |
| | | /FL = gb: AF016582.1 gb: NM_001274.1 | | | | |
| 201422_at | interferon, gamma- | gb: NM_006332.1 /DEF = Homo sapiens | IFI30 | −1.4 | 1.1 | 1.1 |
| | inducible protein | interferon, gamma-inducible protein 30 | | | | |
| | 30 | (IFI30), mRNA. /FEA = mRNA /GEN = IFI30 | | | | |
| | | /PROD = interferon, gamma-inducible | | | | |
| | | protein 30 /DB_XREF = gi: 5453695 | | | | |
| | | /UG = Hs.14623 interferon, gamma- | | | | |
| | | inducible protein 30 /FL = gb: J03909.1 | | | | |
| | | gb: NM_006332.1 gb: AF097362.1 | | | | |
| 202779_s_at | ubiquitin- | gb: NM_014501.1 /DEF = Homo sapiens | UBE2S | 1.1 | −1.5 | −1.2 |
| | conjugating | ubiquitin carrier protein (E2-EPF), mRNA. | | | | |
| | enzyme E2S | /FEA = mRNA /GEN = E2-EPF | | | | |
| | | /PROD = ubiquitin carrier protein | | | | |
| | | /DB_XREF = gi: 7657045 /UG = Hs.174070 | | | | |
| | | ubiquitin carrier protein /FL = gb: M91670.1 | | | | |
| | | gb: NM_014501.1 | | | | |
| 57540_at | ribokinase | Cluster Incl. AI823980: wj29b03.x1 Homo | RBKS | −59.7 | −1.0 | −1.0 |
| | | sapiens cDNA, 3 end /clone = IMAGE- | | | | |
| | | 2404205 /clone_end = 3′ /gb = AI823980 | | | | |
| | | /gi = 5444651 /ug = Hs.11916 /len = 620 | | | | |
| 204068_at | serine/threonine | gb: NM_006281.1 /DEF = Homo sapiens | STK3 | 51.1 | −1.0 | −1.0 |
| | kinase 3 (STE20 | serinethreonine kinase 3 (Ste20, yeast | | | | |
| | homolog, yeast) | homolog) (STK3), mRNA. /FEA = mRNA | | | | |
| | | /GEN = STK3 /PROD = serinethreonine | | | | |
| | | kinase 3 (Ste20, yeasthomolog) | | | | |
| | | /DB_XREF = gi: 5454093 /UG = Hs.166684 | | | | |
| | | serinethreonine kinase 3 (Ste20, yeast | | | | |
| | | homolog) /FL = gb: U26424.1 gb: U60206.1 | | | | |
| | | gb: NM_006281.1 | | | | |
| 218559_s_at | v-maf | gb: NM_005461.1 /DEF = Homo sapiens | MAFB | 1.1 | −1.5 | 1.1 |
| | musculoaponeurotic | Kreisler (mouse) maf-related leucine | | | | |
| | fibrosarcoma | zipper homolog (KRML), mRNA. | | | | |
| | oncogene homolog | /FEA = mRNA /GEN = KRML | | | | |
| | B (avian) | /PROD = Kreisler (mouse) maf-related | | | | |
| | | leucine zipperhomolog | | | | |
| | | /DB_XREF = gi: 4885446 /UG = Hs.169487 | | | | |
| | | Kreisler (mouse) maf-related leucine | | | | |
| | | zipper homolog /FL = gb: AF134157.1 | | | | |
| | | gb: NM_005461.1 | | | | |
| 208107_s_at | exonuclease NEF- | gb: NM_030941.1 /DEF = Homo sapiens | | −1.0 | −1.0 | −1.0 |
| | sp | exonuclease NEF-sp (LOC81691), mRNA. | | | | |
| | | /FEA = mRNA /GEN = LOC81691 | | | | |
| | | /PROD = exonuclease NEF-sp | | | | |
| | | /DB_XREF = gi: 13569912 | | | | |
| | | /FL = gb: NM_030941.1 | | | | |
| 210512_s_at | vascular | gb: AF022375.1 /DEF = Homo sapiens | VEGF | 80.6 | −73.5 | −1.2 |
| | endothelial growth | vascular endothelial growth factor mRNA, | | | | |
| | factor | complete cds. /FEA = mRNA | | | | |
| | | /PROD = vascular endothelial growth factor | | | | |
| | | /DB_XREF = gi: 3719220 /UG = Hs.73793 | | | | |
| | | vascular endothelial growth factor | | | | |
| | | /FL = gb: M32977.1 gb: AF022375.1 | | | | |
| | | gb: NM_003376.1 gb: AB021221.1 | | | | |
| | | gb: AF091352.1 | | | | |
| 200783_s_at | stathmin | gb: NM_005563.2 /DEF = Homo sapiens | STMN1 | −1.2 | 1.6 | 1.1 |
| | 1/oncoprotein 18 | leukemia-associated phosphoprotein p18 | | | | |
| | | (stathmin) (LAP18), mRNA. /FEA = mRNA | | | | |
| | | /GEN = LAP18 /PROD = leukemia- | | | | |
| | | associated phosphoprotein p18 | | | | |
| | | /DB_XREF = gi: 13518023 /UG = Hs.81915 | | | | |
| | | leukemia-associated phosphoprotein p18 | | | | |
| | | (stathmin) /FL = gb: NM_005563.2 | | | | |
| | | gb: J04991.1 | | | | |
| 202107_s_at | MCM2 | gb: NM_004526.1 /DEF = Homo sapiens | MCM2 | 1.1 | −1.5 | −1.5 |
| | minichromosome | minichromosome maintenance deficient | | | | |
| | maintenance | (S. cerevisiae) 2 (mitotin) (MCM2), mRNA. | | | | |
| | deficient 2, mitotin | /FEA = mRNA /GEN = MCM2 | | | | |
| | (S. cerevisiae) | /PROD = minichromosome maintenance | | | | |
| | | deficient (S. cerevisiae) 2 (mitotin) | | | | |
| | | /DB_XREF = gi: 4758705 /UG = Hs.57101 | | | | |
| | | minichromosome maintenance deficient | | | | |
| | | (S. cerevisiae) 2 (mitotin) | | | | |
| | | /FL = gb: D83987.1 gb: NM_004526.1 | | | | |
| 213566_at | ribonuclease, | Consensus includes gb: NM_005615.1 | RNASE6 | 1.2 | −1.2 | 1.7 |
| | RNase A family, k6 | /DEF = Homo sapiens ribonuclease, RNase | | | | |
| | | A family, k6 (RNASE6), mRNA. | | | | |
| | | /FEA = CDS /GEN = RNASE6 | | | | |
| | | /PROD = ribonuclease, RNase A family, k6 | | | | |
| | | /DB_XREF = gi: 5032044 /UG = Hs.23262 | | | | |
| | | ribonuclease, RNase A family, k6 | | | | |
| | | /FL = gb: NM_005615.1 | | | | |
| 205240_at | G-protein signalling | gb: NM_013296.1 /DEF = Homo sapiens | GPSM2 | −1.0 | −1.0 | −1.0 |
| | modulator 2 (AGS3 | LGN protein (HSU54999), mRNA. | | | | |
| | like, C. elegans) | /FEA = mRNA /GEN = HSU54999 | | | | |
| | | /PROD = LGN protein | | | | |
| | | /DB_XREF = gi: 9558734 /UG = Hs.278338 | | | | |
| | | LGN protein /FL = gb: U54999.1 | | | | |
| | | gb: NM_013296.1 | | | | |
| 208498_s_at | amylase, alpha 2B; | gb: NM_004038.1 /DEF = Homo sapiens | AMY2B | 2.0 | −1.3 | 1.1 |
| | pancreatic | amylase, alpha 1A; salivary (AMY1A), | | | | |
| | | mRNA. /FEA = CDS /GEN = AMY1A | | | | |
| | | /PROD = amylase, alpha 1A; salivary | | | | |
| | | /DB_XREF = gi: 4757749 /UG = Hs.274376 | | | | |
| | | amylase, alpha 1A; salivary | | | | |
| | | /FL = gb: NM_004038.1 | | | | |
| 202241_at | tribbles homolog 1 | gb: NM_025195.1 /DEF = Homo sapiens | TRIB1 | 1.4 | −1.3 | 1.1 |
| | (Drosophila) | phosphoprotein regulated by mitogenic | | | | |
| | | pathways (C8FW), mRNA. /FEA = mRNA | | | | |
| | | /GEN = C8FW /PROD = G-protein-coupled | | | | |
| | | receptor induced protein | | | | |
| | | /DB_XREF = gi: 13399327 /UG = Hs.7837 | | | | |
| | | phosphoprotein regulated by mitogenic | | | | |
| | | pathways /FL = gb: AF205437.1 | | | | |
| | | gb: NM_025195.1 | | | | |
| 219947_at | C-type (calcium | gb: NM_016184.1 /DEF = Homo sapiens C- | CLECSF6 | 1.5 | −1.2 | −1.8 |
| | dependent, | type (calcium dependent, carbohydrate- | | | | |
| | carbohydrate- | recognition domain) lectin, superfamily | | | | |
| | recognition | member 6 (CLECSF6), mRNA. | | | | |
| | domain) lectin, | /FEA = mRNA /GEN = CLECSF6 /PROD = C- | | | | |
| | superfamily | type (calcium dependent, carbohydrate- | | | | |
| | member 6 | recognition domain) lectin, | | | | |
| | | superfamilymember 6 | | | | |
| | | /DB_XREF = gi: 7705337 /UG = Hs.115515 C- | | | | |
| | | type (calcium dependent, carbohydrate- | | | | |
| | | recognition domain) lectin, superfamily | | | | |
| | | member 6 /FL = gb: AF109146.1 | | | | |
| | | gb: NM_016184.1 gb: AF067800.1 | | | | |
| 204787_at | Ig superfamily | gb: NM_007268.1 /DEF = Homo sapiens Ig | VSIG4 | 1.1 | 1.0 | 1.2 |
| | protein | superfamily protein (Z39IG), mRNA. | | | | |
| | | /FEA = mRNA /GEN = Z39IG /PROD = Ig | | | | |
| | | superfamily protein | | | | |
| | | /DB_XREF = gi: 6005957 /UG = Hs.8904 Ig | | | | |
| | | superfamily protein /FL = gb: NM_007268.1 | | | | |
| 204860_s_at | baculoviral IAP | Consensus includes gb: AI817801 | BIRC1 | 1.2 | 1.0 | 1.1 |
| | repeat-containing 1 | /FEA = EST /DB_XREF = gi: 5436880 | | | | |
| | | /DB_XREF = est: wk40d12.x1 | | | | |
| | | /CLONE = IMAGE: 2417879 /UG = Hs.79019 | | | | |
| | | baculoviral IAP repeat-containing 1 | | | | |
| | | /FL = gb: U19251.1 gb: NM_004536.1 | | | | |
| 205547_s_at | transgelin | gb: NM_003186.2 /DEF = Homo sapiens | TAGLN | 1.4 | 1.0 | 1.1 |
| | | transgelin (TAGLN), mRNA. /FEA = mRNA | | | | |
| | | /GEN = TAGLN /PROD = transgelin | | | | |
| | | /DB_XREF = gi: 12621918 /UG = Hs.75777 | | | | |
| | | transgelin /FL = gb: NM_003186.2 | | | | |
| | | gb: M95787.1 gb: D17409.1 | | | | |
| 218755_at | kinesin family | gb: NM_005733.1 /DEF = Homo sapiens | KIF20A | −1.0 | −1.0 | −1.0 |
| | member 20A | RAB6 interacting, kinesin-like | | | | |
| | | (rabkinesin6) (RAB6KIFL), mRNA. | | | | |
| | | /FEA = mRNA /GEN = RAB6KIFL | | | | |
| | | /PROD = RAB6 interacting, kinesin-like | | | | |
| | | (rabkinesin6) /DB_XREF = gi: 5032012 | | | | |
| | | /UG = Hs.73625 RAB6 interacting, kinesin- | | | | |
| | | like (rabkinesin6) /FL = gb: AF070672.1 | | | | |
| | | gb: AF153329.1 gb: NM_005733.1 | | | | |
| 207906_at | interleukin 3 | gb: NM_000588.1 /DEF = Homo sapiens | IL3 | −1.0 | −1.0 | −1.0 |
| | (colony-stimulating | interleukin 3 (colony-stimulating factor, | | | | |
| | factor, multiple) | multiple) (IL3), mRNA. /FEA = mRNA | | | | |
| | | /GEN = IL3 /PROD = interleukin 3 (colony- | | | | |
| | | stimulating factor, multiple) | | | | |
| | | /DB_XREF = gi: 4504666 /UG = Hs.694 | | | | |
| | | interleukin 3 (colony-stimulating factor, | | | | |
| | | multiple) /FL = gb: M17115.1 gb: M14743.1 | | | | |
| | | gb: M20137.1 gb: NM_000588.1 | | | | |
| 210004_at | oxidised low | gb: AF035776.1 /DEF = Homo sapiens | OLR1 | 1.2 | −1.6 | −2.0 |
| | density lipoprotein | oxidized low-density lipoprotein receptor | | | | |
| | (lectin-like) | mRNA, complete cds. /FEA = mRNA | | | | |
| | receptor 1 | /PROD = oxidized low-density lipoprotein | | | | |
| | | receptor /DB_XREF = gi: 3941299 | | | | |
| | | /UG = Hs.77729 oxidised low density | | | | |
| | | lipoprotein (lectin-like) receptor 1 | | | | |
| | | /FL = gb: AB010710.1 gb: AF035776.1 | | | | |
| | | gb: NM_002543.1 | | | | |
| 221419_s_at | Similar to | gb: NM_013307.1 /DEF = Homo sapiens | | −1.5 | 1.0 | −1.1 |
| | ribosomal protein | non-functional folate binding protein | | | | |
| | L13a; 60S | (HSAF000381), mRNA. /FEA = CDS | | | | |
| | ribosomal protein | /GEN = HSAF000381 /PROD = non- | | | | |
| | L13a; 23 kD highly | functional folate binding protein | | | | |
| | basic protein | /DB_XREF = gi: 7019412 | | | | |
| | (LOC399810), | /FL = gb: NM_013307.1 | | | | |
| | mRNA [BLAST] | | | | | |
| 218436_at | endoplasmic | gb: NM_022464.1 /DEF = Homo sapiens | SIL1 | 115.9 | −1.8 | −1.7 |
| | reticulum | endoplasmic reticulum chaperone SIL1, | | | | |
| | chaperone SIL1, | homolog of yeast (SIL1), mRNA. | | | | |
| | homolog of yeast | /FEA = mRNA /GEN = SIL1 | | | | |
| | | /PROD = endoplasmic reticulum chaperone | | | | |
| | | SIL1, homolog ofyeast | | | | |
| | | /DB_XREF = gi: 11968008 /UG = Hs.297875 | | | | |
| | | endoplasmic reticulum chaperone SIL1, | | | | |
| | | homolog of yeast /FL = gb: NM_022464.1 | | | | |
| 205727_at | telomerase- | gb: NM_007110.1 /DEF = Homo sapiens | TEP1 | −1.0 | −1.0 | −1.0 |
| | associated protein 1 | telomerase-associated protein 1 (TEP1), | | | | |
| | | mRNA. /FEA = mRNA /GEN = TEP1 | | | | |
| | | /PROD = telomerase-associated protein 1 | | | | |
| | | /DB_XREF = gi: 6005893 /UG = Hs.232070 | | | | |
| | | telomerase-associated protein 1 | | | | |
| | | /FL = gb: U86136.1 gb: NM_007110.1 | | | | |
| 218831_s_at | Fc fragment of IgG, | gb: NM_004107.1 /DEF = Homo sapiens Fc | FCGRT | −1.2 | −1.2 | 1.0 |
| | receptor, | fragment of IgG, receptor, transporter, | | | | |
| | transporter, alpha | alpha (FCGRT), mRNA. /FEA = mRNA | | | | |
| | | /GEN = FCGRT /PROD = Fc fragment of | | | | |
| | | IgG, receptor, transporter, alpha | | | | |
| | | /DB_XREF = gi: 4758345 /UG = Hs.111903 | | | | |
| | | Fc fragment of IgG, receptor, transporter, | | | | |
| | | alpha /FL = gb: NM_004107.1 gb: U12255.1 | | | | |
| 217963_s_at | nerve growth factor | gb: NM_014380.1 /DEF = Homo sapiens | NGFRAP1 | 1.2 | 1.0 | 1.3 |
| | receptor | p75NTR-associated cell death executor; | | | | |
| | (TNFRSF16) | ovarian granulosa cell protein (13 kD) | | | | |
| | associated protein 1 | (DXS6984E), mRNA. /FEA = mRNA | | | | |
| | | /GEN = DXS6984E /PROD = p75NTR- | | | | |
| | | associated cell death executor; | | | | |
| | | ovariangranulosa cell protein (13 kD) | | | | |
| | | /DB_XREF = gi: 7657043 /UG = Hs.17775 | | | | |
| | | p75NTR-associated cell death executor; | | | | |
| | | ovarian granulosa cell protein (13 kD) | | | | |
| | | /FL = gb: NM_014380.1 gb: AF187064.1 | | | | |
| 209642_at | BUB1 budding | gb: AF043294.2 /DEF = Homo sapiens | BUB1 | −1.0 | −50.6 | −52.7 |
| | uninhibited by | putative mitotic checkpoint kinase mRNA, | | | | |
| | benzimidazoles 1 | complete cds. /FEA = mRNA | | | | |
| | homolog (yeast) | /PROD = putative mitotic checkpoint kinase | | | | |
| | | /DB_XREF = gi: 6970210 /UG = Hs.98658 | | | | |
| | | budding uninhibited by benzimidazoles 1 | | | | |
| | | (yeast homolog) /FL = gb: AF047471.1 | | | | |
| | | gb: AF053305.1 gb: AF046078.1 | | | | |
| | | gb: NM_004336.1 gb: AF043294.2 | | | | |
| 203418_at | cyclin A2 | gb: NM_001237.1 /DEF = Homo sapiens | CCNA2 | −1.0 | −1.0 | −72.9 |
| | | cyclin A2 (CCNA2), mRNA. /FEA = mRNA | | | | |
| | | /GEN = CCNA2 /PROD = cyclin A | | | | |
| | | /DB_XREF = gi: 4502612 /UG = Hs.85137 | | | | |
| | | cyclin A2 /FL = gb: NM_001237.1 | | | | |
| 203755_at | BUB1 budding | gb: NM_001211.2 /DEF = Homo sapiens | BUB1B | 1.1 | −1.3 | −1.1 |
| | uninhibited by | budding uninhibited by benzimidazoles 1 | | | | |
| | benzimidazoles 1 | (yeast homolog), beta (BUB1B), mRNA. | | | | |
| | homolog beta | /FEA = mRNA /GEN = BUB1B | | | | |
| | (yeast) | /PROD = budding uninhibited by | | | | |
| | | benzimidazoles 1 (yeasthomolog), beta | | | | |
| | | /DB_XREF = gi: 5729749 /UG = Hs.36708 | | | | |
| | | budding uninhibited by benzimidazoles 1 | | | | |
| | | (yeast homolog), beta /FL = gb: AF053306.1 | | | | |
| | | gb: AF035933.1 gb: AF068760.1 | | | | |
| | | gb: AF046918.1 gb: AF107297.1 | | | | |
| | | gb: AF046079.2 gb: NM_001211.2 | | | | |
| 205582_s_at | gamma- | gb: NM_004121.1 /DEF = Homo sapiens | GGTLA1 | −1.0 | −1.0 | −55.9 |
| | glutamyltransferase- | gamma-glutamyltransferase-like activity 1 | | | | |
| | like activity 1 | (GGTLA1), mRNA. /FEA = mRNA | | | | |
| | | /GEN = GGTLA1 /PROD = gamma- | | | | |
| | | glutamyltransferase-like activity 1 | | | | |
| | | /DB_XREF = gi: 4758431 /UG = Hs.1675 | | | | |
| | | gamma-glutamyltransferase-like activity 1 | | | | |
| | | /FL = gb: M64099.1 gb: NM_004121.1 | | | | |
| 206420_at | immunoglobulin | gb: NM_005849.1 /DEF = Homo sapiens | IGSF6 | −1.2 | −1.4 | −1.3 |
| | superfamily, | immunoglobulin superfamily, member 6 | | | | |
| | member 6 | (IGSF6), mRNA. /FEA = mRNA | | | | |
| | | /GEN = IGSF6 /PROD = immunoglobulin | | | | |
| | | superfamily, member 6 | | | | |
| | | /DB_XREF = gi: 5031672 /UG = Hs.135194 | | | | |
| | | immunoglobulin superfamily, member 6 | | | | |
| | | /FL = gb: NM_005849.1 | | | | |
| 204072_s_at | hypothetical | gb: NM_023037.1 /DEF = Homo sapiens | 13CDNA73 | 1.3 | 1.2 | −1.5 |
| | protein CG003 | putative gene product (13CDNA73), | | | | |
| | | mRNA. /FEA = mRNA /GEN = 13CDNA73 | | | | |
| | | /PROD = putative gene product | | | | |
| | | /DB_XREF = gi: 12957487 /UG = Hs.181304 | | | | |
| | | putative gene product | | | | |
| | | /FL = gb: NM_023037.1 | | | | |
| 201798_s_at | fer-1-like 3, | gb: NM_013451.1 /DEF = Homo sapiens fer- | FER1L3 | −1.3 | −1.7 | −2.0 |
| | myoferlin (C. elegans) | 1 (C. elegans)-like 3 (myoferlin) (FER1L3), | | | | |
| | | mRNA. /FEA = mRNA /GEN = FER1L3 | | | | |
| | | /PROD = fer-1 (C. elegans)-like 3 (myoferlin) | | | | |
| | | /DB_XREF = gi: 7305052 /UG = Hs.234680 | | | | |
| | | fer-1 (C. elegans)-like 3 (myoferlin) | | | | |
| | | /FL = gb: AF182316.1 gb: NM_013451.1 | | | | |
| 203213_at | cell division cycle | Consensus includes gb: AL524035 | CDC2 | −1.0 | −1.0 | −1.0 |
| | 2, G1 to S and G2 | /FEA = EST /DB_XREF = gi: 12787528 | | | | |
| | to M | /DB_XREF = est: AL524035 | | | | |
| | | /CLONE = CS0DC003YN06 (3 prime) | | | | |
| | | /UG = Hs.184572 cell division cycle 2, G1 | | | | |
| | | to S and G2 to M /FL = gb: NM_001786.1 | | | | |
| 212681_at | erythrocyte | Consensus includes gb: AI770004 | EPB41L3 | 1.6 | 1.1 | −1.1 |
| | membrane protein | /FEA = EST /DB_XREF = gi: 5236513 | | | | |
| | band 4.1-like 3 | /DB_XREF = est: wh66d06.x1 | | | | |
| | | /CLONE = IMAGE: 2385707 | | | | |
| | | /UG = Hs.103839 erythrocyte membrane | | | | |
| | | protein band 4.1-like 3 | | | | |
| 205167_s_at | cell division cycle | gb: NM_001790.2 /DEF = Homo sapiens cell | CDC25C | −1.0 | −1.0 | −1.0 |
| | 25C | division cycle 25C (CDC25C), transcript | | | | |
| | | variant 1, mRNA. /FEA = mRNA | | | | |
| | | /GEN = CDC25C /PROD = cell division cycle | | | | |
| | | 25C protein, isoform a | | | | |
| | | /DB_XREF = gi: 12408659 /UG = Hs.656 cell | | | | |
| | | division cycle 25C /FL = gb: NM_001790.2 | | | | |
| | | gb: M34065.1 | | | | |
| 204033_at | thyroid hormone | gb: NM_004237.1 /DEF = Homo sapiens | TRIP13 | −67.9 | 1.3 | 1.0 |
| | receptor interactor | thyroid hormone receptor interactor 13 | | | | |
| | 13 | (TRIP13), mRNA. /FEA = mRNA | | | | |
| | | /GEN = TRIP13 /PROD = thyroid hormone | | | | |
| | | receptor interactor 13 | | | | |
| | | /DB_XREF = gi: 11321606 /UG = Hs.6566 | | | | |
| | | thyroid hormone receptor interactor 13 | | | | |
| | | /FL = gb: NM_004237.1 gb: BC000404.1 | | | | |
| | | gb: U96131.1 | | | | |
| 201110_s_at | thrombospondin 1 | gb: NM_003246.1 /DEF = Homo sapiens | THBS1 | 1.9 | 86.1 | 61.4 |
| | | thrombospondin 1 (THBS1), mRNA. | | | | |
| | | /FEA = mRNA /GEN = THBS1 | | | | |
| | | /PROD = thrombospondin 1 | | | | |
| | | /DB_XREF = gi: 4507484 /UG = Hs.87409 | | | | |
| | | thrombospondin 1 /FL = gb: NM_003246.1 | | | | |
| 210052_s_at | TPX2, microtubule- | gb: AF098158.1 /DEF = Homo sapiens | TPX2 | −61.8 | −73.1 | −83.2 |
| | associated protein | restricted expressed proliferation | | | | |
| | homolog (Xenopus | associated protein 100 mRNA, complete | | | | |
| | laevis) | cds. /FEA = mRNA /PROD = restricted | | | | |
| | | expressed proliferation associatedprotein | | | | |
| | | 100 /DB_XREF = gi: 6073830 /UG = Hs.9329 | | | | |
| | | chromosome 20 open reading frame 1 | | | | |
| | | /FL = gb: NM_012112.1 gb: BC004136.1 | | | | |
| | | gb: AB024704.1 gb: AF146731.1 | | | | |
| | | gb: AF098158.1 gb: AB027467.1 | | | | |
| 202870_s_at | CDC20 cell | gb: NM_001255.1 /DEF = Homo sapiens | CDC20 | −1.0 | −1.0 | −63.0 |
| | division cycle 20 | CDC20 (cell division cycle 20, S. cerevisiae, | | | | |
| | homolog (S. cerevisiae) | homolog) (CDC20), mRNA. | | | | |
| | | /FEA = mRNA /GEN = CDC20 /PROD = cell | | | | |
| | | division cycle 20 /DB_XREF = gi: 4557436 | | | | |
| | | /UG = Hs.82906 CDC20 (cell division cycle | | | | |
| | | 20, S. cerevisiae, homolog) | | | | |
| | | /FL = gb: BC001088.1 gb: AF099644.1 | | | | |
| | | gb: NM_001255.1 gb: U05340.1 | | | | |
| 203005_at | lymphotoxin beta | gb: NM_002342.1 /DEF = Homo sapiens | LTBR | 1.2 | 1.5 | 1.2 |
| | receptor (TNFR | lymphotoxin beta receptor (TNFR | | | | |
| | superfamily, | superfamily, member 3 (LTBR), mRNA. | | | | |
| | member 3) | /FEA = mRNA /GEN = LTBR | | | | |
| | | /PROD = lymphotoxin B receptor | | | | |
| | | /DB_XREF = gi: 4505038 /UG = Hs.1116 | | | | |
| | | lymphotoxin beta receptor (TNFR | | | | |
| | | superfamily, member 3 /FL = gb: L04270.1 | | | | |
| | | gb: NM_002342.1 | | | | |
| 219607_s_at | membrane- | gb: NM_024021.1 /DEF = Homo sapiens | MS4A4A | −1.0 | 1.0 | 2.0 |
| | spanning 4- | membrane-spanning 4-domains, subfamily | | | | |
| | domains subfamily | A, member 4 (MS4A4), mRNA. | | | | |
| | A, member 4 | /FEA = mRNA /GEN = MS4A4 | | | | |
| | | /PROD = membrane-spanning 4-domains, | | | | |
| | | subfamily A, member4 | | | | |
| | | /DB_XREF = gi: 13430865 /UG = Hs.325960 | | | | |
| | | membrane-spanning 4-domains, subfamily | | | | |
| | | A, member 4 /FL = gb: AB013102.1 | | | | |
| | | gb: NM_024021.1 gb: AF068288.1 | | | | |
| | | gb: NM_016650.1 | | | | |
| 204887_s_at | polo-like kinase 4 | gb: NM_014264.1 /DEF = Homo sapiens | PLK4 | −1.0 | −1.0 | −1.2 |
| | (Drosophila) | serinethreonine kinase 18 (STK18), | | | | |
| | | mRNA. /FEA = mRNA /GEN = STK18 | | | | |
| | | /PROD = serinethreonine kinase 18 | | | | |
| | | /DB_XREF = gi: 7657626 /UG = Hs.172052 | | | | |
| | | serinethreonine kinase 18 | | | | |
| | | /FL = gb: NM_014264.1 | | | | |
| 204159_at | cyclin-dependent | gb: NM_001262.1 /DEF = Homo sapiens | CDKN2C | −1.0 | −1.0 | 52.9 |
| | kinase inhibitor 2C | cyclin-dependent kinase inhibitor 2C (p18, | | | | |
| | (p18, inhibits | inhibits CDK4) (CDKN2C), mRNA. | | | | |
| | CDK4) | /FEA = mRNA /GEN = CDKN2C | | | | |
| | | /PROD = cyclin-dependent kinase inhibitor | | | | |
| | | 2C (p18, inhibits CDK4) | | | | |
| | | /DB_XREF = gi: 4502750 /UG = Hs.4854 | | | | |
| | | cyclin-dependent kinase inhibitor 2C (p18, | | | | |
| | | inhibits CDK4) /FL = gb: AF041248.1 | | | | |
| | | gb: NM_001262.1 | | | | |
| 220832_at | toll-like receptor 8 | gb: NM_016610.1 /DEF = Homo sapiens | TLR8 | 1.4 | −2.0 | −95.0 |
| | | Toll-like receptor 8 (LOC51311), mRNA. | | | | |
| | | /FEA = mRNA /GEN = LOC51311 | | | | |
| | | /PROD = Toll-like receptor 8 | | | | |
| | | /DB_XREF = gi: 7706147 /UG = Hs.272410 | | | | |
| | | Toll-like receptor 8 /FL = gb: AF246971.1 | | | | |
| | | gb: NM_016610.1 gb: AF245703.1 | | | | |
| 204112_s_at | histamine N- | gb: NM_006895.1 /DEF = Homo sapiens | HNMT | 1.1 | −1.2 | 1.1 |
| | methyltransferase | histamine N-methyltransferase (HNMT), | | | | |
| | | mRNA. /FEA = mRNA /GEN = HNMT | | | | |
| | | /PROD = histamine N-methyltransferase | | | | |
| | | /DB_XREF = gi: 5901969 /UG = Hs.81182 | | | | |
| | | histamine N-methyltransferase | | | | |
| | | /FL = gb: U08092.1 gb: D16224.1 | | | | |
| | | gb: NM_006895.1 | | | | |
| 206148_at | interleukin 3 | gb: NM_002183.1 /DEF = Homo sapiens | IL3RA | −1.0 | −1.0 | −1.0 |
| | receptor, alpha | interleukin 3 receptor, alpha (low affinity) | | | | |
| | (low affinity) | (IL3RA), mRNA. /FEA = mRNA | | | | |
| | | /GEN = IL3RA /PROD = interleukin 3 | | | | |
| | | receptor, alpha (low affinity) | | | | |
| | | /DB_XREF = gi: 13324709 /UG = Hs.172689 | | | | |
| | | interleukin 3 receptor, alpha (low affinity) | | | | |
| | | /FL = gb: NM_002183.1 gb: M74782.1 | | | | |
| 202087_s_at | cathepsin L | gb: NM_001912.1 /DEF = Homo sapiens | CTSL | 1.2 | −1.5 | 1.4 |
| | | cathepsin L (CTSL), mRNA. /FEA = mRNA | | | | |
| | | /GEN = CTSL /PROD = cathepsin L | | | | |
| | | /DB_XREF = gi: 4503154 /UG = Hs.78056 | | | | |
| | | cathepsin L /FL = gb: NM_001912.1 | | | | |
| 218308_at | transforming, | gb: NM_006342.1 /DEF = Homo sapiens | TACC3 | 1.1 | −1.1 | 1.0 |
| | acidic coiled-coil | transforming, acidic coiled-coil containing | | | | |
| | containing protein 3 | protein 3 (TACC3), mRNA. /FEA = mRNA | | | | |
| | | /GEN = TACC3 /PROD = transforming acidic | | | | |
| | | coiled-coil containingprotein 3 | | | | |
| | | /DB_XREF = gi: 5454101 /UG = Hs.104019 | | | | |
| | | transforming, acidic coiled-coil containing | | | | |
| | | protein 3 /FL = gb: AF093543.1 | | | | |
| | | gb: NM_006342.1 | | | | |
| 208322_s_at | sialyltransferase | gb: NM_003033.1 /DEF = Homo sapiens | SIAT4A | 1.1 | −1.2 | 1.2 |
| | 4A (beta- | sialyltransferase 4A (beta-galactosidase | | | | |
| | galactoside alpha- | alpha-2,3-sialytransferase) (SIAT4A), | | | | |
| | 2,3- | mRNA. /FEA = mRNA /GEN = SIAT4A | | | | |
| | sialyltransferase) | /PROD = sialyltransferase 4A (beta- | | | | |
| | | galactosidasealpha-2,3-sialytransferase) | | | | |
| | | /DB_XREF = gi: 4506950 /UG = Hs.301698 | | | | |
| | | sialyltransferase 4A (beta-galactosidase | | | | |
| | | alpha-2,3-sialytransferase) | | | | |
| | | /FL = gb: L13972.1 gb: NM_003033.1 | | | | |
| | | gb: L29555.1 | | | | |
| 213226_at | exosome | Consensus includes gb: AI346350 | CCNA2 | −1.3 | −1.4 | −69.4 |
| | component 9 | /FEA = EST /DB_XREF = gi: 4083556 | | | | |
| | | /DB_XREF = est: qp50c06.x1 | | | | |
| | | /CLONE = IMAGE: 1926442 /UG = Hs.91728 | | | | |
| | | polymyositisscleroderma autoantigen 1 | | | | |
| | | (75 kD) | | | | |
| 205345_at | BRCA1 associated | gb: NM_000465.1 /DEF = Homo sapiens | BARD1 | 79.0 | −1.3 | −1.2 |
| | RING domain 1 | BRCA1 associated RING domain 1 | | | | |
| | | (BARD1), mRNA. /FEA = mRNA | | | | |
| | | /GEN = BARD1 /PROD = BRCA1 associated | | | | |
| | | RING domain 1 /DB_XREF = gi: 4557348 | | | | |
| | | /UG = Hs.54089 BRCA1 associated RING | | | | |
| | | domain 1 /FL = gb: U76638.1 | | | | |
| | | gb: NM_000465.1 | | | | |
| 203485_at | reticulon 1 | gb: NM_021136.1 /DEF = Homo sapiens | RTN1 | 1.3 | −1.3 | −1.7 |
| | | reticulon 1 (RTN1), mRNA. /FEA = mRNA | | | | |
| | | /GEN = RTN1 /PROD = reticulon 1 | | | | |
| | | /DB_XREF = gi: 10863934 /UG = Hs.99947 | | | | |
| | | reticulon 1 /FL = gb: NM_021136.1 | | | | |
| | | gb: L10333.1 gb: L10334.1 | | | | |
| 211336_x_at | leukocyte | gb: AF009007.1 /DEF = Homo sapiens | LILRB1 | −1.1 | −1.2 | 1.0 |
| | immunoglobulin- | immunoglobulin-like transcript 2c mRNA, | | | | |
| | like receptor, | complete cds. /FEA = mRNA | | | | |
| | subfamily B (with | /PROD = immunoglobulin-like transcript 2c | | | | |
| | TM and ITIM | /DB_XREF = gi: 2660705 /UG = Hs.204040 | | | | |
| | domains), member 1 | leukocyte immunoglobulin-like receptor, | | | | |
| | | subfamily B (with TM and ITIM domains), | | | | |
| | | member 1 /FL = gb: AF009007.1 | | | | |
| 206978_at | Transcribed | gb: NM_000647.2 /DEF = Homo sapiens | CCR2 | 1.2 | −1.4 | −1.6 |
| | sequences | chemokine (C-C motif) receptor 2 (CCR2), | | | | |
| | | mRNA. /FEA = mRNA /GEN = CCR2 | | | | |
| | | /PROD = chemokine (C-C motif) receptor 2 | | | | |
| | | /DB_XREF = gi: 4827072 /UG = Hs.395 | | | | |
| | | chemokine (C-C motif) receptor 2 | | | | |
| | | /FL = gb: U03882.1 gb: NM_000647.2 | | | | |
| 200853_at | H2A histone family, | gb: NM_002106.1 /DEF = Homo sapiens | | 1.3 | −1.3 | −1.3 |
| | member Z | H2A histone family, member Z (H2AFZ), | | | | |
| | | mRNA. /FEA = mRNA /GEN = H2AFZ | | | | |
| | | /PROD = H2A histone family, member Z | | | | |
| | | /DB_XREF = gi: 4504254 /UG = Hs.119192 | | | | |
| | | H2A histone family, member Z | | | | |
| | | /FL = gb: M37583.1 gb: NM_002106.1 | | | | |
| 204558_at | RAD54-like (S. cerevisiae) | gb: NM_003579.1 /DEF = Homo sapiens | RAD54L | −1.0 | −1.0 | −1.0 |
| | | RAD54 (S. cerevisiae)-like (RAD54L), | | | | |
| | | mRNA. /FEA = mRNA /GEN = RAD54L | | | | |
| | | /PROD = RAD54 (S. cerevisiae)-like | | | | |
| | | /DB_XREF = gi: 4506396 /UG = Hs.66718 | | | | |
| | | RAD54 (S. cerevisiae)-like | | | | |
| | | /FL = gb: NM_003579.1 | | | | |
| 218662_s_at | chromosome | gb: NM_022346.1 /DEF = Homo sapiens | HCAP-G | −1.0 | −1.0 | −51.5 |
| | condensation | chromosome condensation protein G | | | | |
| | protein G | (HCAP-G), mRNA. /FEA = mRNA | | | | |
| | | /GEN = HCAP-G /PROD = chromosome | | | | |
| | | condensation protein G | | | | |
| | | /DB_XREF = gi: 11641252 /UG = Hs.193602 | | | | |
| | | chromosome condensation protein G | | | | |
| | | /FL = gb: AF235023.1 gb: NM_022346.1 | | | | |
| | | gb: AF331796.1 gb: BC000827.1 | | | | |
| | | gb: AB013299.1 | | | | |
| 209531_at | glutathione | gb: BC001453.1 /DEF = Homo sapiens, | GSTZ1 | −109.4 | −1.2 | 1.1 |
| | transferase zeta 1 | glutathione transferase zeta 1 | | | | |
| | (maleylacetoacetate | (maleylacetoacetate isomerase), clone | | | | |
| | isomerase) | MGC: 2029, mRNA, complete cds. | | | | |
| | | /FEA = mRNA /PROD = glutathione | | | | |
| | | transferase zeta 1(maleylacetoacetate | | | | |
| | | isomerase) /DB_XREF = gi: 12655190 | | | | |
| | | /UG = Hs.26403 glutathione transferase | | | | |
| | | zeta 1 (maleylacetoacetate isomerase) | | | | |
| | | /FL = gb: BC001453.1 gb: U86529.1 | | | | |
| | | gb: NM_001513.1 | | | | |
| 211519_s_at | kinesin family | gb: AY026505.1 /DEF = Homo sapiens | KIF2C | −1.4 | −1.1 | 1.1 |
| | member 2C | testis specific mitotic centromere- | | | | |
| | | associated kinesin mRNA, complete cds. | | | | |
| | | /FEA = mRNA /PROD = testis specific mitotic | | | | |
| | | centromere-associatedkinesin | | | | |
| | | /DB_XREF = gi: 13432054 /UG = Hs.69360 | | | | |
| | | kinesin-like 6 (mitotic centromere- | | | | |
| | | associated kinesin) /FL = gb: AY026505.1 | | | | |
| 202954_at | ubiquitin- | gb: NM_007019.1 /DEF = Homo sapiens | UBE2C | 1.1 | 1.0 | −1.1 |
| | conjugating | ubiquitin carrier protein E2-C (UBCH10), | | | | |
| | enzyme E2C | mRNA. /FEA = mRNA /GEN = UBCH10 | | | | |
| | | /PROD = ubiquitin carrier protein E2-C | | | | |
| | | /DB_XREF = gi: 5902145 /UG = Hs.93002 | | | | |
| | | ubiquitin carrier protein E2-C | | | | |
| | | /FL = gb: U73379.1 gb: NM_007019.1 | | | | |
| 203232_s_at | spinocerebellar | gb: NM_000332.1 /DEF = Homo sapiens | ATXN1 | 1.7 | 1.2 | −1.4 |
| | ataxia 1 | spinocerebellar ataxia 1 | | | | |
| | (olivopontocerebellar | (olivopontocerebellar ataxia 1, autosomal | | | | |
| | ataxia 1, | dominant, ataxin 1) (SCA1), mRNA. | | | | |
| | autosomal | /FEA = mRNA /GEN = SCA1 /PROD = ataxin | | | | |
| | dominant, ataxin 1) | 1 /DB_XREF = gi: 4506792 /UG = Hs.74520 | | | | |
| | | spinocerebellar ataxia 1 | | | | |
| | | (olivopontocerebellar ataxia 1, autosomal | | | | |
| | | dominant, ataxin 1) /FL = gb: NM_000332.1 | | | | |
| 204798_at | v-myb | gb: NM_005375.1 /DEF = Homo sapiens v- | MYB | 1.4 | 1.1 | −1.4 |
| | myeloblastosis | myb avian myeloblastosis viral oncogene | | | | |
| | viral oncogene | homolog (MYB), mRNA. /FEA = mRNA | | | | |
| | homolog (avian) | /GEN = MYB /PROD = v-myb avian | | | | |
| | | myeloblastosis viral oncogenehomolog | | | | |
| | | /DB_XREF = gi: 4885496 /UG = Hs.1334 v- | | | | |
| | | myb avian myeloblastosis viral oncogene | | | | |
| | | homolog /FL = gb: M15024.1 | | | | |
| | | gb: AF104863.1 gb: NM_005375.1 | | | | |
| 203887_s_at | thrombomodulin | gb: NM_000361.1 /DEF = Homo sapiens | THBD | −1.2 | 1.1 | 52.6 |
| | | thrombomodulin (THBD), mRNA. | | | | |
| | | /FEA = mRNA /GEN = THBD | | | | |
| | | /PROD = thrombomodulin | | | | |
| | | /DB_XREF = gi: 4507482 /UG = Hs.2030 | | | | |
| | | thrombomodulin /FL = gb: M16552.1 | | | | |
| | | gb: NM_000361.1 | | | | |
| 204962_s_at | centromere protein | gb: NM_001809.2 /DEF = Homo sapiens | CENPA | −1.0 | −1.0 | −52.3 |
| | A, 17 kDa | centromere protein A (17 kD) (CENPA), | | | | |
| | | mRNA. /FEA = mRNA /GEN = CENPA | | | | |
| | | /PROD = centromere protein A | | | | |
| | | /DB_XREF = gi: 4585861 /UG = Hs.1594 | | | | |
| | | centromere protein A (17 kD) | | | | |
| | | /FL = gb: NM_001809.2 gb: U14518.1 | | | | |
| 203923_s_at | cytochrome b-245, | gb: NM_000397.2 /DEF = Homo sapiens | CYBB | 1.1 | −1.2 | −1.3 |
| | beta polypeptide | cytochrome b-245, beta polypeptide | | | | |
| | (chronic | (chronic granulomatous disease) (CYBB), | | | | |
| | granulomatous | mRNA. /FEA = mRNA /GEN = CYBB | | | | |
| | disease) | /PROD = cytochrome b-245, beta | | | | |
| | | polypeptide (chronicgranulomatous | | | | |
| | | disease) /DB_XREF = gi: 6996020 | | | | |
| | | /UG = Hs.88974 cytochrome b-245, beta | | | | |
| | | polypeptide (chronic granulomatous | | | | |
| | | disease) /FL = gb: NM_000397.2 | | | | |
| 201809_s_at | endoglin (Osler- | gb: NM_000118.1 /DEF = Homo sapiens | ENG | 1.2 | −1.4 | 1.2 |
| | Rendu-Weber | endoglin (Osler-Rendu-Weber syndrome | | | | |
| | syndrome 1) | 1) (ENG), mRNA. /FEA = mRNA | | | | |
| | | /GEN = ENG /PROD = endoglin precursor | | | | |
| | | /DB_XREF = gi: 4557554 /UG = Hs.76753 | | | | |
| | | endoglin (Osler-Rendu-Weber syndrome | | | | |
| | | 1) /FL = gb: NM_000118.1 | | | | |
| 214560_at | formyl peptide | Consensus includes gb: NM_002030.2 | FPRL2 | −1.3 | −1.2 | 1.0 |
| | receptor-like 2 | /DEF = Homo sapiens formyl peptide | | | | |
| | | receptor-like 2 (FPRL2), mRNA. | | | | |
| | | /FEA = CDS /GEN = FPRL2 /PROD = formyl | | | | |
| | | peptide receptor-like 2 | | | | |
| | | /DB_XREF = gi: 4758401 /UG = Hs.158314 | | | | |
| | | formyl peptide receptor-like 2 | | | | |
| | | /FL = gb: M76673.1 gb: NM_002030.2 | | | | |
| 218039_at | nucleolar and | gb: NM_016359.1 /DEF = Homo sapiens | NUSAP1 | 1.2 | −1.2 | −1.4 |
| | spindle associated | clone HQ0310 PRO0310p1 (LOC51203), | | | | |
| | protein 1 | mRNA. /FEA = mRNA /GEN = LOC51203 | | | | |
| | | /PROD = clone HQ0310 PRO0310p1 | | | | |
| | | /DB_XREF = gi: 7705950 /UG = Hs.279905 | | | | |
| | | clone HQ0310 PRO0310p1 | | | | |
| | | /FL = gb: AF305711.1 gb: BC001308.1 | | | | |
| | | gb: AF290612.1 gb: AF090915.1 | | | | |
| | | gb: NM_016359.1 | | | | |
| 213754_s_at | tripartite motif- | Consensus includes gb: AW613203 | PAIP1 | 69.5 | 1.0 | 1.2 |
| | containing 26 | /FEA = EST /DB_XREF = gi: 7318389 | | | | |
| | | /DB_XREF = est: hh67h11.x1 | | | | |
| | | /CLONE = IMAGE: 2967909 | | | | |
| | | /UG = Hs.109643 polyadenylate binding | | | | |
| | | protein-interacting protein 1 | | | | |
| 218264_at | BRCA2 and | gb: NM_016567.1 /DEF = Homo sapiens | BCCIP | 53.2 | 50.0 | −1.0 |
| | CDKN1A | cdk inhibitor p21 binding protein (TOK-1), | | | | |
| | interacting protein | mRNA. /FEA = mRNA /GEN = TOK-1 | | | | |
| | | /PROD = cdk inhibitor p21 binding protein | | | | |
| | | /DB_XREF = gi: 7706580 /UG = Hs.279862 | | | | |
| | | cdk inhibitor p21 binding protein | | | | |
| | | /FL = gb: AB040450.1 gb: NM_016567.1 | | | | |
| 219211_at | ubiquitin specific | gb: NM_017414.1 /DEF = Homo sapiens | USP18 | −1.0 | −183.7 | 1.0 |
| | protease 18 | ubiquitin specific protease 18 (USP18), | | | | |
| | | mRNA. /FEA = mRNA /GEN = USP18 | | | | |
| | | /PROD = ubiquitin specific protease 18 | | | | |
| | | /DB_XREF = gi: 8394518 /UG = Hs.38260 | | | | |
| | | ubiquitin specific protease 18 | | | | |
| | | /FL = gb: AL136690.1 gb: AF176642.2 | | | | |
| | | gb: NM_017414.1 | | | | |
| 204638_at | acid phosphatase | gb: NM_001611.2 /DEF = Homo sapiens | ACP5 | 1.0 | −1.3 | −1.1 |
| | 5, tartrate resistant | acid phosphatase 5, tartrate resistant | | | | |
| | | (ACP5), mRNA. /FEA = mRNA | | | | |
| | | /GEN = ACP5 /PROD = tartrate resistant | | | | |
| | | acid phosphatase 5 precursor | | | | |
| | | /DB_XREF = gi: 6138970 /UG = Hs.1211 acid | | | | |
| | | phosphatase 5, tartrate resistant | | | | |
| | | /FL = gb: J04430.1 gb: NM_001611.2 | | | | |
| 206461_x_at | metallothionein 1H | gb: NM_005951.1 /DEF = Homo sapiens | MT1H | 1.2 | −1.9 | −1.2 |
| | | metallothionein 1H (MT1H), mRNA. | | | | |
| | | /FEA = mRNA /GEN = MT1H | | | | |
| | | /PROD = metallothionein 1H | | | | |
| | | /DB_XREF = gi: 10835084 /UG = Hs.2667 | | | | |
| | | metallothionein 1H /FL = gb: NM_005951.1 | | | | |
| 204641_at | NIMA (never in | gb: NM_002497.1 /DEF = Homo sapiens | NEK2 | −1.0 | −1.0 | −1.0 |
| | mitosis gene a)- | NIMA (never in mitosis gene a)-related | | | | |
| | related kinase 2 | kinase 2 (NEK2), mRNA. /FEA = mRNA | | | | |
| | | /GEN = NEK2 /PROD = NIMA (never in | | | | |
| | | mitosis gene a)-related kinase 2 | | | | |
| | | /DB_XREF = gi: 4505372 /UG = Hs.153704 | | | | |
| | | NIMA (never in mitosis gene a)-related | | | | |
| | | kinase 2 /FL = gb: NM_002497.1 | | | | |
| | | gb: U11050.1 | | | | |
| 201041_s_at | dual specificity | gb: NM_004417.2 /DEF = Homo sapiens | DUSP1 | 1.4 | −1.1 | 1.0 |
| | phosphatase 1 | dual specificity phosphatase 1 (DUSP1), | | | | |
| | | mRNA. /FEA = mRNA /GEN = DUSP1 | | | | |
| | | /PROD = dual specificity phosphatase 1 | | | | |
| | | /DB_XREF = gi: 7108342 /UG = Hs.171695 | | | | |
| | | dual specificity phosphatase 1 | | | | |
| | | /FL = gb: NM_004417.2 | | | | |
| 204531_s_at | breast cancer 1, | gb: NM_007295.1 /DEF = Homo sapiens | BRCA1 | −1.0 | −1.0 | −1.0 |
| | early onset | breast cancer 1, early onset (BRCA1), | | | | |
| | | transcript variant BRCA1b, mRNA. | | | | |
| | | /FEA = mRNA /GEN = BRCA1 | | | | |
| | | /PROD = breast cancer 1, early onset | | | | |
| | | /DB_XREF = gi: 6552300 /UG = Hs.194143 | | | | |
| | | breast cancer 1, early onset | | | | |
| | | /FL = gb: NM_007295.1 | | | | |
| 202218_s_at | fatty acid | gb: NM_004265.1 /DEF = Homo sapiens | FADS2 | −1.4 | 1.1 | −2.0 |
| | desaturase 2 | delta-6 fatty acid desaturase (FADSD6), | | | | |
| | | mRNA. /FEA = mRNA /GEN = FADSD6 | | | | |
| | | /PROD = delta-6 fatty acid desaturase | | | | |
| | | /DB_XREF = gi: 4758333 /UG = Hs.184641 | | | | |
| | | fatty acid desaturase 2 | | | | |
| | | /FL = gb: AF084559.1 gb: AF126799.1 | | | | |
| | | gb: NM_004265.1 | | | | |
| 201292_at | topoisomerase | Consensus includes gb: AL561834 | TOP2A | −1.0 | −1.0 | −1.0 |
| | (DNA) II alpha | /FEA = EST /DB_XREF = gi: 12909658 | | | | |
| | 170 kDa | /DB_XREF = est: AL561834 | | | | |
| | | /CLONE = CS0DB005YC15 (3 prime) | | | | |
| | | /UG = Hs.156346 topoisomerase (DNA) II | | | | |
| | | alpha (170 kD) /FL = gb: J04088.1 | | | | |
| | | gb: NM_001067.1 | | | | |
| 212298_at | neuropilin 1 | Consensus includes gb: BE620457 | NRP1 | 1.6 | 1.4 | 1.2 |
| | | /FEA = EST /DB_XREF = gi: 9891395 | | | | |
| | | /DB_XREF = est: 601483690F1 | | | | |
| | | /CLONE = IMAGE: 3886055 /UG = Hs.69285 | | | | |
| | | neuropilin 1 /FL = gb: AF018956.1 gb: AF016050.1 gb: NM_003873.1 | | | | |
| 204249_s_at | LIM domain only 2 | gb: NM_005574.2 /DEF = Homo sapiens | LMO2 | 1.0 | −1.5 | −1.3 |
| | (rhombotin-like 1) | LIM domain only 2 (rhombotin-like 1) | | | | |
| | | (LMO2), mRNA. /FEA = mRNA | | | | |
| | | /GEN = LMO2 /PROD = LIM domain only 2 | | | | |
| | | /DB_XREF = gi: 6633806 /UG = Hs.184585 | | | | |
| | | LIM domain only 2 (rhombotin-like 1) | | | | |
| | | /FL = gb: NM_005574.2 | | | | |
| 203167_at | tissue inhibitor of | gb: NM_003255.2 /DEF = Homo sapiens | TIMP2 | 1.2 | −1.9 | 1.1 |
| | metalloproteinase 2 | tissue inhibitor of metalloproteinase 2 | | | | |
| | | (TIMP2), mRNA. /FEA = mRNA | | | | |
| | | /GEN = TIMP2 /PROD = tissue inhibitor of | | | | |
| | | metalloproteinase 2precursor | | | | |
| | | /DB_XREF = gi: 9257247 /UG = Hs.325495 | | | | |
| | | tissue inhibitor of metalloproteinase 2 | | | | |
| | | /FL = gb: M32304.1 gb: J05593.1 | | | | |
| | | gb: NM_003255.2 | | | | |
| 214637_at | oncostatin M | Consensus includes gb: BG437034 | OSM | −1.1 | −1.0 | −1.0 |
| | | /FEA = EST /DB_XREF = gi: 13343540 | | | | |
| | | /DB_XREF = est: 602488622F1 | | | | |
| | | /CLONE = IMAGE: 4620821 | | | | |
| | | /UG = Hs.248156 oncostatin M | | | | |
| | | /FL = gb: NM_020530.1 | | | | |
| 203535_at | S100 calcium | gb: NM_002965.2 /DEF = Homo sapiens | S100A9 | −1.1 | −1.4 | −1.5 |
| | binding protein A9 | S100 calcium-binding protein A9 | | | | |
| | (calgranulin B) | (calgranulin B) (S100A9), mRNA. | | | | |
| | | /FEA = mRNA /GEN = S100A9 /PROD = S100 | | | | |
| | | calcium-binding protein A9 | | | | |
| | | /DB_XREF = gi: 9845520 /UG = Hs.112405 | | | | |
| | | S100 calcium-binding protein A9 | | | | |
| | | (calgranulin B) /FL = gb: M26311.1 | | | | |
| | | gb: NM_002965.2 | | | | |
| 221463_at | chemokine (C-C | gb: NM_002991.1 /DEF = Homo sapiens | CCL24 | 2.4 | −1.0 | −56.9 |
| | motif) ligand 24 | small inducible cytokine subfamily A (Cys- | | | | |
| | | Cys), member 24 (SCYA24), mRNA. | | | | |
| | | /FEA = CDS /GEN = SCYA24 /PROD = small | | | | |
| | | inducible cytokine subfamily A (Cys- | | | | |
| | | Cys), member 24 /DB_XREF = gi: 4506838 | | | | |
| | | /UG = Hs.247838 small inducible cytokine | | | | |
| | | subfamily A (Cys-Cys), member 24 | | | | |
| | | /FL = gb: U85768.1 gb: NM_002991.1 | | | | |
| 204194_at | BTB and CNC | gb: NM_001186.1 /DEF = Homo sapiens | BACH1 | 1.5 | −1.3 | −1.3 |
| | homology 1, basic | BTB and CNC homology 1, basic leucine | | | | |
| | leucine zipper | zipper transcription factor 1 (BACH1), | | | | |
| | transcription factor 1 | mRNA. /FEA = mRNA /GEN = BACH1 | | | | |
| | | /PROD = BTB and CNC homology 1, basic | | | | |
| | | leucine zippertranscription factor 1 | | | | |
| | | /DB_XREF = gi: 4502352 /UG = Hs.154276 | | | | |
| | | BTB and CNC homology 1, basic leucine | | | | |
| | | zipper transcription factor 1 | | | | |
| | | /FL = gb: AB002803.1 gb: NM_001186.1 | | | | |
| 210784_x_at | leukocyte | gb: AF009634.1 /DEF = Homo sapiens | LILRB3 | 1.0 | −1.2 | −1.2 |
| | immunoglobulin- | clone 17.6 immunoglobulin-like transcript | | | | |
| | like receptor, | 5 protein mRNA, complete cds. | | | | |
| | subfamily B (with | /FEA = mRNA /PROD = immunoglobulin-like | | | | |
| | TM and ITIM | transcript 5 protein /DB_XREF = gi: 2662427 | | | | |
| | domains), member 3 | /UG = Hs.105928 leukocyte immunoglobulin | | | | |
| | | like receptor, subfamily B (with TM and | | | | |
| | | ITIM domains), member 3 | | | | |
| | | /FL = gb: AF009634.1 gb: AF009636.1 | | | | |
| | | gb: AF009640.1 | | | | |
| 201785_at | ribonuclease, | gb: NM_002933.1 /DEF = Homo sapiens | RNASE1 | 1.0 | 1.2 | 2.2 |
| | RNase A family, 1 | ribonuclease, RNase A family, 1 | | | | |
| | (pancreatic) | (pancreatic) (RNASE1), mRNA. | | | | |
| | | /FEA = mRNA /GEN = RNASE1 | | | | |
| | | /PROD = ribonuclease, RNase A family, 1 | | | | |
| | | (pancreatic) /DB_XREF = gi: 4506546 | | | | |
| | | /UG = Hs.78224 ribonuclease, RNase A | | | | |
| | | family, 1 (pancreatic) /FL = gb: BC005324.1 | | | | |
| | | gb: NM_002933.1 gb: D26129.1 | | | | |
| 214146_s_at | pro-platelet basic | Consensus includes gb: R64130 | PPBP | 1.5 | 1.1 | 1.6 |
| | protein (chemokine | /FEA = EST /DB_XREF = gi: 836009 | | | | |
| | (C—X—C motif) | /DB_XREF = est: yi18h03.s1 | | | | |
| | ligand 7) | /CLONE = IMAGE: 139637 /UG = Hs.2164 | | | | |
| | | pro-platelet basic protein (includes platelet | | | | |
| | | basic protein, beta-thromboglobulin, | | | | |
| | | connective tissue-activating peptide III, | | | | |
| | | neutrophil-activating peptide-2) | | | | |
| 206214_at | phospholipase A2, | gb: NM_005084.1 /DEF = Homo sapiens | PLA2G7 | 1.2 | −1.3 | 1.1 |
| | group VII (platelet- | phospholipase A2, group VII (platelet- | | | | |
| | activating factor | activating factor acetylhydrolase, plasma) | | | | |
| | acetylhydrolase, | (PLA2G7), mRNA. /FEA = mRNA | | | | |
| | plasma) | /GEN = PLA2G7 /PROD = phospholipase | | | | |
| | | A2, group VII (platelet-activatingfactor | | | | |
| | | acetylhydrolase, plasma) | | | | |
| | | /DB_XREF = gi: 4826883 /UG = Hs.93304 | | | | |
| | | phospholipase A2, group VII (platelet- | | | | |
| | | activating factor acetylhydrolase, plasma) | | | | |
| | | /FL = gb: U24577.1 gb: NM_005084.1 | | | | |
| | | gb: U20157.1 | | | | |
| 218355_at | kinesin family member | gb: NM_012310.2 /DEF = Homo | KIF4A | −1.0 | −1.0 | −1.0 |
| | | sapiens kinesin family member 4A | | | | |
| | | (KIF4A), mRNA. /FEA = mRNA | | | | |
| | | /GEN = KIF4A /PROD = kinesin | | | | |
| | | family member 4 | | | | |
| | | /DB_XREF = gi: 7305204 | | | | |
| | | /UG = Hs.279766 kinesin family | | | | |
| | | member 4A /FL = gb: AF179308.1 | | | | |
| | | gb: AF071592.2 gb: NM_012310.2 | | | | |
| 216950_s_at | Fc fragment of IgG, hi [symbol] | Consensus includes gb: X14355.1 | FCGR1A | 1.0 | −1.1 | −1.2 |
| | | /DEF = Human mRNA for high | | | | |
| | | affinity Fc receptor (FcRI) b form. | | | | |
| | | /FEA = mRNA /DB_XREF = gi: 31333 | | | | |
| | | /UG = Hs.77424 Fc fragment of | | | | |
| | | IgG, high affinity Ia, receptor for | | | | |
| | | (CD64) | | | | |
| 209464_at | aurora kinase B | gb: AB011446.1 /DEF = Homo | AURKB | −1.0 | −1.0 | −1.1 |
| | | sapiens mRNA for Aik2, complete | | | | |
| | | cds. /FEA = mRNA /GEN = aik2 | | | | |
| | | /PROD = Aik2 | | | | |
| | | /DB_XREF = gi: 5688865 | | | | |
| | | /UG = Hs.180655 serinethreonine | | | | |
| | | kinase 12 /FL = gb: BC000442.1 | | | | |
| | | gb: AF004022.1 gb: AF008552.1 | | | | |
| | | gb: AB011450.1 gb: AF015254.1 | | | | |
| | | gb: NM_004217.1 gb: AB011446.1 | | | | |
| 202191_s_at | growth arrest-specific | Consensus includes gb: BE439987 | GAS7 | 1.2 | −1.4 | −1.1 |
| | | /FEA = EST /DB_XREF = gi: 9439470 | | | | |
| | | /DB_XREF = est: HTM1-745F | | | | |
| | | /UG = Hs.226133 growth arrest- | | | | |
| | | specific 7 /FL = gb: AB007854.1 | | | | |
| | | gb: NM_005890.1 | | | | |
| 205769_at | solute carrier family 27 | gb: NM_003645.1 /DEF = Homo | SLC27A2 | −1.0 | −1.0 | −1.0 |
| | | sapiens fatty-acid-Coenzyme A | | | | |
| | | ligase, very long-chain 1 | | | | |
| | | (FACVL1), mRNA. /FEA = mRNA | | | | |
| | | /GEN = FACVL1 /PROD = very long- | | | | |
| | | chain fatty-acid-Coenzyme A | | | | |
| | | ligase 1 /DB_XREF = gi: 4503652 | | | | |
| | | /UG = Hs.11729 fatty-acid- | | | | |
| | | Coenzyme A ligase, very long- | | | | |
| | | chain 1 /FL = gb: D88308.1 | | | | |
| | | gb: AF096290.1 gb: NM_003645.1 | | | | |
| 209714_s_at | cyclin-dependent kinas | gb: AF213033.1 /DEF = Homo | CDKN3 | −1.3 | −1.0 | −1.2 |
| | | sapiens isolate BX-01 cyclin- | | | | |
| | | dependent kinase associated | | | | |
| | | protein phosphatase mRNA, | | | | |
| | | complete cds. /FEA = mRNA | | | | |
| | | /PROD = cyclin-dependent kinase | | | | |
| | | associated proteinphosphatase | | | | |
| | | /DB_XREF = gi: 12734643 | | | | |
| | | /UG = Hs.84113 cyclin-dependent | | | | |
| | | kinase inhibitor 3 (CDK2- | | | | |
| | | associated dual specificity | | | | |
| | | phosphatase) /FL = gb: AF213033.1 | | | | |
| | | gb: AF213034.1 gb: AF213035.1 | | | | |
| | | gb: AF213036.1 gb: AF213037.1 | | | | |
| | | gb: AF213038.1 gb: AF213039.1 | | | | |
| | | gb: AF213040.1 gb: AF213041.1 | | | | |
| | | gb: AF213042.1 gb: AF213044.1 | | | | |
| | | gb: AF213046.1 gb: AF213047.1 | | | | |
| | | gb: AF213048.1 gb: AF213049.1 | | | | |
| | | gb: AF213050.1 gb: AF213051.1 | | | | |
| | | gb: AF213052.1 gb: AF213053.1 | | | | |
| | | gb: U02681.1 gb: L25876.1 | | | | |
| | | gb: NM_005192.1 gb: L27711.1 | | | | |
| 203145_at | sperm associated anti [symbol] | gb: NM_006461.1 /DEF = Homo | SPAG5 | 1.1 | 1.3 | 94.7 |
| | | sapiens mitotic spindle coiled-coil | | | | |
| | | related protein (DEEPEST), | | | | |
| | | mRNA. /FEA = mRNA | | | | |
| | | /GEN = DEEPEST /PROD = mitotic | | | | |
| | | spindle coiled-coil related protein | | | | |
| | | /DB_XREF = gi: 5453631 | | | | |
| | | /UG = Hs.16244 mitotic spindle | | | | |
| | | coiled-coil related protein | | | | |
| | | /FL = gb: BC000322.1 | | | | |
| | | gb: AF063308.1 gb: NM_006461.1 | | | | |
| 205119_s_at | formyl peptide recepto | gb: NM_002029.1 /DEF = Homo | | 1.3 | −1.0 | −1.4 |
| | | sapiens formyl peptide receptor 1 | | | | |
| | | (FPR1), mRNA. /FEA = mRNA | | | | |
| | | /GEN = FPR1 /PROD = formyl | | | | |
| | | peptide receptor 1 | | | | |
| | | /DB_XREF = gi: 4503778 | | | | |
| | | /UG = Hs.753 formyl peptide | | | | |
| | | receptor 1 /FL = gb: BC005315.1 | | | | |
| | | gb: M60626.1 gb: M60627.1 | | | | |
| | | gb: NM_002029.1 | | | | |
| 204759_at | chromosome condens [symbol] | gb: NM_001268.1 /DEF = Homo | CHC1L | 1.6 | −1.0 | 1.2 |
| | | sapiens chromosome | | | | |
| | | condensation 1-like (CHC1L), | | | | |
| | | mRNA. /FEA = mRNA | | | | |
| | | /GEN = CHC1L /PROD = RCC1-like | | | | |
| | | G exchanging factor RLG | | | | |
| | | /DB_XREF = gi: 4557444 | | | | |
| | | /UG = Hs.27007 chromosome | | | | |
| | | condensation 1-like | | | | |
| | | /FL = gb: AF060219.1 | | | | |
| | | gb: NM_001268.1 | | | | |
| 201666_at | tissue inhibitor of meta | gb: NM_003254.1 /DEF = Homo | TIMP1 | 1.0 | −1.3 | 1.0 |
| | | sapiens tissue inhibitor of | | | | |
| | | metalloproteinase 1 (erythroid | | | | |
| | | potentiating activity, collagenase | | | | |
| | | inhibitor) (TIMP1), mRNA. | | | | |
| | | /FEA = mRNA /GEN = TIMP1 | | | | |
| | | /PROD = tissue inhibitor of | | | | |
| | | metalloproteinase 1precursor | | | | |
| | | /DB_XREF = gi: 4507508 | | | | |
| | | /UG = Hs.5831 tissue inhibitor of | | | | |
| | | metalloproteinase 1 (erythroid | | | | |
| | | potentiating activity, collagenase | | | | |
| | | inhibitor) /FL = gb: BC000866.1 | | | | |
| | | gb: M12670.1 gb: M59906.1 | | | | |
| | | gb: NM_003254.1 | | | | |
| 217996_at | pleckstrin homology-lik | Consensus includes gb: AA576961 | PHLDA1 | 1.2 | 63.7 | −66.0 |
| | | /FEA = EST /DB_XREF = gi: 2354435 | | | | |
| | | /DB_XREF = est: nm82d08.s1 | | | | |
| | | /CLONE = IMAGE: 1074735 | | | | |
| | | /UG = Hs.82101 pleckstrin | | | | |
| | | homology-like domain, family A, | | | | |
| | | member 1 /FL = gb: NM_007350.1 | | | | |
| 209709_s_at | hyaluronan-mediated r | gb: U29343.1 /DEF = Homo sapiens | HMMR | −71.2 | −1.0 | −74.3 |
| | | hyaluronan receptor (RHAMM) | | | | |
| | | mRNA, complete cds. | | | | |
| | | /FEA = mRNA /GEN = RHAMM | | | | |
| | | /PROD = hyaluronan receptor | | | | |
| | | /DB_XREF = gi: 2959555 | | | | |
| | | /UG = Hs. 72550 hyaluronan- | | | | |
| | | mediated motility receptor | | | | |
| | | (RHAMM) /FL = gb: U29343.1 | | | | |
| | | gb: AF032862.1 gb: NM_012484.1 | | | | |
| 202388_at | regulator of G-protein [symbol] | gb: NM_002923.1 /DEF = Homo | RGS2 | 1.4 | 1.2 | 1.3 |
| | | sapiens regulator of G-protein | | | | |
| | | signalling 2, 24 kD (RGS2), mRNA. | | | | |
| | | /FEA = mRNA /GEN = RGS2 | | | | |
| | | /PROD = regulator of G-protein | | | | |
| | | signalling 2, 24 kD | | | | |
| | | /DB_XREF = gi: 4506516 | | | | |
| | | /UG = Hs.78944 regulator of G- | | | | |
| | | protein signalling 2, 24 kD | | | | |
| | | /FL = gb: L13463.1 | | | | |
| | | gb: NM_002923.1 | | | | |
| 204092_s_at | serine/threonine kinas [symbol] | gb: NM_003600.1 /DEF = Homo | STK6 | −1.0 | −60.9 | 55.8 |
| | | sapiens serinethreonine kinase 15 | | | | |
| | | (STK15), mRNA. /FEA = mRNA | | | | |
| | | /GEN = STK15 | | | | |
| | | /PROD = serinethreonine kinase 15 | | | | |
| | | /DB_XREF = gi: 4507274 | | | | |
| | | /UG = Hs.48915 serinethreonine | | | | |
| | | kinase 15 /FL = gb: AF008551.1 | | | | |
| | | gb: AF011468.1 gb: NM_003600.1 | | | | |
| 204446_s_at | arachidonate 5-lipoxyg | gb: NM_000698.1 /DEF = Homo | ALOX5 | 1.2 | −1.3 | −1.3 |
| | | sapiens arachidonate 5- | | | | |
| | | lipoxygenase (ALOX5), mRNA. | | | | |
| | | /FEA = mRNA /GEN = ALOX5 | | | | |
| | | /PROD = arachidonate 5- | | | | |
| | | lipoxygenase | | | | |
| | | /DB_XREF = gi: 4502056 | | | | |
| | | /UG = Hs.89499 arachidonate 5- | | | | |
| | | lipoxygenase /FL = gb: J03600.1 | | | | |
| | | gb: J03571.1 gb: NM_000698.1 | | | | |
| 203126_at | inositol(myo)-1(or 4)- [symbol] | gb: NM_014214.1 /DEF = Homo | IMPA2 | −1.0 | −1.7 | 1.0 |
| | | sapiens inositol(myo)-1(or 4)- | | | | |
| | | monophosphatase 2 (IMPA2), | | | | |
| | | mRNA. /FEA = mRNA | | | | |
| | | /GEN = IMPA2 | | | | |
| | | /PROD = inositol(myo)-1(or 4)- | | | | |
| | | monophosphatase 2 | | | | |
| | | /DB_XREF = gi: 7657235 | | | | |
| | | /UG = Hs.5753 inositol(myo)-1(or 4)- | | | | |
| | | monophosphatase 2 | | | | |
| | | /FL = gb: AF200432.1 | | | | |
| | | gb: NM_014214.1 gb: AF014398.2 | | | | |
| 208581_x_at | metallothionein 1X | gb: NM_005952.1 /DEF = Homo | MT1X | 1.2 | −1.4 | −1.5 |
| | | sapiens metallothionein 1X | | | | |
| | | (MT1X), mRNA. /FEA = CDS | | | | |
| | | /GEN = MT1X | | | | |
| | | /PROD = metallothionein 1X | | | | |
| | | /DB_XREF = gi: 10835231 | | | | |
| | | /UG = Hs.278462 metallothionein | | | | |
| | | 1X /FL = gb: NM_005952.1 | | | | |
| 219890_at | C-type (calcium depen [symbol] | gb: NM_013252.1 /DEF = Homo | CLECSF5 | 1.5 | −1.0 | −1.2 |
| | | sapiens C-type (calcium | | | | |
| | | dependent, carbohydrate- | | | | |
| | | recognition domain) lectin, | | | | |
| | | superfamily member 5 | | | | |
| | | (CLECSF5), mRNA. /FEA = mRNA | | | | |
| | | /GEN = CLECSF5 /PROD = C-type | | | | |
| | | (calcium dependent, carbohydrate- | | | | |
| | | recognition domain) lectin, | | | | |
| | | superfamilymember 5 | | | | |
| | | /DB_XREF = gi: 10281668 | | | | |
| | | /UG = Hs.126355 C-type (calcium | | | | |
| | | dependent, carbohydrate- | | | | |
| | | recognition domain) lectin, | | | | |
| | | superfamily member 5 | | | | |
| | | /FL = gb: NM_013252.1 | | | | |
| | | gb: AF139768.1 | | | | |
| 209773_s_at | ribonucleotide reducta [symbol] | gb: BC001886.1 /DEF = Homo | RRM2 | −1.9 | −1.5 | −1.5 |
| | | sapiens, ribonucleotide reductase | | | | |
| | | M2 polypeptide, clone MGC: 1551, | | | | |
| | | mRNA, complete cds. | | | | |
| | | /FEA = mRNA | | | | |
| | | /PROD = ribonucleotide reductase | | | | |
| | | M2 polypeptide | | | | |
| | | /DB_XREF = gi: 12804874 | | | | |
| | | /UG = Hs.75319 ribonucleotide | | | | |
| | | reductase M2 polypeptide | | | | |
| | | /FL = gb: BC001886.1 | | | | |
| 202705_at | cyclin B2 | gb: NM_004701.2 /DEF = Homo | CCNB2 | −1.0 | −1.3 | 1.0 |
| | | sapiens cyclin B2 (CCNB2), | | | | |
| | | mRNA. /FEA = mRNA | | | | |
| | | /GEN = CCNB2 /PROD = cyclin B2 | | | | |
| | | /DB_XREF = gi: 10938017 | | | | |
| | | /UG = Hs.194698 cyclin B2 | | | | |
| | | /FL = gb: NM_004701.2 | | | | |
| | | gb: AF002822.1 gb: AB020981.1 | | | | |
| | | gb: AL080146.1 | | | | |
| 205098_at | chemokine (C-C motif) | Consensus includes gb: AI421071 | | −1.1 | −1.7 | −1.1 |
| | | /FEA = EST /DB_XREF = gi: 4267002 | | | | |
| | | /DB_XREF = est: tf24a02.x1 | | | | |
| | | /CLONE = IMAGE: 2097098 | | | | |
| | | /UG = Hs.301921 chemokine (C-C | | | | |
| | | motif) receptor 1 /FL = gb: L09230.1 | | | | |
| | | gb: L10918.1 gb: NM_001295.1 | | | | |
| 204438_at | mannose receptor, C t [symbol] | gb: NM_002438.1 /DEF = Homo | MRC1 | 1.1 | −1.5 | −1.6 |
| | | sapiens mannose receptor, C type | | | | |
| | | 1 (MRC1), mRNA. /FEA = mRNA | | | | |
| | | /GEN = MRC1 /PROD = mannose | | | | |
| | | receptor, C type 1 | | | | |
| | | /DB_XREF = gi: 4505244 | | | | |
| | | /UG = Hs.75182 mannose receptor, | | | | |
| | | C type 1 /FL = gb: J05550.1 | | | | |
| | | gb: NM_002438.1 | | | | |
| 201438_at | collagen, type VI, alpha [symbol] | gb: NM_004369.1 /DEF = Homo | COL6A3 | −1.0 | −1.0 | −1.0 |
| | | sapiens collagen, type VI, alpha 3 | | | | |
| | | (COL6A3), mRNA. /FEA = mRNA | | | | |
| | | /GEN = COL6A3 /PROD = collagen, | | | | |
| | | type VI, alpha 3 | | | | |
| | | /DB_XREF = gi: 4758027 | | | | |
| | | /UG = Hs.80988 collagen, type VI, | | | | |
| | | alpha 3 /FL = gb: NM_004369.1 | | | | |
| 207067_s_at | histidine decarboxylase [symbol] | gb: NM_002112.1 /DEF = Homo | HDC | −1.3 | −1.0 | −1.2 |
| | | sapiens histidine decarboxylase | | | | |
| | | (HDC), mRNA. /FEA = mRNA | | | | |
| | | /GEN = HDC /PROD = histidine | | | | |
| | | decarboxylase | | | | |
| | | /DB_XREF = gi: 4504364 | | | | |
| | | /UG = Hs.1481 histidine | | | | |
| | | decarboxylase /FL = gb: M60445.1 | | | | |
| | | gb: NM_002112.1 | | | | |
| 220088_at | G protein-coupled rece [symbol] | gb: NM_001736.1 /DEF = Homo | C5R1 | 1.2 | −1.1 | 1.1 |
| | | sapiens complement component 5 | | | | |
| | | receptor 1 (C5a ligand) (C5R1), | | | | |
| | | mRNA. /FEA = mRNA /GEN = C5R1 | | | | |
| | | /PROD = complement component 5 | | | | |
| | | receptor 1 (C5a ligand) | | | | |
| | | /DB_XREF = gi: 4502508 | | | | |
| | | /UG = Hs.2161 complement | | | | |
| | | component 5 receptor 1 (C5a | | | | |
| | | ligand) /FL = gb: M62505.1 | | | | |
| | | gb: NM_001736.1 | | | | |
| 204614_at | serine (or cysteine) pro [symbol] | gb: NM_002575.1 /DEF = Homo | SERPINB2 | 1.4 | 1.2 | 1.3 |
| | | sapiens serine (or cysteine) | | | | |
| | | proteinase inhibitor, clade B | | | | |
| | | (ovalbumin), member 2 | | | | |
| | | (SERPINB2), mRNA. | | | | |
| | | /FEA = mRNA /GEN = SERPINB2 | | | | |
| | | /PROD = serine (or cysteine) | | | | |
| | | proteinase inhibitor, cladeB | | | | |
| | | (ovalbumin), member 2 | | | | |
| | | /DB_XREF = gi: 4505594 | | | | |
| | | /UG = Hs.75716 serine (or | | | | |
| | | cysteine) proteinase inhibitor, | | | | |
| | | clade B (ovalbumin), member 2 | | | | |
| | | /FL = gb: J02685.1 gb: J03603.1 | | | | |
| | | gb: M18082.1 gb: NM_002575.1 | | | | |
| 204039_at | CCAAT/enhancer bind [symbol] | gb: NM_004364.1 /DEF = Homo | CEBPA | 1.3 | −1.1 | −1.1 |
| | | sapiens CCAATenhancer binding | | | | |
| | | protein (CEBP), alpha (CEBPA), | | | | |
| | | mRNA. /FEA = mRNA | | | | |
| | | /GEN = CEBPA | | | | |
| | | /PROD = CCAATenhancer binding | | | | |
| | | protein (CEBP), alpha | | | | |
| | | /DB_XREF = gi: 4757971 | | | | |
| | | /UG = Hs.76171 CCAATenhancer | | | | |
| | | binding protein (CEBP), alpha | | | | |
| | | /FL = gb: NM_004364.1 | | | | |
| 205786_s_at | integrin, alpha M (com [symbol] | gb: NM_000632.2 /DEF = Homo | ITGAM | 1.0 | −1.2 | −1.6 |
| | | sapiens integrin, alpha M | | | | |
| | | (complement component receptor | | | | |
| | | 3, alpha; also known as CD11b | | | | |
| | | (p170), macrophage antigen alpha | | | | |
| | | polypeptide) (ITGAM), mRNA. | | | | |
| | | /FEA = mRNA /GEN = ITGAM | | | | |
| | | /PROD = integrin alpha M precursor | | | | |
| | | /DB_XREF = gi: 6006013 | | | | |
| | | /UG = Hs.172631 integrin, alpha M | | | | |
| | | (complement component receptor | | | | |
| | | 3, alpha; also known as CD11b | | | | |
| | | (p170), macrophage antigen alpha | | | | |
| | | polypeptide) /FL = gb: J03925.1 | | | | |
| | | gb: NM_000632.2 | | | | |
| 201739_at | serum/glucocorticoid re [symbol] | gb: NM_005627.1 /DEF = Homo | SGK | 1.3 | 1.0 | 1.0 |
| | | sapiens serumglucocorticoid | | | | |
| | | regulated kinase (SGK), mRNA. | | | | |
| | | /FEA = mRNA /GEN = SGK | | | | |
| | | /PROD = serumglucocorticoid | | | | |
| | | regulated kinase | | | | |
| | | /DB_XREF = gi: 5032090 | | | | |
| | | /UG = Hs.296323 | | | | |
| | | serumglucocorticoid regulated | | | | |
| | | kinase /FL = gb: BC001263.1 | | | | |
| | | gb: NM_005627.1 gb: AF153609.1 | | | | |
| 209949_at | neutrophil cytosolic fac [symbol] | gb: BC001606.1 /DEF = Homo | NCF2 | 1.2 | −1.4 | −1.5 |
| | | sapiens, Similar to neutrophil | | | | |
| | | cytosolic factor 2 (65 kD, chronic | | | | |
| | | granulomatous disease, | | | | |
| | | autosomal 2), clone MGC: 2275, | | | | |
| | | mRNA, complete cds. | | | | |
| | | /FEA = mRNA /PROD = Similar to | | | | |
| | | neutrophil cytosolic factor 2 | | | | |
| | | (65 kD, chronic granulomatous | | | | |
| | | disease, autosomal 2) | | | | |
| | | /DB_XREF = gi: 12804408 | | | | |
| | | /UG = Hs.949 neutrophil cytosolic | | | | |
| | | factor 2 (65 kD, chronic | | | | |
| | | granulomatous disease, | | | | |
| | | autosomal 2) /FL = gb: BC001606.1 | | | | |
| | | gb: M32011.1 gb: NM_000433.1 | | | | |
| 221210_s_at | N-acetylneuraminate p [symbol] | gb: NM_030769.1 /DEF = Homo | NPL | 1.5 | −1.1 | 1.1 |
| | | sapiens hypothetical protein | | | | |
| | | similar to swine acylneuraminate | | | | |
| | | lyase (C1ORF13), mRNA. | | | | |
| | | /FEA = mRNA /GEN = C1ORF13 | | | | |
| | | /PROD = hypothetical protein | | | | |
| | | similar to swineacylneuraminate | | | | |
| | | lyase /DB_XREF = gi: 13540532 | | | | |
| | | /FL = gb: NM_030769.1 | | | | |
| 203554_x_at | pituitary tumor-transfor [symbol] | gb: NM_004219.2 /DEF = Homo | PTTG1 | −1.5 | −1.2 | −1.2 |
| | | sapiens pituitary tumor- | | | | |
| | | transforming 1 (PTTG1), mRNA. | | | | |
| | | /FEA = mRNA /GEN = PTTG1 | | | | |
| | | /PROD = pituitary tumor- | | | | |
| | | transforming protein 1 | | | | |
| | | /DB_XREF = gi: 11038651 | | | | |
| | | /UG = Hs.252587 pituitary tumor- | | | | |
| | | transforming 1 | | | | |
| | | /FL = gb: NM_004219.2 | | | | |
| | | gb: AF095287.1 gb: AF062649.1 | | | | |
| | | gb: AF075242.1 | | | | |
| 210184_at | integrin, alpha X (antig [symbol] | gb: M81695.1 /DEF = H. sapiens | ITGAX | 1.1 | −1.0 | −1.2 |
| | | leukocyte adhesion glycoprotein | | | | |
| | | p150, 95 mRNA, complete cds. | | | | |
| | | /FEA = mRNA /GEN = ITGAX | | | | |
| | | /DB_XREF = gi: 487829 | | | | |
| | | /UG = Hs.51077 integrin, alpha X | | | | |
| | | (antigen CD11C (p150), alpha | | | | |
| | | polypeptide) /FL = gb: M81695.1 | | | | |
| | | gb: NM_000887.2 | | | | |
| 208079_s_at | serine/threonine kinase [symbol] | gb: NM_003158.1 /DEF = Homo | STK6 | −1.0 | −1.0 | 1.1 |
| | | sapiens serinethreonine kinase 6 | | | | |
| | | (STK6), mRNA. /FEA = mRNA | | | | |
| | | /GEN = STK6 | | | | |
| | | /PROD = serinethreonine kinase 6 | | | | |
| | | /DB_XREF = gi: 4507278 | | | | |
| | | /UG = Hs.250822 serinethreonine | | | | |
| | | kinase 6 /FL = gb: D84212.1 | | | | |
| | | gb: NM_003158.1 | | | | |
| 202240_at | polo-like kinase 1 (Dro [symbol] | gb: NM_005030.1 /DEF = Homo | PLK1 | −78.4 | 61.4 | −1.8 |
| | | sapiens polo (Drosophia)-like | | | | |
| | | kinase (PLK), mRNA. | | | | |
| | | /FEA = mRNA /GEN = PLK | | | | |
| | | /PROD = polo (Drosophia)-like | | | | |
| | | kinase /DB_XREF = gi: 4826915 | | | | |
| | | /UG = Hs.77597 polo (Drosophia)- | | | | |
| | | like kinase /FL = gb: BC002369.1 | | | | |
| | | gb: BC003002.1 gb: U01038.1 | | | | |
| | | gb: L19559.1 gb: NM_005030.1 | | | | |
| 217762_s_at | RAB31, member RAS | Consensus includes gb: BE789881 | RAB31 | 1.3 | −1.3 | −1.1 |
| | | /FEA = EST | | | | |
| | | /DB_XREF = gi: 10211079 | | | | |
| | | /DB_XREF = est: 601476750F1 | | | | |
| | | /CLONE = IMAGE: 3879493 | | | | |
| | | /UG = Hs.223025 RAB31, member | | | | |
| | | RAS oncogene family | | | | |
| | | /FL = gb: AF234995.1 | | | | |
| | | gb: BC001148.1 gb: U59877.1 | | | | |
| | | gb: U57091.1 gb: NM_006868.1 | | | | |
| | | gb: AF183421.1 | | | | |
| 215925_s_at | CD72 antigen | Consensus includes | CD72 | 69.7 | −1.4 | 1.4 |
| | | gb: AF283777.2 /DEF = Homo | | | | |
| | | sapiens clone TCBAP0702 mRNA | | | | |
| | | sequence. /FEA = mRNA | | | | |
| | | /DB_XREF = gi: 10281735 | | | | |
| | | /UG = Hs.116481 CD72 antigen | | | | |
| 201422_at | interferon, gamma-ind [symbol] | gb: NM_006332.1 /DEF = Homo | IFI30 | −1.4 | −1.1 | −1.1 |
| | | sapiens interferon, gamma- | | | | |
| | | inducible protein 30 (IFI30), | | | | |
| | | mRNA. /FEA = mRNA /GEN = IFI30 | | | | |
| | | /PROD = interferon, gamma- | | | | |
| | | inducible protein 30 | | | | |
| | | /DB_XREF = gi: 5453695 | | | | |
| | | /UG = Hs.14623 interferon, gamma- | | | | |
| | | inducible protein 30 | | | | |
| | | /FL = gb: J03909.1 gb: NM_006332.1 | | | | |
| | | gb: AF097362.1 | | | | |
| 218559_s_at | v-maf musculoaponeu [symbol] | gb: NM_005461.1 /DEF = Homo | MAFB | 1.1 | −1.5 | 1.1 |
| | | sapiens Kreisler (mouse) maf- | | | | |
| | | related leucine zipper homolog | | | | |
| | | (KRML), mRNA. /FEA = mRNA | | | | |
| | | /GEN = KRML /PROD = Kreisler | | | | |
| | | (mouse) maf-related leucine | | | | |
| | | zipperhomolog | | | | |
| | | /DB_XREF = gi: 4885446 | | | | |
| | | /UG = Hs.169487 Kreisler (mouse) | | | | |
| | | maf-related leucine zipper | | | | |
| | | homolog /FL = gb: AF134157.1 | | | | |
| | | gb: NM_005461.1 | | | | |
| 202241_at | tribbles homolog 1 (Dr [symbol] | gb: NM_025195.1 /DEF = Homo | TRIB1 | 1.4 | −1.3 | 1.1 |
| | | sapiens phosphoprotein regulated | | | | |
| | | by mitogenic pathways (C8FW), | | | | |
| | | mRNA. /FEA = mRNA | | | | |
| | | /GEN = C8FW /PROD = G-protein- | | | | |
| | | coupled receptor induced protein | | | | |
| | | /DB_XREF = gi: 13399327 | | | | |
| | | /UG = Hs.7837 phosphoprotein | | | | |
| | | regulated by mitogenic pathways | | | | |
| | | /FL = gb: AF205437.1 | | | | |
| | | gb: NM_025195.1 | | | | |
| 210004_at | oxidised low density lip [symbol] | gb: AF035776.1 /DEF = Homo | OLR1 | 1.2 | −1.6 | −2.0 |
| | | sapiens oxidized low-density | | | | |
| | | lipoprotein receptor mRNA, | | | | |
| | | complete cds. /FEA = mRNA | | | | |
| | | /PROD = oxidized low-density | | | | |
| | | lipoprotein receptor | | | | |
| | | /DB_XREF = gi: 3941299 | | | | |
| | | /UG = Hs.77729 oxidised low | | | | |
| | | density lipoprotein (lectin-like) | | | | |
| | | receptor 1 /FL = gb: AB010710.1 | | | | |
| | | gb: AF035776.1 gb: NM_002543.1 | | | | |
| 203755_at | BUB1 budding uninhib [symbol] | gb: NM_001211.2 /DEF = Homo | BUB1B | −1.1 | −1.3 | −1.1 |
| | | sapiens budding uninhibited by | | | | |
| | | benzimidazoles 1 (yeast | | | | |
| | | homolog), beta (BUB1B), mRNA. | | | | |
| | | /FEA = mRNA /GEN = BUB1B | | | | |
| | | /PROD = budding uninhibited by | | | | |
| | | benzimidazoles 1 (yeasthomolog), | | | | |
| | | beta /DB_XREF = gi: 5729749 | | | | |
| | | /UG = Hs.36708 budding | | | | |
| | | uninhibited by benzimidazoles 1 | | | | |
| | | (yeast homolog), beta | | | | |
| | | /FL = gb: AF053306.1 | | | | |
| | | gb: AF035933.1 gb: AF068760.1 | | | | |
| | | gb: AF046918.1 gb: AF107297.1 | | | | |
| | | gb: AF046079.2 gb: NM_001211.2 | | | | |
| 204072_s_at | hypothetical protein C [symbol] | gb: NM_023037.1 /DEF = Homo | 13CDNA73 | 1.3 | 1.2 | −1.5 |
| | | sapiens putative gene product | | | | |
| | | (13CDNA73), mRNA. | | | | |
| | | /FEA = mRNA /GEN = 13CDNA73 | | | | |
| | | /PROD = putative gene product | | | | |
| | | /DB_XREF = gi: 12957487 | | | | |
| | | /UG = Hs.181304 putative gene | | | | |
| | | product /FL = gb: NM_023037.1 | | | | |
| 202870_s_at | CDC20 cell division cy [symbol] | gb: NM_001255.1 /DEF = Homo | CDC20 | −1.0 | −1.0 | −63.0 |
| | | sapiens CDC20 (cell division cycle | | | | |
| | | 20, S. cerevisiae, homolog) | | | | |
| | | (CDC20), mRNA. /FEA = mRNA | | | | |
| | | /GEN = CDC20 /PROD = cell | | | | |
| | | division cycle 20 | | | | |
| | | /DB_XREF = gi: 4557436 | | | | |
| | | /UG = Hs.82906 CDC20 (cell | | | | |
| | | division cycle 20, S. cerevisiae, | | | | |
| | | homolog) /FL = gb: BC001088.1 | | | | |
| | | gb: AF099644.1 gb: NM_001255.1 | | | | |
| | | gb: U05340.1 | | | | |
| 220832_at | toll-like receptor 8 | gb: NM_016610.1 /DEF = Homo | TLR8 | 1.4 | −2.0 | −95.0 |
| | | sapiens Toll-like receptor 8 | | | | |
| | | (LOC51311), mRNA. /FEA = mRNA | | | | |
| | | /GEN = LOC51311 /PROD = Toll-like | | | | |
| | | receptor 8 /DB_XREF = gi: 7706147 | | | | |
| | | /UG = Hs.272410 Toll-like receptor | | | | |
| | | 8 /FL = gb: AF246971.1 | | | | |
| | | gb: NM_016610.1 gb: AF245703.1 | | | | |
| 211519_s_at | kinesin family member | gb: AY026505.1 /DEF = Homo | KIF2C | −1.4 | −1.1 | −1.1 |
| | | sapiens testis specific mitotic | | | | |
| | | centromere-associated kinesin | | | | |
| | | mRNA, complete cds. | | | | |
| | | /FEA = mRNA /PROD = testis | | | | |
| | | specific mitotic centromere- | | | | |
| | | associatedkinesin | | | | |
| | | /DB_XREF = gi: 13432054 | | | | |
| | | /UG = Hs.69360 kinesin-like 6 | | | | |
| | | (mitotic centromere-associated | | | | |
| | | kinesin) /FL = gb: AY026505.1 | | | | |
| 202954_at | ubiquitin-conjugating e [symbol] | gb: NM_007019.1 /DEF = Homo | UBE2C | −1.1 | 1.0 | −1.1 |
| | | sapiens ubiquitin carrier protein E2 | | | | |
| | | C (UBCH10), mRNA. /FEA = mRNA | | | | |
| | | /GEN = UBCH10 /PROD = ubiquitin | | | | |
| | | carrier protein E2-C | | | | |
| | | /DB_XREF = gi: 5902145 | | | | |
| | | /UG = Hs.93002 ubiquitin carrier | | | | |
| | | protein E2-C /FL = gb: U73379.1 | | | | |
| | | gb: NM_007019.1 | | | | |
| 204798_at | v-myb myeloblastosis [symbol] | gb: NM_005375.1 /DEF = Homo | MYB | 1.4 | −1.1 | −1.4 |
| | | sapiens v-myb avian | | | | |
| | | myeloblastosis viral oncogene | | | | |
| | | homolog (MYB), mRNA. | | | | |
| | | /FEA = mRNA /GEN = MYB | | | | |
| | | /PROD = v-myb avian | | | | |
| | | myeloblastosis viral | | | | |
| | | oncogenehomolog | | | | |
| | | /DB_XREF = gi: 4885496 | | | | |
| | | /UG = Hs.1334 v-myb avian | | | | |
| | | myeloblastosis viral oncogene | | | | |
| | | homolog /FL = gb: M15024.1 | | | | |
| | | gb: AF104863.1 gb: NM_005375.1 | | | | |
| 204962_s_at | centromere protein A, | gb: NM_001809.2 /DEF = Homo | CENPA | −1.0 | −1.0 | −52.3 |
| | | sapiens centromere protein A | | | | |
| | | (17 kD) (CENPA), mRNA. | | | | |
| | | /FEA = mRNA /GEN = CENPA | | | | |
| | | /PROD = centromere protein A | | | | |
| | | /DB_XREF = gi: 4585861 | | | | |
| | | /UG = Hs.1594 centromere protein | | | | |
| | | A (17 kD) /FL = gb: NM_001809.2 | | | | |
| | | gb: U14518.1 | | | | |
| 203923_s_at | cytochrome b-245, bet [symbol] | gb: NM_000397.2 /DEF = Homo | CYBB | 1.1 | −1.2 | −1.3 |
| | | sapiens cytochrome b-245, beta | | | | |
| | | polypeptide (chronic | | | | |
| | | granulomatous disease) (CYBB), | | | | |
| | | mRNA. /FEA = mRNA | | | | |
| | | /GEN = CYBB /PROD = cytochrome | | | | |
| | | b-245, beta polypeptide | | | | |
| | | (chronicgranulomatous disease) | | | | |
| | | /DB_XREF = gi: 6996020 | | | | |
| | | /UG = Hs.88974 cytochrome b-245, | | | | |
| | | beta polypeptide (chronic | | | | |
| | | granulomatous disease) | | | | |
| | | /FL = gb: NM_000397.2 | | | | |
| 218039_at | nucleolar and spindle [symbol] | gb: NM_016359.1 /DEF = Homo | NUSAP1 | 1.2 | −1.2 | −1.4 |
| | | sapiens clone HQ0310 | | | | |
| | | PRO0310p1 (LOC51203), mRNA. | | | | |
| | | /FEA = mRNA /GEN = LOC51203 | | | | |
| | | /PROD = clone HQ0310 | | | | |
| | | PRO0310p1 | | | | |
| | | /DB_XREF = gi: 7705950 | | | | |
| | | /UG = Hs.279905 clone HQ0310 | | | | |
| | | PRO0310p1 /FL = gb: AF305711.1 | | | | |
| | | gb: BC001308.1 gb: AF290612.1 | | | | |
| | | gb: AF090915.1 gb: NM_016359.1 | | | | |
| 206461_x_at | metallothionein 1H | gb: NM_005951.1 /DEF = Homo | MT1H | 1.2 | −1.9 | −1.2 |
| | | sapiens metallothionein 1H | | | | |
| | | (MT1H), mRNA. /FEA = mRNA | | | | |
| | | /GEN = MT1H | | | | |
| | | /PROD = metallothionein 1H | | | | |
| | | /DB_XREF = gi: 10835084 | | | | |
| | | /UG = Hs.2667 metallothionein 1H | | | | |
| | | /FL = gb: NM_005951.1 | | | | |
| 203535_at | S100 calcium binding [symbol] | gb: NM_002965.2 /DEF = Homo | S100A9 | −1.1 | −1.4 | −1.5 |
| | | sapiens S100 calcium-binding | | | | |
| | | protein A9 (calgranulin B) | | | | |
| | | (S100A9), mRNA. /FEA = mRNA | | | | |
| | | /GEN = S100A9 /PROD = S100 calcium-binding protein A9 | | | | |
| | | /DB_XREF = gi: 9845520 | | | | |
| | | /UG = Hs.112405 S100 calcium- | | | | |
| | | binding protein A9 (calgranulin B) | | | | |
| | | /FL = gb: M26311.1 | | | | |
| | | gb: NM_002965.2 | | | | |
| 218355_at | kinesin family | gb: NM_012310.2 /DEF = Homo sapiens kinesin | KIF4A | −1.0 | −1.0 | −1.0 |
| | member 4A | family member 4A (KIF4A), mRNA. /FEA = mRNA | | | | |
| | | /GEN = KIF4A /PROD = kinesin family member 4 | | | | |
| | | /DB_XREF = gi: 7305204 /UG = Hs.279766 kinesin | | | | |
| | | family member 4A /FL = gb: AF179308.1 | | | | |
| | | gb: AF071592.2 gb: NM_012310.2 | | | | |
| 216950_s_at | Fc fragment of | Consensus includes gb: X14355.1 /DEF = Human | FCGR1A | 1.0 | −1.1 | −1.2 |
| | IgG, high affinity | mRNA for high affinity Fc receptor (FcRI) b form. | | | | |
| | Ia, receptor for | /FEA = mRNA /DB_XREF = gi: 31333 | | | | |
| | (CD64) | /UG = Hs.77424 Fc fragment of IgG, high affinity | | | | |
| | | Ia, receptor for (CD64) | | | | |
| 202191_s_at | growth arrest- | Consensus includes gb: BE439987 /FEA = EST | GAS7 | 1.2 | −1.4 | −1.1 |
| | specific 7 | /DB_XREF = gi: 9439470 /DB_XREF = est: HTM1- | | | | |
| | | 745F /UG = Hs.226133 growth arrest-specific 7 | | | | |
| | | /FL = gb: AB007854.1 gb: NM_005890.1 | | | | |
| 205769_at | solute carrier | gb: NM_003645.1 /DEF = Homo sapiens fatty-acid- | SLC27A2 | −1.0 | −1.0 | −1.0 |
| | family 27 (fatty | Coenzyme A ligase, very long-chain 1 (FACVL1), | | | | |
| | acid transporter), | mRNA. /FEA = mRNA /GEN = FACVL1 | | | | |
| | member 2 | /PROD = very long-chain fatty-acid-Coenzyme A | | | | |
| | | ligase 1 /DB_XREF = gi: 4503652 /UG = Hs.11729 | | | | |
| | | fatty-acid-Coenzyme A ligase, very long-chain 1 | | | | |
| | | /FL = gb: D88308.1 gb: AF096290.1 | | | | |
| | | gb: NM_003645.1 | | | | |
| 209714_s_at | cyclin-dependent | gb: AF213033.1 /DEF = Homo sapiens isolate BX- | CDKN3 | −1.3 | −1.0 | −1.2 |
| | kinase inhibitor 3 | 01 cyclin-dependent kinase associated protein | | | | |
| | (CDK2- | phosphatase mRNA, complete cds. | | | | |
| | associated dual | /FEA = mRNA /PROD = cyclin-dependent kinase | | | | |
| | specificity | associated proteinphosphatase | | | | |
| | phosphatase) | /DB_XREF = gi: 12734643 /UG = Hs.84113 cyclin- | | | | |
| | | dependent kinase inhibitor 3 (CDK2-associated | | | | |
| | | dual specificity phosphatase) | | | | |
| | | /FL = gb: AF213033.1 gb: AF213034.1 | | | | |
| | | gb: AF213035.1 gb: AF213036.1 gb: AF213037.1 | | | | |
| | | gb: AF213038.1 gb: AF213039.1 gb: AF213040.1 | | | | |
| | | gb: AF213041.1 gb: AF213042.1 gb: AF213044.1 | | | | |
| | | gb: AF213046.1 gb: AF213047.1 gb: AF213048.1 | | | | |
| | | gb: AF213049.1 gb: AF213050.1 gb: AF213051.1 | | | | |
| | | gb: AF213052.1 gb: AF213053.1 gb: U02681.1 | | | | |
| | | gb: L25876.1 gb: NM_005192.1 gb: L27711.1 | | | | |
| 203145_at | sperm associated | gb: NM_006461.1 /DEF = Homo sapiens mitotic | SPAG5 | 1.1 | 1.3 | 94.7 |
| | antigen 5 | spindle coiled-coil related protein (DEEPEST), | | | | |
| | | mRNA. /FEA = mRNA /GEN = DEEPEST | | | | |
| | | /PROD = mitotic spindle coiled-coil related protein | | | | |
| | | /DB_XREF = gi: 5453631 /UG = Hs.16244 mitotic | | | | |
| | | spindle coiled-coil related protein | | | | |
| | | /FL = gb: BC000322.1 gb: AF063308.1 | | | | |
| | | gb: NM_006461.1 | | | | |
| 205119_s_at | formyl peptide | gb: NM_002029.1 /DEF = Homo sapiens formyl | | 1.3 | −1.0 | −1.4 |
| | receptor 1 | peptide receptor 1 (FPR1), mRNA. /FEA = mRNA | | | | |
| | | /GEN = FPR1 /PROD = formyl peptide receptor 1 | | | | |
| | | /DB_XREF = gi: 4503778 /UG = Hs.753 formyl | | | | |
| | | peptide receptor 1 /FL = gb: BC005315.1 | | | | |
| | | gb: M60626.1 gb: M60627.1 gb: NM_002029.1 | | | | |
| 204759_at | chromosome | gb: NM_001268.1 /DEF = Homo sapiens | CHC1L | 1.6 | −1.0 | 1.2 |
| | condensation 1- | chromosome condensation 1-like (CHC1L), | | | | |
| | like | mRNA. /FEA = mRNA /GEN = CHC1L | | | | |
| | | /PROD = RCC1-like G exchanging factor RLG | | | | |
| | | /DB_XREF = gi: 4557444 /UG = Hs.27007 | | | | |
| | | chromosome condensation 1-like | | | | |
| | | /FL = gb: AF060219.1 gb: NM_001268.1 | | | | |
| 201666_at | tissue inhibitor of | gb: NM_003254.1 /DEF = Homo sapiens tissue | TIMP1 | 1.0 | −1.3 | 1.0 |
| | metalloproteinase | inhibitor of metalloproteinase 1 (erythroid | | | | |
| | 1 (erythroid | potentiating activity, collagenase inhibitor) | | | | |
| | potentiating | (TIMP1), mRNA. /FEA = mRNA /GEN = TIMP1 | | | | |
| | activity, | /PROD = tissue inhibitor of metalloproteinase | | | | |
| | collagenase | 1 precursor /DB_XREF = gi: 4507508 /UG = Hs.5831 tissue inhibitor of | | | | |
| | inhibitor) | metalloproteinase 1 (erythroid | | | | |
| | | potentiating activity, collagenase inhibitor) | | | | |
| | | /FL = gb: BC000866.1 gb: M12670.1 gb: M59906.1 | | | | |
| | | gb: NM_003254.1 | | | | |
| 217996_at | pleckstrin | Consensus includes gb: AA576961 /FEA = EST | PHLDA1 | 1.2 | 63.7 | −66.0 |
| | homology-like | /DB_XREF = gi: 2354435 | | | | |
| | domain, family A, | /DB_XREF = est: nm82d08.s1 | | | | |
| | member 1 | /CLONE = IMAGE: 1074735 /UG = Hs.82101 | | | | |
| | | pleckstrin homology-like domain, family A, | | | | |
| | | member 1 /FL = gb: NM_007350.1 | | | | |
| 202388_at | regulator of G- | gb: NM_002923.1 /DEF = Homo sapiens regulator | RGS2 | 1.4 | 1.2 | 1.3 |
| | protein signalling | of G-protein signalling 2, 24 kD (RGS2), mRNA. | | | | |
| | 2, 24 kDa | /FEA = mRNA /GEN = RGS2 /PROD = regulator of | | | | |
| | | G-protein signalling 2, 24 kD | | | | |
| | | /DB_XREF = gi: 4506516 /UG = Hs.78944 regulator | | | | |
| | | of G-protein signalling 2, 24 kD /FL = gb: L13463.1 | | | | |
| | | gb: NM_002923.1 | | | | |
| 204446_s_at | arachidonate 5- | gb: NM_000698.1 /DEF = Homo sapiens | ALOX5 | 1.2 | −1.3 | −1.3 |
| | lipoxygenase | arachidonate 5-lipoxygenase (ALOX5), mRNA. | | | | |
| | | /FEA = mRNA /GEN = ALOX5 | | | | |
| | | /PROD = arachidonate 5-lipoxygenase | | | | |
| | | /DB_XREF = gi: 4502056 /UG = Hs.89499 | | | | |
| | | arachidonate 5-lipoxygenase /FL = gb: J03600.1 | | | | |
| | | gb: J03571.1 gb: NM_000698.1 | | | | |
| 203126_at | inositol(myo)-1(or | gb: NM_014214.1 /DEF = Homo sapiens | IMPA2 | −1.0 | −1.7 | 1.0 |
| | 4)- | inositol(myo)-1(or 4)-monophosphatase 2 | | | | |
| | monophosphatase 2 | (IMPA2), mRNA. /FEA = mRNA /GEN = IMPA2 | | | | |
| | | /PROD = inositol(myo)-1(or 4)-monophosphatase | | | | |
| | | 2 /DB_XREF = gi: 7657235 /UG = Hs.5753 | | | | |
| | | inositol(myo)-1(or 4)-monophosphatase 2 | | | | |
| | | /FL = gb: AF200432.1 gb: NM_014214.1 | | | | |
| | | gb: AF014398.2 | | | | |
| 208581_x_at | metallothionein | gb: NM_005952.1 /DEF = Homo sapiens | MT1X | 1.2 | −1.4 | −1.5 |
| | 1X | metallothionein 1X (MT1X), mRNA. /FEA = CDS | | | | |
| | | /GEN = MT1X /PROD = metallothionein 1X | | | | |
| | | /DB_XREF = gi: 10835231 /UG = Hs.278462 | | | | |
| | | metallothionein 1X /FL = gb: NM_005952.1 | | | | |
| 219890_at | C-type (calcium | gb: NM_013252.1 /DEF = Homo sapiens C-type | CLECSF5 | 1.5 | −1.0 | −1.2 |
| | dependent, | (calcium dependent, carbohydrate-recognition | | | | |
| | carbohydrate- | domain) lectin, superfamily member 5 | | | | |
| | recognition | (CLECSF5), mRNA. /FEA = mRNA | | | | |
| | domain) lectin, | /GEN = CLECSF5 /PROD = C-type (calcium | | | | |
| | superfamily | dependent, carbohydrate-recognition domain) | | | | |
| | member 5 | lectin, superfamilymember 5 | | | | |
| | | /DB_XREF = gi: 10281668 /UG = Hs.126355 C-type | | | | |
| | | (calcium dependent, carbohydrate-recognition | | | | |
| | | domain) lectin, superfamily member 5 | | | | |
| | | /FL = gb: NM_013252.1 gb: AF139768.1 | | | | |
| 209773_s_at | ribonucleotide | gb: BC001886.1 /DEF = Homo sapiens, | RRM2 | −1.9 | −1.5 | −1.5 |
| | reductase M2 | ribonucleotide reductase M2 polypeptide, clone | | | | |
| | polypeptide | MGC: 1551, mRNA, complete cds. /FEA = mRNA | | | | |
| | | /PROD = ribonucleotide reductase M2 polypeptide | | | | |
| | | /DB_XREF = gi: 12804874 /UG = Hs.75319 | | | | |
| | | ribonucleotide reductase M2 polypeptide | | | | |
| | | /FL = gb: BC001886.1 | | | | |
| 205098_at | chemokine (C-C | Consensus includes gb: AI421071 /FEA = EST | | −1.1 | −1.7 | −1.1 |
| | motif) receptor 1 | /DB_XREF = gi: 4267002 | | | | |
| | | /DB_XREF = est: tf24a02.x1 | | | | |
| | | /CLONE = IMAGE: 2097098 /UG = Hs.301921 | | | | |
| | | chemokine (C-C motif) receptor 1 | | | | |
| | | /FL = gb: L09230.1 gb: L10918.1 gb: NM_001295.1 | | | | |
| 201438_at | collagen, type VI, | gb: NM_004369.1 /DEF = Homo sapiens collagen, | COL6A3 | −1.0 | −1.0 | −1.0 |
| | alpha 3 | type VI, alpha 3 (COL6A3), mRNA. /FEA = mRNA | | | | |
| | | /GEN = COL6A3 /PROD = collagen, type VI, alpha | | | | |
| | | 3 /DB_XREF = gi: 4758027 /UG = Hs.80988 | | | | |
| | | collagen, type VI, alpha 3 /FL = gb: NM_004369.1 | | | | |
| 207067_s_at | histidine | gb: NM_002112.1 /DEF = Homo sapiens histidine | HDC | −1.3 | −1.0 | −1.2 |
| | decarboxylase | decarboxylase (HDC), mRNA. /FEA = mRNA | | | | |
| | | /GEN = HDC /PROD = histidine decarboxylase | | | | |
| | | /DB_XREF = gi: 4504364 /UG = Hs.1481 histidine | | | | |
| | | decarboxylase /FL = gb: M60445.1 | | | | |
| | | gb: NM_002112.1 | | | | |
| 204614_at | serine (or | gb: NM_002575.1 /DEF = Homo sapiens serine (or | SERPINB2 | 1.4 | 1.2 | 1.3 |
| | cysteine) | cysteine) proteinase inhibitor, clade B | | | | |
| | proteinase | (ovalbumin), member 2 (SERPINB2), mRNA. | | | | |
| | inhibitor, clade B | /FEA = mRNA /GEN = SERPINB2 /PROD = serine | | | | |
| | (ovalbumin), | (or cysteine) proteinase inhibitor, cladeB | | | | |
| | member 2 | (ovalbumin), member 2 /DB_XREF = gi: 4505594 | | | | |
| | | /UG = Hs.75716 serine (or cysteine) proteinase | | | | |
| | | inhibitor, clade B (ovalbumin), member 2 | | | | |
| | | /FL = gb: J02685.1 gb: J03603.1 gb: M18082.1 | | | | |
| | | gb: NM_002575.1 | | | | |
| 205786_s_at | integrin, alpha M | gb: NM_000632.2 /DEF = Homo sapiens integrin, | ITGAM | 1.0 | −1.2 | −1.6 |
| | (complement | alpha M (complement component receptor 3, | | | | |
| | component | alpha; also known as CD11b (p170), | | | | |
| | receptor 3, alpha; | macrophage antigen alpha polypeptide) | | | | |
| | also known as | (ITGAM), mRNA. /FEA = mRNA /GEN = ITGAM | | | | |
| | CD11b (p170), | /PROD = integrin alpha M precursor | | | | |
| | macrophage | /DB_XREF = gi: 6006013 /UG = Hs.172631 integrin, | | | | |
| | antigen alpha | alpha M (complement component receptor 3, | | | | |
| | polypeptide) | alpha; also known as CD11b (p170), | | | | |
| | | macrophage antigen alpha polypeptide) | | | | |
| | | /FL = gb: J03925.1 gb: NM_000632.2 | | | | |
| 201739_at | serum/glucocorticoid | gb: NM_005627.1 /DEF = Homo sapiens | SGK | 1.3 | 1.0 | 1.0 |
| | regulated | serumglucocorticoid regulated kinase (SGK), | | | | |
| | kinase | mRNA. /FEA = mRNA /GEN = SGK | | | | |
| | | /PROD = serumglucocorticoid regulated kinase | | | | |
| | | /DB_XREF = gi: 5032090 /UG = Hs.296323 | | | | |
| | | serumglucocorticoid regulated kinase | | | | |
| | | /FL = gb: BC001263.1 gb: NM_005627.1 | | | | |
| | | gb: AF153609.1 | | | | |
| 209949_at | neutrophil | gb: BC001606.1 /DEF = Homo sapiens, Similar to | NCF2 | 1.2 | −1.4 | −1.5 |
| | cytosolic factor 2 | neutrophil cytosolic factor 2 (65 kD, chronic | | | | |
| | (65 kDa, chronic | granulomatous disease, autosomal 2), clone | | | | |
| | granulomatous | MGC: 2275, mRNA, complete cds. /FEA = mRNA | | | | |
| | disease, | /PROD = Similar to neutrophil cytosolic factor 2 | | | | |
| | autosomal 2) | (65 kD, chronic granulomatous disease, | | | | |
| | | autosomal 2) /DB_XREF = gi: 12804408 | | | | |
| | | /UG = Hs.949 neutrophil cytosolic factor 2 (65 kD, | | | | |
| | | chronic granulomatous disease, autosomal 2) | | | | |
| | | /FL = gb: BC001606.1 gb: M32011.1 | | | | |
| | | gb: NM_000433.1 | | | | |
| 221210_s_at | N- | gb: NM_030769.1 /DEF = Homo sapiens | NPL | 1.5 | −1.1 | 1.1 |
| | acetylneuraminate | hypothetical protein similar to swine | | | | |
| | pyruvate lyase | acylneuraminate lyase (C1ORF13), mRNA. | | | | |
| | (dihydrodipicolinate | /FEA = mRNA /GEN = C1ORF13 | | | | |
| | synthase) | /PROD = hypothetical protein similar to | | | | |
| | | swineacylneuraminate lyase | | | | |
| | | /DB_XREF = gi: 13540532 /FL = gb: NM_030769.1 | | | | |
| 210184_at | integrin, alpha X | gb: M81695.1 /DEF = H. sapiens leukocyte | ITGAX | 1.1 | −1.0 | −1.2 |
| | (antigen CD11C | adhesion glycoprotein p150, 95 mRNA, complete | | | | |
| | (p150), alpha | cds. /FEA = mRNA /GEN = ITGAX | | | | |
| | polypeptide) | /DB_XREF = gi: 487829 /UG = Hs.51077 integrin, | | | | |
| | | alpha X (antigen CD11C (p150), alpha polypeptide) /FL = gb: M81695.1 | | | | |
| | | gb: NM_000887.2 | | | | |
| 217762_s_at | RAB31, member | Consensus includes gb: BE789881 /FEA = EST | RAB31 | 1.3 | −1.3 | −1.1 |
| | RAS oncogene | /DB_XREF = gi: 10211079 | | | | |
| | family | /DB_XREF = est: 601476750F1 | | | | |
| | | /CLONE = IMAGE: 3879493 /UG = Hs.223025 | | | | |
| | | RAB31, member RAS oncogene family | | | | |
| | | /FL = gb: AF234995.1 gb: BC001148.1 | | | | |
| | | gb: U59877.1 gb: U57091.1 gb: NM_006868.1 | | | | |
| | | gb: AF183421.1 | | | | |
| 215925_s_at | CD72 antigen | Consensus includes gb: AF283777.2 | CD72 | 69.7 | −1.4 | 1.4 |
| | | /DEF = Homo sapiens clone TCBAP0702 mRNA | | | | |
| | | sequence. /FEA = mRNA | | | | |
| | | /DB_XREF = gi: 10281735 /UG = Hs.116481 CD72 | | | | |
| | | antigen | | | | |
| 201422_at | interferon, | gb: NM_006332.1 /DEF = Homo sapiens | IFI30 | −1.4 | −1.1 | −1.1 |
| | gamma-inducible | interferon, gamma-inducible protein 30 (IFI30), | | | | |
| | protein 30 | mRNA. /FEA = mRNA /GEN = IFI30 | | | | |
| | | /PROD = interferon, gamma-inducible protein 30 | | | | |
| | | /DB_XREF = gi: 5453695 /UG = Hs.14623 | | | | |
| | | interferon, gamma-inducible protein 30 | | | | |
| | | /FL = gb: J03909.1 gb: NM_006332.1 | | | | |
| | | gb: AF097362.1 | | | | |
| 218559_s_at | v-maf | gb: NM_005461.1 /DEF = Homo sapiens Kreisler | MAFB | 1.1 | −1.5 | 1.1 |
| | musculoaponeurotic | (mouse) maf-related leucine zipper homolog | | | | |
| | fibrosarcoma | (KRML), mRNA. /FEA = mRNA /GEN = KRML | | | | |
| | oncogene | /PROD = Kreisler (mouse) maf-related leucine | | | | |
| | homolog B | zipperhomolog /DB_XREF = gi: 4885446 | | | | |
| | (avian) | /UG = Hs.169487 Kreisler (mouse) maf-related | | | | |
| | | leucine zipper homolog /FL = gb: AF134157.1 gb: NM_005461.1 | | | | |
| 202241_at | tribbles homolog | gb: NM_025195.1 /DEF = Homo sapiens | TRIB1 | 1.4 | −1.3 | 1.1 |
| | 1 (Drosophila) | phosphoprotein regulated by mitogenic pathways | | | | |
| | | (C8FW), mRNA. /FEA = mRNA /GEN = C8FW | | | | |
| | | /PROD = G-protein-coupled receptor induced | | | | |
| | | protein /DB_XREF = gi: 13399327 /UG = Hs.7837 | | | | |
| | | phosphoprotein regulated by mitogenic pathways | | | | |
| | | /FL = gb: AF205437.1 gb: NM_025195.1 | | | | |
| 204798_at | v-myb | gb: NM_005375.1 /DEF = Homo sapiens v-myb | MYB | 1.4 | −1.1 | −1.4 |
| | myeloblastosis | avian myeloblastosis viral oncogene homolog | | | | |
| | viral oncogene | (MYB), mRNA. /FEA = mRNA /GEN = MYB | | | | |
| | homolog (avian) | /PROD = v-myb avian myeloblastosis viral | | | | |
| | | oncogenehomolog /DB_XREF = gi: 4885496 | | | | |
| | | /UG = Hs.1334 v-myb avian myeloblastosis viral | | | | |
| | | oncogene homolog /FL = gb: M15024.1 | | | | |
| | | gb: AF104863.1 gb: NM_005375.1 | | | | |
| 203923_s_at | cytochrome b- | gb: NM_000397.2 /DEF = Homo sapiens | CYBB | 1.1 | −1.2 | −1.3 |
| | 245, beta | cytochrome b-245, beta polypeptide (chronic | | | | |
| | polypeptide | granulomatous disease) (CYBB), mRNA. | | | | |
| | (chronic | /FEA = mRNA /GEN = CYBB /PROD = cytochrome | | | | |
| | granulomatous | b-245, beta polypeptide (chronicgranulomatous | | | | |
| | disease) | disease) /DB_XREF = gi: 6996020 /UG = Hs.88974 | | | | |
| | | cytochrome b-245, beta polypeptide (chronic | | | | |
| | | granulomatous disease) /FL = gb: NM_000397.2 | | | | |
| 218039_at | nucleolar and | gb: NM_016359.1 /DEF = Homo sapiens clone | NUSAP1 | 1.2 | −1.2 | −1.4 |
| | spindle | HQ0310 PRO0310p1 (LOC51203), mRNA. | | | | |
| | associated | /FEA = mRNA /GEN = LOC51203 /PROD = clone | | | | |
| | protein 1 | HQ0310 PRO0310p1 /DB_XREF = gi: 7705950 | | | | |
| | | /UG = Hs.279905 clone HQ0310 PRO0310p1 | | | | |
| | | /FL = gb: AF305711.1 gb: BC001308.1 | | | | |
| | | gb: AF290612.1 gb: AF090915.1 | | | | |
| | | gb: NM_016359.1 | | | | |
| 206461_x_at | metallothionein | gb: NM_005951.1 /DEF = Homo sapiens | MT1H | 1.2 | −1.9 | −1.2 |
| | 1H | metallothionein 1H (MT1H), mRNA. /FEA = mRNA | | | | |
| | | /GEN = MT1H /PROD = metallothionein 1H | | | | |
| | | /DB_XREF = gi: 10835084 /UG = Hs.2667 | | | | |
| | | metallothionein 1H /FL = gb: NM_005951.1 | | | | |
| 203535_at | S100 calcium | gb: NM_002965.2 /DEF = Homo sapiens S100 | S100A9 | −1.1 | −1.4 | −1.5 |
| | binding protein A9 | calcium-binding protein A9 (calgranulin B) | | | | |
| | (calgranulin B) | (S100A9), mRNA. /FEA = mRNA /GEN = S100A9 | | | | |
| | | /PROD = S100 calcium-binding protein A9 | | | | |
| | | /DB_XREF = gi: 9845520 /UG = Hs.112405 S100 | | | | |
| | | calcium-binding protein A9 (calgranulin B) | | | | |
| | | /FL = gb: M26311.1 gb: NM_002965.2 | | | | |
| 204415_at | interferon, alpha- | gb: NM_022873.1 /DEF = Homo sapiens | G1P3 | −1.1 | −3.5 | 1.4 |
| | inducible protein | interferon, alpha-inducible protein (clone IFI-6- | | | | |
| | (clone IFI-6-16) | 16) (G1P3), transcript variant 3, mRNA. | | | | |
| | | /FEA = mRNA /GEN = G1P3 /PROD = interferon | | | | |
| | | induced 6-16 protein, isoform c | | | | |
| | | /DB_XREF = gi: 13259549 /UG = Hs.265827 | | | | |
| | | interferon, alpha-inducible protein (clone IFI-6- | | | | |
| | | 16) /FL = gb: NM_022872.1 gb: NM_022873.1 | | | | |
| | | gb: NM_002038.2 | | | | |
| 205483_s_at | interferon, alpha- | gb: NM_005101.1 /DEF = Homo sapiens | G1P2 | 1.0 | −9.9 | −1.1 |
| | inducible protein | interferon-stimulated protein, 15 kDa (ISG15), | | | | |
| | (clone IFI-15K) | mRNA. /FEA = mRNA /GEN = ISG15 | | | | |
| | | /PROD = interferon-stimulated protein, 15 kDa | | | | |
| | | /DB_XREF = gi: 4826773 /UG = Hs.833 interferon-stimulated protein, | | | | |
| | | 15 kDa /FL = gb: M13755.1 | | | | |
| | | gb: NM_005101.1 | | | | |
| 203595_s_at | interferon-induced | Consensus includes gb: N47725 /FEA = EST | IFIT5 | 1.5 | −2.2 | 1.0 |
| | protein with | /DB_XREF = gi: 1188891 | | | | |
| | tetratricopeptide | /DB_XREF = est: yy92h11.s1 | | | | |
| | repeats 5 | /CLONE = IMAGE: 281061 /UG = Hs.27610 | | | | |
| | | retinoic acid- and interferon-inducible protein | | | | |
| | | (58 kD) /FL = gb: U34605.1 gb: NM_012420.1 | | | | |
| 203153_at | interferon-induced | gb: NM_001548.1 /DEF = Homo sapiens | | −1.0 | −1084.5 | −1.0 |
| | protein with | interferon-induced protein with tetratricopeptide | | | | |
| | tetratricopeptide | repeats 1 (IFIT1), mRNA. /FEA = mRNA | | | | |
| | repeats 1 | /GEN = IFIT1 /PROD = interferon-induced protein | | | | |
| | | withtetratricopeptide repeats 1 | | | | |
| | | /DB_XREF = gi: 4504584 /UG = Hs.20315 | | | | |
| | | interferon-induced protein with tetratricopeptide | | | | |
| | | repeats 1 /FL = gb: M24594.1 gb: NM_001548.1 | | | | |
| 214453_s_at | interferon-induced | Consensus includes gb: NM_006417.1 | IFI44 | 1.4 | −4.9 | 1.1 |
| | protein 44 | /DEF = Homo sapiens interferon-induced, | | | | |
| | | hepatitis C-associated microtubular aggregate | | | | |
| | | protein (44 kD) (MTAP44), mRNA. /FEA = CDS | | | | |
| | | /GEN = MTAP44 /PROD = interferon-induced, | | | | |
| | | hepatitis C-associatedmicrotubular aggregate | | | | |
| | | protein (44 kD) /DB_XREF = gi: 5453743 | | | | |
| | | /UG = Hs.82316 interferon-induced, hepatitis C- | | | | |
| | | associated microtubular aggregate protein | | | | |
| | | (44 kD) /FL = gb: NM_006417.1 | | | | |
| 202086_at | myxovirus (influenza | gb: NM_002462.1 /DEF = Homo sapiens | MX1 | 1.1 | −7.3 | −1.2 |
| | virus) resistance 1, | myxovirus (influenza) resistance 1, homolog of | | | | |
| | interferon-inducible | murine (interferon-inducible protein p78) (MX1), | | | | |
| | protein p78 (mouse) | mRNA. /FEA = mRNA /GEN = MX1 | | | | |
| | | /PROD = myxovirus (influenza) resistance 1, | | | | |
| | | homolog ofmurine (interferon-inducible protein | | | | |
| | | p78) /DB_XREF = gi: 4505290 /UG = Hs.76391 | | | | |
| | | myxovirus (influenza) resistance 1, homolog of | | | | |
| | | murine (interferon-inducible protein p78) | | | | |
| | | /FL = gb: M30817.1 gb: M33882.1 | | | | |
| | | gb: NM_002462.1 | | | | |
| 216598_s_at | chemokine (C-C | Consensus includes gb: S69738.1 /DEF = MCP- | CCL2 | −1.3 | −3.1 | −2.4 |
| | motif) ligand 2 | 1 = monocyte chemotactic protein human, aortic | | | | |
| | | endothelial cells, mRNA, 661 nt. /FEA = mRNA | | | | |
| | | /GEN = MCP-1 /PROD = MCP-1 | | | | |
| | | /DB_XREF = gi: 545464 /UG = Hs.303649 small | | | | |
| | | inducible cytokine A2 (monocyte chemotactic | | | | |
| | | protein 1, homologous to mouse Sig-je) | | | | |
| 204533_at | chemokine (C—X—C | gb: NM_001565.1 /DEF = Homo sapiens small | CXCL10 | −1.2 | −4.9 | −3.9 |
| | motif) ligand 10 | inducible cytokine subfamily B (Cys-X-Cys), | | | | |
| | | member 10 (SCYB10), mRNA. /FEA = mRNA | | | | |
| | | /GEN = SCYB10 /PROD = interferon gamma- | | | | |
| | | induced precursor /DB_XREF = gi: 4504700 | | | | |
| | | /UG = Hs.2248 small inducible cytokine | | | | |
| | | subfamily B (Cys-X-Cys), member 10 | | | | |
| | | /FL = gb: NM_001565.1 | | | | |
| 203915_at | chemokine (C—X—C | gb: NM_002416.1 /DEF = Homo sapiens | CXCL9 | −1.2 | −1.7 | −3.2 |
| | motif) ligand 9 | monokine induced by gamma interferon (MIG), | | | | |
| | | mRNA. /FEA = mRNA /GEN = MIG | | | | |
| | | /PROD = monokine induced by gamma | | | | |
| | | interferon /DB_XREF = gi: 4505186 | | | | |
| | | /UG = Hs.77367 monokine induced by gamma | | | | |
| | | interferon /FL = gb: NM_002416.1 | | | | |
| 209714_s_at | cyclin-dependent | gb: AF213033.1 /DEF = Homo sapiens isolate | CDKN3 | −1.3 | −1.0 | −1.2 |
| | kinase inhibitor 3 | BX-01 cyclin-dependent kinase associated | | | | |
| | (CDK2-associated | protein phosphatase mRNA, complete cds. | | | | |
| | dual specificity | /FEA = mRNA /PROD = cyclin-dependent kinase | | | | |
| | phosphatase) | associated proteinphosphatase | | | | |
| | | /DB_XREF = gi: 12734643 /UG = Hs.84113 | | | | |
| | | cyclin-dependent kinase inhibitor 3 (CDK2- | | | | |
| | | associated dual specificity phosphatase) | | | | |
| | | /FL = gb: AF213033.1 gb: AF213034.1 | | | | |
| | | gb: AF213035.1 gb: AF213036.1 | | | | |
| | | gb: AF213037.1 gb: AF213038.1 | | | | |
| | | gb: AF213039.1 gb: AF213040.1 | | | | |
| | | gb: AF213041.1 gb: AF213042.1 | | | | |
| | | gb: AF213044.1 gb: AF213046.1 | | | | |
| | | gb: AF213047.1 gb: AF213048.1 | | | | |
| | | gb: AF213049.1 gb: AF213050.1 | | | | |
| | | gb: AF213051.1 gb: AF213052.1 | | | | |
| | | gb: AF213053.1 gb: U02681.1 gb: L25876.1 | | | | |
| | | gb: NM_005192.1 gb: L27711.1 | | | | |
| 204170_s_at | CDC28 protein | gb: NM_001827.1 /DEF = Homo sapiens | CKS2 | −1.1 | −1.7 | −1.9 |
| | kinase regulatory | CDC28 protein kinase 2 (CKS2), mRNA. | | | | |
| | subunit 2 | /FEA = mRNA /GEN = CKS2 /PROD = CDC28 | | | | |
| | | protein kinase 2 /DB_XREF = gi: 4502858 | | | | |
| | | /UG = Hs.83758 CDC28 protein kinase 2 | | | | |
| | | /FL = gb: NM_001827.1 | | | | |
| 203213_at | cell division cycle | Consensus includes gb: AL524035 /FEA = EST | CDC2 | −1.0 | −1.0 | −1.0 |
| | 2, G1 to S and G2 | /DB_XREF = gi: 12787528 | | | | |
| | to M | /DB_XREF = est: AL524035 | | | | |
| | | /CLONE = CS0DC003YN06 (3 prime) | | | | |
| | | /UG = Hs.184572 cell division cycle 2, G1 to S | | | | |
| | | and G2 to M /FL = gb: NM_001786.1 | | | | |
| 201292_at | topoisomerase | Consensus includes gb: AL561834 /FEA = EST | TOP2A | −1.0 | −1.0 | −1.0 |
| | (DNA) II alpha | /DB_XREF = gi: 12909658 | | | | |
| | 170 kDa | /DB_XREF = est: AL561834 | | | | |
| | | /CLONE = CS0DB005YC15 (3 prime) | | | | |
| | | /UG = Hs.156346 topoisomerase (DNA) II | | | | |
| | | alpha (170 kD) /FL = gb: J04088.1 | | | | |
| | | gb: NM_001067.1 | | | | |

[symbol]

indicates data missing or illegible when filed

1-9. (canceled)

10. A nucleic acid array of comprising at least 4 distinct nucleic acid sequences selected from the group consisting of the polynucleotides listed in any one of Gene Tables I, V, VI, VII or Table IV, immobilized on a surface at discrete and known positions, wherein said nucleic acids hybridize to nucleic acids in a sample of a subject that are either up-regulated or down-regulated in response to inhibition of IMPDH.

11-13. (canceled)

14. The nucleic acid array of claim 10, wherein the surface is selected from the group consisting of a metal, silicon, a polymer plastic, paper, ceramic, quartz, gallium arsenide, metal, metalloid, cellulose, celluose acetate, nitrocellulose, and a glass.

15. (canceled)

16. The nucleic acid array of claim 10, further comprising at least one control spot consisting of one or more nucleic acids that are known not to be modulated with IMPDH inhibition.

17. (canceled)

18. The nucleic acid array of claim 10, wherein said surface comprises a plurality of microarrays separated from each other with a hydrophobic polymer strip.

19. (canceled)

20. A set of polynucleotides for use in the detection of IMPDH inhibition, wherein said polynucleotides hybridize to 4 to 314 genes selected from the group consisting of the genes set forth in any one or more of Gene Table I, Gene Table II, Gene Table III, Gene Table IV, Gene Table V, Gene Table VI, Gene Table VII and Gene Table VIII wherein the expression of each said polynucleotides is either up- or down-regulated in response to inhibition of IMPDH.

21-26. (canceled)

27. A method for predicting whether a candidate IMPDH inhibitory agent will produce a therapeutic effect in a subject comprising: contacting a biological sample from said subject with said candidate IMPDH inhibitory agent and determining the expression level in said biological sample of four or more prognostic genes selected from the group consisting of the genes set forth in one or more of Gene Table I, Gene Table II, Gene Table III, Gene Table IV, Gene Table V, Gene Table VI, Gene Table VII and Gene Table VIII, wherein a modulation of the expression level of the four or more of genes is similar to the modulation resulting from contacting the biological sample with VX-944 and is indicative that said agent is a therapeutically effective IMPDH inhibitory agent.

28-29. (canceled)

30. The method of claim 27, wherein said subject is human.

31. The method of claim 27, wherein said biological sample is a sample from a cancer patient, and wherein said cancer is selected from the group consisting of breast cancer, ovarian cancer, gastric cancer, colorectal cancer, prostate cancer, pancreatic cancer, and lung cancer.

32. The method of claim 27, wherein the modulation of expression in response to said candidate IMPDH inhibitory agent is similar to the modulation in expression seen in response to contacting said biological sample with VX-944.

33. The method of claim 27, wherein said biological sample is a tissue sample comprising cancer cells.

34. The method of claim 27, wherein said tissue is fixed, paraffin-embedded, fresh, or frozen.

35. The method of claim 34, where the tissue is from a biopsy.

36. (canceled)

37. The method of claim 27, wherein the expression level of said prognostic genes is determined by PCR, immunohistochemistry or in situ hybridization.

38-39. (canceled)

40. The method of claim 27, wherein the assay for the measurement of said prognostic genes or their expression products is provided in the form of a kit or kits.

41. A method of preparing a prognostic profile for a subject's response to an IMPDH inhibitor comprising the steps of:
(a) exposing ex vivo cells of said subject to an IMPDH inhibitor;
(b) subjecting RNA extracted from the cells of step (a) to gene expression profiling;
(c) determining the expression levels of at least four genes selected from the group consisting of the genes set forth in one or more of Gene Table I, Gene Table II, Gene Table III, Gene Table IV, Gene Table V, Gene Table VI, Gene Table VII and Gene Table VIII in said cells; and
(d) comparing the expression levels obtained in step (c) to expression levels obtained in the absence of said IMPDH inhibitor, wherein a modulation of the expression level of said four or more of genes in response to said IMPDH inhibitor indicates that said subject is likely to be responsive to said inhibitor.

42. A method of preparing a prognostic profile for a subject's response to an IMPDH inhibitor comprising the steps of:
(a) administering an IMPDH inhibitor to said subject;
(b) subjecting RNA extracted from cells of said subject to gene expression profiling;
(c) determining the expression level of at least four genes selected from the group consisting of the genes set forth in one or more of Gene Table I, Gene Table II, Gene Table III, Gene Table IV, Gene Table V, Gene Table VI, Gene Table VII and Gene Table VIII in said cells of said subject; and
(d) comparing the expression levels obtained in step (c) to expression levels obtained in the absence of said IMPDH inhibitor, wherein a modulation of the expression level of said four or more of genes in response to said IMPDH inhibitor administration indicates that said IMPDH inhibitor will likely predict the subject's response to said IMPDH inhibitor administration.

43. (canceled)

44. The method of claim 41 or 42 wherein said cells are cancer cells obtained from said subject.

45. The method of claim 44, wherein said cancer cells are selected from the group consisting of breast cancer cells, ovarian cancer cells, hematological cancer cells, gastric cancer cells, colorectal cancer cells, pancreatic cancer cells, and lung cancer cells.

46-48. (canceled)

49. The method of claim 41 or 42 wherein said expression profile is compiled into a report that includes recommendation for a treatment for said subject with an IMPDH inhibitor.

50. The method of claim 41 or 42 wherein if altered expression of one or more of the genes selected from the genes set forth in Gene Tables 1, II, III, IV, V, VI, VII, and VIII, or a corresponding expression product is determined, said report includes a prediction that said subject is a suitable candidate for IMPDH inhibition-based therapy.

51. The method of claim 42, further comprising the step of treating said patient with an IMPDH inhibitory agent.

52. The method of claim 42, wherein the IMPDH inhibitory agent is identified according to the method of claim 27.

53-55. (canceled)
